(19) United States
(12) Patent Application Publication
(10) Pub. No.: US 2023/0265479 A1
(43) Pub. Date: Aug. 24, 2023

(54) ARTIFICIAL EUKARYOTIC EXPRESSION SYSTEM WITH ENHANCED PERFORMANCES

(71) Applicant: EUKARYS, Évry-Courcouronnes (FR)

(72) Inventor: Philippe JAIS, Issy-Les-Moulineaux (FR)

(73) Assignee: EUKARYS, Évry-Courcouronnes (FR)

(21) Appl. No.: 18/017,967

(22) PCT Filed: Aug. 3, 2021
(86) PCT No.: PCT/EP2021/071698

§ 371 Date: Jan. 25, 2023

(30) Foreign Application Priority Data
Aug. 4, 2020 (EP) 20305899.5

(51) Int. Cl. C12P 21/02; C12N 9/16; C12N 9/10; C12N 9/12; C12N 15/85; C12N 15/52

(52) U.S. Cl. CPC C12P 21/02; C12N 9/16; C12Y 301/03016; C12N 9/104; C12N 9/1247; C12N 15/85; C12N 15/52; C12N 2800/107

(57) ABSTRACT

The present invention concerns a method for expressing a recombinant DNA molecule in a eukaryotic host cell, comprising the steps of:
(a) expressing or introducing at least one chimeric protein, in said host cell, wherein said chimeric protein comprises:
   (i) at least one catalytic domain of a capping enzyme, in particular selected in the group consisting of cap-0 canonical capping enzymes, cap-0 non-canonical capping enzymes, cap-1 capping enzymes and cap-2 capping enzymes; and
   (ii) at least one catalytic domain of a DNA-dependent RNA polymerase, in particular a bacteriophage DNA-dependent RNA polymerase,
(b) constitutively or transiently downregulating the phosphorylation level of subunit a of translation initiation factor eIF2 (eIF2α) in said host cell.

The invention also concerns an isolated nucleic acid molecule or a set of nucleic acid molecules, comprising or consisting of (1) at least one nucleic acid sequence encoding a chimeric protein comprising at least one catalytic domain of a capping enzyme; and at least one catalytic domain of a DNA-dependent RNA polymerase; and (2) at least one nucleic acid sequence downregulating the phosphorylation level of eIF2α in a eukaryotic host cell or encoding a polypeptide downregulating said phosphorylation level; and (3) optionally, at least one nucleic acid sequence encoding a poly(A) polymerase, as well as vectors, kits and cells comprising said nucleic acid molecule or set, and different uses and applications thereof.

The present invention relates to an artificial eukaryotic expression system whose performance has been improved by inhibiting the phosphorylation or increasing the dephosphorylation of the eukaryotic Initiation Factor 2 (eIF2), which is a key regulator of initiation of translation.

### BACKGROUND

Eukaryotic expression is very widely used in the life sciences, biotechnology and medicine. Different expression vectors for gene transfer into eukaryotic cells have been developed, for both in vivo and in vitro applications, especially non-viral vectors and more preferably vectors not using the RNA transcription system of eukaryotic cells, but some bacteriophage DNA-dependent RNA polymerases, which have a higher processivity than the eukaryotic RNA polymerases. However, the level of transgene expression obtained by these vectors is usually insufficient or modest. One reason for this low expression level is the lack of a capping structure of the transcribed RNA.

With a view to overcoming this lack of capping structure of the transcribed RNA, the present inventor has first developed an artificial expression system, namely a chimeric protein, for efficient transgenesis in eukaryotic cells, which autonomously generates capped mRNA molecules, in particular in the cytoplasm of said eukaryotic cells. This first generation of the artificial expression system is disclosed in WO2011/128444 and in Jais et al. 2019 and comprises a chimeric enzyme, with a catalytic domain of a capping enzyme and a catalytic domain of a DNA-dependent RNA polymerase (Jais, Decroly et al. 2019). This artificial system is known as C3P3 (acronym of cytoplasmic chimeric capping-prone phage polymerase) and is detailed in the examples.

A second type of modification required to increase the level of expression relies on the elongation of the polyadenylation (poly(A)) tail of the neo-synthesized mRNA. The second generation of the artificial expression system developed by the inventor and also described in the examples thus comprises an activity of poly(A)-polymerase, and is disclosed in WO2019/020811.

Surprisingly, the inventor has however detected abnormalities in the polysomal profile in cells expressing artificial expression system comprising a chimeric enzyme with at least one catalytic domain of a capping enzyme and at least one catalytic domain of a DNA-dependent RNA polymerase together with a catalytic domain of a poly(A) polymerase. Said abnormalities reflect a translation initiation defect in these cells.

It was particularly surprising to detect this abnormal polysomal profile on 5′-capped polyadenylated RNA transcripts, since the polysomal profile was found normal when the same expression system was used without the catalytic domain of a poly(A) polymerase, which suggested the absence of translation initiation defect (Jais, Decroly et al. 2019).

The inventor has now found that, although the system already described synthesizes capped RNA molecules (first generation of the artificial expression system) or capped RNA molecules with an extended poly(A) tail (second generation of the artificial expression system), capable of being translated by the eukaryotic host cell machinery, the translation of these RNA molecules is suboptimal in the eukaryotic cells, i.e. is abnormally low given the amount of corresponding RNA molecules, which is attributable to defects in the initiation of translation. The inventor has unexpectedly shown that this defect was already present al low level with the first generation of the artificial expression system, limiting the translation rate, even though no anomaly could be detected by polysomal profiling analysis with that system.

There is thus an important need in the field of the invention to improve the translation efficiency of the artificial expression system.

Early Eukaryotic Translation

Initiation

In the eukaryotes, translation is the process in which ribosomes in the cytoplasm or attached to the ER (endoplasmic reticulum) synthesize proteins after the process transcription of DNA to RNA.

Translation is initiated by the binding of the eukaryotic initiation factor 4E (eIF4E) to the capping at the 5′-end of an mRNA molecule, which assembles with other initiation factors (i.e. eIF4A, eIF4E, and eIF4G), and then recruits the 43S preinitiation complex. This ribonucleoprotein complex contains the small ribosomal subunit (40S) bound by the initiation factors eIF1, eIF1A, eIF3, eIF5 and the active eIF2-Met-tRNAiMet-GTP ternary complex (eIF2-TC). This eIF2-TC ternary complex consists of GTP, Met-tRNAᵢ^{Met} tRNA, which a methionine-charged initiator distinct from other methionine-charged tRNAs used for elongation of the polypeptide chain, and eIF2 heterotrimer αβγ.

Scanning

The resulting 48S initiation complex moves along the mRNA chain toward its 3′-end, followed by a linear scanning 5′ to 3′ direction process, until it reaches an initiation codon in Kozak consensus sequence. Pairing between the AUG initiation codon and the Met-tRNAᵢ^{Met} anticodon elicits hydrolysis of the GTP by eIF2, which requires the GTPase activating protein eIF5. The resulting signal induces the dissociation of several factors, including eIF2, from the small ribosomal subunit. This leads to the association of the large 60S subunit and the formation of the complete 80S ribosome, which can start translation elongation.

Once the initiation phase is completed, eIF2 is released from the ribosome bound to GDP as an inactive binary complex. With the help of the guanine nucleotide exchange factor eIF2B, the GDP in eIF2 is exchanged for a GTP and the ternary complex reforms for a new round of translation initiation.

Regulation

Translation initiation is foremostly regulated though eIF2 activity, which consists of three subunits, α (also called subunit 1, EIF2S1; UniProtKB/Uniprot accession number P05198), β (subunit 2, EIF2S2), and γ (subunit 3, EIF2S3) (FIG. 4). The serine 52 residue (historically known as Ser51) of eIF2α subunit is conserved can be phosphorylated by various kinases. The phosphorylation of eIF2α increases its affinity for the guanosine nucleotide exchange factor eIF2B, which is responsible for the GDP-GTP exchange, but only when eIF2 is non-phosphorylated (Yang and Hinnebusch 1996, Pavitt, Ramaiah et al. 1998). In addition, since the cellular concentration of eIF2B is much lower than that of eIF2, even a small amount of phosphorylated eIF2α can completely abolish eIF2B activity by sequestration, and thereby results in the decrease of unphosphorylated eIF2α to its active (GTP-bound) state. The phosphorylation of eIF2α stops the initiation of translation which is no longer possible in the absence of the ternary complex available. Noticeably, due to their specific 5′ untranslated regions, the expression of some mammalian genes evade translational arrest triggered by eIF2 phosphorylation, including ATF4 (Vattem and Wek 2004), PPP1R15A (Lee, Cevallos et al. 2009), and DDIT3 (Jousse, Bruhat et al. 2001). Some other cellular factors have an important role in the initiation of translation, and more specifically in the loading of the charged aminoacyl-tRNA Met-tRNAᵢ^{Met} onto the 43S preinitiation complex. First the tRNAᵢ^{Met}, which is 76-nucleotide aminoacyl-tRNA with eight residues that have additional base modifications. Second, the methionyl tRNA synthetase (MetRS, also named MARS1; UniProtKB/Uniprot accession number P56192) that catalyzes the ligation of methionine to the tRNAᵢ^{Met} moiety. The resulting Met-tRNAᵢ^{Met} is then specifically bound by eIF2 to form a stable ternary complex with GTP (Levin, Kyner et al. 1973). The overexpression of these factors which could potentially increase the translation initiation rate, and thereby increase the level of expression by the present artificial expression system C3P3.

Polysome Profiling Assay

Polysome profiling is a technique used to study the association of mRNAs with ribosomes from cell lysate, in which the translation is halted via the addition of cycloheximide. Centrifugation of this lysate on a sucrose gradient allows separation of the small 40S and 60S large ribosomal subunits, monosomes that are composed of one ribosome residing on an mRNA, while the polysomes are made up of several ribosomes attached to an mRNA. The resulting profile is analyzed by optical density (O.D.) at 254 nm and consists of a series of peaks of the various ribosomal components.

Due to the fact that this technique is considered to be highly reproducible and sensible, polysome profiling is regarded as the reference method to study the overall translation functional activity in cells (Chasse, Boulben et al. 2017). This technique is particularly sensitive to any alteration in the initiation of translation. More specifically, this method is well adapted for analyzing eIF2 kinases and translational control in the unfolded protein response (Dey, Baird et al. 2010, Teske, Baird et al. 2011, Baird, Palam et al. 2014, Andreev, O'Connor et al. 2015, Knutsen, Rødland et al. 2015).

eIF2α Kinases

Phosphorylation of the eIF2α depends of four distinct eIF2α kinases in vertebrates. Each kinase is activated by stimulatory factors that bind to its regulatory domains, then promote the active state dimer configuration of their catalytic kinase domains:
EIF2AK1 (UniProtKB/Uniprot accession number Q9BQI3), also called heme-regulated inhibitor (HRI), is activated during heme deficiency by the release of heme from its kinase insert domains (Chen, Throop et al. 1991).
EIF2AK2 (UniProtKB/Uniprot accession number P19525), also named Protein kinase R (PKR) is activated during a viral infection by the binding of double-stranded RNA (dsRNA) to its double-stranded RNA (dsRNA) binding domains (Levin, Petryshyn et al. 1980, Meurs, Chong et al. 1990). EIF2AK2 is a key effector of the antiviral type-I interferon response, whose inhibition is critical for the artificial C3P3 expression system as detailed below.
EIF2AK3 (UniProtKB/Uniprot accession number Q9NZJ5), also denominated PKR-like endoplasmic reticulum (ER) kinase (PERK), is activated during ER stress by the release of its luminal ER domain bound with the protein HSPA5 (Harding, Zhang et al. 1999, Bertalotti, Zhang et al. 2000). EIF2AK3 is a key effector of the unfolded protein response, whose repression is also important for increased efficacy of the C3P3 system as shown below.
EIF2AK4 (UniProtKB/Uniprot accession number Q9P2K8), also named General control non-depressible 2 (GCN2) is activated under amino acid deprivation by the binding of uncharged tRNA to the regulatory domains (Dever, Feng et al. 1992, Zhang, McGrath et al. 2002).

Unfolded Protein Response

Mechanism

The unfolded protein response (UPR) is a cellular stress response related to the endoplasmic reticulum (ER) stress (Hetz and Papa 2018). The unfolded protein response is activated in response to an accumulation of unfolded or misfolded proteins in the lumen of the ER. As stated above, the activation of unfolded protein response is responsible for inhibition translation initiation though phosphorylation of eIF2α by EIF2AK3 kinase.

The term protein folding incorporates all the processes involved in the production of a protein after the nascent polypeptides have become synthesized by the ribosomes. The proteins destined to be secreted or sorted to other cell organelles carry an N-terminal signal sequence that will interact with a signal recognition particle (SRP). The SRP will lead the ribosome-mRNA-polypeptide complex to the ER membrane. Once docked, the protein continues translation, with the resultant strand being fed through the polypeptide translocator directly into the ER. Protein folding commences as soon as the polypeptide enters to the luminal environment, even as translation of the remaining polypeptide continues.

Protein folding steps involve a range of enzymes and molecular chaperones to coordinate and regulate reactions, in addition to a range of substrates required for the reactions to take place. The most important of these is N-linked glycosylation and disulfide bond formation, which is the main means by which the cell monitors protein folding. The misfolding protein becomes characteristically devoid of glucose residues, targeting it for identification and re-glycosylation by the enzyme UGGT (UniProtKB/Uniprot accession number Q0WL80). If this fails to restore the normal folding process, the misfolded protein is guided through ER-associated degradation. The chaperone EDEM guides the retrotranslocation of the misfolded protein back into the cytosol. where it enters the ubiquitin-proteasome pathway, as it is tagged by multiple ubiquitin molecules, targeting it for degradation by cytosolic proteasomes.

EIF2AK3 Kinase

Eukaryotic translation initiation factor 2-alpha kinase 3, also known as protein kinase R (PKR)-like ER kinase (PERK), is a key effector of the unfolded protein response.

EIF2AK3 is a type I membrane protein located in the ER, where it is induced by stress caused by misfolded proteins (Harding, Zhang et al. 1999). EIF2AK3 mediates the translational control arm of the unfolded protein response through phosphorylation of eIF2α (Harding, Zhang et al. 1999, Bertolotti, Zhang et al. 2000).

Activation of EIF2AK3 is under the control of the ER chaperone HSPA5 (also named BIP or GRP78), a heat shock protein 70 kDa family member, which senses the accumulation of misfolded proteins. Under nonstress conditions, HSPA5 binds the amino-terminal portion of EIF2AK3 located in the ER lumen, thereby repressing the eIF2 kinase activity, and therefore the phosphorylation of eIF2α by EIF2AK3. When misfolded proteins accumulate, HSPA5 binds the exposed hydrophobic residues of the misfolded protein that would normally be buried inside properly folded proteins, and thereby dissociates from EIF2AK3. This leads to the activation of this serine/threonine protein kinase by dimerization and trans-autophosphorylation, which in turn induces eIF2α phosphorylation and reduced translational initiation. Ultimately, such inhibition of protein translation decreases the influx of nascent polypeptides into the overloaded ER secretory pathway.

EIF2AK3 can be inhibited by various means including various viral proteins such as 3a (UniProtKB/Uniprot accession number P59632) from SARS Coronavirus (Minakshi, Padhan et al. 2009) or other polypeptides, chemical compounds (e.g. GSK2606414/CAS 1337531-89-1, AMGPERK44/CAS 1883548-84-2), nucleic acids (e.g. siRNA, shRNA, miRNA, antisense or ribozyme). Host-cell EIF2AK3 can be also knocked-out by various gene editing technologies, such as ZFNs, TALENs, and CRISPR-Cas9 system and its derivatives.

Mutant and Viral Proteins

Noticeably, several dominant negative mutants of EIF2AK3 have been characterized, such as the K618A that abolishes the ability of the protein to undergo autophosphorylation and to phosphorylate eIF2α, as well as the PERKΔC mutant produced by removing amino acids 582-1,081 of that contains the kinase domain the protein (Harding, Zhang et al. 1999). Such mutants can be used for EIF2AK3 inhibition and consequently eIF2α phosphorylation.

Type-I Interferon Response

Classification of Interferons

Interferons (IFNs) are signaling proteins, which belong to the class of cytokines and are produced and released by host cells in response to the presence of several viruses. They can be classified in three types according to their receptors:
Type I IFNs include in humans 13 IFNα genes, and only one type of IFNβ, IFNω, IFNε and IFNκ. They are mostly non-glycosylated proteins of 165-200-plus amino-acids, which share homologies that range from 30-85% within a species. They interact with IFN receptor that consists of subunits I and II (IFNAR1 and IFNAR2). The production of type I IFN-α is inhibited by another cytokine known as Interleukin-10.
Type II IFN only includes IFNγ, which binds to IFNGR1 and IFNGR2. It is released by natural killer (NK) and activated T-cells, and, even though it has direct antiviral activity, its main role is shaping the adaptive immune response,
Type III interferons includes four IFNλ subtypes (i.e. IFNλ1, IFNλ2, IFNλ3 and IFNλ4), which have similar activities as type I IFNs. However, they bind the heterodimeric receptor consisting of IFNLR1 and IL10RB subunits whose expression is restricted to specific cell types, such as epithelial cells. It appears that the functions of IFNλ are very close to those of type I IFNs, as they induce an unspecialized state of antiviral resistance, use the same transduction pathway, and also induce the expression of EIF2AK2 (Doyle, Schreckhise et al. 2006, Marcello, Grakoui et al. 2006). Therefore, the term type I interferon response also encompasses the effects of class III interferons hereinafter.

Pattern Recognition Receptors Sensors

The response to interferon is initiated by sensor proteins named Pattern Recognition Receptors (PRRs). Based on their localization, PRRs may be divided into membrane-bound PRRs and cytoplasmic PRRs. Membrane-bound PRRs are named Toll like receptors (TLRs) and are single-pass membrane-spanning receptors usually expressed on immune cells such as macrophages and dendritic cells. They recognize structurally conserved molecules derived from pathogens. Ten functional members of the TLR family have been described in humans so far. A set of them, TLR3 (UniProtKB/Uniprot accession number O15455), TLR7 (UniProtKB/Uniprot accession number Q9NYK1), TLR8 (UniProtKB/Uniprot accession number Q9NR97), and TLR9 (UniProtKB/Uniprot accession number Q9NR96), scan the extracellular and endosomal space for the detection of RNA and DNA, thereby detecting viral genomes from lysed virus particles outside the cell (Kawai and Akira 2006). Other TLRs bind other pathogen components such as bacterial proteins, lipoproteins, or peptidoglycans, as well as bacterial ribosomal RNA sequence and small molecules such as lipoteichoic acid or lipopolysaccharides.

Several cytosolic sensors have been described, of which two types have been characterized: the RIG-I-like receptors (RNA sensing retinoic acid inducible gene, i.e. RIG-I, MDA5, IFIIH1, and LGP2) and the cGAS DNA sensor, all of which detect viral genomes in the cytoplasm. RIG-I (UniProtKB/Uniprot accession number O95786) recognizes tri-phosphate and di-phosphate at the end of a dsRNA (double-stranded RNA) stem, a hallmark of the viral RNAs of the majority of the RNA viruses (Pichlmair, Schulz et al. 2006). MDA5 (also named IFIT1; UniProtKB/Uniprot accession number Q96C10) senses long dsRNAs, which are believed to represent replicative intermediates for many RNA viruses (Kato, Takeuchi et al. 2006). LGP2 is a protein structurally related to both RIG-I and MDA5 that appears to be a cofactor in viral RNA sensing through a still not completely clear mechanism that most likely involves making the viral RNA more accessible to RIG-I or MDA5 (Venkataraman, Valdes et al. 2007). For DNA viruses, the presence of cytoplasmic DNA associated with their infection is the trigger for IFN induction. The cellular sensor cGAS (UniProtKB/Uniprot accession number Q8N884) becomes activated when it binds to cytoplasmic DNA from DNA viruses, which synthesizes the dinucleotide cGAMP (i.e. cyclic GMP-AMP) that stimulates the IFN inducing cascade (Li, Wu et al. 2013).

IFN Typed Signaling Pathway and Effectors

Upon interaction with their specific PAMP, TLRs homo- or heterodimerize and interact with the MyD88 (UniProtKB/Uniprot accession number Q99836) and TIR-domain-containing adapter-inducing interferon-β (TRIF; UniProtKB/Uniprot accession number Q8IUC6) adapters to initiate the downstream signaling interferon cascade. The cellular sensors also recruit STING (DNA-sensing; UniProtKB/Uniprot accession number Q86WV6) and MAVS (RNA-sensing; UniProtKB/Uniprot accession number Q7Z434) adapters, which also participate in the initiation of the downstream signaling interferon cascade.

The activation of the membrane and intracellular sensors lead to the activation of specific serine/threonine kinases, such as TBK1 (UniProtKB/Uniprot accession number Q9UHD2) and IKKε (UniProtKB/Uniprot accession number Q14164). They activate by phosphorylation IFN Regulatory Factor (IRF) family, such as IRF3 (UniProtKB/Uniprot accession number Q14653) and IRF7 (UniProtKB/Uniprot accession number Q92985). They are found in an inactive cytoplasmic form that upon serine/threonine phosphorylation homo- or hetero-dimerize, then translocate to the nucleus to activates the transcription of IFNα and β, as well as other interferon-induced genes.

Once secreted, type I IFNα and β signal through their receptor IFNAR in a paracrine and autocrine manner. IFNAR is a heteromeric cell surface receptor composed of two subunits, referred to as the low affinity subunit, IFNAR1 (UniProtKB/Uniprot accession number P17181), and the high affinity subunit, IFNAR2 (UniProtKB/Uniprot accession number P48551). Upon binding of type I IFNs, IFNAR activates the JAK-STAT signaling pathway. In this pathway, JAKs, such as JAK1 (UniProtKB/Uniprot accession number P23458) and TYK2 (UniProtKB/Uniprot accession number P29597), associate with IFN receptors and, following receptor engagement with IFN, phosphorylate both STAT1 (UniProtKB/Uniprot accession number P42224) and STAT2 (UniProtKB/Uniprot accession number P52630).

Phosphorylated STATs interact with interferon regulatory factor IRF9 (UniProtKB/Uniprot accession number Q00978) and forms the interferon-stimulated gene factor 3 (ISGF3) complex, which translocate into the cell nucleus and binds to specific nucleotide sequences called IFN-stimulated response elements (ISREs) in the promoters of many genes, known as IFN stimulated genes ISGs.

Up to a thousand ISGs have been characterized but the function of only a few is known. Some of these genes encode for important effectors of the type-I interferon response in addition to EIF2AK2, including MX1 (UniProtKB/Uniprot accession number P20591), OAS1 (UniProtKB/Uniprot accession number P00973), RNASEL (UniProtKB/Uniprot accession number Q05823), APOBEC3G (UniProtKB/Uniprot accession number Q9HC16), TRIMS (UniProtKB/Uniprot accession number Q0PF16), ISG15 (UniProtKB/Uniprot accession number P05161), ADAR (UniProtKB/Uniprot accession number P55265), IFITM1 (UniProtKB/Uniprot accession number P13164), IFITM2 (UniProtKB/Uniprot accession number Q01629), IFITM3 (UniProtKB/Uniprot accession number Q01628), BST2 (UniProtKB/Uniprot accession number Q10589), RSAD2 (UniProtKB/Uniprot accession number Q8WXG1), and IFIT1 (UniProtKB/Uniprot accession number P09914).

Viral Proteins

Several viral proteins can antagonize the IFN system, and very often inhibit the IFN-mediated antiviral response at multiple steps. Some examples are given according the main protein targeted:
DDX58 (also named RIG-I; UniProtKB/Uniprot accession number O95786) can be inhibited by NS2 (UniProtKB/Uniprot accession number O42038) from Respiratory syncytial virus (Ling, Tran et al. 2009, Masatani, Ito et al. 2010), NS1 (UniProtKB/Uniprot accession number P03496) from Influenza A (Gack, Albrecht et al. 2009), Z protein (UniProtKB/Uniprot accession number Q6UY71) from virus New world arenaviruses (Fan, Briese et al. 2010), and VP35 (UniProtKB/Uniprot accession number Q05127) from Zaire Ebolavirus (Cardenas, Loo et al. 2006).
IFIH1 (also named MDA5; UniProtKB/Uniprot accession number Q9BYX4) can be inhibited by V protein (UniProtKB/Uniprot accession number O55777) from paramyxovirus (Childs, Andrejeva et al. 2009).
MAVS (UniProtKB/Uniprot accession number Q7Z434) can be inhibited by ABC polyprotein (UniProtKB/Uniprot accession number P08617) from Hepatitis A (Yang, Liang et al. 2007), X protein (UniProtKB/Uniprot accession number Q7TDY3) from Hepatitis B virus (Wei, Ni et al. 2010), NS3/4A (UniProtKB/Uniprot accession number P27958) from Hepatitis C virus (Li, Sun et al. 2005), PB1-F2 protein (UniProtKB/Uniprot accession number P0C0U1) from Influenza virus A (Varga, Grant et al. 2012).
TBK1 (UniProtKB/Uniprot accession number Q9UHD2) can be inhibited by ICP34.5 (UniProtKB/Uniprot accession number P08353) from Herpes virus 1 (Ma, Jin et al. 2012), ORF11 (UniProtKB/Uniprot accession number C9DRI5) from murine gamma-herpesvirus 68 (Kang, Cheong et al. 2014), and C6 protein (UniProtKB/Uniprot accession number P17362) from Vaccinia virus (Unterholzner, Sumner et al. 2011).
TRAF2 (UniProtKB/Uniprot accession number Q12933) and/or TRAF3 (UniProtKB/Uniprot accession number Q13114) proteins can be inhibited by NS5A (UniProtKB/Uniprot accession number P27958) from hepatitis C virus (Park, Choi et al. 2003), VP4 (UniProtKB/Uniprot accession number A2T3T2) from rotavirus (LaMonica, Kocer et al. 2001), LHDAg (UniProtKB/Uniprot accession number P29996) from hepatitis delta virus (Park, Oh et al. 2009), vFLIP (UniProtKB/Uniprot accession number P88961) from Kaposi's sarcoma herpesvirus (Guasparri, Wu et al. 2006), Gn protein (UniProtKB/Uniprot accession number P08668) from Hantavirus (Alff, Sen et al. 2008), M protein (P59596) from SARS-Cov coronavirus (Siu, Kok et al. 2009), and LMP1 (UniProtKB/Uniprot accession number P03230) from herpes virus (Wu, Xie et al. 2005).
IRF3 (UniProtKB/Uniprot accession number Q14653) can be inhibited by bICP0 from bovine herpesvirus 1 (Saira, Zhou et al. 2007), BGLF4 kinase from Epstein-Barr virus (Wang, Doong et al. 2009), Npro from Bovine Viral Diarrhea Virus (Seago, Hilton et al. 2007, Peterhans and Schweizer 2013), NSP1 from rotavirus (Barro and Patton 2005), PLpro from SARS-CoV coronavirus (Devaraj, Wang et al. 2007), LANA-1 from Kaposi sarcoma-associated herpesvirus (Cloutier and Flamand 2010), E6 from human Papillomavirus 16 (Ronco, Karpova et al. 1998), and L protein from Theiler's virus (Spiegel, Pichlmair et al. 2005).
IRF7 (UniProtKB/Uniprot accession number Q92985) can be inhibited by VP35 (UniProtKB/Uniprot accession number Q05127) from Ebola Zaire virus (Chang, Kubota et al. 2009), BZLF-1 from (UniProtKB/Uniprot accession number P03206) from Epstein-Barr virus (Hahn, Huye et al. 2005), ORF45 (UniProtKB/Uniprot accession number F5HDE4) from human herpesvirus 8 (Zhu, King et al. 2002), NSP1 (UniProtKB/Uniprot accession number Q99FX5) from rotavirus (Barro and Patton 2007), and ML protein from (UniProtKB/Uniprot accession number Q80A33) Thogoto virus (Buettner, Vogt et al. 2010).
STAT1 (UniProtKB/Uniprot accession number P42224) can be inhibited by P protein (UniProtKB/Uniprot accession number Q91K91) from Nipah virus (Ciancanelli, Volchkova et al. 2009), E1A protein (UniProtKB/Uniprot accession number P03255) from adenovirus (Look, Roswit et al. 1998), V protein (UniProtKB/Uniprot accession number P0C774) from measles virus (Caignard, Bourai et al. 2009), V protein (UniProtKB/Uniprot accession number P30928) from Mumps virus (Kubota, Yokosawa et al. 2002), P protein (UniProtKB/Uniprot accession number P16286) from rabies virus (Chelbi-Alix, Vidy et al. 2006), C protein (UniProtKB/Uniprot accession number P04862) from Sendai virus (Garcin, Marq et al. 2002), and V protein (UniProtKB/Uniprot accession number P11207) from PIV5 (Didcock, Young et al. 1999, Precious, Carlos et al. 2007).
STAT2 (UniProtKB/Uniprot accession number P52630) can be inhibited by NS5 (UniProtKB/Uniprot accession number P17763) from Dengue virus polyprotein (Ashour, Laurent-Rolle et al. 2009), V protein (UniProtKB/Uniprot accession number P19847) from Human Parainfluenza Virus 2 (Parisien, Lau et al. 2001), V protein (UniProtKB/Uniprot accession number Q997F2) from Nipah virus polyprotein (Rodriguez, Parisien et al. 2002), V protein (UniProtKB/Uniprot accession number P0C774) from measles virus (Ramachandran, Parisien et al. 2008), P phosphoprotein (UniProtKB/Uniprot accession number P16286) from Rabies virus (Brzozka, Finke et al. 2006), and NS1 (UniProtKB/Uniprot accession number P0DOE9) from human respiratory syncytial virus (Elliott, Lynch et al. 2007).
IRF9 (UniProtKB/Uniprot accession number Q00978) can be inhibited by E7 (UniProtKB/Uniprot accession number P03129) from human papillomavirus (Barnard and McMillan 1999), and μ2 protein (UniProtKB/Uniprot accession number Q00335) from reovirus (Zurney, Kobayashi et al. 2009).
TYK2 (UniProtKB/Uniprot accession number P29597) can be inhibited by LMP-1 (UniProtKB/Uniprot accession number P03230) from Epstein-Barr virus (Geiger and Martin 2006), E6 (UniProtKB/Uniprot accession number Q9QNP8) from human papillomavirus-18 (Li, Labrecque et al. 1999), and NS5 (UniProtKB/Uniprot accession number P27395) from Japanese encephalitis virus polyprotein (Lin, Chang et al. 2006).
JAK1 (UniProtKB/Uniprot accession number P23458) can be inhibited by VP40 protein (UniProtKB/Uniprot accession number P35260) from the Marburg virus (Valmas and Basler 2011), and T antigen (UniProtKB/Uniprot accession number P03071) from polyomavirus (Weihua, Ramanujam et al. 1998).
IFNAR1/IFNAR2 (UniProtKB/Uniprot accession number P17181 and P48551) can be inhibited by 3a protein (UniProtKB/Uniprot accession number P59632) from SARS-Cov coronavirus (Minakshi, Padhan et al. 2009), K3 (UniProtKB/Uniprot accession number P90495) and K5 proteins (UniProtKB/Uniprot accession number P90489) from Kaposi's sarcoma herpesvirus (Li, Means et al. 2007).
EIF2AK2 (UniProtKB/Uniprot accession number P19525) can be inhibited by E3L protein (UniProtKB/Uniprot accession number P21081) from vaccinia virus (Davies, Chang et al. 1993), K3L protein (UniProtKB/Uniprot accession number P18378) from vaccinia virus (Davies, Chang et al. 1993), NSs protein (UniProtKB/Uniprot accession number P21698) from Rift Valley fever virus (Habjan, Pichlmair et al. 2009), σ3 protein (UniProtKB/Uniprot accession number P07939) from orthoreovirus (Imani and Jacobs 1988), NS1 protein (UniProtKB/Uniprot accession number P03496) from Influenza A (Bergmann, Garcia-Sastre et al. 2000) or B viruses (UniProtKB/Uniprot accession number P03502) (Dauber, Schneider et al. 2006), NSP3 (UniProtKB/Uniprot accession number Q85015) from porcine rotavirus (Langland, Pettiford et al. 1994), Us11 from Herpes simplex 1 virus (Khoo, Perez et al. 2002), E6 from human papillomavirus 16 (Hebner, Wilson et al. 2006), TRS1 from human cytomegalovirus (Hakki, Marshall et al. 2011), pm142-pm143 from mouse cytomegalovirus (Child and Gebelle 2009), TFV 27R from Iridovirus (Essbauer, Bremont et al. 2001), C8L from swinepox virus (Kawagishi-Kobayashi, Cao et al. 2000), E1B and E4 from human adenovirus C (Spurgeon and Ornelles 2009), and N protein from Respiratory syncytial virus (Groskreutz, Babor et al. 2010), ORF4a from Middle East respiratory syndrome coronavirus (MERS CoV; (Khan, Tahir Khan et al. 2020).
IFIT1 (UniProtKB/Uniprot accession number P09914) can be inhibited by the secondary structural motifs within the 5′-UTR of the genomic RNA from alphaviruses (Hyde, Gardner et al. 2014, Reynaud, Kim et al. 2015).

EIF2AK2 Kinase

EIF2AK2 (eukaryotic translation initiation factor 2-alpha kinase 2; also known as Protein kinase RNA-activated or PKR), is a key effector of type-I interferon response.

EIF2AK2 is a nuclear and cytoplasmic protein, which contains an N-terminal dsRNA binding domain (dsRBD) and a C-terminal protein domain, that gives it kinase and pro-apoptotic functions. The dsRBD consists of two tandem copies of a conserved double stranded RNA binding motif, dsRBM1 and dsRBM2. These two domains, which are separated by a protein region with no distinct structure or function.

EIF2AK2 is activated by dsRNA, which can be formed as a result of transcription of viral DNA or RNA templates in both sense and antisense directions or DNA- or RNA-containing inverted repeats, which upon transcription fold into dsRNA hairpins. dsRNA is considered as a hallmark of infection and replication of dsRNA and single-stranded RNA (ssRNA) viruses, except for retroviruses. The production of dsDNA is also observed in cases of infection by cytoplasmic DNA viruses.

The binding of dsRNA to EIF2AK2 induces the dimerization of its dsRBD and subsequently its auto-phosphorylation. Once active, EIF2AK2 can phosphorylate the eukaryotic translation initiation factor eIF2α, which inhibits further cellular mRNA translation, thereby preventing viral protein synthesis.

EIF2AK2 also activates the transcription factor NFkB (UniProtKB/Uniprot accession number P19838), by phosphorylating its inhibitory subunit, IkkB (UniProtKB/Uniprot accession number O14920). Activated NFkB upregulates the expression of Interferon cytokines, which work to spread the antiviral signal locally. EIF2AK2 also activates tumor suppressor PP2A heterodimer (UniProtKB/Uniprot accession number P67775 and P62714) which regulates the cell cycle and the metabolism. Through complex mechanisms, active EIF2AK2 is able to induce cellular apoptosis, to prevent further viral spread. EIF2AK2 can be inhibited by various means including the viral proteins listed above or other polypeptides, chemical compounds (e.g. CAS 608512-97-6 or C16/GW-506033X), nucleic acids (e.g. siRNA, shRNA, miRNA, antisense or ribozyme). Host-cell EIF2AK2 can be also knocked-out by various gene editing technologies, such as ZFNs, TALENs, and CRISPR-Cas9 system and its derivatives. In addition, artificial inductors for EIF2AK2 degradation can be generated using the proteolysis targeting chimera system, also abbreviated PROTAC (Sakamoto, Kim et al. 2001). PROTACs consist of two covalently linked protein-binding molecules: one capable of engaging an E3 ubiquitin ligase, and another that binds to a target protein meant for degradation. By analogy with PROTACs that are heterobifunctional small molecules, chimeric proteins that can drive the degradation of target proteins by ubiquitination are commonly called biological PROTACS or BIO-PROTACs (Lim, Khoo et al. 2020). Such a technology can be easily adapted for the decay of EIF2AK2 or other EIF2αkinases by fusion of stimuli-binding domain (i.e. dsRNA-binding domain for EIF2AK2) to an E3 ligase. The construction thus generated allows the degradation of the EIF2αkinases targeted by ubiquitination.

### SUMMARY OF THE INVENTION

The inventor has unexpectedly found that the translation of RNA molecules produced by the artificial expression system already described in WO2011/12844 and WO2019/020811 is partially inhibited in eukaryotic cells by a type-I interferon and/or by unfolded protein responses, which are both responsible for a translational arrest triggered by eIF2 phosphorylation.

This finding was particularly unexpected because the polysomal profile obtained with first generation of the artificial expression system consisting of the fusion of a capping enzyme with DNA-dependent RNA polymerase was normal, which suggested the absence of translation initiation defect (Jais, Decroly et al. 2019). Surprisingly, the present inventor has discovered that the polysomal profile obtained with second generation of the artificial expression system, which consisted of a tethered poly(A) polymerase and the artificial expression system of the first generation, was massively disturbed. The profile obtained with this second generation polysomal profile was featured by reduced 40S and 60S ribosome peaks, massive increase the 80S monosome peak formed by the assembly of the 40S and 60S ribosome subunits, and frank decrease of the polysome peaks (Example 1).

The inventor has moreover demonstrated that the translation of RNA molecules produced by the artificial expression system can be greatly increased by inhibiting some of the cellular mechanisms of defense, especially those triggering the phosphorylation of eIF2α (or EIF2α in the following), such as the type-I interferon and unfolded protein responses.

By inhibiting those responses leading to the phosphorylation of EIF2α, the inventor has unexpectedly found that the level of translation rate of mRNA molecules produced by artificial expression systems in eukaryotic cells, such as those described in WO2011/12844 and WO2019/020811, can be increased by at least 30%, preferably at least 50%, and even more, as much as at least 5-fold.

Thus, in one aspect, the invention relates to a method for expressing a recombinant DNA molecule or mRNA molecule in a eukaryotic host cell, comprising the steps of:
(a) expressing or introducing at least one chimeric protein or enzyme, into said host cell, wherein said chimeric protein or enzyme comprises:
   (i) at least one catalytic domain of a capping enzyme, in particular selected in the group consisting of cap-0 canonical capping enzymes, cap-0 non-canonical capping enzymes, cap-1 capping enzymes and cap-2 capping enzymes; and
   (ii) at least one catalytic domain of a DNA-dependent RNA polymerase, in particular a bacteriophage DNA-dependent RNA polymerase; and
(b) constitutively or transiently downregulating the phosphorylation level of subunit a of translation initiation factor eIF2 (eIF2α) in said host cell.

According to a second aspect, the present invention is also directed to a eukaryotic host cell for the expression of a recombinant protein, wherein the phosphorylation level of eIF2α is constitutively or transiently downregulated in said cell, and wherein said cell comprises at least one nucleic acid molecule encoding at least one chimeric protein or enzyme comprising:
(i) at least one catalytic domain of a capping enzyme as disclosed above; and
(ii) at least one catalytic domain of a DNA-dependent RNA polymerase.

According to still another aspect, the invention is directed to a nucleic acid molecule or a set of nucleic acid molecules, comprising or consisting of:
(a) at least one nucleic acid sequence encoding a chimeric protein or enzyme comprising:
   (i) at least one catalytic domain of a capping enzyme; and
   (ii) at least one catalytic domain of a DNA-dependent RNA polymerase; and
(b) at least one nucleic acid sequence downregulating the phosphorylation level of eIF2α in a eukaryotic host cell, or encoding a compound downregulating said phosphorylation level.

According to still another aspect, the invention is also directed to a kit for the production of a recombinant protein of interest, comprising or consisting of:
(a) an isolated nucleic acid molecule or a set of isolated nucleic acid molecules encoding a chimeric protein or enzyme comprising:
   (i) at least one catalytic domain of a capping enzyme; and
   (ii) at least one catalytic domain of a DNA-dependent RNA polymerase; and
(b) at least one compound capable of downregulating the phosphorylation level of eIF2α in a eukaryotic host cell, or a nucleic acid molecule encoding such a compound.

The invention also concerns vectors comprising or consisting of the isolated nucleic acid molecule or set of isolated nucleic acid molecules as mentioned above, as well as polyproteins, polypeptides or sets of polypeptides encoded by said isolated nucleic acid molecule or set of isolated nucleic acid molecules as mentioned above.

The invention also concerns different uses, processes and applications of the methods, isolated nucleic acid molecules or set of isolated nucleic acid molecules, vectors, kits, polyproteins, polypeptide and sets of polypeptides as described, especially methods of treatment, method for generating an immune response, methods or use in gene therapy, method for bioproduction, and other methods which will be detailed in the following.

### Definitions

As used herein, the term “chimeric protein/enzyme” refers to a protein/enzyme that is not a native protein/enzyme found in the nature (that is non-natural). Accordingly, a chimeric protein/enzyme may comprise domains, especially catalytic domains, that are derived from different sources (e.g. from different proteins/enzymes) or domains derived from the same source (e.g. from the same protein), but arranged in a different manner than that found in nature. In particular, the chimeric protein/enzyme according to the invention is a monomeric or oligomeric non-natural protein/enzyme.

The term “chimeric enzyme” encompasses monomeric (i.e. single-unit) enzyme but also oligomeric (i.e. multi-unit) enzyme, in particular hetero-oligomeric enzyme.

The term “catalytic domain” of an enzyme relates to a protein domain, which is necessary and sufficient, in particular in its three-dimensional structure, to assure the enzymatic function. The term “catalytic domain” encompasses catalytic domain of wild-type or mutant enzyme.

The term “domain” defines distinct functional and/or structural building blocks and elements in a protein which folds and functions independently.

As used herein, the term “monomeric protein” relates to a single-unit protein that consists of only one polypeptide chain.

As used herein, the term “oligomeric protein” refers to a multi-unit enzyme that consists of at least two polypeptide chains, linked together covalently or noncovalently.

As used herein, the term “polyprotein” refers to a protein, usually large in size, encoded by a single open reading frame, but either cleaved by the action of exogenous or cellular endopeptidases, or produced into multiple proteins by the action of the ribosome skipping motif such as 2A sequences.

As used herein, the term “fusion protein” relates to artificial proteins created through the joining of two or more proteins or protein domains that originally coded for separate proteins. Translation of this fusion gene results in a single or multiple polypeptides with functional properties derived from each of the original proteins.

As used herein, the terms «link» and «bound» encompass covalent and non-covalent linkage. As used herein, the term “capping enzyme” refers to any enzyme able to add a ^{m7}GpppN₁N₂ cap at 5′-end of mRNA (where Ni and N2 are the ultimate and penultimate bases of the mRNA) and/or to modify the ultimate or penultimate bases of a RNA sequence, including cap-0 canonical or non-canonical capping enzymes and cap-1 or cap-2 nucleoside 2′ methyltransferases, N6-methyl-adenosine transferase.

As used herein, the term “cap-0 canonical capping enzymes” refers to enzymes able to add cap-0 structure at the 5′end of RNA molecules by involving a series of three enzymatic reactions: RNA triphosphatase (RTPase) that removes the γ phosphate residue of 5′ triphosphate end of nascent pre-mRNA to diphosphate ppRNA, RNA guanylyltransferase (GTase) that transfers GMP from GTP to the diphosphate ppRNA nascent RNA terminus, and RNA N⁷-guanine methyltransferase (N7-MTase) that adds a methyl residue on nitrogen 7 of guanine to the GpppRNA cap.

As used herein, the term “cap-0 non canonical capping enzymes” refers to enzymes able to add a cap-0 structure at the 5′ end of RNA molecules but by an enzymatic process which differs from the canonical enzymatic process.

As used herein, the term “cap-1 capping enzymes” refers to enzymes able to add cap-1 structure at the 5′ ends of RNA molecules, namely a 2′-O-methylation at the ultimate base of the 5′-ends of the RNA molecules.

As used herein, the term “cap-2 capping enzymes” refers to enzymes able to add cap-2 structure at the 5′ ends of RNA molecules, namely a 2′-O-methylation at the penultimate base of the 5′-ends of the RNA molecules.

As used herein, the term “5′-end RNA processing enzyme other than cap-0, cap-1 and cap-2 capping enzymes” relates to enzymes able to modify the ultimate or penultimate bases of a mRNA sequence, other than cap-0, cap-1 and cap-2 capping enzymes, including N6-methyl-adenosine transferase and enzymes able to add 2,2,7-trimethylguanosine (TMG) and 2,7-trimethylguanosine (DMG) cap modifications at the 5′end of RNA molecules.

As used herein, the term “DNA-dependent RNA polymerase” (RNAPs) relates to nucleotidyl transferases that synthesize complementary strand of RNA from a single- or double-stranded DNA template in the 5′-3′ direction.

As used herein, the term “poly(A) polymerase” relates to any enzyme able to catalyze the non-templated addition of adenosine residues from ATP onto the 3′ end of RNA molecules.

The term “noncanonical poly(A) polymerases”, refers to enzymes, that do not have a tripartite structure involving a N-terminal nucleotidyltransferase (NT) catalytic domain, a central domain, and a C-terminal domain corresponding to the RNA binding domain (RBD).

As used herein, the term “protein-RNA tethering system” refers to a system wherein a protein (or a peptide) recognizes and specifically binds (with high affinity) via its RNA-binding domain to a specific RNA element consisting of a specific RNA sequence and/or structure, therefore making possible to tether this protein (or peptide) with this RNA element.

As used herein, the term “gene editing” relates to all technologies allowing the stable modification of the cell genome. They include in particular zinc finger nucleases (ZFNs), transcription activator-like effector nucleases (TALENs), engineered meganucleases and CRISPR-Cas9 system and its derivatives.

As used herein, the term “in cellulo” relates to cellular work or experiment done in single cells of more complex organisms, such as mammalian cultured cells.

As used herein, the term “orthogonal” designate biological systems whose basic structures are independent and generally originates from different species.

As used herein the term “the RNA element of a protein-RNA tethering system which specifically binds to said RNA-binding domain” relates to an RNA sequence, usually forming a stem-loop, which is able to bind with high affinity to the corresponding RNA-binding domain of a protein-RNA tethering system.

As used herein the term “endogenous DNA-dependent RNA polymerase” relates to the endogenous DNA-dependent RNA polymerase of said host cell. When the host cell is a eukaryotic cell, said endogenous DNA-dependent RNA polymerase is the RNA polymerase II.

As used herein the term “endogenous capping enzyme” refers to the endogenous capping enzyme of said host cell.

### DETAILED DESCRIPTION OF THE INVENTION

Inhibition of eIF2α Phosphorylation

Overview

As described hereinafter, the inhibition of phosphorylation of eIF2α or the increase of its dephosphorylation, which can be operated by several means, are critical for increased expression level by an artificial expression system such as the C3P3 expression system, especially when the system comprises a catalytic domain of a poly(A) polymerase.

According to a first aspect, the invention relates to a method for expressing a recombinant DNA molecule in a eukaryotic host cell, comprising the steps of:
(a) expressing or introducing at least one chimeric protein or enzyme, into said host cell, wherein said chimeric protein or enzyme comprises:
   (i) at least one catalytic domain of a capping enzyme, in particular selected in the group consisting of cap-0 canonical capping enzymes, cap-0 non-canonical capping enzymes, cap-1 capping enzymes and cap-2 capping enzymes; and
   (ii) at least one catalytic domain of a DNA-dependent RNA polymerase, in particular a bacteriophage DNA-dependent RNA polymerase; and
(b) constitutively or transiently downregulating the phosphorylation level of subunit a of translation initiation factor eIF2 (eIF2α) in said host cell.

Downregulating the phosphorylation level of eIF2α may comprise or consist in the impairment, or inhibition of a pathway leading to phosphorylation of eIF2α, and/or in the activation, or stimulation of a pathway leading to dephosphorylation of eIF2α.

In a variant of this method also covered by the invention, at least one chimeric protein or enzyme is expressed or introduced into a eukaryotic host cell already modified, preferably constitutively modified, to downregulate or know-out by gene editing the phosphorylation level of eIF2α. Such a modification can be obtained, for example by genetically impairing at least one gene encoding a protein involved in the pathway leading to phosphorylation of eIF2α, or by silencing one or more of these genes, for example by expressing siRNAs as disclosed in the experimental section, or by editing such a gene, or by expressing inhibitors, antagonists or competitive agonists of the corresponding proteins.

Particularly preferred cells are cells defective for at least one of the enzymes involved in type-I interferon response or in the unfolded protein response, e.g. defective for at least one of the genes coding for EIF2AK2 (NCBI GenBank accession number NM_002759), EIF2AK3 (NCBI GenBank accession number NM_004836), DDX58 (also named RIG-I; NCBI GenBank accession number NM_014314), IFIH1 (also named MDA5; NCBI GenBank accession number NM_022168), MAVS (NCBI GenBank accession number NM_020746), IFNAR1 (NCBI GenBank accession number NM_000629), IFNAR2 (NCBI GenBank accession number NM_207584), IRF3 (NCBI GenBank accession number NM_001571), IRF7 (NCBI GenBank accession number NM_004030), IFNB1 (NCBI GenBank accession number NM_002176), TBK1 (NCBI GenBank accession number NM_013254.4), TRAF2 (NCBI GenBank accession number NM_021138.4), TRAF3 (NCBI GenBank accession number NM_145725.3), IFIT1 (NCBI GenBank accession number NM_001548.5) or a protein of the JAK-STAT pathway, in particular JAK1 (NCBI GenBank accession number NM_002227), STAT1 (NCBI GenBank accession number NM_139266), STAT2 (NCBI GenBank accession number NM_005419), TYK2 (NCBI GenBank accession number NM_003331) and IRF9 (NCBI GenBank accession number NM_006084). Potentially, the cells may be defective for at least two genes, i.e. two genes or more coding from proteins involved directly or indirectly in the phosphorylation of EIF2α. In such a case, step (b) of the method recited above has already been carried out and the variant method of the invention only comprise step (b), namely the introduction or expression of the chimeric protein in said specific host cells, already modified with regard to its EIF2αphosphorylation level.

According to some aspects of the invention, the method or its variant is used in vitro (e.g. for in vitro protein synthesis or recombinant proteins) or in cellulo (e.g. for bioproduction of recombinant proteins from the recombinant DNA using cultured mammalian cell lines), or ex vivo (e.g. for cellular immunotherapy with CAR-T cells). The method can thus be used for example in cultured eukaryotic cells or in isolated eukaryotic cells. The method can also be used in vivo, especially in a eukaryotic organism, especially a mammal, more preferably human, e.g. for synthetic gene therapy or synthetic gene vaccination.

The step (a) of expressing or introducing the chimeric protein or enzyme and the step (b) of downregulating the phosphorylation level of eIF2α can be carried out in any order, simultaneously or sequentially, except for the variant of the method, where step (b) has been carried out before the beginning of the variant method. According to a preferred embodiment, the phosphorylation level of eIF2α is downregulated at least once and preferably as soon as the chimeric protein or enzyme is expressed or introduced into the host cell.

The host cell comprises or is transformed to comprise the recombinant DNA molecule or mRNA molecule to be expressed, which can be constitutively or transiently introduced into said cell, preferably but not necessarily in the cytoplasm of said cell. The method of the invention thus allows said recombinant DNA or mRNA molecule to be efficiently expressed, namely efficiently transcribed and translated, within the host cell.

In the context of the present invention, expression of a DNA refers mainly to its transcription and translation into protein or polypeptide.

The host cell may be any eukaryotic cell, especially yeast cells or animal cells, such as vertebrate cells, mammalian cells, primate cells and/or human cells. The host cell is preferably not a human embryonic stem cell.

The chimeric protein or enzyme is advantageously, but not necessarily, expressed or introduced into the cytoplasm of the host cell. Indeed, according to this specific embodiment, the recombinant DNA can be transcribed in the cytoplasm without interfering with the transcription machinery of the host cell.

According to one aspect, the phosphorylation level of eIF2α is constitutively downregulated or knocked-out by gene editing in the host cell. This downregulation may advantageously be obtained by using a host cell which has already been genetically modified to impair the pathway leading to phosphorylation of eIF2α or to activate a pathway leading to dephosphorylation of eIF2α, corresponding to the variant of the method mentioned above.

According to another embodiment, the phosphorylation level of eIF2α is only transiently downregulated. As used herein the term “downregulation of the phosphorylation level of EIF2α” relates to a decrease of at least 20%, particularly at least 35%, at least 50% and more particularly at least 65%, at least 80%, at least 90% of the phosphorylation level of EIF2α, with respect to the phosphorylation level of EIF2α in the absence of said step of downregulation. In particular, downregulating the phosphorylation level of eIF2α consists in reducing the level of phosphorylated eIF2α with respect to a host cell which does not comprise this step of downregulation, and in which phosphorylated eIF2α is present in the cell, for instance triggered by a stimulus such as the presence of artificially transcribed mRNA, generated by the artificial expression system C3P3, in particular C3P3-G1 or C3P3-G2 (WO2011/128444, Jais et al. 2019 or WO2019/020811). The phosphorylation level of EIF2αcan be measured by any technique known to the skilled reader, especially one of the techniques disclosed in the section below detailing different EIF2 phosphorylation assays.

The downregulation of the phosphorylation level of eIF2α is preferably obtained by modulating the activity and/or expression of a target host cell protein involved in the regulation of the phosphorylation level of eIF2α. This downregulation can rely on inhibition of the activity or decreasing the expression of host cell proteins involved in the phosphorylation of eIF2α, either activating or increasing expression or activity of proteins host cells involved in the dephosphorylation of eIF2α, such as for example the catalytic subunit of the serine/threonine eIF2α phosphatase 1 alpha (human protein UniProtKB/Uniprot accession number P62136, also named PPP1CA), as well as its viral or host-cell regulatory subunits including PPP1R15 (also known as GADD34, UniProtKB/Uniprot accession number ID O75807), DP71L(s) or DP71L(I) from African swine fever virus (UniProtKB/Uniprot accession number Q65212 and P0C755, respectively) and ICP34.5 from human Herpes-simplex virus-1 (UniProtKB/Uniprot accession number P36313). The modulation of these target host cell gives rise to a downregulation of the phosphorylation level of EIF2α of at least 20%, as mentioned above.

The modulation of at least one target host cell protein involved in the regulation of the phosphorylation level of eIF2α may be obtained by any means known to the skilled reader. Such a modulation, in case of inhibition or downregulation of a target host cell protein involved in the phosphorylation of eIF2α can for example be obtained by using small molecules or introducing or expressing a siRNA targeting said host cell protein, or a shRNA or miRNA. Illustrations of the siRNA embodiment are described in the example section of the application. Inhibition can also be obtained by expression or introduction of an antisense, ribozymes or catalytic DNA targeting the DNA or RNA corresponding to the host cell protein. Such modulation of a target host cell protein involved in the phosphorylation of eIF2α can also be carried out using a modulator polypeptide, i.e. a polypeptide modulating the activity or the expression of a target host cell protein involved in the regulation of the phosphorylation level of eIF2α. The downregulation of a target host cell protein involved in the phosphorylation of eIF2α can also be achieved using a proteolysis targeting chimera protein, as described above, e.g. a biological proteolysis targeting chimera system (BIO-PROTAC).

eIF2 phosphorylation assays are well known to the skilled reader, for defining host cell proteins to be targeted and appropriate modulators.

The host cell protein to be targeted can be a protein involved in the type-I interferon response, or in the unfolded protein response. More generally, the host cell protein to be targeted is known to be involved in the eukaryotic eIF2 response.

Examples of potential target host cell proteins are those mentioned in the introductive section and include: EIF2AK2, EIF2AK3, DDX58, IFIH1, MAVS, IFNAR1, IFNAR2, IRF3, IRF7, IFNB1, TBK1, TRAF2, TRAF3, IFIT1 or proteins of the JAK-STAT pathway, in particular JAK1, STAT1, STAT2, TYK2 and IRF9. Preferred host cell targets are EIF2AK2 and EIF2AK3, which are directly involved in the phosphorylation of eIF2α. According to an embodiment, step (b) is carried out by introducing or expressed in the cell a compound which is an inhibitor of one these proteins, especially an inhibitor of EIF2AK2 and EIF2AK3.

Deletion or mutation of the corresponding gene encoding the target host cell protein may also be obtained by targeted gene editing, by using systems such a TALEN or CRISPR, well known to the skilled reader. Such methods are perfectly suited also for the variant method of the invention.

According to another aspect of the present invention, the downregulation of the phosphorylation level of eIF2α is advantageously carried out by introducing a compound downregulating the phosphorylation level of eIF2α, especially a compound inhibiting a host cell protein responsible, directly or indirectly, for the phosphorylation of eIF2α. Said compound can be for example a chemical compound, a siRNA, shRNA, miRNA, an antisense, a ribozyme or a polypeptide. When said compound can be expressed from a nucleic acid molecule, the compound downregulating the phosphorylation level of eIF2α can either be introduced directly, or can be expressed in the cell from a nucleic acid molecule.

The invention is clearly not limited to the introduction or expression of a single compound downregulating the phosphorylation level of eIF2α, and encompasses the introduction or expression of at least two different compounds or means for downregulating the phosphorylation level of eIF2α, either targeting the same host cell protein, or preferably targeting different host cell proteins, such as different host cell proteins from the type-I interferon pathway, or from the unfolded protein response.

According to another aspect of the present invention, the downregulation of the phosphorylation level of eIF2α is advantageously carried out by introducing into the host cell at least one polypeptide, or introducing a nucleic acid molecule encoding such a polypeptide, wherein said polypeptide directly or indirectly downregulates the phosphorylation level of eIF2α. The polypeptide preferably modulates the activity or expression of a target host cell protein involved in the regulation of the phosphorylation level of eIF2α. In the following, such a polypeptide is referred to as “a modulator polypeptide”. Such a modulator polypeptide can be introduced into the host cell, or alternatively a nucleic acid molecule encoding said modulator polypeptide is introduced and the modulator polypeptide is expressed from this introduced nucleic acid molecule. Both embodiments are within the scope of the invention.

A compound according to the invention downregulating the phosphorylation level of eIF2α is preferably an inhibitor of at least one of the following host cell targets: EIF2AK2, EIF2AK3, DDX58, IFIH1, MAVS, IFNAR1, IFNAR2, IRF3, IRF7, IFNB1, TBK1, TRAF2, TRAF3, IFIT, a type-I interferon protein or a protein of the JAK-STAT pathway, in particular JAK1, TYK2, STAT1, STAT2 and IRF9.

The nucleic acid molecule encoding said modulator polypeptide may be introduced transiently, for example as a plasmid, as an expression vector or as an artificial chromosome, or constitutively, for example by recombination or integration into the host cell genome. In the variant method disclosed above, this step has already been carried out and the host cell comprises such a polypeptide or a nucleic acid molecule encoding said polypeptide, preferably constitutively.

The main elements of the eukaryotic expression system according to one aspect of the present invention are thus as follows:
at least one catalytic domain of a capping enzyme,
at least one catalytic domain of a DNA-dependent RNA polymerase; and
at least one means of downregulating the phosphorylation level of eIF2α in a host cell, preferably a modulator polypeptide,
   wherein the catalytic domain of a capping enzyme and the catalytic domain of a DNA-dependent RNA polymerase are as defined herein, and essentially as described in WO2011/128444 and WO2019/020811.

These different elements, as well as additional optional elements can be added, especially a catalytic domain of a poly(A) polymerase, will be described in the following. It is to be understood that the following description of these key elements and of the optional ones, is applicable to all the aspects of the invention mentioned above, namely the different methods, cells, nucleic acid molecules, kit, polypeptides and uses of the invention.

eIF2 Phosphorylation Assays

eIF2 phosphorylation assays are well known to the skilled reader, and can be used for defining host cell proteins to be targeted and appropriate modulators.

Phosphorylation of EIF2 at Ser52 can be assayed by sandwich ELISA (Enzyme Linked Immunosorbent Assay) using microplates coated with a polyclonal antibody, whereas an unbound antibody is raised against the Ser52 phospho-EIF2α(Hong, Nam et al. 2016). Following incubation of microplates with cell lysates and the unbound antibody, Ser52 phospho-EIF2αprotein by can be detected by various methods. First, by colorimetry using a Ser52 phospho-EIF2αantibody conjugated to horseradish peroxidase or alkaline phosphatase, and using a chromogenic substrate such as TMB (3,3′,5,5′-tetramethylbenzidine) or ABTS (2,2′-azino-bis(3-ethylbenzothiazoline-6-sulphonic acid). Second, by chemiluminescence using a Ser52 phospho-EIF2αmonoclonal antibody conjugated to horseradish peroxidase or alkaline phosphatase, and using enhanced luminol-based chemiluminescent (ECL) substrate. Third, by Förster Resonance Energy Transfer (FRET) using two specific antibodies conjugated with lanthanide as donor (e.g. europium (Eu3+) or terbium (Tb2+)) and short-lived fluorophore acceptors (e.g. XL665 or d2) fluorophores, followed by excitation and detection at the appropriate wavelengths. Four, by fluorescence using antibodies conjugated to horseradish peroxidase or alkaline phosphatase, and using fluorescent substrates such as MUP (4-Methylumbelliferyl phosphate) disodium salt.

Ser52 phosphorylation of EIF2 can be also assayed by Western-blotting (Knutsen, Rødland et al. 2015). Cell lysates are loaded on SDS-PAGE gels and the proteins can be analyzed by Western-blotting using phospho-EIF2αspecific antibodies and antibodies raised against non-phosphorylated epitope of eIF2α as control (Berlanga, Ventoso et al. 2006). Example 2(c) describes an assay of phosphorylation of eIF2α by Western-Blotting.

Still another eIF2 phosphorylation assay is based on a system consisting of conjugated beads (Eglen, Reisine et al. 2008, Pytel, Seyb et al. 2014). The donor beads are usually coated with streptavidin to capture a biotinylated antibody directed against a specific phospho-epitope of eIF2α and contain a photosensitizing agent (e.g. phthalocyanine). When irradiated at 680 nm, this photosensitizing agent releases of singlet oxygen molecules from the ambient oxygen that triggers a cascade of energy transfer in the acceptor beads. The acceptor beads are coated with Protein A to capture the second antibody, which is directed against another non-phosphorylated epitope of eIF2. They usually contain three chemical dyes (e.g. thioxene, anthracene and rubrene). Thioxene reacts initially with singlet oxygen to produce light energy, which is subsequently transferred to anthracene and thence to rubrene, then finally emits light at wavelengths of 520-620 nm. In the presence of Ser52 phosphorylated EIF2, the two antibodies bring the donor and acceptor beads close together, resulting in the emission of light, which is directly proportional to the amount of phosphoprotein present in the sample.

EIF2 phosphorylation can be also detected by polysomal profiling, which is a method of global analysis of cell translation that separates translated mRNAs on a sucrose gradient according to the number of bound ribosomes. The typical polysome profile observed when eIF2 is hyperphosphorylated consists of a decrease in 40S, 60S peaks and polysomes, while the peak of the 80S monosome, formed by the assembly of the two subunits, is increased (Dey, Baird et al. 2010, Teske, Baird et al. 2011, Baird, Palam et al. 2014, Andreev, O'Connor et al. 2015, Knutsen, Rødland et al. 2015).

Modulator Polypeptide

According to a preferred embodiment of the present invention, the means for downregulating the phosphorylation level of eIF2α in a host cell is a compound having this activity, and more preferably a modulator polypeptide.

Said modulator polypeptide may be introduced simultaneously with the chimeric protein comprising at least one catalytic domain of a capping enzyme and at least one catalytic domain of a DNA-dependent RNA polymerase. According to an even preferred embodiment, the modulator polypeptide directly or indirectly downregulating the phosphorylation level of eIF2α is part of the chimeric protein of the invention. In such a case, as detailed below and in the examples of the invention, the chimeric protein/enzyme is preferably a polyprotein.

The target host cell protein involved in the regulation of the phosphorylation level of eIF2α which is to be modulated by the modulator polypeptide is preferably a host cell protein involved in the type-I interferon response or in the unfolded protein response of the host cell, and more generally increasing directly or indirectly the phosphorylation rate of eIF2 to increase translation initiation. In one aspect, the modulator polypeptide modulates the activity or expression of a target host cell protein which is activated by dsRNA such as, for example, EIF2AK2.

Potential target host cell proteins which can advantageously be modulated according to the invention are EIF2AK2, EIF2AK3, DDX58, IFIH1, MAVS, IFNAR1, IFNAR2, IRF3, IRF7, IFNB1, TBK1, TRAF2, TRAF3, IFIT or a protein of the JAK-STAT pathway, in particular JAK1, STAT1, STAT2, TYK2 and IRF9, or protein phosphatase 1 PP1 or a subunit thereof, in particular PPP1CA or PPP1R15.

Preferred host cell targets are EIF2AK2 and EIF2AK3, which are directly involved in the phosphorylation of eIF2α.

A polypeptide modulator is thus preferably an inhibitor of at least one of the following host cell targets: EIF2AK2, EIF2AK3, DDX58, IFIH1, MAVS, IFNAR1, IFNAR2, IRF3, IRF7, IFNB1, a type-I interferon protein or a protein of the JAK-STAT pathway, in particular JAK1, TYK2, STAT1, STAT2 and IRF9, preferably an inhibitor of EIF2AK2 or EIF2AK3. Inhibitors of EIF2AK2 are particularly preferred. Still another polypeptide modulator is preferably an activator of eIF2α dephosphorylation including the serine/threonine-protein phosphatase PP1-alpha catalytic subunit PPP1CA, or its viral or host-cell regulatory proteins including PPP1R15, DP71L(s), DP71L(I) and ICP34.5, or a protein with at least 40% amino acid sequence identity with one of said proteins, or a biologically active fragment thereof. Preferably, the percentage of amino acid sequence identity is at least 50%, preferably at least 60% or at least 70%, and most preferably 80% or more, and even preferably 85% or 90% or more, e.g. at least 95% or at least 99% sequence identity.

The polypeptide modulating under appropriate conditions the activity or expression of a target host cell protein involved in the regulation of the phosphorylation level of eIF2α is advantageously a viral protein or is derived from such a viral protein. In order to escape the defense mechanism of the host cells, viruses have indeed developed different strategies to circumvent the translation arrest generally triggered by the defense mechanism of eukaryotic host cells, inter alia through the expression of viral proteins known to interfere with the regulation of the phosphorylation level of eIF2α.

According to such an embodiment, the modulator polypeptide preferably consists in, comprises or is derived from a viral protein inhibiting EIF2AK2 activity. Such a viral protein may be selected from the long and short isoform of E3L of vaccinia virus (UniProtKB/Uniprot accession number ID P21081-1 and UniProtKB/Uniprot accession number ID P21081-2 respectively), NSs protein (UniProtKB/Uniprot accession number ID P21698) from Rift Valley fever virus, N^{PRO} (N-terminal autoprotease; UniProtKB/Uniprot accession number ID Q6Y4U2) from Bovine Viral Diarrhea Virus, V protein (UniProtKB/Uniprot accession number ID P11207) from parainfluenza virus type 5, ICP34.5 from human Herpes-simplex virus-1, NS1 protein (UniProtKB/Uniprot accession number P03496) from Influenza A virus, NS1 protein from human respiratory syncytial virus (UniProtKB/Uniprot accession number ID O42083), K3L protein (UniProtKB/Uniprot accession number ID P18378) of vaccinia virus, DP71L, especially DP71(s) and DP71L(I) from African swine fever virus, VP35 from Zaire Ebolavirus (UniProtKB/Uniprot accession number ID Q05127), VP40 protein (UniProtKB/Uniprot accession number ID P35260) from the Marburg virus, LMP-1 protein (UniProtKB/Uniprot accession number ID P03230) from Epstein-Barr virus, μ2 protein (UniProtKB/Uniprot accession number ID Q00335) from reovirus, B18R secreted protein from vaccinia virus (UniProtKB/Uniprot accession number ID P25213) and ORF4a from Middle East respiratory syndrome coronavirus (MERS CoV; (Khan, Tahir Khan et al. 2020). The modulator polypeptide thus consists in, comprises or is derived from at least one of said viral proteins, or a protein with at least 40% amino acid sequence identity with one of said viral proteins, or a biologically active fragment thereof. Preferably, the percentage of amino acid sequence identity is at least 50%, preferably at least 60% or at least 70%, and most preferably 80% or more, and even preferably 85% or 90% or more, e.g. at least 95% or at least 99% sequence identity.

As referenced herein, percent (%) identity or % sequence identity with respect to a particular sequence, or a specified portion thereof, may be defined as the percentage of nucleotides or amino acids in the candidate sequence that are identical with the nucleotides or amino acids in the subject sequence (or specified portion thereof), after aligning the full length of the particular sequence, or the specified portion thereof, with the subject sequence and introducing gaps, if necessary, to achieve the maximum percent sequence identity. Sequence identity values can be obtained, in particular, by using the BLAST 2.0 suite of programs using default parameters (Altschul, Madden et al. 1997). Software for performing BLAST analyses is publicly available, e.g., through the National Center for Biotechnology-Information (world wide web at ncbi.nlm.nih.gov/).

By biologically active fragment, it is understood a fragment of these proteins, preferably comprising at least 10 amino acids, and which exhibit the same functional activity of said viral proteins, or of a functional subunit of these viral proteins, for example the functional subunit binding dsRNA.

Advantageously, the modulator polypeptide may consist or comprise or be derived from different subunits of different viral proteins, i.e. be a chimeric or artificial modulator polypeptide.

In an embodiment, step (b) of the method of the invention increases the level of expression of a target recombinant DNA in the host cell by at least 10%, still preferably at least 15% or 20%, even more preferably by at least 50%. The increase is measured by comparing the level of expression of a target recombinant DNA in a host cell in which step (a) is carried out, with and without step (b).

The increase may be measured using a reporter recombinant DNA, i.e. a luciferase gene.

Furthermore, certain modulator polypeptides, for instance certain viral proteins, have a more or less marked modulatory effect on their target (e.g. inhibitory or activating effect) depending on the cell type or species of the cell in which they are expressed. Accordingly, the modulator polypeptide is advantageously selected to have a significant modulatory effect in the particular cell type or species in which they are expressed. This can be made for instance by assessing the level of expression of a recombinant DNA, e.g. a reporter DNA such as firefly luciferase, in a host cell expressing an expression system such as the C3P3-G1 system and/or the C3P3-G2 system, with or without the modulatory polypeptide. The modulator polypeptide preferably increases the level of expression of a target recombinant DNA, e.g. a firefly luciferase reporter DNA, in the host cell by at least 10%, still preferably at least 15% or 20%, even more preferably by at least 50%.

The modulator polypeptides can also be selected based on their modulatory activity in a given cell type, e.g. a cell line such as HEK293. In one embodiment the modulator polypeptide increases the level of expression of a target recombinant DNA, e.g. a firefly luciferase reporter gene, in a HEK293 host cell expressing the C3P3-G1 system and/or the C3P3-G2 system, preferably by at least 10%, still preferably at least 15% or 20%.

According to specific embodiments, the modulator polypeptide is an eIF2AK2 inhibitor comprising at least one Zα domain and at least one dsRNA-binding domain, e.g. for one or more viral proteins, in particular a Zα domain from E3L of vaccinia virus or from mammalian preferably human ADAR1 operably linked to at least one dsRNA-binding domain, in particular a dsRNA-binding domain from Influenza A virus NS1 protein, from mammalian EIF2AK2, from Flock House virus B2 protein or from orthoreovirus σ3 protein.

Particularly preferred combinations are the Zα domain from E3L of vaccinia virus operably linked to the dsRNA-binding domain from Influenza A virus NS1 and the Zα domain from E3L of vaccinia virus operably linked to the dsRNA-binding domain from mammalian EIF2AK2 proteins. By operably linked it is to be understood that both domains are fused or covalently linked, or non-covalently linked, however preserving the eIF2AK2 inhibition capacity. It is indeed known that, taken in isolation, the Zα domain as well as the dsRNA-binding domain do not inhibit eIF2AK2 activity and that linkage of both domains is necessary for eIF2AK2 inhibition.

When the Zα domain and the dsRNA-binding domain are fused, a linker may be inserted between both domains, for example the Gly4Ser or G4S (i.e. 4 glycine followed by a serine) linker, more preferably (G4S)2.

Examples of different combinations are illustrated in the examples, and correspond to the following modulator polypeptides:
Zα domain from human ADAR1, fused to the dsRNA-binding domain from E3L of vaccinia virus, through a linker: pADAR1-Zα/(G4S)2/E3L-dsDNA (SEQ ID NO. 6) and the corresponding nucleotide sequence SEQ ID NO. 5.
Zα domain from E3L of vaccinia virus, fused to the dsRNA-binding domain from Influenza A virus NS1: pE3L-Zα/NS1-dsDNA (SEQ ID NO. 8) and the corresponding nucleotide sequence SEQ ID NO. 7.
Zα domain from E3L of vaccinia virus, fused to the dsRNA-binding domain from Flock House virus B2 protein: pE3L-Zα/B2-dsDNA (SEQ ID NO. 10) and the corresponding nucleotide sequence SEQ ID NO. 9.
Zα domain from E3L of vaccinia virus, fused to the dsRNA-binding domain from human EIF2AK2 protein: pE3L-Zα/hEIF2AK2-dsDNA (SEQ ID NO. 12) and the corresponding nucleotide sequence SEQ ID NO. 11.
Zα domain from E3L of vaccinia virus, fused to the dsRNA-binding domain from orthoreovirus σ3 protein: pE3L-Zα/σ3-dsDNA (SEQ ID NO. 14) and the corresponding nucleotide sequence SEQ ID NO. 13.

Other combinations may readily be envisaged by a skilled person, on the basis of the different domains of the viral proteins known as EIF2AK2 inhibitors, especially those listed above.

Moreover, some of the naturally occurring proteins inhibiting eIF2AK2 are known to dimerize or oligomerize. When chimeric modulator polypeptides are designed, derived from these naturally occurring proteins, a further element promoting dimerization or oligomerization, especially homodimerization, is thus advantageously inserted in the construct. An artificial modulator polypeptide according to the invention may therefore further comprise at least one oligomerization domain operably linked to said at least one of said Zα domain and said dsRNA-binding domain, in order to promote oligomerization of the modulator polypeptide. Suitable examples are the leucine-zipper motif, the super leucine zipper motif s-ZIP or the GCN4-pVg leucine zipper; they are illustrated in the example section. Such an oligomerization domain is preferably an homodimerization domain, and even more preferably a domain promoting homodimerization in parallel orientation.

Examples of different chimeric modulator polypeptides are illustrated in the examples, namely:
Zα domain from E3L of vaccinia virus, fused to the dsRNA-binding domain from Influenza A virus NS1, fused by a (G4S)2 linker to a super leucine zipper: pE3L-Zα/NS1-dsDNA/(G4S)2/LZ-sZIP (SEQ ID NO. 16) and the corresponding nucleotide sequence SEQ ID NO. 15;
Zα domain from E3L of vaccinia virus, fused to the dsRNA-binding domain from Influenza A virus NS1, fused by a (G4S)2 linker to a GCN4-pVg leucine zipper pE3L-Zα/NS1-dsDNA/(G4S)2/GCN4 (SEQ ID NO. 18) and the corresponding nucleotide sequence SEQ ID NO. 17.

In a preferred embodiment of the invention, the modulator polypeptide, inhibiting eIF2AK2, consists in or comprises the sequence SEQ ID NO:16, or a sequence having at least 40% sequence identity with SEQ ID NO:16, preferably at least 50%, or 60%, preferably at least 70%, and most preferably 80% or more, and even preferably 85% or 90% or more, e.g. at least 95%, and having the expected activity of eIF2AK2 inhibition.

According to another embodiment, the modulator polypeptide is or contain an activator of the dephosphorylation of eIF2α, such as PPP1CA the eukaryotic catalytic subunit of the serine/threonine-protein phosphatase PP1-alpha (UniProtKB/Uniprot accession number ID P62136) or its viral and host-cell regulators such as PPP1R15 UniProtKB/Uniprot accession number ID O75807), DP71L(s) and DP71L(I) isoforms from African swine fever virus (UniProtKB/Uniprot accession number Q65212 and P0C755, respectively) or a biologically active fragment thereof, or a protein derivative having at least 40% amino acid sequence identity with said protein or biologically active fragment thereof, preserving the biological activity of dephosphorylating eIF2α protein. The percentage of amino acid sequence identity is advantageously greater than 40%, as described above, namely greater than 50%, 60%, 70%, 80%, 90%, 95% or 99%.

According to still another embodiment, the modulator polypeptide is an inactive mutant of a host cell protein involved in the regulation of the phosphorylation level of eIF2α. Particularly preferred inactive mutants are inactive mutants from EIF2AK2 or EIF2AK3, or from a biologically active fragment thereof. Advantageously, said inactive mutants are capable of dimerizing with their wildtype counterparts thereby competing with the homodimerized wildtype proteins. Potential inactive mutants are the K296R mutant of the human EIF2AK2 and biologically active fragments thereof.

Still another type of inhibitor consists of dsRNA binding domain from EIF2AK2 deleted of its carboxy-terminal kinase domain. This artificial protein is able to dimerize with the wild-type EIF2AK2 protein, which forms inactive dimers. This results in an inhibition of activity by dominant negative effect. Mutants with increased homo dimerization capacity can also be considered as F41A, K60A, K64E, K150A, and K154E, which show enhanced dimerization activity increased affinity and dimerization capacity (Patel, Stanton et al. 1996, Patel and Sen 1998). Generally, any derivatives of said dsRNA binding domain which maintain the homo-dimerization activity are also encompassed, in particular those having at least 40% amino acid sequence identity with said dsRNA binding domain from EIF2AK2 or biologically active fragment thereof, preserving the biological activity of dephosphorylating eIF2α protein. The percentage of amino acid sequence identity is advantageously of at least 40%, preferably at least 50%, 60%, 70%, 80%, 90%, 95% or 99%. The dsRNA binding domain is preferably from a mammalian EIF2AK2, in particular from the human EIF2AK2 (UniProtKB/Uniprot accession number P19525, residues 2-167).

Artificial inhibitors of EIF2αkinases consisting of artificial chimeric proteins can be also used to drive the ubiquitination of EIF2αkinases, and thereby their degradation by the 28S proteasome. Due to their functional analogy with the heterobifunctional small molecules called PROTACs (proteolysis-targeting chimeras), these chimeric proteins are often called biological PROTACs or BIO-PROTACs (Lim, Khoo et al. 2020). Several examples of such chimeric proteins have been reported by fusion of interacting domain of the targeted protein with specific subunit domains of multimeric E3 ligases, such as F-box proteins from Skp1-Cul1-F-box complex (Zhou, Bogacki et al. 2000, Su, Ishikawa et al. 2003, Lim, Khoo et al. 2020), BTB proteins from the Cul3-BTB complex (Lim, Khoo et al. 2020) or CHIP protein from CHIP-Ubc13-Uev1a complex (Portnoff, Stephens et al. 2014). Other examples were obtained by engineering the E2 ubiquitin-conjugating enzymes (Gosink and Vierstra 1995).

Such a technology can be easily adapted specifically target the degradation of EIF2AK2 by construction of chimeric proteins comprising:
a. A polypeptide capable of selectively binding to EIF2AK2, such as
   dsRNA-binding region from EIF2AK2 protein deleted of its carboxyl-terminal kinase domain. This region of the human EIF2AK2 protein contains two dsRNA binding motifs separated by a short spacer; or
   Orthologous dsRNA binding domains (e.g. dsRNA-binding domain of E3L protein from vaccinia virus); or
   Single-chain antibodies (e.g. nanobodies or ScFv) raised against EIF2AK2;
b. Fused to specific domains from multimeric E3 ligases, such as:
   Skp1-interacting domains from BTRCP (UniProtKB/Uniprot accession number Q9Y297), FBW7 (UniProtKB/Uniprot accession number Q969H0), SPK2 (UniProtKB/Uniprot accession number Q13309), which is part of the Rbx1-Cul1-Spk1-F-box protein complexes,
   Elongin BC-interacting domains from VHL (UniProtKB/Uniprot accession number Q9Y297), which is part of the Rbx1-Cul2/5-Spk1-Elongin BC-VHL complex,
   Cullin3-interacting domains from SPOP (UniProtKB/Uniprot accession number O43791), which is part of the Rbx1-Cul3-BTB proteins complexes,
   DDB1-interacting domains from CRBN (UniProtKB/Uniprot accession number Q96SW2) or DDB2 (UniProtKB/Uniprot accession number Q92466), which is part of the Rbx1-Cul4A-CRBN-DDB1 complex,
   Elongin BC-interacting domains from SOCS2 (UniProtKB/Uniprot accession number O14508), which is part of the Rbx2-Cul2/5-Elongin BC-SOCS-box protein complex,
   U-box interacting domain and coiled-coil dimerization domain from STUB1 (also named CHIP, UniProtKB/Uniprot accession number Q9UNE7), which is part of the CHIP-Ubc13-Uev1a complex,
   CUL1-interacting domain from Skp1 (UniProtKB/Uniprot accession number P63208), which is part of the Rbx1-Cul2-Spk1-F-box protein complexes.

The dsRNA-binding region from EIF2AK2 protein may be a wild-type or a mutant dsRNA-binding region. The EIF2AK2 protein is preferably a mammalian EIF2AK2, more preferably a human EIF2AK2. The downregulation of the phosphorylation levels of eIF2α can be advantageously effected by modulating two different pathways leading to the downregulation of the phosphorylation levels of eIF2α.

Therefore, according to preferred embodiments, the invention comprises the use of at least two compounds modulating the phosphorylation level of EIF2α, preferably two compounds downregulating different host cell targets involved on the regulation of said phosphorylation level. It has been indeed unexpectedly demonstrated by the inventors that such a combination may have a supra-additive effect. Particularly preferred combinations are:
one Zα domain and at least one dsRNA-binding domain for one or more viral proteins, in particular a Zα domain from E3L of vaccinia virus or from mammalian preferably human ADAR1 operably linked to at least one dsRNA-binding domain, in particular a dsRNA-binding domain from Influenza A virus NS1 protein, from mammalian EIF2AK2, from Flock House virus B2 protein or from orthoreovirus σ3 protein, in combination with
the N(pro) from Bovine Viral Diarrhea Virus that targets IRF3, the NSs from Rift Valley fever virus that promotes EIF2AK2 proteasomal degradation, or 5′UTR from Sindbis viral genome that antagonize IFIT1.

In another embodiment, the invention comprises the downregulation of the phosphorylation levels of eIF2α, by modulating two different pathways, namely by inhibiting the phosphorylation of eIF2α, preferably by inhibiting EIF2AK2, and by activating the dephosphorylation of eIF2α. It has been demonstrated by the inventor that acting simultaneously on the phosphorylation eIF2α (by inhibition) and on the dephosphorylation of eIF2α (by activation) has a supra-additive effect or synergic effect. A synergy between the two modes of action was unexpected since both targeted pathways modulate the same target, i.e. eIF2α and particularly its phosphorylation. In particular, the present inventor has surprisingly demonstrated that the following combination of eIF2α phosphorylation modulators has supra-additive effect, comprising:
dsRNA-binding domain from EIF2AK2 protein deleted of its carboxyl-terminal kinase domain,
optionally, a ubiquitin-interacting domain from multimeric E3 ligases including, but not limited to BTRCP, FBW7, SPK2, VHL, SPOP, CRBN, SOCS2, STUB1 or SPK1, and;
an activator of eIF2α dephosphorylation including the serine/threonine-protein phosphatase PP1-alpha catalytic subunit PPP1CA, or its viral or host-cell regulatory proteins including the host-cell PPP1R15, DP71L(s) and DP71L(I) from African swine fever virus, and ICP34.5 from human Herpes-simplex virus-1.

In a preferred embodiment, the inhibition of the phosphorylation of eIF2α can be obtained efficiently by the expression of the chimeric protein described in Example 7 and following, which repress kinase EIF2AK2 and stimulate the dephosphorylation of eIF2α.

At any step, the ability of a modulator polypeptide to downregulate the phosphorylation level of eIF2α, or the ability of a combination of modulators to provide a supra-additive effect can be easily confirmed by a skilled person using well-known eIF2 phosphorylation assays.

In a variant also covered by this invention, step (b) may be replaced, or combined with a step of modulating, in particular stimulating eIF2 activity such as to increase the translation initiation rate, and thereby increase the level of expression by the artificial expression system according to the invention. Accordingly, in one aspect, this invention relates to a method for expressing a recombinant DNA molecule in a eukaryotic host cell, comprising the steps of:
(a) expressing or introducing at least one chimeric protein, in said host cell, wherein said chimeric protein comprises:
   at least one catalytic domain of a capping enzyme, in particular selected in the group consisting of cap-0 canonical capping enzymes, cap-0 non-canonical capping enzymes, cap-1 capping enzymes and cap-2 capping enzymes; and
   at least one catalytic domain of a DNA-dependent RNA polymerase, in particular a bacteriophage DNA-dependent RNA polymerase,
(b) constitutively or transiently stimulating eIF2 activity in said host cell.

This variant also concerns the various embodiments of the invention, in particular isolated nucleic acid molecule(s), chimeric polyprotein, polypeptide or set of polypeptides, vectors, kits, compositions, in particular pharmaceutical compositions, uses and methods described in the present specification. EIF2 activity may be stimulated by overexpressing a component of the eIF2-Met-tRNAᵢ^{Met}-GTP ternary complex (eIF2-TC), which deliver Met-tRNAᵢ^{Met} to the 40S ribosomal subunit. EIF2 activity may also be stimulated by overexpressing and/or activating, or by silencing and/or inhibiting a pathway regulating expression of a component of the eIF2-Met-tRNAᵢ^{Met}-GTP ternary complex (eIF2-TC). In some embodiments, eIF2 activity may be modulated by constitutively or transiently overexpressing a compound involved in the regulation of production of Met-tRNAᵢ^{Met}, in particular tRNAᵢ^{Met} and/or the methionyl tRNA synthetase (MetRS, also named MARS1; UniProtKB/Uniprot accession number P56192).

Single or Set of Nucleic Acid Molecules

The invention also relates to an isolated nucleic acid molecule or a group or set of isolated nucleic acid molecules, said nucleic acid molecule(s) encoding the different elements of the invention as already defined, namely:
at least one catalytic domain of a capping enzyme,
at least one catalytic domain of a DNA-dependent RNA polymerase,
at least one means of downregulating the phosphorylation level of eIF2α, preferably a modulator polypeptide,
and preferably also at least one catalytic domain of a poly(A) polymerase, preferably tethered through a N-peptide or any tethering peptide from an RNA-protein tethering system.

The isolated nucleic acid molecule or the group of isolated nucleic acid molecules comprises or consists in:
(a) at least one nucleic acid sequence encoding a chimeric protein comprising:
   (i) at least one catalytic domain of a capping enzyme; and
   (ii) at least one catalytic domain of a DNA-dependent RNA polymerase; and
(b) at least one nucleic acid sequence downregulating the phosphorylation level of eIF2α in a eukaryotic host cell or encoding a molecule downregulating said phosphorylation level, preferably encoding a modulator polypeptide, and,
(c) optionally, at least one nucleic acid sequence encoding a poly(A) polymerase, preferably tethered through a N-peptide.

Sequence (b) may inter alia be a sequence coding for inter alia a ribozyme, a siRNA, a shRNA, a miRNA, an antisense or a modulator polypeptide, capable of modulating the activity or the expression of a target host cell involved in the phosphorylation level of eIF2α.

The preferred embodiments of these different elements have already been disclosed in the context of the other aspects of the invention and are not all repeated here.

It is thus preferred that the modulator polypeptide modulates a target host cell protein involved in the type-I interferon (IFN-I) response, or in the unfolded protein response of the host cell. Said host cell protein is advantageously selected from the list of EIF2AK2, EIF2AK3, DDX58, IFIH1, MAVS, IFNAR1, IFNAR2, IRF3, IRF7, IFNB1, a type-I interferon protein or a protein of the JAK-STAT pathway, in particular JAK1, TYK2, STAT1, STAT2 and IRF9, or protein phosphatase 1 PP1 or a subunit thereof, in particular PPP1CA or PPP1R15.

In a preferred embodiment, and as already detailed, the modulator polypeptide is selected from:
a viral protein selected from E3L of vaccinia virus, NSs from Rift Valley fever virus, N^{PRO} from Bovine Viral Diarrhea Virus, V protein from parainfluenza virus type 5, ICP34.5 from human Herpes-simplex virus-1, NS1 from influenza A virus, K3L from vaccinia virus, DP71L from African swine fever virus in particular isoforms DP71L(s) and DP71L(I), VP35 from Zaire Ebolavirus, VP40 from Marburg virus, LMP-1 from Epstein-Barr virus, μ2 from reovirus, B18R of vaccinia virus and ORF4a from Middle East respiratory syndrome coronavirus, a protein with at least 40% amino acid sequence identity with one of E3L of vaccinia virus, NSs from Rift Valley fever virus, N^{PRO} from Bovine Viral Diarrhea Virus, V protein from parainfluenza virus type 5, ICP34.5 from human Herpes-simplex virus-1, NS1 from influenza A virus, K3L from vaccinia virus, DP71L from African swine fever virus, in particular isoforms DP71L(s) and DP71L(I), VP35 from Zaire Ebolavirus, VP40 from Marburg virus, LMP-1 from Epstein-Barr virus, μ2 from reovirus B18R of vaccinia virus and ORF4a from Middle East respiratory syndrome coronavirus, or a biologically active fragment thereof;
PPP1CA catalytic subunit and its regulatory proteins, in particular its host-cell regulatory proteins such as the eukaryotic protein PPP1R15 or a biologically active fragment thereof;
an inactive mutant of a host cell protein involved in the regulation of the phosphorylation level of eIF2α, in particular selected from EIF2AK2 or EIF2AK3 or a biologically active fragment thereof, in particular the K296R mutant of the human EIF2AK2, or the dsRNA binding domain from EIF2AK2 deleted of its carboxy-terminal kinase domain or a biologically active fragment thereof.

Preferably, the modulator polypeptide is an eIF2AK2 inhibitor, especially an eIF2AK2 inhibitor comprising at least one Zα domain, in particular a Zα domain from E3L of vaccinia virus or human ADAR1 operably linked to at least one dsRNA-binding domain, in particular a dsRNA-binding domain from Influenza A virus NS1 protein, mammalian EIF2AK2, Flock House virus B2 protein, orthoreovirus σ3 protein, preferably selected from Influenza A virus NS1 and mammalian EIF2AK2 proteins. In another preferred embodiment, the preferred polypeptide comprises a ubiquitin-interacting domain from a multimeric E3 ligase including, but not limited to, BTRCP, FBW7, SPK2, VHL, SPOP, CRBN, SOCS2, STUB1 or SPK1.

A preferred eIF2AK2 inhibitor comprises the amino acid sequence set forth in SEQ ID NO. 16; or an amino acid sequence with at least 40% amino acid sequence identity with SEQ ID NO. 16, or at least 50%, at least 60%, at least 70%, at least 80%, at least 90%, at least 95% or at least 98%, or at least 99%; or a biologically active fragment thereof as already described.

In one embodiment, the isolated nucleic acid molecule(s) comprise(s) the nucleic acid sequences detailed above fused in frame, in particular in the order (a), (c) and (b) (from 5′ to 3′ terminus). The isolated nucleic acid molecule(s) thus advantageously comprise(s) from the 5′-terminus to the 3′-terminus:
optionally one nucleic acid sequence encoding a catalytic domain of a poly(A) polymerase, preferably tethered through a N-peptide from lambdoid viruses;
one nucleic acid sequence encoding the modulator polypeptide, capable of downregulating the phosphorylation level of eIF2α;
one nucleic acid sequence encoding a chimeric protein comprising:
   (i) at least one catalytic domain of a capping enzyme; and
   (ii) at least one catalytic domain of a DNA-dependent RNA polymerase; and

Such a molecule and the preferred embodiments of each element have already been described in the context of the other aspects of the invention.

Such a single nucleic acid molecule has the advantage of facilitating the subunit assembly, since there is only a single open-reading frame, and allow higher expression rates (as illustrated in WO2019/020811).

In one embodiment, the isolated nucleic acid molecule or the set of isolated nucleic acid molecules comprise at least one nucleic acid sequence encoding a ribosome skipping motif.

Sequences coding for a linker may also be inserted between the different elements, in place of sequences encoding a ribosome skipping motif.

Accordingly, in a particularly preferred embodiment applicable to all the aspects of the invention, the nucleic acid molecule or the set of nucleic acid molecules comprises from the 5′-terminus to the 3′-terminus at least:
N-peptide from the lambda bacteriophage (Genbank AAA32249.1) SEQ ID NO. 29 and SEQ ID NO. 30;
Mutant S605A/S48A/S654A/KK656-657RR of the mouse poly(A) polymerase a isoform 1 (PAPOLA, UniProtKB/Uniprot accession number Q61183-1);
Ribosome skipping F2A sequence from the Foot-and-mouth disease virus (Genbank AAT01770.1);
Zα domain of E3L from the vaccinia virus;
dsRNA binding domain from NS1 from the influenza virus;
Leucine zipper sZIP;
Ribosome skipping F2A sequence from the Foot-and-mouth disease virus (Genbank AAT01770.1);
African Swine Fever Virus capping enzyme NP868R (UniProtKB/Uniprot accession number P32094.1);
Peptide linker (G₄S)₂ (SEQ ID NO. 34); and
Mutant R551S K1E DNA-dependent RNA polymerase from bacteriophage K1E (UniProtKB/Uniprot accession number Q2WC24).

This molecule is illustrated in the example and corresponds to SEQ ID NO. 19. The encoded protein sequence corresponds to SEQ ID NO. 20.

In another preferred embodiment applicable to all the aspects of the invention, the nucleic acid molecule or the set of nucleic acid molecules comprises from the 5′-terminus to the 3′-terminus at least:
dsRNA binding domain from human EIF2AK2 (UniProtKB/Uniprot accession number P19525, residues 2-167);
Peptide linker (G4S)2;
DP71L(I) from the African Swine Fever virus (UniProtKB/Uniprot accession number P0C755);
Ribosome skipping F2A sequence from the Foot-and-mouth disease virus (Genbank AAT01770.1);
N-peptide from the lambda bacteriophage (Genbank AAA32249.1) SEQ ID NO. 29 and SEQ ID NO. 30;
Mutant S605A/S48A/S654A/KK656-657RR of the mouse poly(A) polymerase a isoform 1 (PAPOLA, UniProtKB/Uniprot accession number Q61183-1);
Ribosome skipping F2A sequence from the Foot-and-mouth disease virus (Genbank AAT01770.1);
African Swine Fever Virus capping enzyme NP868R (UniProtKB/Uniprot accession number P32094.1);
Peptide linker (G₄S)₂; and
Mutant R551S K1E DNA-dependent RNA polymerase from bacteriophage K1E (UniProtKB/Uniprot accession number Q2WC24).

This molecule is illustrated in the example and corresponds to SEQ ID NO. 35. The encoded protein sequence corresponds to SEQ ID NO. 36.

Accordingly, in one embodiment, step (b) of the method according to the invention comprises introducing, into said host cell, a polypeptide comprising, the sequence of SEQ ID NO. 20 or SEQ ID NO. 36 or a sequence with at least 40% identity to SEQ ID NO. 20 or SEQ ID NO. 36, or a nucleic acid sequence encoding said polypeptide, wherein said polypeptide is capable of downregulating the phosphorylation level of eIF2α. Preferably, the percentage of amino acid sequence identity is at least 50%, preferably at least 60% or at least 70%, and most preferably 80% or more, and even preferably 85% or 90% or more, e.g. at least 95% or at least 99% sequence identity.

The nucleic acid molecule(s) according to the invention can be operably linked to at least one, preferably all promoters selected from the group consisting of:
a promoter for a eukaryotic DNA-dependent RNA polymerase, preferably for RNA polymerase II;
a promoter for said catalytic domain of a DNA-dependent RNA polymerase of the chimeric enzyme of the invention.

The link of the nucleic acid to a promoter for a eukaryotic DNA-dependent RNA polymerase, preferably for RNA polymerase II has notably the advantage that when the chimeric enzyme of the invention is expressed in a eukaryotic host cell, the expression of the chimeric enzymes is driven by the eukaryotic RNA polymerase, preferably the RNA polymerase II. These chimeric enzymes, in turn, can initiate transcription of the transgene. If tissue-specific RNA polymerase II promoters are used, the chimeric enzyme of the invention can be selectively expressed in the targeted tissues/cells.

Said promoter can be a constitutive promoter or an inducible promoter well known by one skilled in the art. The promoter can be developmentally regulated, inducible or tissue specific.

The invention also relates to a vector comprising the nucleic acid molecule or the set of nucleic acid molecules according to the invention. Said vector can be suitable for semi-stable or stable expression.

The invention also relates to a group of vectors comprising said group of isolated nucleic acid molecules according to the invention.

Particularly said vector according to the invention is a cloning or an expression vector.

Preferred vectors are plasmids or engineered dsDNA molecules such as MIDGE (Schakowski, Gorschluter et al. 2001) or DNA minicircles (Ronald, Cusso et al. 2013).

The invention also relates to a host cell comprising a nucleic acid molecule according to the invention or a vector according to the invention or a group of vectors according to the invention. The host cell according to the invention can be useful for large-scale protein production. The host cell is an eukaryotic host cell, especially a mammalian host cell, such as a human host cell.

Such a host-cell may thus comprise at least one nucleic acid sequence encoding the modulator polypeptide, capable of downregulating the phosphorylation level of eIF2α; the phosphorylation level of eIF2α is thus downregulated in this host cell; this embodiment thus corresponds to an already detailed embodiment, in a preceding section.

The invention also relates to a genetically engineered non-human eukaryotic organism, which expresses a chimeric enzyme or polyprotein encoded by the nucleic acid molecule or the group of isolated nucleic acid molecules according to the invention, or the vector or group of vectors according to the invention, in particular a chimeric enzyme or chimeric polyprotein according to the invention. Said non-human eukaryotic organism may be any non-human animals, plants, fungi or unicellular organism, including yeasts or protozoa, preferably a non-human mammal. In such a genetically engineered non-human eukaryotic organism, the phosphorylation level of eIF2α is downregulated, either constitutively or transiently, for example by transformation with a nucleic acid molecule encoding a modulator polypeptide as already described.

C3P3-G3 Expression System and its Components

Overview

The C3P3 (acronym of cytoplasmic chimeric capping-prone phage polymerase) is an artificial expression system, which relies on two components:
A DNA-dependent RNA polymerase, which has been developed by molecular engineering. This artificial enzyme synthesizes the mRNA chain and performs eukaryotic-like modifications requested for mRNA translation and other biological functions. The structure of these different generations of C3P3 enzyme is described below.
Specific DNA templates, which consists of the C3P3 promoter followed by a C3P3 promoter, which a 18-24 bp sequence, followed 5′-untranslated region (5′UTR typically from the human β-globin gene), the open-reading frame of the target gene, 3′-untranslated region (3′UTR), artificial adenosine track typically of 40 adenosine residues and a self-cleaving ribozyme (typically, the genomic ribozyme from of the hepatitis D virus), and terminated by the transcription stop (typically from bacteriophage T7 φ10). From the 2^{nd} generation of the system, sequences allowing an mRNA-protein interaction are added in the 3′UTR region. These sequences, typically four repeats in tandem or more of BoxBl or BoxBr of bacteriophage lambdoid family, make it possible to recruit the poly(A) polymerase from the C3P3 enzyme, which is fused to the N peptide of bacteriophage λ.

The structure of different generations of the C3P3 enzyme, which all consists of a single open-reading-frame, are as follows:
C3P3-G1, which allows the synthesis of mRNA with cap-0 modification at 5′-end, was described elsewhere (Jais 2011, Jais 2011, Jais 2017). The C3P3-G1 enzyme consists of the following fusion from the N- to C-terminus (SEQ ID NO. 1 and SEQ ID NO. 2; FIG. 8A): African Swine Fever Virus capping enzyme NP868R (UniProtKB/Uniprot accession number P32094.1), flexible (G4S)2 linker (SEQ ID NO. 34), and mutant R551S K1E DNA-dependent RNA polymerase from bacteriophage K1E (UniProtKB/Uniprot accession number Q2WC24). Noticeably, NP868R contains the three enzymatic domains requested to generate a cap0: 5′-triphosphatase, guanylyltransferase and N7-guanine methyltransferase.
C3P3-G2, which allows the post-transcriptional polyadenylation at 3′-end of the target mRNA in addition to cap-0 modification, has been described elsewhere with slight modifications (Jais 2017). The C3P3-G2 enzyme consists of the following fusion from the N- to C-terminus (SEQ ID NO. 3 and SEQ ID NO. 4; FIG. 8B): N-peptide from the λ bacteriophage that binds at high affinity to the BoxBL sequences from the target mRNA (Genbank AAA32249.1), mutant S605A/S48A/S654A/KK656-657RR mouse poly(A) polymerase a isoform 1 (PAPOLA, UniProtKB/Uniprot accession number Q61183-1), ribosome skipping F2A sequence from the Foot-and-mouth disease virus that allows ribosome skipping (Genbank AAT01770.1), African Swine Fever Virus capping enzyme NP868R (UniProtKB/Uniprot accession number P32094.1), flexible (G4S)2 linker, and mutant R551S K1E DNA-dependent RNA polymerase from bacteriophage K1E (UniProtKB/Uniprot accession number Q2WC24). The mouse poly(A) polymerase a of C3P3-G2 has been modified by the deletion of its SUMOylation signal involved in its nuclear addressing, as well as the inactivation of several putative phosphorylation sites by the cdc2 protein kinase.

C3P3-G3a, which is detailed Example 6, comprises from the 5′-terminus to the 3′-terminus (SEQ ID NO. 19) or from the N-terminus to C-terminus (SEQ ID NO. 20):
N-peptide from the lambda bacteriophage (Genbank AAA32249.1) SEQ ID NO. 29 and SEQ ID NO. 30;
Mutant S605A/S48A/S654A/KK656-657RR of the mouse poly(A) polymerase a isoform 1 (PAPOLA, UniProtKB/Uniprot accession number Q61183-1);
Ribosome skipping F2A sequence from the Foot-and-mouth disease virus (Genbank AAT01770.1);
Zα domain of E3L from the vaccinia virus;
dsRNA binding domain from NS1 from the influenza virus;
Leucine zipper sZIP;
Ribosome skipping F2A sequence from the Foot-and-mouth disease virus (Genbank AAT01770.1);
African Swine Fever Virus capping enzyme NP868R (UniProtKB/Uniprot accession number P32094.1);
Peptide linker (G₄S)₂ (SEQ ID NO. 34); and
Mutant R551S K1E DNA-dependent RNA polymerase from bacteriophage K1E (UniProtKB/Uniprot accession number Q2WC24).

C3P3-G3f, which is detailed in Example 9 (SEQ ID NO. 35 and SEQ ID NO. 36), comprises from the N-terminus to C-terminus:
dsRNA binding domain from the human EIF2AK2 (UniProtKB/Uniprot accession number P19525, residues 2-167);
Peptide linker (G₄S)₂;
DP71L(I) from the African Swine Fever virus (UniProtKB/Uniprot accession number P0C755),
Ribosome skipping F2A sequence from the Foot-and-mouth disease virus (Genbank AAT01770.1);
N-peptide from the lambda bacteriophage (Genbank AAA32249.1) SEQ ID NO. 29 and SEQ ID NO. 30;
Mutant S605A/S48A/S654A/KK656-657RR of the mouse poly(A) polymerase a isoform 1 (PAPOLA, UniProtKB/Uniprot accession number Q61183-1);
Ribosome skipping F2A sequence from the Foot-and-mouth disease virus (Genbank AAT01770.1);
African Swine Fever Virus capping enzyme NP868R (UniProtKB/Uniprot accession number P32094.1);
Peptide linker (G₄S)₂; and
Mutant R551S K1E DNA-dependent RNA polymerase from bacteriophage K1E (UniProtKB/Uniprot accession number Q2WC24).

Most of the sequences of the C3P3 system described below were also described in details in WO2011/128444 and WO2019/020811. Their assembly to generate the C3P3-G3a and C3P3-G3f systems with the modulator polypeptide is also described below.

Capping Enzymes

Cap-0 canonical capping enzymes are able to add cap-0 structure at the 5′-end of RNA molecules by involving a series of three enzymatic reactions: RNA triphosphatase (RTPase) that removes the γ phosphate residue of 5′ triphosphate end of nascent pre-mRNA to diphosphate ppRNA, RNA guanylyltransferase (GTase) that transfers GMP from GTP to the diphosphate ppRNA nascent RNA terminus, and RNA N⁷-guanine methyltransferase (N7-MTase) that adds a methyl residue on nitrogen 7 of guanine to the GpppRNA cap (Furuichi and Shatkin 2000).

The enzymatic domains of eukaryotic organisms and viruses, which are involved in the canonical formation of cap-0 structure, can be found assembled in a variable number of protein subunits:
Single subunit capping enzymes with all three enzymatic domains, i.e. RTPase, GTase and N7-MTase. Examples of such enzymes are disclosed in WO2011/128444 and WO2019/020811, and include, but are not limited to Acanthamoeba Polyphaga mimivirus capping enzyme R382; ORF3 capping enzyme from yeast Kluyveromyces lactis linear extra-chromosomal episome pGKL2; African swine fever virus NP868R capping enzyme (Pena, Yanez et al. 1993, Jais 2011, Dixon, Chapman et al. 2013, Jais, Decroly et al. 2019) (NCBI ASFV genomic sequence strain BA71 V NC_001659; UniProtKB/Swiss-Prot accession number P32094); and VP4 Bluetongue virus capping enzyme;
Capping enzymes consisting of two subunits which include, but are not limited to those disclosed in WO2011/128444 and WO2019/020811, namely the mammalian capping enzymes that consists of the RNGTT subunit having both RTPase and GTase enzymatic activities and RNMT having N7-MTase enzymatic activity; the vaccinia capping enzyme that consists of the D1R gene product having RTPase, GTase and N7-MTase enzymatic domains and D12L gene product that has no intrinsic enzymatic activity but enhances drastically the RNA N7-MTase activity of the D1 R subunit;
Capping enzymes that consist of three subunits, such Saccharomyces cerevisiae CET1 with RTPase, CEG1 with GTase, and ABD1 having N7-MTase catalytic activities.

The chimeric protein/enzyme of the artificial expression system C3P3, which is expressed or introduced at step (a) of the method of the invention as described above, comprise:
(i) at least one catalytic domain of a capping enzyme containing:
   at least one catalytic domain of an RNA triphosphatase;
   at least one catalytic domain of a guanylyltransferase; and
   at least one catalytic domain of a N7-guanine methyltransferase;
(ii) at least one catalytic domain of a DNA-dependent RNA polymerase,
   in particular, wherein at least one of said catalytic domains is a catalytic domain of a cap-0 canonical capping enzyme, more particularly of a virus cap-0 canonical capping enzyme. As specified above, the chimeric enzyme or protein also comprises at least one catalytic domain of a DNA-dependent RNA polymerase, in particular a bacteriophage DNA-dependent RNA polymerase.

Said catalytic domains of an RNA triphosphatase, of a guanylyltransferase, of a N⁷-guanine methyltransferase, can be from the same or from different proteins.

Preferably, said catalytic domains of an RNA triphosphatase, of a guanylyltransferase, of a N⁷-guanine methyltransferase are from one or several cytoplasmic enzymes, which have advantageously relatively simple structure and well-characterized enzymatic activities. Thus, in particular, said catalytic domains of an RNA triphosphatase, of a guanylyltransferase, of a N⁷-guanine methyltransferase can be catalytic domains of one or several virus capping enzymes, or of capping enzymes of cytoplasmic episomes.

In one embodiment, said catalytic domains of a RNA triphosphatase, of a guanylyltransferase, of a N⁷-guanine methyltransferase are from one or several virus capping enzymes, in particular selected in the group consisting of the vaccinia virus capping enzyme, bluetongue virus capping enzyme, bamboo mosaic virus capping enzyme, African swine fever virus capping enzyme, Acanthamoeba polyphaga mimivirus capping enzyme, Organic Lake phycodnavirus 1 (OLPV1) capping enzyme, Organic Lake phycodnavirus 2 (OLPV2) capping enzyme, Phaeocystis globosa virus capping enzyme, Chrysochromulina ericina virus capping enzyme and mutants or derivatives thereof which are able respectively to remove the γ phosphate residue of 5′ triphosphate end of nascent pre-mRNA to diphosphate or transfer GMP from GTP to the diphosphate nascent RNA terminus or add a methyl residue on azote 7 of guanine to the GpppN cap, more particularly of the African swine fever virus capping enzyme and mutants or derivatives thereof which are able respectively to remove the γ phosphate residue of 5′ triphosphate end of nascent pre-mRNA to diphosphate or transfer GMP from GTP to the diphosphate nascent RNA terminus or add a methyl residue on azote 7 of guanine to the GpppN cap (Pena, Yanez et al. 1993, Dixon, Chapman et al. 2013, Jais, Decroly et al. 2019).

In one embodiment of the chimeric enzyme or protein according to the invention, said catalytic domain of an RNA triphosphatase, said catalytic domain of a guanylyltransferase and said catalytic domain of a N⁷-guanine methyltransferase, are included in a monomer, i.e. in one polypeptide. For example, said monomer can be a monomeric capping enzyme or a monomeric chimeric enzyme according to the invention.

Said monomeric capping enzyme can be a monomeric virus capping enzyme, in particular selected in the group consisting of the bluetongue virus capping enzyme, bamboo mosaic virus capping enzyme, African swine fever virus capping enzyme, Acanthamoeba polyphaga mimivirus capping enzyme, OLPV1 capping enzyme, OLPV2 capping enzyme, Phaeocystis globosa virus capping enzyme, Chrysochromulina ericina virus capping enzyme and mutants and derivatives thereof which are able to add a ^{m7}GpppN cap at the 5′-terminal end of RNA molecules and, more particularly of the African swine fever virus capping enzyme and mutants and derivatives thereof which are able to add a ^{m7}GpppN cap at the 5′-terminal end of RNA molecules, and even more particularly the African swine fever virus capping enzyme

The chimeric protein or enzyme according to the invention can also further comprise a domain, which enhances the activity of at least one catalytic domain of the chimeric protein or enzyme of the invention, in particular of at least one catalytic domain of a capping enzyme, more particularly of at least one catalytic domain selected in the group consisting of a catalytic domain of a RNA triphosphatase, a catalytic domain of a guanylyltransferase, a catalytic domain of a N⁷-guanine methyltransferase, preferably of a N⁷-guanine methyltransferase.

For example, said domain, which enhances the activity of at least one catalytic domain of the chimeric enzyme of the invention, can be a 31-kDa subunit encoded by the vaccinia virus D12L gene (genomic sequence NC_006998.1; Gene3707515; UniProtKB/Uniprot accession number YP_232999.1), which has no intrinsic enzymatic activity, but enhances drastically the RNA N⁷-guanine methyltransferase activity of the D1 R subunit of the vaccinia mRNA capping enzyme.

In one embodiment, the chimeric protein or enzyme according to the invention further comprises at least one catalytic domain of a cap-1 or cap-2 capping enzymes, as already defined.

In a further embodiment, the chimeric enzyme according to the invention further comprises at least one catalytic domain of a 5′-end RNA processing enzyme other than cap-0, cap-1 and cap-2 capping enzymes.

In a preferred embodiment, the chimeric protein or enzyme according to the invention comprise the wild-type African swine fever virus capping enzyme and mutants or derivatives thereof which are able respectively to remove the γ phosphate residue of 5′ triphosphate end of nascent pre-mRNA to diphosphate or transfer GMP from GTP to the diphosphate nascent RNA terminus or add a methyl residue on azote 7 of guanine to the GpppN cap (Pena, Yanez et al. 1993, Dixon, Chapman et al. 2013, Jais, Decroly et al. 2019).

DNA-Dependent RNA Polymerases

The chimeric protein or enzyme according to the invention also comprises at least one catalytic domain of a DNA-dependent RNA polymerase as described in WO2011/128444.

Preferably, said catalytic domain of a DNA-dependent RNA polymerase is a catalytic domain of an enzyme, which has a relatively simple structure and more preferably, which has characterized genomic enzymatic regulation elements (i.e. promoter and transcription termination signal). Thus, in particular, said catalytic domain of a DNA-dependent RNA polymerase can be a catalytic domain of a bacteriophage DNA-dependent RNA polymerase, of a bacterial DNA-dependent RNA polymerase or of a DNA-dependent RNA polymerase of various eukaryotic organelles.

In one embodiment, said catalytic domain of a DNA-dependent RNA polymerase is a catalytic domain of a bacteriophage DNA-dependent RNA polymerase. The bacteriophage DNA-dependent RNA polymerases have notably the advantage that they optimize the levels of transgene expression, in particular by having a higher processivity than the eukaryotic RNA polymerases. The bacteriophage

DNA-dependent RNA polymerases have also a much simpler structure than most nuclear eukaryotic RNA polymerases, which consist of multiple subunits (e.g. RNA polymerase II) and transcription factors. Most of the bacteriophage DNA-dependent RNA polymerases characterized so far are single-subunit enzymes, which require no accessory proteins for initiation, elongation, or termination of transcription (Chen and Schneider 2005). Several of these enzymes, which are named for the bacteriophages from which they have been cloned, have also well-characterized regulation genomic elements (i.e. promoter and termination signals), which are important for transgenesis.

Said catalytic domain of a bacteriophage DNA-dependent RNA polymerase can be a catalytic domain of a bacteriophage DNA-dependent RNA polymerase, in particular selected in the group consisting of the T7 RNA polymerase (NCBI GenBank accession number genomic sequence NC_001 604; Gene 1261 050; UniProtKB/Uniprot accession number P00573), T3 RNA polymerase (NCBI GenBank accession number genomic sequence NC_003298; Gene 927437; UniProtKB/Uniprot accession number Q778M8), K1E RNA polymerase (NCBI GenBank accession number genome sequence AM084415.1, UniProtKB/Uniprot accession number Q2WC24), K1.5 RNA polymerase (NCBI GenBank accession number genome sequence AY370674.1, NCBI GenBank accession number YP_654105.1), K11 RNA polymerase (NCBI GenBank accession number genomic K11 RNAP sequence NC_004665; Gene 1258850; UniProtKB/Uniprot accession number Q859H5), phiA1122 RNA polymerase (NCBI GenBank accession number genomic sequence NC_004777; Gene 1733944; UniProtKB/Uniprot accession number protein Q858N4), phiYeo3-12 RNA polymerase (NCBI GenBank accession number genomic sequence NC_001271; Gene 1262422; UniProtKB/Uniprot accession number Q9T145) and gh-1 RNA polymerase (NCBI GenBank accession number genomic sequence NC_004665; Gene 1258850; UniProtKB/Uniprot accession number protein Q859H5), SP6 RNA polymerase (NCBI GenBank accession number genomic sequence NC_004831; Gene 1481778; UniProtKB/Uniprot accession number protein Q7Y5R1), and mutants or derivatives thereof, which are able to synthesize single-stranded RNA complementary in sequence to the double-stranded DNA template in the 5′-3′ direction, more particularly the T7 RNA polymerase, the T3 RNA polymerase, SP6 RNA polymerase, K1.5 RNA polymerase and K1E RNA polymerase and mutants or derivatives thereof, which are able to synthesize single-stranded RNA complementary in sequence to the double-stranded DNA template in the 5′-3′ direction.

The catalytic domain of a DNA-dependent RNA polymerase can be the one of the wild-type of the K1E or K1.5 RNA polymerase but also of mutants of the K1E or K1.5 RNA polymerases, which are able to synthesize single-stranded RNA complementary in sequence to the double-stranded DNA template in the 5′-3′ direction, even with high processivity. For example, said mutants can be selected in the group consisting of the K1E RNA polymerase mutants R551 (Jais, Decroly et al. 2019), F644A, Q649S, G645A, R627S, 181 OS, D812E (Makarova, Makarov et al. 1995), and K631M (Osumi-Davis, de Aguilera et al. 1992, Osumi-Davis, Sreerama et al. 1994), in particular R551S mutant.

Preferably, said catalytic domain of the DNA-dependent RNA-polymerase of the chimeric enzyme according to the invention is from different enzymes than those of the host cell to prevent the competition between the endogenous gene transcription and said DNA sequence transcription.

Poly(A) Polymerases

As described in the present specification the chimeric protein or enzyme according to the invention comprises optionally at least one catalytic domain of a poly(A) polymerase, preferably fused to at least one RNA-binding domain of a protein-RNA tethering system.

In one embodiment, said catalytic domain of a poly(A) polymerase is a catalytic domain of a canonical poly(A) polymerase including mammalian (such as PAPOLA, PAPOLB, PAPOLG), yeast, (such as Saccharomyces cerevisiae PAP1, Schizosaccharomyces pombe PLA1, Candida albicans PAP, Pneumocystis carinii PAP), protozoan, viral and bacterial canonical poly(A) polymerases.

In particular, said catalytic domain of a poly(A) polymerase is a cytoplasmic canonical poly(A) polymerase, more particularly selected in the group consisting of:
Mammalian cytoplasmic poly(A) polymerase including PAPOLB (human and mouse PAPOLB, UniProtKB/Uniprot accession number Q9NRJ5 and Q9WVP6, respectively), which is at least in part located in the cytoplasmic compartment (Kashiwabara, Tsuruta et al. 2016); mutants of PAPOLA (human and mouse PAPOLA, UniProtKB/Uniprot accession number P51003 and Q61 183, respectively) wherein mutation or deletion of the nuclear localization signal can relocate the nuclear enzyme to the cytoplasm (Raabe, Murthy et al. 1994, Vethantham, Rao et al. 2008), and mutants of PAPOLG (human and mouse PAPOLG, UniProtKB/Uniprot accession number Q9BWT3 and Q6PCL9, respectively) wherein mutation or deletion of the nuclear localization signal is likely to relocate the nuclear enzyme to the cytoplasm (Kyriakopoulou, Nordvarg et al. 2001),
Yeast or protozoan poly(A) polymerases, e.g. mutants of Saccharomyces cerevisiae PAP1, UniProtKB/Uniprot accession number P29468; Schizosaccharomyces pombe PLA1, UniProtKB/Uniprot accession number Q10295), Candida albicans PAP (UniProtKB/Swiss-Prot accession number Q9UW26), Pneumocystis carinii PAP (also named Pneumocystis jiroveci; UniProtKB/Uniprot accession number A0A0W4ZDF2), wherein mutation or deletion of the nuclear localization signal is likely to relocate the nuclear enzyme to the cytoplasm, as well as other psychrotrophic, mesophilic, thermophilic or hyperthermophilic yeast or protozoan strains,
Viral poly(A) polymerases, including the heterodimeric vaccinia virus poly(A) polymerase that consists of the VP55 catalytic subunit (UniProtKB/Uniprot accession number strain Western Reserve P23371) and VP39 that acts as a processivity factor (UniProtKB/Uniprot accession number strain Western Reserve P07617), other poxvirus poly(A) polymerases (e.g. Cowpox virus, Monkeypox virus or Camelpox virus), African Swine Fever Virus (C475L, UniProtKB/Uniprot accession number A0A0A1E081), Acanthamoeba polyphaga mimivirus R341 (UniProtKB/Uniprot accession number E3VZZ8) and the Megavirus chilensis Mg561 poly(A) polymerases (NCBI GenBank accession number: YP_004894612), Moumouvirus (NCBI GenBank accession number AEX62700), Mamavirus (NCBI GenBank accession number AEQ60527), Cafeteria roenbergensis BV-PW1 virus (NCBI GenBank accession number YP_003969918), Megavirus Iba (NCBI GenBank accession number AGD92490), Yellowstone lake mimivirus (NCBI GenBank accession number YP_0091741 12), Chrysochromulina ericina virus (NCBI GenBank accession number YP_009173345), organic lake phycodnavirus 1 (NCBI GenBank accession number ADX05881), organic lake phycodnavirus 2 (NCBI GenBank accession number ADX06298), Faustovirus (NCBI GenBank accession number AMN83802) and Phaeocystis globose virus (NCBI GenBank accession number YP_008052392), and mutants or derivatives thereof which are able to catalyze the non-templated addition of adenosine residues from ATP onto the 3′ end of RNA molecules.

In another embodiment, said catalytic domain of a poly(A) polymerase is a catalytic domain of a non-canonical poly(A) polymerase, in particular of a cytoplasmic non-canonical poly(A) polymerase.

In preferred embodiment, said poly(A) polymerase is the mouse PAPOLA (UniProtKB/Uniprot accession number Q61183) containing KK656-657RR mutation to inactivate the nuclear localization signal (Raabe, Murthy et al. 1994, Vethantham, Rao et al. 2008), together with the S605A/S48A/S654A mutations to inactivate putative phosphorylation sites by the cdc2 protein kinase, which repress PAPOLA activity during the M phase of the cell cycle (Raabe, Bollum et al. 1991). Alternatively or in addition, mouse PAPOLA also contains the F1001 mutation, which increases the processivity of the enzyme (Raabe, Murthy et al. 1994).

Protein-RNA Tethering Systems

As described in the present specification, some enzymatic domains of the chimeric protein according to the invention should be preferably fused to at least one RNA-binding domain of a protein-RNA tethering system.

Protein-RNA tethering system refers to a system wherein a protein (or a peptide) recognizes and specifically binds (with high affinity) via its RNA-binding domain to a specific RNA element consisting of a specific RNA sequence and/or structure, therefore making possible to tether this protein (or peptide) with this RNA element. The specific binding between the protein (or the peptide) via its RNA binding domain and the specific RNA element implies that the protein (or peptide) and the specific RNA element interact with high affinity. Interaction with high affinity includes interaction with an affinity of about 10⁻⁶ M or stronger, in particular at least 10⁻⁷M, at least 10⁻⁸M, at least 10⁻⁹M and more particularly at least 10⁻¹⁰M

The RNA-protein affinity can be determined by various methods, well known by one skilled in the art. These methods include, but are not limited to, steady-state fluorescence or electrophoretic measurements, RNA electrophoretic mobility shift assay. The RNA-binding domain of a protein-RNA tethering system make possible to tether a specific protein (or peptide) via said RNA-binding domain with this specific RNA element of a target mRNA.

The Protein-RNA tethering systems from bacteriophages characterized so far belong either to the lambdoid virus family (i.e. λ, P22, Φ21, HK97 and 933W viruses) or the MS2-related family (i.e. MS2, and R17). The MS2 coat protein and the R17 coat protein recognize and interact with high affinity with specific RNA elements consisting of stem-loop RNA structures. The entire or nearly entire MS2/R17 proteins are needed for proficient binding to the tethered RNA, since multiple amino-acid residues spread in these proteins are involved in stem-loop interaction (Valegard, Murray et al. 1997). The lambdoid N antitermination protein-RNA tethering systems recognize and interact with high affinity with specific RNA elements consisting of BoxBl and BoxBr stem loop RNA structures (Das 1993, Greenblatt, Nodwell et al. 1993, Friedman and Court 1995). Importantly, the 18- to 22-amino-acid region from the N-terminal sequences of the lambdoid N-proteins bind to cognate RNA sequences with an affinity and specificity similar to that of the full-length N-proteins. Other well-characterized protein-RNA tethering systems are disclosed in WO2019/020811.

In one embodiment, said RNA-binding domain is a bacteriophage RNA-binding domain of a bacteriophage protein selected in the group consisting of: the MS2 virus coat protein (NCBI GenBank accession number NC_001417.2, UniProtKB/Uniprot accession number P03612), the R17 virus coat protein (NCBI GenBank accession numbers EF108465.1, UniProtKB/Uniprot accession number P69170) and the lambdoid viruses N antitermination proteins and mutants and derivatives thereof which are able to recognize and interact with high affinity with specific RNA element, more particularly the lambdoid viruses N antitermination proteins selected from the group consisting of the N antitermination protein from lambda virus (NCBI GenBank accession number NC_001416.1, complete genome sequence; UniProtKB/Uniprot accession number P03045), phi21 virus N antitermination protein (NCBI GenBank accession number AH007390.1, partial genome sequence; UniProtKB/Uniprot accession number P07243), HK97 virus N antitermination protein-RNA tethering system (NCBI GenBank accession number NC_002167.1, complete genome sequence; NCBI GenBank accession number protein accession number NP_037732.1) and p22 virus N antitermination protein (particularly NCBI GenBank accession number sequence (NCBI GenBank accession number NC_002371.2, complete genome sequence; UniProtKB/Uniprot accession number P04891), and even more particularly the N antitermination protein from lambda virus (NCBI GenBank accession number NC_001416.1, complete genome sequence; UniProtKB/Uniprot accession number P03045).

In another embodiment, a poly(A) polymerase or at least its catalytic domain is linked to the MS2 virus coat protein (NCBI GenBank accession number NC_00141 7.2, UniProtKB/Uniprot accession number P03612) or its isolate the R17 virus coat protein (NCBI GenBank accession numbers EF108465.1, UniProtKB/Uniprot accession number P69170), or a mutant or derivative thereof which is able to recognize and interact with high affinity to the specific stem-loop RNA element.

In still another embodiment, a poly(A) polymerase or at least its catalytic domain is linked to any one of the N-peptides of lambda family bacteriophages that consists of amino acids at position 1 to 22, in particular 1 to 18, of the lambda virus N antitermination protein (NCBI GenBank accession number NC_001416.1, complete genome sequence; UniProtKB/Uniprot accession number P03045), or of phi21 virus N antitermination protein (NCBI GenBank accession number AH007390.1, partial genome sequence; UniProtKB/Uniprot accession number P07243), or of HK97 virus N antitermination protein-RNA tethering system (NCBI GenBank accession number NC_002167.1, complete genome sequence; NCBI GenBank accession number protein accession number NP_037732.1) or of P22 virus N antitermination protein (NCBI GenBank accession number NC_002371.2, complete genome sequence; UniProtKB/Uniprot accession number P04891) or a mutant or derivative thereof, which are able to recognize and bind with high affinity to the specific RNA element consisting of BoxBl and BoxBr stem loop RNA structures.

In a preferred embodiment, a poly(A) polymerase or at least its catalytic domain is linked to the N-peptide consisting of amino acids at position 1 to 22 of the lambda virus N antitermination protein (NCBI GenBank accession number NC_001416.1, complete genome sequence; UniProtKB/Uniprot accession number P03045), which is able to recognize and interact with high affinity to the specific RNA element consisting of BoxBl (SEQ ID NO. 31) and BoxBr (SEQ ID NO. 32) stem loop RNA structures, in particular SEQ ID NO. 30, preferably encoded by SEQ ID NO. 29.

Linking Peptides

As also described in WO2011/128444 and WO2019/020811, the different coding sequences that makes up the chimeric protein may be fused in-frame together though linking peptides.

Linking peptide has the advantage of generating fusion proteins in which steric hindrance is minimized and enough space is provided for the components of the fusion protein to remain in their native conformation.

In one embodiment, the modules of the system according to the invention, i.e. the different catalytic domains and polypeptides of the system according to the invention, in particular of the chimeric enzyme/protein or the chimeric polyprotein, can be assembled, fused, or bound directly or indirectly by a linking peptide, particularly by a linking peptide of formula Gly₄, (Gly₄Ser)₁, (SEQ ID NO. 33), (Gly₄Ser)₂ (SEQ ID NO. 34) or (Gly₄Ser)₄, more particularly of formula (Gly₄Ser)₂ and (Gly₄Ser)₄, and even more particularly (Gly₄Ser)₂, also abbreviated (G4S)2.

Said linking peptide of the invention can be selected from the group consisting of:
peptides of formula (GlyₘSerₚ)ₙ, in which:
   m represents an integer from 0 to 12, in particular from 1 to 8, and more particularly from 3 to 6 and even more particularly 4;
   p represents an integer from 0 to 6, in particular from 0 to 5, more particularly from 0 to 3 and more particularly 1; and
   n represents an integer from 0 to 30, in particular from 0 to 12, more particularly from 0 to 8 and even more particularly between 1 and 6 inclusive;

Other types of peptide linkers can be also considered to generate chimeric enzymes according to the invention, as those described previously in the patent application WO 2011/128444.

In a preferred embodiment, the C-terminal end of one of said catalytic domains of a capping enzyme is fused to the N-terminal end of said catalytic domain of a DNA-dependent RNA polymerase through peptides of formula Gly₄, (Gly₄Ser), (Gly₄Ser)₂ and (Gly₄Ser)₄, preferably (Gly₄Ser)₂.

In still another embodiment, the said polypeptide capable of modulating the activity or expression of a target host cell protein involved in the regulation of the phosphorylation level of eIF2α are linked by a linking peptide to said chimeric enzyme and/or to said poly(A) polymerase.

Leucine Zippers

The leucine zippers, which are dimeric coiled-coil protein structures composed of two or more amphipathic α-helices that interact with each other, are commonly used to homo- or hetero-dimerize/multimerize proteins. Each helix consists of repeats of seven amino acids, in which the first amino-acid (residue a) is hydrophobic, the fourth (residue d) is usually a leucine, while the other residues are polar. Several types of leucine-zippers have been described, allowing non-covalent assembly in parallel or anti-parallel orientations of a variable number of identical or different peptide subunits.

In one embodiment, the different catalytic domains and polypeptides of the system according to the invention, in particular of the chimeric enzyme/protein or the chimeric polyprotein, can homodimerize in parallel orientation by the super leucine zipper (sLZ), which has a long coiled coil helix (Harbury, Zhang et al. 1993, Harbury, Kim et al. 1994), or homotetramerize in antiparallel orientation by the GCN4-pVg leucine zipper (Pack, Kujau et al. 1993, Pluckthun and Pack 1997), and even more preferably by the sLZ leucine zipper.

Other types of leucine zippers can be also considered to generate chimeric enzymes according to the invention, as those described previously in the patent applications WO2011/128444 and WO2019/020811.

In a preferred embodiment, a sZIP leucine zippers forms heterodimers in parallel orientation of said E3L-Zα/NS1-dsDNA/(G4S)2/SZIP polypeptide, which is capable of decreasing the phosphorylation level of eIF2α.

Ribosome Skipping Sequences

Ribosome skipping motif is an alternate mechanism of translation in which a specific viral peptide prevents the ribosome from covalently linking a new inserted amino-acid, and let it continue translation.

This results in apparent co-translational cleavage of the target polyprotein.

Said ribosome skipping motif is selected in the group consisting of the 2A sequences from the Foot-and-mouth disease virus Aphtovirus (UniProtKB/Swiss-Prot AAT01756), Avisivirua A (UniProtKB/Swiss-Prot M4PJD6), Duck hepatitis A Avihepatovirus (UniProtKB/Swiss-Prot Q0ZQM1), Encephalomyocarditis Cardiovirus (UniProtKB/Swiss-Prot Q66765), Cosavirus A (UniProtKB/Swiss-Prot B8XTP8), Equine rhinitis B Erbovirus 1 (UniProtKB/Swiss-Prot Q66776), Seneca Valley Erbovirus (UniProtKB/Swiss-Prot Q155Z9), Hunnivirus A (UniProtKB/Swiss-Prot F4YYF3), Kunsagivirus A (UniProtKB/Swiss-Prot S4VD62), Mischivirus A (UniProtKB/Swiss-Prot I3VR62), Mosavirus A2 (UniProtKB/Swiss-Prot X2L6K2), Pasivirus A1 (UniProtKB/Swiss-Prot I6YQK4), Porcine teschovirus 1 (UniProtKB/Swiss-Prot Q9WJ28), Infectious flacherie Iflavirus (UniProtKB/Swiss-Prot O70710), Thosea asigna Betatetravirus (UniProtKB/Swiss-Prot Q9YK87), Cricket paralysis Cripavirus (UniProtKB/Swiss-Prot Q9IJX4), Human rotavirus C (UniProtKB/Swiss-Prot Q9PY95), and Lymantria dispar cypovirus 1 (UniProtKB/Swiss-Prot Q911D7),

In particular, said nucleic acid sequence encoding a ribosome skipping motif is selected in the group consisting of the 2A sequences from the Foot-and-mouth disease virus Aphtovirus (also designated as F2A, UniProtKB/Swiss-Prot AAT01756) or Porcine teschovirus 1 (also designated as P2A, UniProtKB/Swiss-Prot Q9WJ28), or even more preferably the F2A leucine-zipper from the Foot-and-mouth disease Aphtovirus.

Other types of ribosome-skipping sequences can be also considered, as those described previously in the patent application WO2019/020811.

In one preferred embodiment, said nucleic acid sequence encoding a ribosome-skipping motif can be localized after the sequence encoding said catalytic domain of a poly(A) polymerase and after the coding sequence of said modulator polypeptides.

Modulator Polypeptides

As previously mentioned, the polypeptide capable of modulating the activity or the expression of a target host cell protein involved in the regulation of the phosphorylation level of eIF2α, can be introduced as a polypeptide into the host cell, or can be expressed from a nucleic acid molecule introduced into the host cell. Both embodiments are modulator polypeptides according to the invention, capable of modulating the activity or the expression of a target host cell protein applicable to the invention.

In the embodiment corresponding to the introduction of the modulator polypeptide encoded by a nucleic acid molecule, it is preferred that the chimeric protein/enzyme comprising at least one catalytic domain of a capping enzyme and at least one catalytic domain of a DNA-dependent RNA polymerase is also introduced as a nucleic acid molecule to be expressed.

In a preferred embodiment, the nucleic acid molecule encoding the chimeric protein/enzyme and the nucleic acid molecule encoding the modulator polypeptide are introduced simultaneously. They may advantageously be part of the same nucleic acid molecule or may be on a set of nucleic acid molecules which can be introduced simultaneously, or not.

In the case of a single nucleic acid molecule, the method of the invention comprises introducing into the host cell a nucleic acid molecule comprising:
a. at least one nucleic acid sequence encoding a chimeric protein as defined previously, namely comprising at least one catalytic domain of a capping enzyme and at least one catalytic domain of a DNA-dependent RNA polymerase; and
b. at least one nucleic acid sequence encoding a polypeptide capable of modulating the activity or expression of a target host cell protein involved in the regulation of the phosphorylation level of eIF2α as defined above, namely a modulator polypeptide.

The nucleic acid molecule may comprise more than one, for instance two nucleic acid sequences, each encoding a modulator polypeptide, preferably acting on different pathways leading to the downregulation of the phosphorylation level of eIF2α, e.g. an inhibitor of EIF2AK2 and an activator of eIF2α dephosphorylation.

According to this embodiment, the introduced nucleic acid molecule thus comprises the sequences encoding the chimeric protein, as well as the sequences encoding the modulator polypeptide, which may be linked operably to different promoters.

The inventor has moreover unexpectedly demonstrated that both nucleic acid sequences a. and b. may not only be part of the same nucleic acid molecule, but they may even be part of the same Open Reading Frame (ORF). In such a case, the nucleic acid molecule encoding the chimeric protein/enzyme also comprises a sequence encoding the modulator polypeptide, thus giving rise to a single chimeric polyprotein, comprising at least one catalytic domain of a capping enzyme, at least one catalytic domain of a DNA-dependent RNA polymerase and at least one polypeptide capable of modulating the activity of a target host cell protein involved in the regulation of the phosphorylation level of eIF2α. This embodiment is illustrated in the example 6 and following.

As detailed previously, the chimeric polyprotein may advantageously also comprise at least one catalytic domain of a poly(A) polymerase, linked to at least one RNA-binding domain of a protein-RNA tethering system, especially a catalytic domain of a poly(A) polymerase tethered through a lambdoid N-peptide or a sequence of similar function.

The different domains of this chimeric polyprotein can be separated by linkers, as more specifically detailed below. Alternatively, the sequences coding these different domains may also be separated by ribosome skipping motifs. Both linkers and ribosome skipping motifs may be used according to the invention. It is preferred in this respect that the nucleic acid molecule encoding the modulator polypeptide as defined above be separated from the sequences coding for the different catalytic domains, especially from the sequence coding for the capping enzyme and DNA-dependent RNA polymerase, and from the poly(A) polymerase, by ribosome skipping motifs. Definition and additional details regarding these motifs are disclosed below.

The invention thus encompasses methods wherein steps (a) and (b) as disclosed above are carried out simultaneously, and correspond to the introduction into the host cell of at least one nucleic acid sequence encoding the chimeric protein comprising the catalytic domain of a capping enzyme and the catalytic domain of a DNA-dependent RNA polymerase, one nucleic acid sequence encoding said polypeptide capable of downregulating the phosphorylation level of eIF2α and preferably also one nucleic acid sequence encoding a poly(A) polymerase tethered through a lambdoid N-peptide or a sequence of similar function, fused in frame on a same nucleic acid molecule by one or more nucleic acid sequences encoding linkers and/or ribosome skipping motifs.

Subcellular Compartmentalization

The different elements of the invention, namely the catalytic domains and polypeptides according to the invention, can be nuclear, localized in a subcellular compartment or cytoplasmic. Addressing the chimeric enzyme/protein or polyprotein according to the invention to the nucleus can achieved by the addition of a signal peptide well known by one skilled in the art, which directs the transport of the enzyme in cells. For example, the chimeric enzyme/protein or polyprotein according to the invention can comprise a nuclear localization signal (NLS), which directs the enzyme/protein or polyprotein to the nucleus. Such NLS is often a unit consisting of five basic, plus-charged amino acids. The NLS can be located anywhere on the peptide chain.

The cytoplasmic localization of the chimeric enzyme/protein or polyprotein according to the invention has the advantage that it optimizes the levels of transgene expression by avoiding the active transfer of large DNA molecules (i.e. transgene) from the cytoplasm to the nucleus of eukaryotic cells and the export of RNA molecules from the nucleus to the cytoplasm. In addition, the expression of the chimeric enzyme/protein or polyprotein according to the invention in the cytoplasm of the host cell prevents its illegitimate transcription of the nuclear genome of the host cell.

These cytoplasmic chimeric enzyme/protein or polyproteins according to the invention can thus be useful to generate a host-independent, eukaryotic DNA expression system that is able to work in the cytoplasm in which significantly higher amounts of transfected DNA are usually found as compared to the nucleus and which circumvent any protein synthesis arrest, which could be triggered by chimeric enzyme/protein according to the invention. In addition, given its role, the modulator polypeptide of the invention is preferably localized in the cytoplasm of the host cell, in order to modulate the phosphorylation level of eIF2α.

The chimeric enzyme/protein or polyprotein according to the invention is therefore preferably a chimeric enzyme/protein or a polyprotein addressed to the cytoplasm. In particular, it does not comprise signal peptide that directs the transport of the enzyme/protein or polyprotein, except to the cytoplasm.

Applications of the Invention

Transient Expression System

The present invention relates to an artificial eukaryotic system for efficiently expressing a recombinant DNA molecule. Such a molecule can be any DNA molecule, which comprises a sequence to be translated, for example a sequence coding for a recombinant protein.

The recombinant DNA may either be introduced into the host cell or system independently; if so, it is preferably linked to its own promoter and to any additional sequences necessary for the initiation of transcription. Preferably said DNA is operatively linked to the promoter for a bacteriophage DNA-dependent RNA polymerase.

Alternatively, the recombinant DNA may be part of a nucleic acid molecule comprising a sequence coding for one of the other elements of the invention, namely part of a nucleic acid molecule comprising one or more of a sequence encoding a catalytic domain of a capping enzyme, a sequence encoding a catalytic domain of a DNA-dependent RNA polymerase, or a sequence encoding a modulator polypeptide. In such a case, the recombinant DNA is not necessarily linked to its own promoter.

Moreover, when a protein-RNA tethering system is used, as described above, the recombinant DNA also comprises the element coding for the RNA target of the RNA-binding domain protein-RNA tethering system. When the RNA-binding domain of a protein-RNA tethering system is the RNA-binding domain of the lambdoid N antitermination protein-RNA tethering systems, the element, which specifically binds to said RNA-binding domain can be a BoxBl and/or a BoxBr stem loop RNA structure (Das 1993, Greenblatt, Nodwell et al. 1993, Friedman and Court 1995), including the elements encoded by SEQ ID NO. 31 and SEQ ID NO. 32. Preferably several repetitions of the RNA-binding domain are inserted in the 3′UTR of the target mRNA, preferably at least 3, preferably 5 or more.

According to an embodiment, the recombinant DNA may also be part of a nucleic acid molecule comprising the sequence coding for a modulator polypeptide, wherein this modulator polypeptide constitutes a second means of downregulating the phosphorylation level of EIF2α, i.e. this recombinant

DNA is to be introduced into a cell which is either already modified to downregulate said phosphorylation level, or which is or will be transformed by another molecule coding for another modulator polypeptide. Alternatively, transient expression can be achieved by using one or several mRNA molecules synthesized in vitro, which comprise a sequence coding for one or more of the elements of the invention, namely one or more mRNA molecules comprising one or more of a sequence encoding a catalytic domain of a capping enzyme, a sequence encoding a catalytic domain of a DNA-dependent RNA polymerase, and/or a sequence encoding a modulator polypeptide.

Kit for Recombinant Protein Production

The invention also relates to a kit for the production of a recombinant protein of interest from a recombinant DNA, especially in an eukaryotic cell such as a primary cell or established cell line. This kit according to the invention may comprise or consist in:
an isolated nucleic acid molecule or a set of isolated nucleic acid molecules encoding a chimeric protein comprising at least one catalytic domain of a capping enzyme; at least one catalytic domain of a DNA-dependent RNA polymerase; and optionally, at least one nucleic acid sequence encoding a poly(A) polymerase preferably tethered through a N-peptide, or any tethering peptide from a protein-RNA tethering system); and
at least one compound capable of downregulating the phosphorylation level of eIF2α in a eukaryotic host cell or a nucleic acid sequence coding for such a compound.

The invention also relates to a kit for in-vitro protein synthesis (IVPS) or coupled transcription/translation. This kit according to the invention may comprise or consist, in addition to its standard components (i.e. cell lysate or extract, phage RNA polymerases), of:
an isolated nucleic acid molecule (i.e. recombinant DNA or mRNA) or a set of isolated nucleic acid molecules encoding a chimeric protein comprising at least one catalytic domain of a capping enzyme; at least one catalytic domain of a DNA-dependent RNA polymerase; and optionally, at least one nucleic acid sequence encoding a poly(A) polymerase preferably tethered through a N-peptide, or any tethering peptide from a protein-RNA tethering system); and
at least one compound capable of downregulating the phosphorylation level of eIF2α in a eukaryotic host cell lysate or extract or a nucleic acid sequence (i.e. recombinant DNA or mRNA) coding for such a compound.

In alternative embodiment, instead of the isolated nucleic acid molecule or set of isolated nucleic acid molecules encoding a chimeric protein, the kit according to the invention may comprise a vector or a set of vectors encoding a chimeric protein of the invention.

The compound capable of downregulating the phosphorylation level of eIF2α in a eukaryotic host cell may be any compound already described in the context of the present invention, it can be a small molecule, siRNA, shRNA, miRNA or a ribozyme targeting a protein involved in the phosphorylation pathway of EIF2α. According to the preferred embodiment of the invention, the compound is a polypeptide modulating the activity or the expression of a target host cell protein involved in the regulation of the phosphorylation level of eIF2α, especially an inhibitor of one of EIF2AK2, EIF2AK3, DDX58, IFIH1, MAVS, IFNAR1, IFNAR2, IRF3, IRF7, IFNB1, JAK1, STAT1, STAT2, TYK2 and IRF9 and/or an activator of eIF2α dephosphorylation, especially one of PPP1CA, PPP1R15A, DP71L(s), DP71L(I) or ICP34.5. The kit may comprise the modulator polypeptide, or a nucleic acid sequence encoding such a modulator polypeptide.

According to another embodiment, instead of an isolated nucleic acid molecule or a set of isolated nucleic acid molecules encoding a chimeric protein of the invention, the kit may comprise a chimeric protein/enzyme comprising at least one catalytic domain of a capping enzyme; and at least one catalytic domain of a DNA-dependent RNA polymerase; and optionally, at least one poly(A) polymerase preferably tethered through a lambdoid N-peptide or any tethering peptide from a protein-RNA tethering system.

According to a preferred embodiment, the kit comprises a single nucleic acid molecule, or a single vector, encoding a chimeric polyprotein according to the invention, thus comprising at least one catalytic domain of a capping enzyme, at least one catalytic domain of a DNA-dependent RNA polymerase, a modulator polypeptide and optionally, at least one poly(A) polymerase preferably tethered through a lambdoid N-peptide or any peptide from a protein-RNA tethering system.

Preferably, the chimeric enzyme or polyprotein, is cytoplasmic; or the nucleic acid molecules and/or vector are for a cytoplasmic expression.

A kit according to the invention may also comprise, or be completed by a recombinant DNA sequence encoding the recombinant protein to be produced. In such a case, the recombinant DNA sequence is preferably covalently linked to at least one sequence encoding the RNA element of said protein-RNA tethering system, which specifically binds to said RNA-binding domain.

Particularly, said kit of the invention comprises a DNA sequence encoding the recombinant protein, which is operatively linked to the promoter for a bacteriophage DNA-dependent RNA polymerase.

According to an embodiment, the kit comprises at least two compounds downregulating the phosphorylation level of eIF2α, preferably targeting different pathways, i.e. either type-I interferon response, or unfolded protein response.

According to said embodiment, the kit of the invention further comprises one or more of the following elements: the N(pro) from Bovine Viral Diarrhea Virus that targets IRF3, or a nucleic acid molecule encoding such an element, the NSs from Rift Valley fever virus that promotes EIF2AK2 proteasomal degradation, or a nucleic acid molecule encoding such an element, or 5′UTR from Sindbis viral genome that antagonize IFIT1.

Particularly, said kit further comprises instructions for use.

The invention also relates to a composition, in particular a kit or a pharmaceutical composition, comprising:
a chimeric enzyme/protein, in particular a cytoplasmic chimeric enzyme/protein, comprising at least one catalytic domain of a capping enzyme and at least one catalytic domain of a DNA-dependent RNA polymerase, particularly of a bacteriophage DNA-dependent RNA polymerase and/or an isolated nucleic acid molecule or a group of isolated nucleic acid molecules encoding said chimeric enzyme/protein; and,
optionally, a poly(A) polymerase, in particular a cytoplasmic poly(A) polymerase, comprising at least one RNA-binding domain of a protein-RNA tethering system, particularly of a bacteriophage protein-RNA tethering system, linked to at least one catalytic domain of said poly(A) polymerase and/or an isolated nucleic acid molecule encoding said poly(A) polymerase; and,
at least one compound capable of downregulating the phosphorylation level of eIF2α in a eukaryotic host cell, and/or an isolated nucleic acid molecule encoding a molecule capable of downregulating the phosphorylation level of eIF2α, particularly of a modulator polypeptide; and optionally,
a recombinant DNA sequence, which is preferably operatively linked to the promoter for said DNA-dependent RNA polymerase and preferably covalently linked to at least one sequence encoding the element interacting with high affinity with said RNA-binding domain;
   said composition being useful for the efficient translation of the recombinant DNA sequence, and thus for the production of a recombinant protein encoded by the recombinant DNA sequence.

According to an embodiment, the invention also relates to a composition (in particular a kit or a pharmaceutical composition) comprising:
a chimeric polyprotein, in particular a cytoplasmic chimeric polyprotein, comprising at least one catalytic domain of capping enzyme, at least one catalytic domain of a DNA-dependent RNA polymerase, particularly of a bacteriophage DNA-dependent RNA polymerase, a poly(A) polymerase, in particular a cytoplasmic poly(A) polymerase, comprising at least one RNA-binding domain of a protein-RNA tethering system, particularly of a bacteriophage protein-RNA tethering system, linked to at least one catalytic domain of said poly(A) polymerase; and a modulator polypeptide, and/or
an isolated nucleic acid molecule or a group of isolated nucleic acid molecules encoding said chimeric polyprotein; and optionally
a recombinant DNA sequence, which is preferably operatively linked to the promoter for said DNA-dependent RNA polymerase and preferably covalently linked to at least one sequence encoding the element interacting with high affinity with said RNA-binding domain;

More particularly, said composition, in particular a kit or a pharmaceutical composition, comprises:
a chimeric enzyme, in particular a cytoplasmic chimeric enzyme, comprising the NP868R capping enzyme, and the K1E DNA-dependent RNA polymerase, particularly linked by the (Gly4Ser)2 linker and/or an isolated nucleic acid molecule or a group of isolated nucleic acid molecules encoding said chimeric enzyme; and
a poly(A) polymerase, in particular a cytoplasmic poly(A) polymerase comprising at least one catalytic domain of a poly(A) polymerase selected in the group consisting of PAP1, PAPOLA, PAPOLB, VP55, C475L, R341 and MG561 poly(A) polymerases and comprising at least one RNA-binding domain of a protein-RNA tethering system linked to at least one catalytic domain of said poly(A) polymerase and/or an isolated nucleic acid molecule encoding said poly(A) polymerase; and
a Zα domain, in particular a Zα domain from E3L of vaccinia virus or from mammalian ADAR1 operably linked to at least one dsRNA-binding domain, in particular a dsRNA-binding domain from Influenza A virus NS1 protein, mammalian EIF2AK2, Flock House virus B2 protein, orthoreovirus σ3 protein, preferably selected from Influenza A virus NS1 and mammalian EIF2AK2 proteins, and/or an isolated nucleic acid molecule encoding said domains, and optionally
a recombinant DNA sequence, which is preferably operatively linked to the promoter for said DNA-dependent RNA polymerase and preferably covalently linked to at least one sequence encoding the element interacting with high affinity with said RNA-binding domain;

In still another preferred composition, in particular a kit or a pharmaceutical composition, comprises:
a chimeric enzyme, in particular a cytoplasmic chimeric enzyme, comprising the NP868R capping enzyme, and the K1E DNA-dependent RNA polymerase, particularly linked by the (Gly4Ser)2 linker and/or an isolated nucleic acid molecule or a group of isolated nucleic acid molecules encoding said chimeric enzyme; and
a poly(A) polymerase, in particular a cytoplasmic poly(A) polymerase comprising at least one catalytic domain of a poly(A) polymerase selected in the group consisting of PAP1, PAPOLA, PAPOLB, VP55, C475L, R341 and MG561 poly(A) polymerases and comprising at least one RNA-binding domain of a protein-RNA tethering system linked to at least one catalytic domain of said poly(A) polymerase and/or an isolated nucleic acid molecule encoding said poly(A) polymerase; and
a polypeptide capable of selectively binding to EIF2AK2, consisting of (a) the dsRNA-binding region from EIF2AK2 protein deleted of its carboxyl-terminal kinase domain, (b) optionally linked to specific domains from multimeric E3 ligases, such as BTRCP, FBW7, SPK2, VHL, SPOP, CRBN, SOCS2, STUB1 or SPK1; and
a domain capable of dephosphorylation of eIF2α including PPP1CA, PPP1R15A, DP71L(s), DP71L(I) or ICP34.5; and
a recombinant DNA sequence, which is preferably operatively linked to the promoter for said DNA-dependent RNA polymerase and preferably covalently linked to at least one sequence encoding the element interacting with high affinity with said RNA-binding domain;
   said composition being useful for the efficient translation of the recombinant DNA sequence, and thus for the production of a recombinant protein encoded by the recombinant DNA sequence.

Advantageously, the kit or the compositions of the invention can be used as an orthogonal gene expression system.

Engineered Cells for Protein Production

The invention is also directed to a eukaryotic host cell for the expression of a recombinant protein, wherein the phosphorylation level of eIF2α is constitutively or transiently downregulated in this cell, especially wherein the pathway of EIF2αphosphorylation has been impaired by any means already described in the context of the present invention. Such a cell according to the invention is further modified to incorporate an artificial eukaryotic expression system, as the systems disclosed in WO2011/128444 and WO2019/020811; this cell thus comprises at least one nucleic acid molecule encoding a chimeric protein comprising at least one catalytic domain of a capping enzyme; and at least one catalytic domain of a DNA-dependent RNA polymerase.

The phosphorylation level of eIF2α is constitutively or transiently downregulated or knocked-out by the different means disclosed previously, inter alia by impairing any component involved in the phosphorylation of eIF2α, or by expressing a protein involved in the de-phosphorylation of eIF2α, or by expressing one or more modulator polypeptides as defined previously, modulating the activity of a target host cell protein involved in the regulation of the phosphorylation level of eIF2α, or by editing genes involved in eIF2 phosphorylation by various gene editing technologies, such as ZFNs, TALENs, and CRISPR-Cas9 system and its derivatives. Such a cell therefore preferably comprises a heterologous nucleic acid sequence encoding at least one modulator polypeptide, thus capable of modulating the expression or activity of a target host cell protein involved in the regulation of the phosphorylation level of eIF2α.

The chimeric protein or enzyme is as defined in the context of the present invention.

The modulator polypeptide is also as described in the preceding sections, especially comprising a Zα domain from E3L of vaccinia virus linked to a dsRNA binding domain from Influenza A virus NS1 protein, preferably also comprising a homodimerization domain such as the super leucine-zipper motif. Still another preferred modulator polypeptide consists of the dsRNA-binding region from EIF2AK2 protein, optionally linked to specific domains from multimeric E3 ligases, and also linked to a domain capable of dephosphorylation of eIF2α.

The different and preferred embodiments are those described in connection with the methods of the invention. It is inter alia particularly preferred that the host cell comprises a nucleic acid molecule encoding a chimeric protein/enzyme, a nucleic acid molecule encoding a modulator polypeptide and optionally also a nucleic acid molecule encoding a catalytic domain of a poly(A) polymerase, tethered through a lambdoid N-peptide or a sequence of similar function. It is particularly preferred that the cell comprises a heterologous nucleic acid molecule comprising, in frame, at least a sequence coding for a catalytic domain of a capping enzyme, at least a sequence coding for a catalytic domain of a DNA-dependent RNA polymerase, and at least one sequence coding for a modulator polypeptide, and preferably also at least one sequence coding for a catalytic domain of a poly(A) polymerase, tethered through a N-peptide. The order of the different units is as described elsewhere in the context of the present invention, and preferably in the following order, from N to C-terminus:
catalytic domain of a poly(A) polymerase tethered through a N-peptide—modulator polypeptide—catalytic domain of a capping enzyme—catalytic domain of a DNA-dependent RNA polymerase; or
modulator polypeptide—catalytic domain of a poly(A) polymerase tethered through a N-peptide—catalytic domain of a capping enzyme—catalytic domain of a DNA-dependent RNA polymerase.

Linkers or ribosome skipping motif are advantageously incorporated in the nucleic acid molecule as already detailed.

In Vivo Gene Expression

The invention also relates to different uses and applications of the expression system of the invention, implying the chimeric protein ensuring the production of RNA molecules with 5′-terminal cap, and preferably with 3′ poly(A) tail; or nucleic acid molecule(s) coding for said chimeric protein, in a eukaryotic cellular environment characterized by a downregulation of the phosphorylation level of EIF2α, for the efficient expression of recombinant DNA molecules in a eukaryotic host cell and thus for the production of recombinant proteins.

As detailed in the previous sections, in a preferred embodiment, the expression system of the invention implies a chimeric polyprotein, ensuring the production of RNA molecules with 5′-terminal cap, and preferably with 3′ poly(A) tail and also ensuring the downregulation of the phosphorylation level of EIF2α, or nucleic acid molecule(s) coding for said polyprotein.

The uses of these expression systems are preferably in vitro or ex vivo, or in cellulo in isolated cells.

The in vivo uses are detailed in a further section.

The invention also relates to the use, particularly in vitro, ex vivo or in cellulo, of a nucleic acid molecule or a set of isolated nucleic acid molecules according to the invention, a vector or a group of vectors according to the invention, a chimeric polyprotein or set of polypeptides according to the invention, a cell according to the invention, a kit according to the invention or a composition according to the invention, in particular a pharmaceutical composition according to the invention for the efficient expression of recombinant DNA molecule in a eukaryotic host cell.

The invention also relates to the use, particularly in vitro, ex vivo or in cellulo, of an isolated nucleic acid molecule or a set of isolated nucleic acid molecules according to the invention, or of a chimeric polyprotein or set of polypeptides of the invention, a vector or a group of vectors according to the invention, a cell according to the invention, a kit according to the invention or a composition according to the invention, in particular a pharmaceutical composition according to the invention for the production of a recombinant protein of interest, in particular a protein of therapeutic interest like antibody or antigens, particularly in eukaryotic systems, such as in in vitro synthesized protein assay or cultured cells.

The invention also relates to an in vitro, ex vivo or in cellulo method for producing a recombinant protein from a recombinant DNA sequence, in a host cell, said method comprising the step of expressing in the host cell an isolated nucleic acid molecule or a set of isolated nucleic acid molecules according to the invention, wherein said recombinant DNA sequence is preferably covalently linked to at least one sequence encoding the RNA element of a protein-RNA tethering system, which specifically binds to the RNA-binding domain of said protein-RNA tethering system.

Particularly, said recombinant DNA sequence is operatively linked to the promoter for a bacteriophage DNA-dependent RNA polymerase or to the promoter for said DNA-dependent RNA polymerase of the chimeric protein of the invention.

Alternatively, the invention also relates to an in vitro or ex vivo method for producing a recombinant protein from a recombinant DNA sequence, in a host cell wherein the phosphorylation level of EIF2α has been previously downregulated, said method comprising the step of expressing in the host cell a chimeric protein, comprising a catalytic domain of capping enzyme, a catalytic domain of a DNA-dependent RNA polymerase, particularly of a bacteriophage DNA-dependent RNA polymerase; and optionally a poly(A) polymerase, in particular a cytoplasmic poly(A) polymerase, comprising at least one RNA-binding domain of a protein-RNA tethering system, wherein said recombinant DNA sequence is covalently linked to at least one sequence encoding the RNA element of said protein-RNA tethering system, which specifically binds to said RNA-binding domain.

In particular, the method according to the invention can further comprise the step of introducing into the host cell said recombinant DNA sequence and/or the nucleic acid molecule(s) according to the invention, using methods well-known to one skilled in the art, like by transfection using calcium phosphate, by electroporation or by mixing a cationic lipid with DNA to produce liposomes.

Therapeutic Applications

The invention also relates to a chimeric polyprotein or set of polypeptides according to the invention, an isolated nucleic acid molecule or a set of nucleic acid molecules according to the invention, a vector according to the invention, for use as a medicament, in particular for the prevention and/or treatment of human or animal pathologies, preferably by means of gene therapy. Preferably, the chimeric polyprotein, set of polypeptides, isolated nucleic acid molecule or a set of nucleic acid molecules, or vector, is for use in combination with a recombinant DNA coding for a protein of therapeutic interest.

The invention also relates to the use of a chimeric polyprotein or set of polypeptides according to the invention, or an isolated nucleic acid molecule or a set of nucleic acid molecules according to the invention, or a vector according to the invention, or a cell according to the invention, or a kit according to the invention, for the preparation of a medicament for the prevention and/or treatment of human or animal pathologies, in particular by means of gene therapy.

The invention also relates to a therapeutic method comprising the administration of a chimeric polyprotein or set of polypeptides according to the invention, an isolated nucleic acid molecule or a set of nucleic acid molecules according to the invention, a vector according to the invention, a cell according to the invention or a kit or pharmaceutical composition according to the invention, in a therapeutically amount to a subject in need thereof. The therapeutic method according to the invention can further comprise the administration of at least one recombinant DNA sequence of interest, wherein said recombinant DNA sequence codes for a recombinant protein of interest, in a therapeutically amount to a subject in need thereof.

Said chimeric polyprotein, polypeptide set of polypeptides, isolated nucleic acid molecule, set of nucleic acid molecules, vector cell, kit or pharmaceutical composition according to the invention can be administrated simultaneously, separately or sequentially of said recombinant DNA sequence of interest, in particular before said DNA sequence of interest.

The pathologies which can be treated are selected from the group consisting of pathologies, which can be improved by the expression of at least one recombinant DNA sequence of interest, such as, but without being limited to cancer, neurodegenerative diseases (e.g. Parkinson's disease, Alzheimer's disease), viral infections (e.g. influenza, HIV, hepatitis), heart disease, diabetes, genetic disorders such as severe combined immune deficiency (ADA-SCID), Chronic Granulomatus Disorder (CGD), hemophilia, congenital blindness, lysosomal storage disease, sickle cell anemia, Huntington's disease and muscular dystrophy. In particular, the pathologies can be selected from the group consisting of cancers and their predisposition (especially breast and colorectal cancers, melanoma), malignant hemopathies (in particular leukemias, Hodgkin's and non-Hodgkin's lymphomas, myeloma), coagulation and primary hemostasis disorders, hemoglobinopathies (especially sickle cell anemia and thalassemia), autoimmune disorders (including systemic lupus erythematosus and scleroderma), cardiovascular pathologies (in particular coronary artery disease, myocardial infarction, restenosis and cardiomyopathy, cardiac rhythm and conduction disorders, and hypertrophic cardiomyopathy), vascular diseases (peripheral artery disease of the lower limbs, critical limb ischemia, Buerger's disease), metabolic disorders (especially type I and type II diabetes mellitus and their complications, dsylipidemia, atherosclerosis and their complications, glycogen storage diseases, phenylketonuria), acute hepatitis regardless of the etiology (e.g. acetaminophen and other drug-induced hepatitis, alcoholic hepatitis, Budd-Chiari disease, veno-occlusive disease, ischemic hepatitis, autoimmune hepatitis, Amanita phalloides intoxication, drug-related hepatotoxicity, Wilson's disease, viral infections [hepatitis A virus, hepatitis B virus, hepatitis E virus, herpes simplex, varicella zoster, yellow fever, parvovirus B19, cytomegalovirus], and Reye's syndrome), chronic liver diseases (including fibrosis or cirrhosis regardless of the etiology, non-alcoholic steato-hepatitis, infectious disorders (including AIDS, influenza flu and other viral diseases); botulism, tetanus and other bacterial disorders; malaria and other parasitic disorders), muscular disorders (including Duchenne muscular dystrophy and Steinert myotonic muscular dystrophy), respiratory diseases (especially cystic fibrosis and alpha-1 antitrypsin deficiency, acute lung injury, pulmonary fibrosis, chronic obstructive pulmonary disease), renal disease (especially polycystic kidney disease, acute renal failure, nephrotoxicity of treatments such as cisplatin, chronic renal failure, diabetic nephropathy, Berger's disease and, nephrotic syndrome), immune deficiency (ADA-SCID), lysosomal storage diseases (Pompe, Niemann-Pick, Gaucher, Fabry), colorectal disorders (including Crohn's disease and ulcerative colitis), ocular disorders especially retinal diseases (especially Leber's amaurosis, retinitis pigmentosa, age related macular degeneration), central nervous system disorders (especially Alzheimer disease, Parkinson disease, multiple sclerosis, Huntington disease, neurofibromatosis, adrenoleukodystrophy, amyotrophic lateral sclerosis, acute spinal cord injury, psychiatric diseases (bipolar disease, schizophrenia and autism), scleroderma, vocal fold scar, and central nervous system disorders, skin and connective tissue disorders (wound repair, Marfan syndrome and psoriasis). The invention also encompasses vaccination.

In a further embodiment, the invention relates to a method of treatment by gene therapy, an isolated nucleic acid molecule or a set of isolated nucleic acid molecules according to the invention, a kit according to the invention, a polyprotein, polypeptide, or set of polypeptides according to the invention, a cell according to the invention and/or a pharmaceutical composition according to the invention, in combination with a nucleic acid molecule encoding a therapeutic agent.

In a further embodiment, the invention is also directed to a method for generating an immune response in a mammal against an antigen, especially for vaccination purposes, comprising administering to said mammal a chimeric polyprotein or set of polypeptides according to the invention, or an isolated nucleic acid molecule or a set of nucleic acid molecules according to the invention, or a vector according to the invention, or a cell according to the invention, or a kit or composition according to the invention, in combination with a recombinant DNA coding for said antigen. The mammal is most preferably a human.

The antigen is any immunogenic antigen, and more preferably an antigen derived from a viral or bacterial constituent.

The invention has a particular advantage in the context of replicon vaccines, which are self-amplifying viral RNA sequences that, in addition to the sequence encoding the antigen of interest, contain all elements necessary for RNA replication (Lundstrom 2016). Several replicons can be engineered from ss(+) and ss(−)RNA viruses, the most commonly used belonging to the Alphavirus family, e.g. Sindbis virus, Semliki Forest virus, and Venezuelan equine encephalitis virus (Vander Veen, Harris et al. 2012, Lundstrom 2019). Although efficient, these self-replicating systems have the drawback of generating dsRNA, which is a mandatory intermediary of replication. As described above, the production of this dsRNA results in an inhibition of translation via the activation of EIF2AK2 and consequently the phosphorylation of EIF2α. The artificial expression system according to the invention overcomes this drawback by reverting EIF2α to an unphosphorylated state, thereby restoring translation.

The invention also relates to a pharmaceutical composition comprising a chimeric polyprotein or a set of polypeptides according to the invention, and/or an isolated nucleic acid molecule according to the invention and/or a set of nucleic acid molecules according to the invention, a vector according to the invention, a cell according to the invention, and/or a kit or composition according to the invention. Preferably, said pharmaceutical composition according to the invention is formulated in a pharmaceutical acceptable carrier. Pharmaceutical acceptable carriers are well known by one skilled in the art.

The pharmaceutical composition according to the invention can further comprise at least one recombinant DNA sequence of interest, coding for protein of therapeutic interest.

Such components are present in the pharmaceutical composition or medicament according to the invention in a therapeutic amount (i.e. therapeutically active and nontoxic amount).

The pharmaceutical composition is for use in any therapeutic application, including gene therapy and vaccination.

The invention also encompasses an isolated nucleic acid molecule or a set of isolated nucleic acid molecules a vector, a polyprotein or a set of polypeptides, a cell or a pharmaceutical composition according to the invention, for use in combination with a nucleic acid molecule encoding an antigen or a therapeutic agent, in a method of treatment or prophylaxis, in particular for gene therapy or for generating an immune response against an antigen in an individual, preferably a human.

The invention also relates to a combination product, which comprises as active ingredients a chimeric polyprotein, set of polypeptides, isolated nucleic acid molecule, set of nucleic acid molecules, vector, cell, kit or pharmaceutical composition according to the invention and at least one recombinant DNA sequence of interest, coding for a recombinant protein of interest, for its use as a medicament, wherein said active ingredients are formulated for separate, simultaneous or sequential administration.

The recombinant DNA sequence of interest may encode a polypeptide of therapeutic interest, which can be selected from a monoclonal antibody or its fragments, a growth factor, a cytokine, a cell or nuclear receptor, a ligand, a coagulation factor, the CFTR protein, insulin, dystrophin, a hormone, an enzyme, an enzyme inhibitor, a polypeptide which has an antineoplastic effect, a polypeptide which is capable of inhibiting a bacterial, parasitic or viral, in particular HIV, infection, an antibody, a toxin, an immunotoxin. The recombinant DNA sequence of interest may also encode an immunogenic protein, with a view to generating an immune response.

### BRIEF DESCRIPTION OF DRAWINGS

FIG. 1: shows the generic map of pC3P3 plasmids. The open-readings frames of the different generations of the C3P3 enzyme are subcloned into the pCMVScript plasmid backbone, downstream to the standard CpG-rich RNA polymerase II-dependent promoter IE1 promoter/enhancer from the human cytomegalovirus (CMV).

FIG. 2: shows the map of the pK1Ep-Luciferase-4xλBoxBr plasmid, which consists of a K1E phage RNA polymerase promoter, 5′-UTR from the human β-globin gene, Kozak consensus sequence followed by the ORF of Firefly luciferase gene, four BoxBL RNA tethering repeats in tandem from the λ bacteriophage, artificial poly[A] track of 40 adenosine residues, followed by a self-cleaving genomic ribozyme from of the hepatitis D virus, and terminated by the bacteriophage T7 φ10 transcription stop.

FIG. 3: illustrates the map of the pCMVScript-Luciferase, which consists of the ORF of Firefly luciferase gene subcloned into the pCMVScript plasmid backbone, downstream to the standard CpG-rich RNA polymerase II-dependent promoter IE1 promoter/enhancer from the human cytomegalovirus (CMV).

FIG. 4: illustrates the regulation of translation initiation by the eIF2, which is a heterotrimer consisting of eIF2α, eIF2β, and eIF2γ subunits. The conserved serine 52 residue the α subunit can be phosphorylated by several kinases, which increase the affinity of eIF2 for eIF2B. Since eIF2B can only exchange GDP for GTP if eIF2α is in its unphosphorylated state, the resulting decrease the activation of unphosphorylated eIF2 to its active GTP-bound state and concomitant decrease in translation initiation rates. Conversely, eIF2α can be dephosphorylated by the catalytic Protein Phosphatase 1 (PP1) subunit (PPP1CA) and its regulatory subunit PPP1R15. The unphosphorylated eIF2 complex can load the methionine-charged initiator tRNA Met-tRNAᵢ^{Met} and GTP though eIF2B guanosine nucleotide exchange factor, then assembles with other initiation factors to form the 43S preinitiation complex.

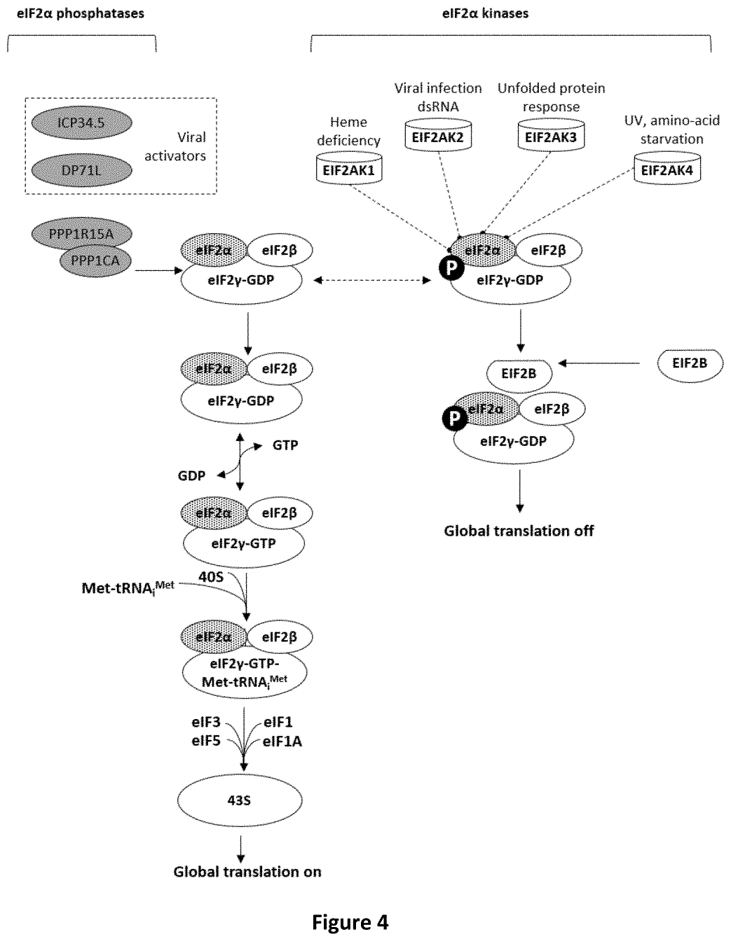

FIG. 5: shows the polysome profile observed in human HEK-293 transfected with the C3P3-G1, C3P3-G2 and C3P3-G3a plasmids, together with the pK1Ep-Luciferase-4xλBoxBr plasmid (solid lines). The profile obtained with the standard RNA polymerase II-dependent pCMVScript-Luciferase plasmid is shown for comparison (dotted line). From left to right, each profile shows the 40S, 60S, and 80S peaks, followed by the polysomes

FIG. 6: Western-blot analysis of eIF2α phosphorylation rate. The top blot is obtained with an anti-human eIF2α antibody which specifically recognizes its phosphorylated form on the Ser52 residue. The bottom blot assays the total human eIF2α using an anti-human eIF2α antibody that binds to the protein whether or not it is phosphorylated.

FIG. 7: illustrates the structure of the test plasmids encoding engineered proteins tested in Example 3. In the first series, the Z domain at the amino terminus extremity of the E3L protein is substituted by other domain with similar function. In the second series, the dsRNA-binding domain at the carboxyl-terminus end of the E3L protein is substituted by other domain with similar function. In the third series, leucine-zippers are added to the carboxyl terminus of the chimeric protein pE3L-Zα/NS1-dsDNA through flexible (G4S)2 linkers for its di/multimerization.

FIG. 8: depicts the structure of the plasmids encoding protein assemblies tested as C3P3-G3 systems as shown in Example 6. The ORF of the artificial protein E3L-Zα/NS1-dsDNA/(G4S)2/SZIP is inserted into the scaffold of the C3P3-G2 enzyme, either at its start of immediately before the Nλ-mPAPOLA block (C3P3-G3d and C3P3-G3e), or within its coding sequence between the Nλ-mPAPOLA and NP868R-(G4S)2-K1ERNAP blocks (C3P3-G3a, C3P3-G3b, and C3P3-G3c).

FIG. 9: depicts the structure of the plasmids encoding protein assemblies tested as further C3P3-G3 systems. The dsRNA-binding domain from the human EIF2AK2 and the DP71L(I) ORF were inserted into the scaffold of the C3P3-G2 enzyme, either at its start of immediately before the Nλ-mPAPOLA block (C3P3-G3f and C3P3-G3g), or within its coding sequence between the Nλ-mPAPOLA and NP868R-(G4S)2-K1ERNAP blocks (C3P3-G3h and C3P3-G3i). In addition, two types of intervening sequences were used, either the flexible (Gly4Ser)2 linker (C3P3-G3f and C3P3-G3h), or the 2A ribosome skipping sequence (C3P3-G3g and C3P3-G3i).

### EXAMPLES

The examples describe different improvements of the C3P3 artificial expression system previously developed by the inventor.

### Example 1: Polysome Profiling LED to the Discovery of Translation Initiation Defect in Human Cells by the C3P3 Artificial Expression System

Objectives

The aim of the present series of experiments was to assess by polysomal profiling whether translation by C3P3 expression system was normal or altered.

Polysome profiling is a method of global analysis of cell translation that separates mRNAs on a sucrose gradient according to the number of bound ribosomes. Due to strong reproducibility and sensitivity, polysome profiling is regarded as the reference method to study the translation process in cells (Chasse, Boulben et al. 2017). This technique is particularly sensitive to any alteration in the initiation of translation. More specifically, this method has been used successfully for analyzing eIF2α kinases effects on translation (Dey, Baird et al. 2010, Teske, Baird et al. 2011, Baird, Palam et al. 2014, Andreev, O'Connor et al. 2015, Knutsen, Rødland et al. 2015).

The profiling of the polysomes obtained in human cultured cells with the 1St generation (C3P3-G1) system was reported (Jais, Decroly et al. 2019). Its absence of detectable anomaly suggested that the translation was normal using this system.

In addition to C3P3-G1, the present inventor has assayed by polysome profiling the 2^{nd} generation (C3P3-G2) and the 3^{rd} generation system CP3-G3a.

Methods

Plasmids

Artificial gene sequences were synthesized and assembled from stepwise PCR using oligonucleotides, cloned and fully sequence verified by GeneArt AG (Regensburg, Germany). The coding sequences of all constructions were optimized for expression in human cells with respect to codon adaptation index (Raab, Graf et al. 2010).

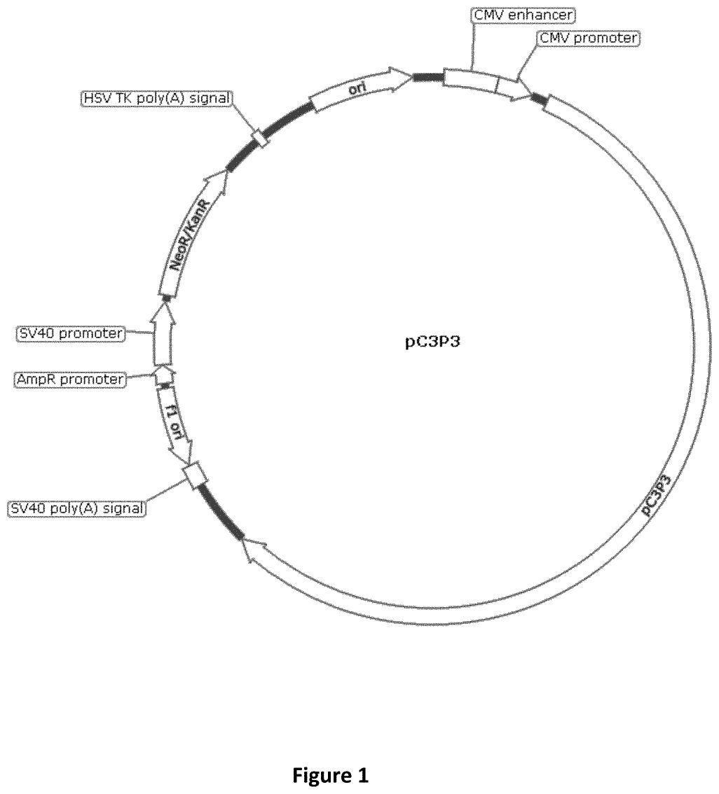

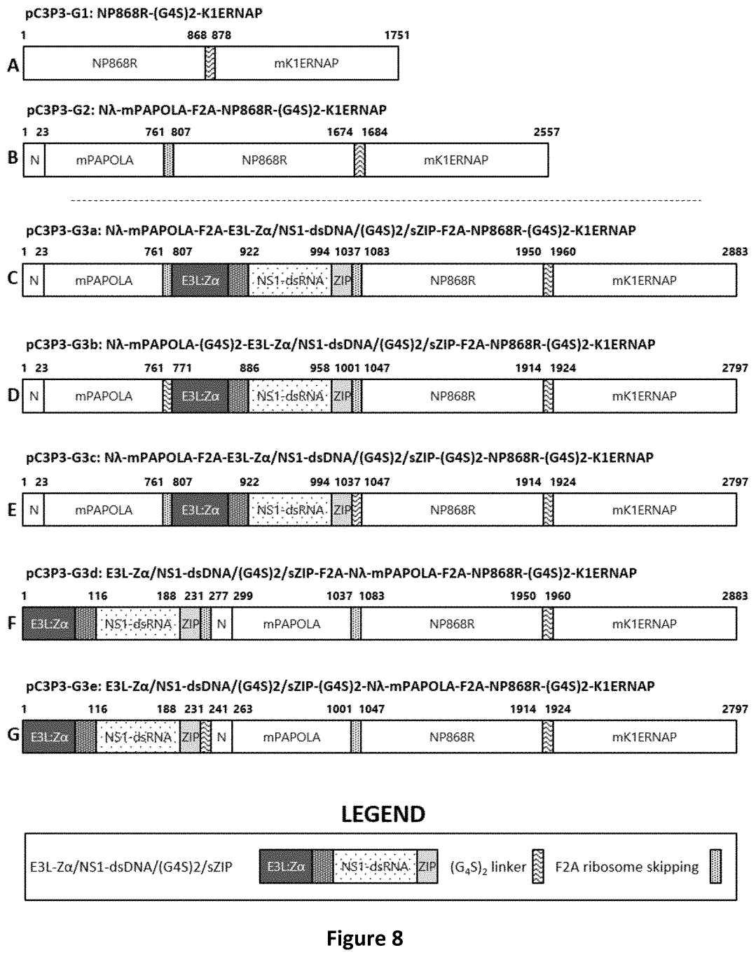

C3P3 enzyme sequences were subcloned into the pCMVScript plasmid backbone (Stratagene, La Jolla, Calif.), downstream to the standard CpG-rich RNA polymerase II-dependent promoter IE1 promoter/enhancer from the human cytomegalovirus (CMV), as shown FIG. 1. The structure of different generations of the C3P3 enzyme were as follows:
C3P3-G1, which allows the synthesis of mRNA with cap-0 modification at 5′-end, was described in WO2011/128444 and elsewhere (Jais 2011, Jais 2011, Jais, Decroly et al. 2019). The C3P3 enzyme consists of the following fusion from the N- to C-terminus (pC3P3-G1 plasmid; SEQ ID NO. 1 and SEQ ID NO. 2; FIG. 8A): African Swine Fever Virus capping enzyme NP868R (UniProtKB/Uniprot accession number P32094.1), flexible (G4S)2 linker, and mutant R551S K1E DNA-dependent RNA polymerase from bacteriophage K1E (UniProtKB/Uniprot accession number Q2WC24).
C3P3-G2, which allows the extension of a polyadenylation mRNA at 3′-end of the target mRNA in addition to cap-0 modification, has been described elsewhere with slight modifications in WO2019/020811 (Jais 2017). The C3P3 enzyme consists of the following fusion from the N- to C-terminus (pC3P3-G2 plasmid; SEQ ID NO. 3 and SEQ ID NO. 4; FIG. 8B): N-peptide from the lambda bacteriophage that binds at high affinity to the BoxBL sequences from the lambda bacteriophage inserted in the 3′UTR of the target mRNA (Genbank AAA32249.1), mutant S605A/S48A/S654A/KK656-657RR mouse poly(A) polymerase a isoform 1 (PAPOLA, UniProtKB/Uniprot accession number Q61183-1), ribosome skipping F2A sequence from the Foot-and-mouth disease virus that allows ribosome skipping (Genbank AAT01770.1), African Swine Fever Virus capping enzyme NP868R (UniProtKB/Uniprot accession number P32094.1), flexible (G4S)2 linker, and mutant R551S K1E DNA-dependent RNA polymerase from bacteriophage K1E (UniProtKB/Uniprot accession number Q2WC24).
A third generation C3P3-G3 enzyme (C3P3-G3a) is described below in Example 6 (pC3P3-G3a plasmid; SEQ ID NO. 19 and SEQ ID NO. 20).

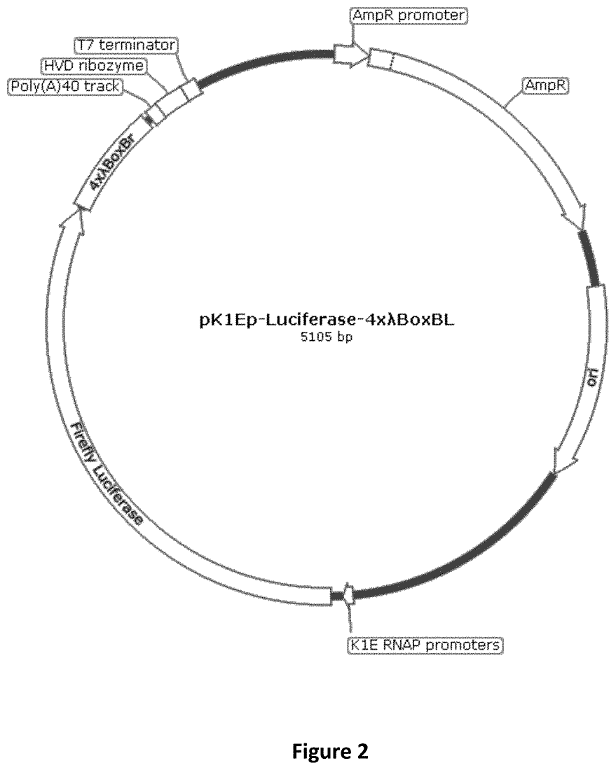

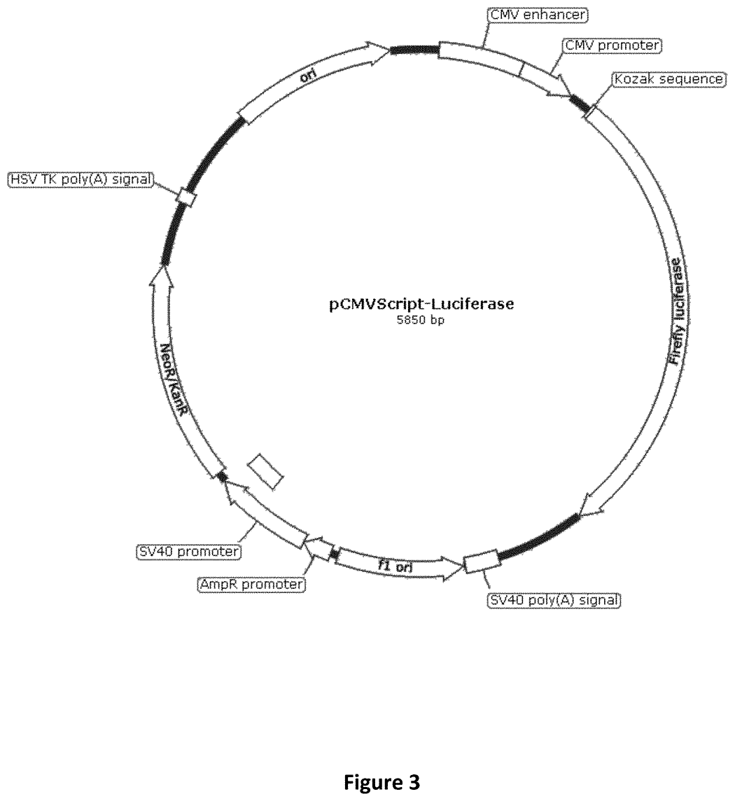

The C3P3 system was used to express the Firefly luciferase test gene (FIG. 2, i.e. pK1Ep-Luciferase-4xλBoxBr), which consists of a K1E phage RNA polymerase promoter transcribed by the C3P3 enzyme, 5′-UTR from the human β-globin gene (Genbank NM_000518.4), Kozak consensus sequence for initiation of translation followed by the ORF of the Photinus pyralis gene (i.e. Firefly luciferase; UniProtKB/Uniprot accession number Q27758) and stop codon, four BoxBL RNA tethering repeats in tandem from the lambda bacteriophage that binds at high affinity to the N-peptide of the C3P3-G2 and C3P3-G3a enzyme (nucleotides 38312-38298 of genomic sequence of Enterobacteria phage lambda KT232076.1), an artificial poly[A] track of 40 adenosine residues, followed by a self-cleaving genomic ribozyme from of the hepatitis D virus, and terminated by the bacteriophage T7 φ10 transcription stop. As a control the Firefly luciferase was expressed by standard nuclear expression system using RNA polymerase II-dependent CMV promoter (FIG. 3). The corresponding pCMVScript-Luciferase plasmid therefore contained the IE1 human CMV promoter/enhancer, Kozak consensus sequence followed by the ORF from Photinus pyralis gene (UniProtKB/Uniprot accession number Q27758), and late SV40 polyadenylation signal.

For standard experiments, the Human Embryonic Kidney 293 (HEK-293, ATCC CRL 1573) were routinely grown at 37° C. in 5% CO₂ atmosphere at 100% relative humidity. Cells were maintained in Dulbecco's Modified Eagle's Medium (DMEM) supplemented with 4 mM L-alanyl-L-glutamine, 10% fetal bovine serum (FBS), 1% non-essential amino-acids, 1% sodium pyruvate, 1% penicillin and streptomycin, and 0.25% fungizone.

Cells were routinely plated in 24-well plates at 1×10⁵ cells per well the day before transfection and transfected at 80% cell confluence. Transient transfection was performed with Lipofectamine 2000 reagent (Invitrogen, Carlsbad, Calif.) according to manufacturer's recommendations. Except otherwise stated, cells were transfected with 2 μl of Lipofectamine 2000 plus 0.8 pg of total plasmid DNA, and were assayed two days after transfection.

Polysome Fractionation

Polysome fractionation of HEK-293 transfected cells was performed as described elsewhere with minor modifications (Verrier and Jean-Jean 2000). A single 75 cm² tissue culture flask of HEK-293 transfected cells was used for each sucrose gradient. The culture medium was removed 24 hours after transfection and replaced with fresh medium. After overnight incubation, the medium was changed again and 2 hours later, cycloheximide at 100 pg/ml was added for 10 min. Cells were washed with PBS, collected by trypsinization, and pelleted. The dry cell pellet was resuspended in 500 μl of lysis buffer (50 mM Tris-HCl at pH 7.4, 300 mM KCl, 10 mM Mg-Acetate, 1 mM DTT, 0.05% Nonidet P40) containing 200 units/ml of SUPERaseln RNAse inhibitor (Invitrogen) and 100 pg/ml of cycloheximide, and lyzed by incubation on ice for 10 min with occasional shaking. Cycloheximide blocks the movement of peptidyl-tRNA from acceptor (aminoacyl) site to the donor (peptidyl) site on ribosomes and locks them onto the mRNA. Nuclei and cell debris were removed by centrifugation at 1,000×g for 10 min and 400 μl of supernatant was layered directly onto a 12 ml 15-50% (w/v) sucrose gradient in 50 mM Tris-Acetate (pH 7.5), 50 mM NH₄Cl, 12 mM MgCl₂ and 1 mM DTT. The gradient was centrifuged at 39,000 rpm in a SW41 Beckman rotor for 2.75 hours at 4° C. After centrifugation, optical density (O. D.) at 254 nm was monitored by pumping the gradient through a Retriever 500 (Teledyne Isco) fraction collector.

Results

Polysome profiling allows global analysis of host-cell translation by separating translated mRNAs on a sucrose gradient according to the number of bound ribosomes. HEK-293 cells expressing the Firefly

Luciferase gene under control of the C3P3 systems (i.e. C3P3-G1, C3P3-G2 and C3P3-G3a) or standard CMV-promoter-based nuclear expression plasmid were compared. Cell lysates are loaded of a 15-50% sucrose gradient. After ultracentrifugation, the gradient is monitored at A254 using a flow cell coupled to a spectrophotometer and then fractionated into equal fractions.

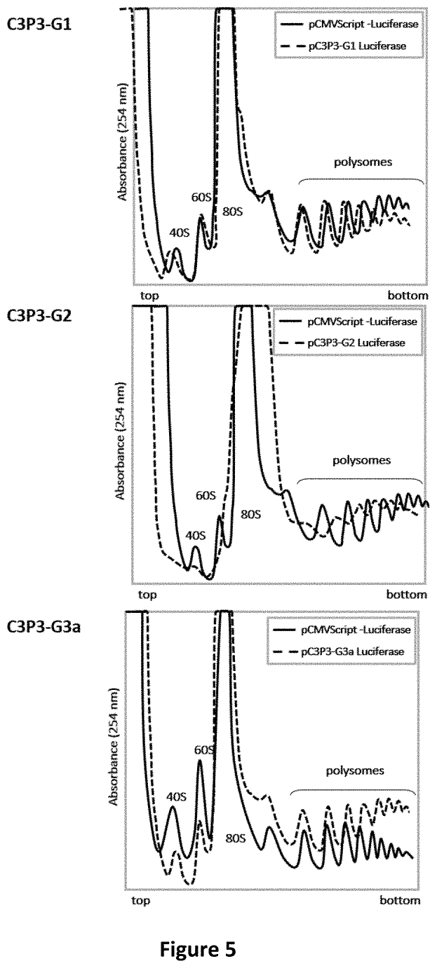

As previously described (Jais 2017), virtually no difference in ribosome distribution patterns was observed between HEK-293 cells transfected with pC3P3-G1/pK1Ep-Luciferase-4xλBoxBr and pCMVScript-Luciferase (FIG. 5a). This finding therefore shows that expression by C3P3-G1 expression system has no detectable effect on global translation of human HEK-293 cells in comparison to standard nuclear expression system as assessed by polysome profile analysis.

Surprisingly, the expression by C3P3-G2 of the Firefly Luciferase has major impact on global translation of human HEK-293 cells (FIG. 5B). Cells transfected pC3P3-G2/pK1Ep-Luciferase-4xλBoxBr showed very reduced 40S and 60S ribosome peaks in comparison of those of control cells transfected with pCMVScript-Luciferase. Moreover, the 80S monosome peak, which is formed during protein synthesis by assembly of the 40S and 60S subunits, was considerably increased, while polysome peaks were reduced. Such patterns are suggestive of a translation initiation defect, and more specifically by eIF2α hyperphosphorylation (Dey, Baird et al. 2010, Baird, Palam et al. 2014, Andreev, O'Connor et al. 2015, Knutsen, Rødland et al. 2015). As described above, several kinases can phosphorylate eIF2α and are activated by various stress signals, including oxidative stress [heme-regulated inhibitor (HRI) or EIF2AK1], dsRNA generated by viral infection [protein kinase double-stranded RNA-dependent (PKR) or EIF2AK2], unfolded protein response activated by endoplasmic reticulum overload [PKR-like ER kinase (PERK) or EIF2AK3], and ROS accumulation or amino acid starvation [general control non-derepressible-2 (GCN2) or EIF2AK4].

The ribosome distribution patterns were also investigated in HEK-293 cells expressing the Firefly Luciferase gene under control of the C3P3-G3a system. As detailed in Example 6, the C3P3-G3a enzyme contains an artificial interferon-inhibitory protein consisting of the Z-α domain from E3L of the vaccinia virus fused the dsRNA-binding domain from NS1 protein of the influenza A virus and terminated by the leucine sZip leucine zipper for homodimerization. The ORF of this artificial protein is inserted in frame into the open-reading of C3P3-G2 enzyme and is separated by 2A ribosome skipping motifs (FIG. 8C). Cells were transfected pC3P3-G3a/pK1Ep-Luciferase-4xλBoxBr as described above. Polysome profile of transfected cells was very similar, although not strictly identical, to that of cells transfected with pCMVScript-Luciferase plasmid (FIG. 5C). These results suggest that blocking the phosphorylation of eIF2α, returns translation to an almost normal state in the cell expressing Firefly Luciferase reporter gene under the control of the C3P3-G3a system.

Conclusions

These experiments on cultured human cells show that the second generation of the C3P3 system, induces a strong defect in the initiation of translation, which can be largely corrected by the C3P3-G3a system. This defect was not visible on the polysome profile of the first-generation C3P3 system. The mechanism of this finding is investigated in the following experiments.

### Example 2 Characterization of the Mechanisms Involved in the Translation Initiation Defect

### Example 2(a) RNA Interference Show that the Translation Initiation Defect Observed with the C3P3-G2 Artificial Expression System is Induced by Type I Interferon and Unfolded Protein Responses

Objectives

The objectives of these experiments were to investigate the mechanisms involved in the translation initiation defect observed in human cultured cells with the C3P3-G2 system. The C3P3-G1 system was also investigated. In addition, key candidate genes that could be involved in such translation initiation defect and could be targeted by viral proteins as selected in Examples 3 and 7, were sought. This was investigated using small interfering RNA (siRNA) to target key cell genes and with Firefly Luciferase reporter gene driven by the artificial C3P3 system expression as a readout.

Methods

Plasmids

The pC3P3-G1, pC3P3-G2 and pK1Ep-Luciferase-4xλBoxBr plasmids were described previously.

siRNA

Small interfering RNA (siRNA) is a class of non-coding dsRNA molecules of 20-25 base pairs in length with 3′-overhangs that operate within the RNA interference (RNAi) pathway to target RNA transcript for destruction.

RNA interference was performed using pools of four siRNA (Dharmacon, Lafayette, Colo., USA). Each pool consists of four chemically siRNA, which were designed in order to reduce off-targets by modifications of the sense strand to prevent interaction with RISC and favor antisense strand uptake, as well as modifications of antisense strand seed region to destabilize off-target activity and enhance target specificity (Birmingham, Anderson et al. 2006, Jackson, Burchard et al. 2006, Anderson, Birmingham et al. 2008).

The following pools of siRNA were targeting the following human genes: EIF2AK2 (NCBI GenBank accession number NM_002759), EIF2AK3 (NCBI GenBank accession number NM_004836), IRF3 (NCBI GenBank accession number NM_001571), IRF7 (NCBI GenBank accession number NM_004030), IRF9 (NCBI GenBank accession number NM_006084), JAK1 (NCBI GenBank accession number NM_002227), STAT1 (NCBI GenBank accession number NM_139266), STAT2 (NCBI GenBank accession number NM_005419), TYK2 (NCBI GenBank accession number NM_003331), DDX58 (NCBI GenBank accession number NM_014314), IFIH1 (NCBI GenBank accession number NM_022168), MAVS (NCBI GenBank accession number NM_020746), IFNAR1 (NCBI GenBank accession number NM_000629), IFNAR2 (NCBI GenBank accession number NM_207584) and IFNB1 (NCBI GenBank accession number NM_002176). A pool of four siRNAs designed and microarray tested for minimal targeting of human genome was used as negative control.

Cell Culture and Transfection

Human cells were cultured and cotransfected with the pC3P3-G1/pK1Ep-Luciferase-4xλBoxBr or pC3P3-G2/pK1Ep-Luciferase-4xλBoxBr plasmids as previously described. For standard luciferase and hSEAP gene reporter expression assays, cells were analyzed 48 hours after transfection. Pools of test and control siRNA were added to transfection reagent and used at a final concentration of 100 nM.

Firefly Luciferase Luminescence and SEAP Colorimetric Assays

Luciferase luminescence was assayed by the Luciferase Assay System (Promega, Madison, Wis.) according to the manufacturer's recommendations. In brief, cells were lysed in Cell Culture Lysis Reagent buffer (CLR), and then centrifuged at 12,000×g for two minutes at 4° C. Luciferase Assay Reagent (Promega; 100 μl/well) diluted at 1:10 was added to supernatant (20 μl/well). Luminescence readout was taken on a Tristar 2 microplate reader (Berthold, Bad Wildbad, Germany) with a read time of one second per well.

In order to normalize for transfection efficacy, cells were transfected with the pORF-eSEAP plasmid (InvivoGen, San Diego, Calif.), which encodes for the human secreted embryonic alkaline phosphatase (hSEAP) driven by the EF-1α/HTLV composite promoter. Enzymatic activity was assayed in cell culture medium using the Quanti-Blue colorimetric enzyme assay kit (InvivoGen). Gene reporter expression was expressed as the ratio of luciferase luminescence (RLU, relative light units) to eSEAP absorbance (OD, optic density).

Statistical Analysis

Student t-test was used for single comparison. For multiple comparisons, one-way ANOVA with

Dunnett's Post Hoc Test was used for testing the between means of the test groups with the control group. All data are presented as are means (n≥4)±standard deviation (SD). Statistical significance was set at P<0.05.

Results

In a first series of experiments, the effects of gene expression inhibition in human HEK-293 cells were investigated with the C3P3-G2 system.

Strong increase of Firefly Luciferase expression was found in cells transfected with EIF2AK2 (relative ratio of 2.68-fold vs. negative control cells, P<0.0001), and at lesser extend EIF2AK3 siRNA (relative ratio of 1.94-fold vs. negative control cells, P<0.0001). Both kinases phosphorylate eIF2α at serine 52 and thereby inhibit translation initiation. EIF2AK2 is activated by dsRNA, which is central trigger of type I-interferon response, while EIF2AK3 is activated by unfolded protein response, which is initiated by ER overload.

The effects of inhibition of key genes involved in type I-interferon response were also investigated using pools of siRNA. Inhibition of DDX58 and IFIH1 by siRNA increases by 2.17-fold and 1.38-fold the expression of Firefly Luciferase, respectively (P<0.0001 for both comparisons). Both factors are cytosolic RNA helicases that sense short and long dsRNA, respectively and trigger type-I interferon production, which in turn induces the expression of EIF2AK2 (Saito and Gale 2008). IRF3 and IFR7 siRNA also increases by 1.70 and 1.36-fold expression of Firefly Luciferase. IRFs are transcription factors that dimerize and translocate to the nucleus following DDX58/RIG-I or IFIH1/MDA5 activation, therefore resulting in type-I interferon response (Honda, Takaoka et al. 2006). The inhibition of interferon β by siRNA also increases the expression of Firefly Luciferase by 1.82-fold. Upon their release, interferons bind their ubiquitous heterodimeric membrane receptor which is composed of two subunits, referred to as the low affinity subunit, IFNAR1, and the high affinity subunit, IFNAR2 (Piehler, Thomas et al. 2012). The inhibition of expression of these subunits by specific siRNA increases by 1.21- and 1.23-fold the expression of Firefly Luciferase. IFN receptors transduce signals via the JAK-STAT pathway, which consists of the Janus tyrosine kinases such as JAK1 and TYK2, which in turn phosphorylate the transcription proteins STAT1 and STAT2 that associate with IRF9 to form a homo- or heterotrimeric transcription complex (Au-Yeung, Mandhana et al. 2013, Platanitis, Demiroz et al. 2019). Noticeably, inhibition of all factors in the JAK-STAT pathway increases the expression of Firefly Luciferase of 1.62-(JAK1), 1.33-(TYK2), 1.21-(STAT1), 1.14-(STAT1), and 1.19-fold (IRF9), respectively.

| | | Firefly Luciferase | |
|---|---|---|---|
| | siRNA | (mean ± SD); | |
| Conditions | reference | relative ratio | P-value |
| pC3P3-G2, pK1E-Luciferase-4xλBoxBL, siRNA:EIF2AK2 | L-003527-00 | 1 456 721 ± 20 541; 2.68 | <0.0001 |
| pC3P3-G2, pK1E-Luciferase-4xλBoxBL, siRNA:EIF2AK3 | L-004883-00 | 1 053 783 ± 21 830; 1.94 | <0.0001 |
| pC3P3-G2, pK1E-Luciferase-4xλBoxBL, siRNA:IRF3 | L-006875-00 | 923 733 ± 40 675; 1.70 | <0.0001 |
| pC3P3-G2, pK1E-Luciferase-4xλBoxBL, siRNA:IRF7 | L-011810-00 | 739 675 ± 24 448; 1.36 | <0.0001 |
| pC3P3-G2, pK1E-Luciferase-4xλBoxBL, siRNA:IRF9 | L-020858-00 | 648 538 ± 11 900; 1.19 | <0.0001 |
| pC3P3-G2, pK1E-Luciferase-4xλBoxBL, siRNA:JAK1 | L-003145-00 | 879 285 ± 30 622; 1.62 | <0.0001 |
| pC3P3-G2, pK1E-Luciferase-4xλBoxBL, siRNA:STAT1 | L-003543-00 | 658 428 ± 11 998; 1.21 | <0.0001 |
| pC3P3-G2, pK1E-Luciferase-4xλBoxBL, siRNA:STAT2 | L-012064-00 | 618 861 ± 9 585; 1.14 | <0.0001 |
| pC3P3-G2, pK1E-Luciferase-4xλBoxBL, siRNA:TYK2 | L-003182-00 | 720 392 ± 18 927; 1.33 | <0.0001 |
| pC3P3-G2, pK1E-Luciferase-4xλBoxBL, siRNA:DDX58 | L-012511-00 | 1 180 432 ± 25 457; 2.17 | <0.0001 |
| pC3P3-G2, pK1E-Luciferase-4xλBoxBL, siRNA:IFIH1 | L-013041-00 | 750 167 ± 7 974; 1.38 | <0.0001 |
| pC3P3-G2, pK1E-Luciferase-4xλBoxBL, siRNA:MAVS | L-024237-00 | 669 935 ± 23 610; 1.23 | <0.0001 |
| pC3P3-G2, pK1E-Luciferase-4xλBoxBL, siRNA:IFNAR1 | L-020209-00 | 651 619 ± 11 100; 1.20 | <0.0001 |
| pC3P3-G2, pK1E-Luciferase-4xλBoxBL, siRNA:IFNAR2 | L-015411-00 | 665 850 ± 17 500; 1.23 | <0.0001 |
| pC3P3-G2, pK1E-Luciferase-4xλBoxBL, siRNA:IFNB1 | L-019656-00 | 988 203 ± 27 864; 1.82 | <0.0001 |
| pC3P3-G2, pK1E-Luciferase-4xλBoxBL, ON-TARGETplus | LP_104738 | 543 084 ± 13 675; 1.00 | |
| Non-targeting Pool | | | |
| pC3P3-G2, empty plasmid, ON-TARGETplus Non-targeting Pool | | 37 847 ± 821; 0.07 | |
| Transfection reagent only | | 26 511 ± 821; 0.05 | |

In a second series of experiments, the effects of gene expression inhibition in human HEK-293 cells were investigated with the C3P3-G1 system with the same methodology as previously. Similar findings were obtained although less marked than with the C3P3-G2 system. Both EIF2AK2 and EIF2AK3 siRNAs increased the expression of Firefly Luciferase, thereby confirming the activation of the type !-interferon, and at lesser extend the unfolded protein responses. Moreover, inhibition of most of the key genes involved in type I-interferon response previously tested significantly increased Firefly Luciferase expression level, the greatest effect being observed with DDX58 siRNA, a key cytoplasmic sensor for dsRNA.

| | | Firefly Luciferase | |
|---|---|---|---|
| | siRNA | (mean ± SD); | |
| Conditions | reference | relative ratio | P-value |
| pC3P3-G1, pK1E-Luciferase-4xλBoxBL, siRNA:EIF2AK2 | L-003527-00 | 1 019 065 ± 14 370; 1.88 | <0.0001 |
| pC3P3-G1, pK1E-Luciferase-4xλBoxBL, siRNA:EIF2AK3 | L-004883-00 | 818 408 ± 16 954; 1.51 | <0.0001 |
| pC3P3-G1, pK1E-Luciferase-4xλBoxBL, siRNA:IRF3 | L-006875-00 | 714 264 ± 31 451; 1.32 | <0.0001 |
| pC3P3-G1, pK1E-Luciferase-4xλBoxBL, siRNA:IRF7 | L-011810-00 | 655 279 ± 21 658; 1.21 | <0.0001 |
| pC3P3-G1, pK1E-Luciferase-4xλBoxBL, siRNA:IRF9 | L-020858-00 | 600 286 ± 10 350; 1.11 | <0.001 |
| pC3P3-G1, pK1E-Luciferase-4xλBoxBL, siRNA:JAK1 | L-003145-00 | 679 860 ± 23 677; 1.25 | <0.0001 |
| pC3P3-G1, pK1E-Luciferase-4xλBoxBL, siRNA:STAT1 | L-003543-00 | 603 668 ± 11 000; 1.11 | <0.0001 |
| pC3P3-G1, pK1E-Luciferase-4xλBoxBL, siRNA:STAT2 | L-012064-00 | 533 121 ± 8 257; 0.98 | NS |
| pC3P3-G1, pK1E-Luciferase-4xλBoxBL, siRNA:TYK2 | L-003182-00 | 651 240 ± 17 110; 1.20 | <0.0001 |
| pC3P3-G1, pK1E-Luciferase-4xλBoxBL, siRNA:DDX58 | L-012511-00 | 908 279 ± 19 588; 1.67 | <0.0001 |
| pC3P3-G1, pK1E-Luciferase-4xλBoxBL, siRNA:IFIH1 | L-013041-00 | 594 715 ± 6 322; 1.10 | <0.001 |
| pC3P3-G1, pK1E-Luciferase-4xλBoxBL, siRNA:MAVS | L-024237-00 | 598 848 ± 21 105; 1.10 | <0.001 |
| pC3P3-G1, pK1E-Luciferase-4xλBoxBL, siRNA:IFNAR1 | L-020209-00 | 585 131 ± 9 967; 1.08 | <0.001 |
| pC3P3-G1, pK1E-Luciferase-4xλBoxBL, siRNA:IFNAR2 | L-015411-00 | 599 730 ± 15 763; 1.10 | <0.001 |
| pC3P3-G1, pK1E-Luciferase-4xλBoxBL, siRNA:IFNB1 | L-019656-00 | 755 455 ± 21 301; 1.39 | <0.0001 |
| pC3P3-G1, pK1E-Luciferase-4xλBoxBL | LP_104738 | 543 084 ± 13 675; 1.00 | <0.0001 |
| pC3P3-G1, empty plasmid | | 37 847 ± 821; 0.00 | |
| Transfection reagent only | | 26 511 ± 822; 0.00 | |

Conclusions

These RNA interference studies confirm that EIF2AK2, an effector of the type I interferon response, and EIF2AK3, a key effector of the unfolded protein response, are both activated by expression of the C3P3-G2, and at lesser extend C3P3-G1 system. Key genes involved in their pathway can be also identified.

### Example 2(b): Repression of EK2AK2 Activity by Small Molecule Increases Expression Levels of Reporter Gene by the C3P3-G2 and C3P3-G1 Systems

Objectives

The present study aims to confirm the activation of EIF2AK2 effectively repress expression levels by the C3P3 system, as suggested by the previous experiments. EIF2AK2 kinase was inhibited by small molecules acting as selective competitive inhibitors. As in the previous experiments, the C3P3-G1 and C3P3-G2 generations of the system were tested.

Methods

Plasmids

The pCMVScript-Luciferase, pC3P3-G1, pC3P3-G2 and pK1Ep-Luciferase-4xλBoxBr plasmids were described previously.

Chemicals

The EIF2AK2/PKR inhibitor CAS 608512-97-6 (Sigma-Aldrich) is an imidazolo-oxindole compound, which inhibits RNA-induced EIF2AK2 autophosphorylation (Jammi, Whitby et al. 2003). CAS 608512-97-6 binds ATP-binding site directed human with an IC50 of 210 nM.

Cell Culture and Transfection

Human cells were cultured and cotransfected with the pC3P3-G1/pK1Ep-Luciferase-4xλBoxBr or pC3P3-G2/pK1Ep-Luciferase-4xλBoxBr plasmids as previously described. For standard luciferase and hSEAP gene reporter expression assays, cells were analyzed 48 hours after transfection. The EIF2AK2 inhibitor was added to culture medium at the time of transfection and tested at concentrations ranging from 10 nM to 10 μM.

Firefly Luciferase Luminescence and SEAP Colorimetric Assays

Firefly luciferase luminescence was assayed from cell lysate as previously described.

Statistical Analysis

Statistical analyses were performed as previously described.

Results

The effects of EIF2AK2 inhibition by CAS 608512-97-6 were investigated in human HEK-293 cells expressing the Firefly Luciferase reporter gene under control of the C3P3-G1 and C3P3-G2 system. A dose-dependent increase of Firefly Luciferase expression was found with the C3P3-G1 (pC3P3-G1/pK1Ep-Luciferase-4xλBoxBr plasmids) and C3P3-G2 (pC3P3-G2/pK1Ep-Luciferase-4xλBoxBr plasmids) expression systems, when cells were treated with the CAS 608512-97-6 EIF2AK2 inhibitor. Maximum efficacy was found at 1 μM with the C3P3-G1 (relative ratio of 1.31-fold vs. negative control cells, P<0.0001) and C3P3-G2 (relative ratio of 1.67-fold vs. negative control cells, P<0.0001) expression systems.

| | Firefly Luciferase (mean ± SD); P-value | |
|---|---|---|
| | vs. no CAS 608512-97-6 (relative ratio) | |
| | pC3P3-G1, pK1Ep(G)- | pC3P3-G2, pK1Ep(G)- |
| | Luciferase-4xλBoxBl | Luciferase-4xλBoxBl |
| no CAS 608512-97-6 | 2 576 402 ± | 6 231 980 ± |
| | 172 961; NA (1.00) | 786 566; NA (1.00) |
| 10 nM CAS 608512-97-6 | 2 708 078 ± | 7 148 341 ± |
| | 159 871; NS (1.05) | 497 010; NS (1.15) |
| 100 nM CAS 608512-97-6 | 3 095 106 ± | 8 113 967 ± |
| | 424 730; <0.01 (1.20) | 451 546; <0.0001 (1.30) |
| 1 μM CAS 608512-97-6 | 3 380 978 ± | 10 387 086 ± |
| | 367 210; <0.0001 (1.31) | 180 533; <0.0001 (1.67) |
| 10 μM CAS 608512-97-6 | 3 228 759 ± | 9 317 870 ± |
| | 235 564; <0.0001 (1.25) | 488 998; <0.0001 (1.50) |
| Lipofectamine only | 829 ± 274 | 1 0427 ± 140 |

The effects of the inhibition of EIK2AK2 by the specific inhibitor CAS 608512-97-6 were also tested with the plasmid pCMVScript-Luciferase which allows conventional nuclear expression of the Firefly Luciferase reporter gene. As shown in the table below, no detectable effect was observed at any concentration ranging from 10 nM to 10 μM. These results suggest that phosphorylation of eIF2α by EIF2AK2 is not a limiting factor for expression by conventional nuclear expression systems.

| | | P-value vs. no |
|---|---|---|
| CAS 608512-97-6 | Firefly Luciferase | CAS 608512-97-6 |
| concentrations | (mean ± SD) | (relative ratio) |
| pCMVScript-Luciferase, no CAS 608512-97-6 | 2 576 402 ± 37 323 | NS (1) |
| pCMVScript-Luciferase, 10 nM CAS 608512-97-6 | 2 490 730 ± 158 348 | NS (0.97) |
| pCMVScript-Luciferase, 100 nM CAS 608512-97-6 | 2 542 067 ± 164 518 | NS (0.99) |
| pCMVScript-Luciferase, 1 μM CAS 608512-97-6 | 2 639 581 ± 64 324 | NS (1.02) |
| pCMVScript-Luciferase, 10 μM CAS 608512-97-6 | 2 640 768 ± 120 057 | NS (1.02) |
| Lipofectamine only | 32 093 ± 8 421 | NA |

Conclusions

Inhibition of EIF2AK2 by the specific antagonist CAS 608512-97-6 increases the expression of a reporter gene under control of first and second generation of the C3P3 artificial expression systems. These findings suggest that the phosphorylation of eIF2α by EIF2AK2 is a limiting factor for expression by the C3P3-G1 and C3P3-G2 systems, but not for conventional nuclear expression systems.

### Example 2(c): Phosphorylation of eIF2α Assessed by Western-Blot

Objectives

The objectives of this experiment were to assay by means of a direct method the level of eIF2 phosphorylation induced by the first- and second-generation expression systems. This was assessed by Western blotting, which makes it possible to quantify the rate of phosphorylated eIF2α protein and the total eIF2α protein.

Methods

HEK-293 cells were transfected as described above with plasmids allowing the expression of the reporter gene Firefly Luciferase by the artificial expression system of the first generation (pC3P3-G1/pK1Ep-Luciferase-4xλBoxBr), second generation (pC3P3-G2/pK1Ep-Luciferase-4xλBoxBr), second generation with dsRNA-binding domain from human EIF2AK2 (pC3P3-G2/phEIF2AK2:DRB/pK1Ep-Luciferase-4xλBoxBr), co-expression of C3P3-G2 and E3L:Zα-NS1:dsDNA-(G4S)2-sZIP as described in Example 5 (pC3P3-G2/pK1Ep-Luciferase-4xλBoxBr/pE3L:Zα-NS1:dsDNA-(G4S)2-sZIP), co-expression of C3P3-G2 and hEIF2AK2:DRB as described in Example 7 (pC3P3-G2/pK1Ep-Luciferase-4xλBoxBr/phEIF2AK2:DRB), and co-expression of C3P3-G2, hEIF2AK2:DRB and DP71L(I) as described in Example 8 (pC3P3-G2/pK1Ep-Luciferase-4xλBoxBr/phEIF2AK2:DRB/pDP71L(I)).

As a positive control, HEK-293 cells were transfected with pools of siRNA against the protein phosphatase 1 catalytic subunit alpha (PPP1CA, Dharmacon LQ-008927-00, NCBI GenBank accession number NM_002708.4) or its regulatory subunit PPP1R15A (GADD34, Dharmacon LQ-004442-02, NCBI GenBank accession number NM_014330.5). Cells treated with non-targeting pool of siRNA was used to assess off-target effects. Pools of test siRNA were added to transfection reagent and used at a final concentration of 100 nM.

Cells were lysed in 200 μl of CLR buffer and lysate was clarified by spinning for 15 sec at 12,000×g at room temperature. Twenty milligrams of total protein were resolved on 4-12% NuPAGE SDS-polyacrylamide gradient gel (Life Technologies, Carlsbad, Calif.), and subjected to western blotting onto nitrocellulose Hybond membrane (GE Healthcare, Pittsburgh, Pa.) overnight at +4° C.

To assess eIF2α phosphorylation, membranes with transferred proteins were blocked with 5% skim milk powder in PBS, then incubated with the rabbit IgG phospho-EIF2S1 (Ser52) polyclonal antibody 44-728G (1:1000; ThermoFisher) raised against human Ser52 phosphorylated eIF2α, then with anti-rabbit IgG-conjugated horseradish peroxidase NA9340V antibody (1:10000; GE Healthcare). Bands were visualized using the SuperSignal West Pico Chemiluminescent Substrate solution (Thermo Scientific) and scanned with the Fusion XPRESS gel imager (Vilber Lourmat, Marne-la-Vallée, France). Molecular weights were determined using the Novex Sharp pre-stained Protein Standard color markers (Thermo Fisher).

For total eIF2α assay, the membranes were then dehybridized, blocked with 5% skim milk powder in PBS, and reprobed with the rabbit IgG EIF2S1 polyclonal antibody AHO1182 raised against total human eIF2α protein (1:500; ThermoFisher), then analyzed by Western blotting as previously described.

Results

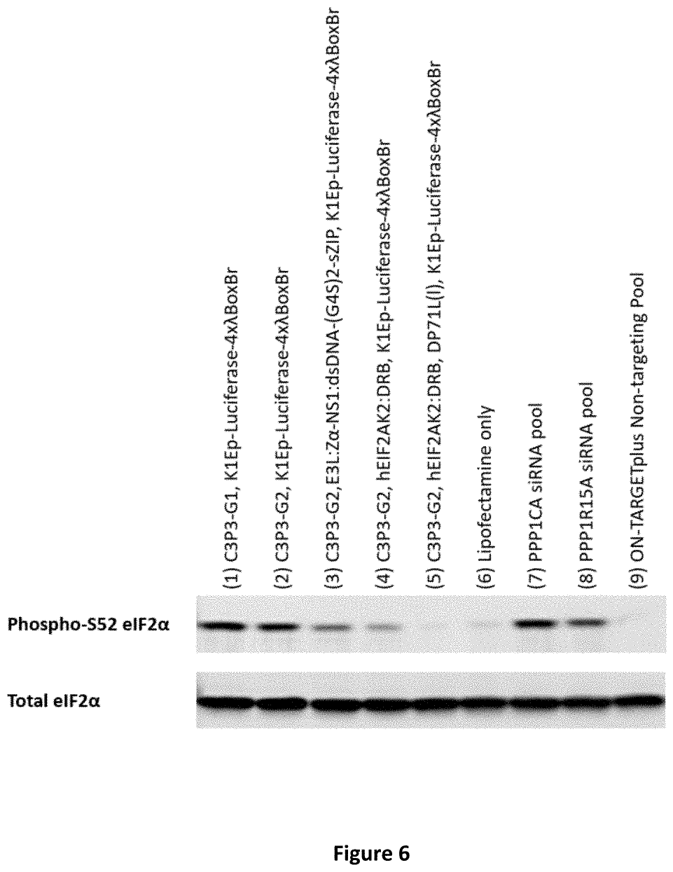

An increased rate of phosphorylation of eIF2α was found in cells transfected with the plasmids of the artificial expression system of the first generation (FIG. 6, track 1 vs. 6) and second generation C3P3 system (track 2 vs. 6), as compared to cells treated with the transfection agent only.

Conversely, the rate of phosphorylation was reduced with the C3P3-G2 expression system when the E3L:Zα-NS1:dsDNA-(G4S)2-sZIP artificial protein was co-expressed (FIG. 6, track 3 vs. 6). Likewise, decrease in the rate of phosphorylation of eIF2α was observed when dsRNA-binding domain from human EIF2AK2 was co-expressed alone (FIG. 6, track 4 vs. 6) or in combination with the DP71L(I) (FIG. 6, track 5 vs. 6).

Finally, pools of siRNA of the catalytic subunit of the phosphatase PPP1CA (FIG. 6, track 7 vs. 9) or of its regulatory subunit PPP1R15A (FIG. 6, track 8 vs. 9), was associated to an increase in the rate of phosphorylation of eIF2α compared to a non-targeting pool of siRNA.

Conclusions

This experiment shows an increase of phosphorylation of eIF2α induced by first- and second-generation artificial expression systems, which can be reserved by protein inhibitors.

### Example 3: Expression of Viral and Host-Cell Proteins and RNA Sequence Triggering the Type-I Interferon Pathway can Increase Expression Levels by the C3P3-G2 and C3P3-G1 Systems

Objectives

The objective of these experiments was to screen for viral proteins and RNA sequences able to inhibit type-I interferon response, which could increase expression of Firefly Luciferase reporter gene driven by the artificial C3P3-G1 and C3P3-G2 systems. This screening phase was secondarily extended to host-cell proteins also involved type-I interferon response.

Plasmids

The pC3P3-G1, pC3P3-G2 and pK1Ep-Luciferase-4xλBoxBr plasmids were described previously. Viral genes known or anticipated to interfere with the host-cell interferon response pathway or other related biological activities were subcloned in the pCMVScript plasmid backbone (Stratagene), following the removal of the T7 φ10 promoter sequence. These corresponding plasmids, designated as p-followed by the name of the ORF, have the following design: 1E1 promoter/enhancer from the human cytomegalovirus (CMV), 5′-untranslated region (5′-UTR), Kozak consensus sequence, selected open-reading frames, 3′-untranslated region (3′-UTR), and SV40 polyadenylation signal.

A first series test plasmids was synthetized, which consists of viral genes known to inhibit host-cell interferon response pathway. These genes have been selected to target host cellular proteins whose inhibition by siRNA has shown the most important effects:
EIF2AK2 was targeted by the long isoform of E3L protein from vaccinia virus (UniProtKB/Uniprot accession number P21081-1; pE3L-1NV) and its short (UniProtKB/Uniprot accession number P21081-2; pE3L-2NV) isoforms (Davies, Chang et al. 1993), NS1 protein (UniProtKB/Uniprot accession number P03496; pNS1/IAV) from Influenza A virus (Bergmann, Garcia-Sastre et al. 2000), K3L protein (UniProtKB/Uniprot accession number P18378; pK3L/VV) from vaccinia virus (Davies, Chang et al. 1993), NSs protein (UniProtKB/Uniprot accession number P21698; pNSs/RVFV) from Rift Valley fever virus, which promotes EIF2AK2 proteasomal degradation (Habjan, Pichlmair et al. 2009), and the dominant-negative mutant K296R of the human EIF2AK2 (UniProtKB/Uniprot accession number P19525; pEIF2AK2:K296R), which is inactive as a result of a mutation in the ATP-binding/phosphotransfer site (Katze, Wambach et al. 1991).
DDX58 was targeted by the VP35 from Zaire Ebolavirus (UniProtKB/Uniprot accession number Q05127; pVP35/EBOV), which can also caps the ends of dsRNA (Kimberlin, Bornholdt et al. 2010, Leung, Prins et al. 2010, Jiang, Ramanathan et al. 2011, Kowalinski, Lunardi et al. 2011),
IRF3 was targeted by the N(pro) (N-terminal autoprotease; UniProtKB/Uniprot accession number Q6Y4U2; pN(pro)/BVDV) from Bovine Viral Diarrhea Virus (Seago, Hilton et al. 2007, Peterhans and Schweizer 2013),
IFNAR1/IFNAR2 heterodimeric receptor was targeted by the B18R secreted protein from vaccinia virus (UniProtKB/Uniprot accession number P25213; pB18R/VV) that binds to type I interferons (Alcami, Symons et al. 2000),
JAK1 was targeted by the VP40 protein (UniProtKB/Uniprot accession number P35260; VP40/MBV) from the Marburg virus (Valmas and Basler 2011),
TYK2 was targeted by the LMP-1 protein (UniProtKB/Uniprot accession number P03230; LMP-1/EBV) from Epstein-Barr virus (Geiger and Martin 2006),
STAT1 was targeted by the V protein (UniProtKB/Uniprot accession number P11207; pV/PIV5) from parainfluenza virus type 5 (Didcock, Young et al. 1999, Precious, Carlos et al. 2007)
STAT2 was targeted by NS1 protein from human respiratory syncytial virus (UniProtKB/Uniprot accession number O42083; NS1/RSV) that mediates its proteasomal degradation (Elliott, Lynch et al. 2007),
IRF9 was targeted by μ2 protein (UniProtKB/Uniprot accession number Q00335; μ2/REOV) from reovirus that induces its nuclear accumulation (Zurney, Kobayashi et al. 2009).

In a second series, viral and host-cell proteins which recruit human PPP1CA (UniProtKB/Uniprot accession number P62136, pPPP1CA), the serine/threonine-protein Phosphatase 1 catalytic subunit, to dephosphorylate eIF2α were tested, including the human PPP1R15A (also known as GADD34, UniProtKB/Uniprot accession number O75807; pPPP1R15A), which the regulatory subunit of PPP1CA (Novoa, Zeng et al. 2001), DP71L(s) from African swine fever virus (UniProtKB/Uniprot accession number Q65212; pDP71L(s)/ASFV) (Afonso, Zsak et al. 1998, Zhang, Moon et al. 2010), and ICP34.5 from human Herpes-simplex virus-1 (UniProtKB/Uniprot accession number P36313; pICP34.5/HVS1) (Goatley, Marron et al. 1999).

In addition, the effects of 5′UTR RNA sequence from alphavirus Sindbis virus (strain ArB7761, Genbank ID MH212167.1) on expression of Firefly Luciferase was investigated by substituting 5′UTR of the pK1Ep-Luciferase-4xλBoxBr plasmid by the 5′UTR sequence from Sindbis virus (strain ArB7761, Genbank ID MH212167.1; pK1Ep-5′UTR/SINV-Luciferase-4xλBoxBr) (Hyde, Gardner et al. 2014, Reynaud, Kim et al. 2015).

Cell Culture and Transfection

Human cells were cultured as previously described. Cells were cotransfected with the pC3P3-G1/pK1Ep-Luciferase-4xλBoxBr or pC3P3-G2/pK1Ep-Luciferase-4xλBoxBr plasmids, together with the test plasmid listed above or an empty dummy plasmid to transfect the same amount of DNA to all conditions.

Firefly Luciferase Luminescence and SEAP Colorimetric Assays

Firefly luciferase luminescence was assayed from cell lysate as previously described.

Statistical Analysis

Statistical analyses were performed as previously described.

Results

In this first series of experiments, the level of expression of the Firefly Luciferase reporter gene expressed by the C3P3-G2 plasmid was investigated in presence several test plasmids encoding for viral proteins known to interfere directly or indirectly with the type-I interferon response.

All test plasmids encoding proteins that target EIF2AK2 statistically significantly increased the level of expression of the Firefly luciferase reporter gene. Greatest increase of nearly 3-fold was observed with the long isoform of E3L from vaccinia virus and at much lesser extend of 1.49-fold with its short isoform (P<0.0001 for both comparisons). This protein is well-characterized competitive inhibitor for the binding of dsRNA to EIF2AK2. NSs protein from Rift Valley fever virus increased by 2.63-fold the expression of the Firefly Luciferase reporter gene (P<0.0001). NS1 protein from Influenza A virus and K3L from vaccinia virus also statistically significantly increased the expression of Firefly Luciferase reporter gene (relative ratio of 2.63-fold and 1.68-fold, P<0.0001). In addition, the dominant-negative mutant K296R of the human EIF2AK2, which is inactive as a result of a mutation in the ATP-binding/phosphotransfer site increased substantially the expression level of Firefly Luciferase reporter gene (relative ratio of 2.63-fold, P<0.0001). This body of evidence confirms the crucial role of eIF2AK2 activation for expression by the artificial C3P3 expression system.

Other viral genes involved in type-I interferon pathway also statistically significantly increased the level of Firefly Luciferase reporter gene expression. They ranged in the following order: pVP35 (RIG-I inhibitor), N^{PRO} (IRF3 inhibitor), VP40 (JAK1 inhibitor), V protein (STAT1 inhibitor), LMP-1 (TYK2 inhibitor), μ2 (IRF9 inhibitor), NS1 from human respiratory syncytial virus (STAT2 inhibitor) and B18R (type I interferons inhibitor).

A second series of candidate genes was tested, which recruit the Protein Phosphatase 1 (PPP1CA) catalytic subunit that dephosphorylate eIF2α at Ser52, thereby reversing the shut-off of protein synthesis induced by eIF2 kinases. The expression of human PPP1R15, a host-cell protein that directs the catalytic PPP1CA subunit to its specific substrate, significantly increased Firefly Luciferase expression by 2.25-fold (P<0.0001). The herpes virus simplex 1 ICP34.5 protein (Mossman and Smiley 2002), which also recruit PPP1CA had similar efficacy to that of PPP1R15 with 2.16-fold increase of Firefly Luciferase expression, whereas DP71L(s) which also recruit PPP1CA has much reduced efficacy of only 1.24-fold (Barber, Netherton et al. 2017).

Finally, we tested the effects of the 5′-UTR from the genomic RNA of the Sindbis alphavirus, which antagonize IFIT1 (Hyde, Gardner et al. 2014, Reynaud, Kim et al. 2015). IFIT1 are effectors induced in response to type-I interferon, which sensors viral RNA that carries a triphosphate group on its 5′-terminus or 5′-cap lacking 2′-O methylation and induces type-I-interferon response (Abbas, Laudenbach et al. 2017). This genomic RNA sequence from the Sindbis alphavirus inserted in the 5′-UTR of the Firefly Luciferase gene reporter plasmid also increased its expression by 2.7-fold (P<0.0001).

| | Firefly Luciferase | |
|---|---|---|
| | (mean ± SD); | |
| Plasmids | relative ratio | P-value |
| pC3P3-G2, pE3L-1/VV, pK1E-Luciferase-4xλBoxBL | 3 312 609 ± 54 621; 2.98 | <0.0001 |
| pC3P3-G2, pK1Ep-5′UTR/SINV-Luciferase-4xλBoxBL | 2 947 342 ± 83 023; 2.66 | <0.0001 |
| pC3P3-G2, pNSs/RVFV, pK1E-Luciferase-4xλBoxBL | 2 924 273 ± 100 916; 2.63 | <0.0001 |
| pC3P3-G2, pEIF2AK2:K296R, pK1E-Luciferase-4xλBoxBL | 2 923 612 ± 91 169; 2.63 | <0.0001 |
| pC3P3-G2, pPPP1R15A, pK1E-Luciferase-4xλBoxBL | 2 498 553 ± 35 329; 2.25 | <0.0001 |
| pC3P3-G2, pICP34.5/HVS1, pK1E-Luciferase-4xλBoxBL | 2 394 434 ± 97 118; 2.16 | <0.0001 |
| pC3P3-G2, pNS1/IAV, pK1E-Luciferase-4xλBoxBL | 2 311 610 ± 74 121; 2.08 | <0.0001 |
| pC3P3-G2, pVP35/EBOV, pK1E-Luciferase-4xλBoxBL | 2 039 576 ± 68 123; 1.84 | <0.0001 |
| pC3P3-G2, pNPRO/BVDV, pK1E-Luciferase-4xλBoxBL | 1 901 080 ± 59 654; 1.71 | <0.0001 |
| pC3P3-G2, pK3L/VV, pK1E-Luciferase-4xλBoxBL | 1 859 375 ± 62 784; 1.68 | <0.0001 |
| pC3P3-G2, VP40/MBV, pK1E-Luciferase-4xλBoxBL | 1 782 322 ± 38 538; 1.61 | <0.0001 |
| pC3P3-G2, pV/PIV5, pK1E-Luciferase-4xλBoxBL | 1 750 866 ± 37 434; 1.58 | <0.0001 |
| pC3P3-G2, LMP-1/EBV, pK1E-Luciferase-4xλBoxBL | 1 721 541 ± 56 310; 1.55 | <0.0001 |
| pC3P3-G2, pE3L-2/VV, pK1E-Luciferase-4xλBoxBL | 1 648 821 ± 59 950; 1.49 | <0.0001 |
| pC3P3-G2, μ2/REOV, pK1E-Luciferase-4xλBoxBL | 1 402 716 ± 48 746; 1.26 | <0.0001 |
| pC3P3-G2, NS1/RSV, pK1E-Luciferase-4xλBoxBL | 1 396 909 ± 50 590; 1.26 | <0.0001 |
| pC3P3-G2, DP71L(s)/ASFV, pK1E-Luciferase-4xλBoxBL | 1 379 443 ± 57 682; 1.24 | <0.0001 |
| pC3P3-G2, pB18R/VV, pK1E-Luciferase-4xλBoxBL | 1 363 247 ± 30 232; 1.23 | <0.0001 |
| pC3P3-G2, pK1E-Luciferase-4xλBoxBL | 1 109 854 ± 8 750; 1.00 | <0.0001 |
| pC3P3-G2, empty plasmid | 13 499 ± 228; 0.01 | <0.0001 |
| Transfection reagent only | 8 890 ± 150; 0.01 | <0.0001 |

The effects of test plasmids driven by the C3P3-G1 system were investigated with the same methodology as above. Similar findings were observed although less marked than with the C3P3-G2 system. All test plasmids encoding proteins that target EIF2AK2, except the short isoform of E3L and K3L, statistically significantly increased the level of expression of the Firefly luciferase reporter gene, with the greatest increase observed with the long isoform of E3L from vaccinia virus (relative ratio of 0.69-fold; P<0.0001).

Most of the previous test genes that target the type-I interferon pathway also statistically significantly increased the expression of the Firefly Luciferase reporter gene, except DP71L(s), LMP-1, NS1, μ2 and B18R. Finally, the 5′-UTR from the genomic RNA of the Sindbis alphaviruses also statistically significantly increased the expression of the Firefly Luciferase reporter gene.

| | Firefly Luciferase | |
|---|---|---|
| | (mean ± SD); | |
| Plasmids | relative ratio | P-value |
| pC3P3-G1, pE3L-1/VV, pK1E-Luciferase-4xλBoxBL | 1 938 320 ± 80 126; 1.69 | <0.0001 |
| pC3P3-G1, pK1Ep-5′UTR/SINV-Luciferase-4xλBoxBL | 1 810 266 ± 22 395; 1.58 | <0.0001 |
| pC3P3-G1, pNSs/RVFV, pK1E-Luciferase-4xλBoxBL | 1 646 400 ± 39 468; 1.44 | <0.0001 |
| pC3P3-G1, pN(pro)/BVDV, pK1E-Luciferase-4xλBoxBL | 1 371 893 ± 23 469; 1.20 | <0.0001 |
| pC3P3-G1, pEIF2AK2:K296R, pK1E-Luciferase-4xλBoxBL | 1 520 092 ± 32 013; 1.33 | <0.0001 |
| pC3P3-G1, pE3L-2/VV, pK1E-Luciferase-4xλBoxBL | 1 209 281 ± 30 214; 1.06 | NS |
| pC3P3-G1, pV/PIV5, pK1E-Luciferase-4xλBoxBL | 1 252 842 ± 42 091; 1.09 | <0.01 |
| pC3P3-G1, pPPP1R15A, pK1E-Luciferase-4xλBoxBL | 1 500 449 ± 56 022; 1.31 | <0.0001 |
| pC3P3-G1, pICP34.5/HVS1, pK1E-Luciferase-4xλBoxBL | 1 423 032 ± 54 867; 1.24 | <0.0001 |
| pC3P3-G1, pNS1/IAV, pK1E-Luciferase-4xλBoxBL | 1 520 613 ± 37 328; 1.33 | <0.0001 |
| pC3P3-G1, pK3L/VV, pK1E-Luciferase-4xλBoxBL | 1 166 878 ± 24 155; 1.02 | NS |
| pC3P3-G1, DP71L(s)/ASFV, pK1E-Luciferase-4xλBoxBL | 1 072 925 ± 14 178; 0.94 | NS |
| pC3P3-G1, pVP35/EBOV, pK1E-Luciferase-4xλBoxBL | 1 418 038 ± 28 761; 1.24 | <0.0001 |
| pC3P3-G1, VP40/MBV, pK1E-Luciferase-4xλBoxBL | 1 339 210 ± 26 474; 1.17 | <0.0001 |
| pC3P3-G1, LMP-1/EBV, pK1E-Luciferase-4xλBoxBL | 1 203 105 ± 32 203; 1.05 | NS |
| pC3P3-G1, NS1/RSV, pK1E-Luciferase-4xλBoxBL | 954 663 ± 20 205; 0.83 | <0.0001 |
| pC3P3-G1, μ2/REOV, pK1E-Luciferase-4xλBoxBL | 1 049 444 ± 40 645; 0.92 | <0.01 |
| pC3P3-G1, pB18R/VV, pK1E-Luciferase-4xλBoxBL | 1 005 633 ± 35 402; 0.88 | <0.0001 |
| pC3P3-G1, pK1E-Luciferase-4xλBoxBL | 1 145 467 ± 47 573; 1.00 | |
| pC3P3-G1, empty plasmid | 10 685 ± 5 716; 0.01 | |
| Transfection reagent only | 10 935 ± 3 918; 0.01 | |

Noticeably, some differences in the activity of these proteins were observed depending on the types of mammalian cell lines tested. For example, a statistically significant efficacy has been observed with K3L from vaccinia virus using the C3P3-G1 and C3P3-G2 systems in monkey kidney cells COS-1 (increased expression of the Firefly Luciferase reporter gene of 1.15 and 1.28-fold with C3P3-G1 and C3P3-G2, respectively; P<0.01 and P<0.0001) and human hepatocellular carcinoma cells HepG2 (increased expression of the Firefly Luciferase reporter gene of 1.29- and 1.43-fold with C3P3-G1 and C3P3-G2, respectively, P<0.0001 for both comparisons). Similarly, a significant increase in expression was observed with DP71L (s) from African swine fever virus in human HeLa cells (increased expression of the Firefly Luciferase reporter gene of 1.12 and 1.26-fold with C3P3-G1 and C3P3-G2, respectively; P<0.05 and P<0.01) and monkey kidney cells COS-1 (increased expression of the Firefly Luciferase reporter gene of 1.30 and 1.42-fold with C3P3-G1 and C3P3-G2 systems, respectively; P<0.0001 for both comparisons). A statistically significant effect of LMP-1 from Epstein-Barr virus was also found with C3P3-G1 and C3P3-G2 systems in the human B myelomonocytic leukemia cell line MV-4-11 (increased expression of the Firefly Luciferase reporter gene of 1.32 and 1.58-fold with C3P3-G1 and C3P3-G2, respectively; P<0.0001 for both comparisons). These results are consistent with previous findings of other investigators, showing that the degree of biological activity of certain viral proteins interacting with the interferon response are dependent on that of the host-cell and therefore differ from one cell type and more generally from one species to another (Langland and Jacobs 2002, Park, Peng et al. 2020).

Conclusions

Experiments show that various viral or cellular proteins, as well as certain viral RNA sequences, involved in the type-I interferon response can significantly increase expression with the artificial C3P3 expression system. The best results were obtained with the long isoform of the E3L protein of vaccinia virus, which was selected for the following protein engineering shown in Example 4.

### Example 4: An Artificial Protein Generated from E3L Protein Scaffold can Increase Expression Levels by the C3P3 System

Objectives

The aim of this series of experiments is to develop an artificial protein using the E3L protein of vaccinia virus as a scaffold, in order to further increase the levels of expression by the C3P3 system.

Methods

Plasmids

The pC3P3-G2 and pK1Ep-Luciferase-4xλBoxBr plasmids were described previously.

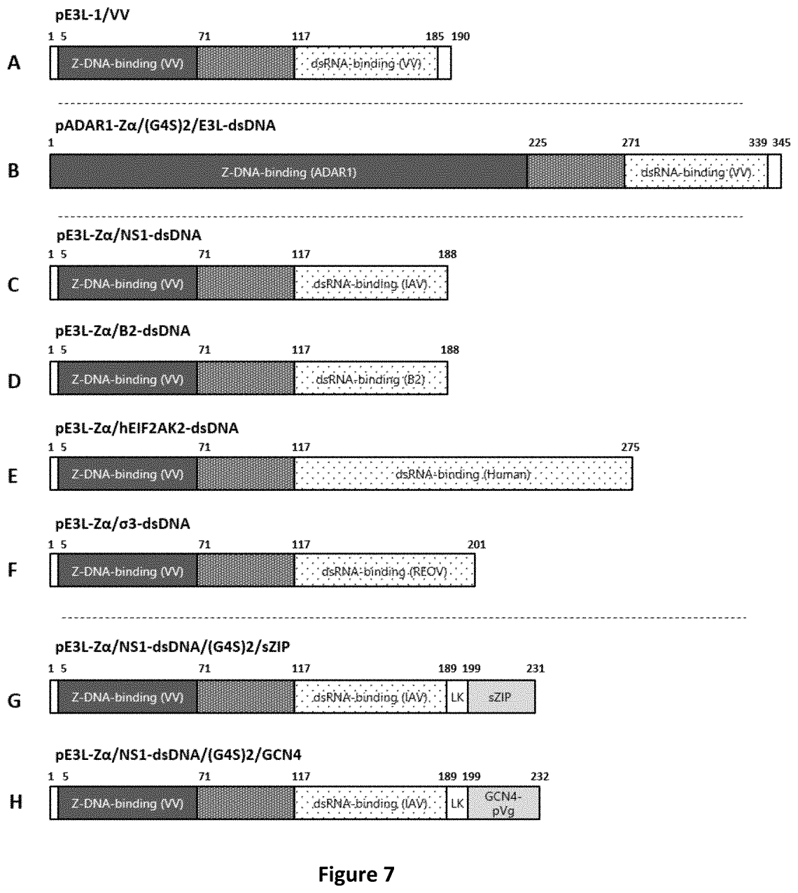

The vaccinia virus E3L protein contains two distinct domains: one Zα binding domain (also named Zα domain) at its amino-terminal extremity and a single dsRNA-binding domain at its carboxy-terminal end (FIG. 7A). These two domains, which are separated by a protein region with no distinct structure or function. In a first series of protein engineering, each of the domains of E3L were substituted by other domains having similar functional activities:
The amino-terminal Zα binding domain was substituted by the Zα binding domain at amino-terminal end of human ADAR1 protein (UniProtKB/Uniprot accession number P55265; Zα domain from human ADAR1, fused to the dsRNA-binding domain from E3L of vaccinia virus, through a linker: pADAR1-Zα/(G4S)2/E3L-dsDNA (SEQ ID NO. 5 and SEQ ID NO. 6; pADAR1-Zα/(G4S)2/E3L-dsDNA plasmid; FIG. 7B), which contains two Zα binding domain in tandem (Schwartz, Rould et al. 1999) though a flexible (G4S)2 linker,
The carboxyl-terminal dsRNA was substituted by several other dsRNA-binding domains from Influenza A virus NS1 protein (UniProtKB/Uniprot accession number P03496; SEQ ID NO. 7 and SEQ ID NO. 8; pE3L-Zα/NS1-dsDNA plasmid; FIG. 7C) (Bergmann, Garcia-Sastre et al. 2000), Flock House virus B2 protein (UniProtKB/Uniprot accession number P68831; SEQ ID NO. 9 and SEQ ID NO. 10; pE3L-Zα/B2-dsDNA plasmid; FIG. 7D) (Lingel, Simon et al. 2005), the amino-terminal region of human EIF2AK2 (UniProtKB/Uniprot accession number P19525; SEQ ID NO. 11 and SEQ ID NO. 12; pE3L-Zα/hEIF2AK2-dsDNA plasmid; FIG. 7E), which contains two dsRNA binding motifs separated by a short spacer (Patel and Sen 1992), and the orthoreovirus structural σ3 protein (UniProtKB/Uniprot accession number P07939; SEQ ID NO. 13 and SEQ ID NO. 14; pE3L-Zα/σ3-dsDNA plasmid; FIG. 7F) (Olland, Jane-Valbuena et al. 2001).

The E3L-Zα/NS1-dsDNA was selected from previous series of experiments and further optimized in a second series of protein engineering. Unlike the wild-type E3L protein, which can dimerize through its carboxyl-terminal region or even to form high order multimers at low ionic strength (Ho and Shuman 1996), the artificial protein E3L-Zα/NS1-dsDNA lacks known dimerization domain. To generate dimerization or multimerization of this protein, two different leucine zippers were introduced at the carboxy-terminal extremity of the artificial E3L-Zα/NS1-dsDNA protein:
Super leucine zipper (sLZ), which can homodimerize in parallel orientation through its super long coiled coil helix (SEQ ID NO. 15 and SEQ ID NO. 16; pE3L-Zα/NS1-dsDNA/(G4S)2/sZIP; FIG. 7G) (Harbury, Zhang et al. 1993, Harbury, Kim et al. 1994),
GCN4-pVg leucine zipper, which can homotetramerize in antiparallel orientation (SEQ ID NO. 17 and SEQ ID NO. 18; pE3L-Zα/NS1-dsDNA/(G4S)2/GCN4; FIG. 7H) (Pack, Kujau et al. 1993, Pluckthun and Pack 1997).

Cell Culture and Transfection

Human cells were cultured and transfected as previously described.

Firefly Luciferase Luminescence and SEAP Colorimetric Assays

Firefly luciferase luminescence was assayed from cell lysate as previously described.

Statistical Analysis

Statistical analyses were performed as previously described.

Results

The vaccinia virus E3L protein contains two distinct domains (FIG. 7A), which are both necessary to inhibit the interferon response (White and Jacobs 2012). First, its amino-terminal region (residues 5-70) contains a Z-DNA-binding domain. Second, the carboxyl-terminal region (residues 117-185) of E3L has typical dsRNA-binding domain, which binds to and sequesters as a homodimer dsRNA synthesized during viral infection (Ho and Shuman 1996). Such binding mask the dsRNA thereby preventing recognition and subsequent activation of EIF2AK2. Finally, a region with no distinct structure or function separates these two functional domains (residues 71-116).

The substitution of the amino-terminal Zα-binding domain of the wild-type E3L protein by the amino-terminal end of human ADAR1 protein, which contains two Zα binding domains, results in a functional protein that increased the expression of the Firefly Luciferase reporter, although at statistically lower level than that of the wild-type E3L protein (relative ratio of 1.74-fold vs. no test plasmid; P<0.0001).

This result is consistent with that of other authors who have shown that the existence of the Zα functional domain is essential for its biological activity of the E3L protein involved in the virulence of the vaccinia virus and could be substituted by the Zα binding domain from ADAR1 (Kim, Muralinath et al. 2003). The dsRNA-binding domain of E3L was then substituted by others from other human and viral proteins. Greatest increased expression levels of the Firefly Luciferase reporter gene were observed with two dsRNA-binding domain substitutions, i.e. NS1 protein from Influenza A virus and human EIF2AK2 (relative ratio of 3.11-fold and 3.02-fold vs. no test plasmid, respectively; P<0.0001 for both comparisons). In addition, the expression levels with these two test plasmids were statistically greater than with the wild-type E3L plasmid (relative ratio of 3.11-fold and 3.02-fold vs. 2.73, respectively; P<0.0001 for both comparisons). The two other substitutions by the B2 and σ3 dsRNA-binding domain were also functional, but to a lesser extent than the wild-type E3L plasmid.

To further optimize the E3L-Zα/NS1-dsDNA, this artificial protein was engineered by grafting leucine zippers at its carboxyl-terminal end through a flexible (G4S)2 linker. Leucine zippers are coiled-coil protein structures composed of two amphipathic α-helices that interact with each other and are commonly used to homo- or hetero-di/multimerize proteins (O'Shea, Klemm et al. 1991). Each helix consists of repeats of seven amino acids, in which the first amino-acid (residue a) is hydrophobic, the fourth (residue d) is usually a Leucine, while the other residues are polar. The super leucine zipper (sZIP), which can form homodimers in parallel orientation, increased significantly the expression of Firefly Luciferase in comparison to the E3L-Zα/NS1-dsDNA protein (relative ratio of 3.62-fold vs. 3.11-fold; P<0.0001). In contrast, the addition of the GCN4-pVg leucine zipper, which forms homotetramers in antiparallel orientation, had no detectable effects in comparison to the E3L-Zα/NS1-dsDNA protein (relative ratio of 3.01-fold vs. 3.11-fold; P=NS).

| | Firefly Luciferase | |
|---|---|---|
| | (mean ± SD); | P-value vs. |
| Plasmids | ratio | pE3L-1 |
| pC3P3-G2, pE3L-1, pK1E- | 3 940 065 ± 76 802; 2.73 | — |
| Luciferase-4xλBoxBL | | |
| pC3P3-G2, pADAR1-Zα/ | 2 382 402 ± 71 147; 1.65 | <0.0001 |
| (G4S)2/E3L-dsDNA, pK1E- | | |
| Luciferase-4xλBoxBL | | |
| pC3P3-G2, pE3L-Zα/ | 4 494 430 ± 90 526; 3.11 | <0.0001 |
| NS1-dsDNA, pK1E- | | |
| Luciferase-4xλBoxBL | | |
| pC3P3-G2, pE3L-Zα/ | 2 518 407 ± 34 326; 1.74 | <0.0001 |
| B2-dsDNA, pK1E- | | |
| Luciferase-4xλBoxBL | | |
| pC3P3-G2, pE3L-Zα/ | 4 358 392 ± 89 250; 3.02 | <0.0001 |
| hEIF2AK2-dsDNA, pK1E- | | |
| Luciferase-4xλBoxBL | | |
| pC3P3-G2, pE3L-Zα/ | 3 132 092 ± 57 352; 2.17 | <0.0001 |
| σ3-dsDNA, pK1E- | | |
| Luciferase-4xλBoxBL | | |
| pC3P3-G2, pE3L-Zα/ | 5 236 071 ± 191 878; 3.62 | <0.0001 |
| NS1-dsDNA/(G4S)2/sZIP, | | |
| pK1E-Luciferase-4xλBoxBL | | |
| pC3P3-G2, pE3L-Zα/ | 4 346 079 ± 179 900; 3.01 | <0.0001 |
| NS1-dsDNA/(G4S)2/GCN4, | | |
| pK1E-Luciferase-4xλBoxBL | | |
| pC3P3-G2, pK1E-Luciferase- | 1 444 957 ± 49 361; 1.00 | <0.0001 |
| 4xλBoxBL | | |
| pC3P3-G2, empty plasmid | 28 979 ± 527; 0.02 | |
| Transfection reagent only | 17 326 ± 385; 0.01 | |

Conclusions

These experiments shown that an artificial protein with greater activity than that of the long isoform of the E3L protein can be engineered. The artificial E3L-Zα/NS1-dsDNA/(G4S)2/sZIP protein was selected for further development.

### Example 5: The Co-Expression of the Artificial E3L-Zα/NS1-dsDNA/(G4S)2/sZIP, Together with Other Proteins or RNA Sequences Tested in Example 3, can Even Increase Expression Levels by the C3P3 Expression System

Objectives

The objective of this series of experiments is to test the additivity of the coexpression of pE3L-Zα/NS1-dsDNA/(G4S)2/sZIP with plasmids encoding other proteins or RNA sequences previously tested.

Methods

Plasmids

All plasmids were described in the above examples.

Cell Culture and Transfection

Human cells were cultured and transfected as previously described.

Firefly Luciferase Luminescence and SEAP Colorimetric Assays

Firefly luciferase luminescence was assayed from cell lysate as previously described.

Statistical Analysis

Statistical analyses were performed as previously described.

Results

A possible additive effect on the expression of the reporter gene Firefly Luciferase expressed by the C3P3-G2 system was tested by co-transfection of the previous test plasmids, together with the plasmid pE3L-Zα/NS1-dsDNA/(G4S)2/sZIP.

The results of the table below show successively the effect of each of the two plasmids transfected separately, then of the two plasmids co-transfected simultaneously. A supra-additive effect (SA) is defined as being at a statistically higher level of expression than by the simple addition of the effects tested separately, i.e. pE3L-Zα/NS1-dsDNA/(G4S)2/sZIP on one hand and test plasmid on the other hand. The infra-additive effect (IA) and the strict additive effects (SA) were defined respectively as a statistically lower or non-different expression compared to the simple addition of the effects tested separately.

Supra-additive effect, was observed with plasmids encoding for the N(pro) from Bovine Viral Diarrhea Virus that target IRF3 (P<0.05), NSs from Rift Valley fever virus that promotes EIF2AK2 proteasomal degradation (P<0.001) and 5′UTR from Sindbis viral genome (P<0.001), together with the pE3L-Zα/NS1-dsDNA/(G4S)2/sZIP plasmid.

Infra-additive (IA) or strict additive (SA) effects, which were defined as on a statistically lower or not different levels of expression than the addition of the individual effects, were observed with all other test plasmids.

| | Firefly Luciferase |
|---|---|
| | (mean ± SD); |
| Plasmids | relative ratio |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, VP40/MBV, pK1E-Luciferase-4xλBoxBL | 2 001 557 ± 43 278; 1.80 |
| 1 + 2) pC3P3-G2, VP40/MBV, pE3L-Zα/ | 3 472 301 ± 75 079; 3.13 (IA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, pEIF2AK2:K296R, pK1E-Luciferase-4xλBoxBL | 2 924 887 ± 97 692; 2.64 |
| 1 + 2) pC3P3-G2, pEIF2AK2:K296R, pE3L-Zα/ | 5 202 600 ± 173 769; 4.69 (IA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, pVP35/EBOV, pK1E-Luciferase-4xλBoxBL | 2 120 069 ± 70 811; 1.91 |
| 1 + 2) pC3P3-G2, pVP35/EBOV, pE3L-Zα/ | 3 679 190 ± 122 886; 3.32 (IA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, pV/PIV5, pK1E-Luciferase-4xλBoxBL | 2 031 546 ± 43 435; 1.83 |
| 1 + 2) pC3P3-G2, pV/PIV5, pE3L-Zα/ | 4 000 943 ± 85 541; 3.60 (IA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, pPPP1R15A, pK1E-Luciferase-4xλBoxBL | 2 642 205 ± 37 360; 2.38 |
| 1 + 2) pC3P3-G2, pPPP1R15A, pE3L-Zα/ | 5 670 692 ± 80 182; 5.11 (IA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, pNSs/RVFV, pK1E-Luciferase-4xλBoxBL | 3 333 228 ± 115 029; 3.00 |
| 1 + 2) pC3P3-G2, pNSs/RVFV, pE3L-Zα/ | 9 000 800 ± 310 617; 8.11 (SA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, pN(pro)/BVDV, pK1E-Luciferase-4xλBoxBL | 2 208 016 ± 69 286; 1.99 |
| 1 + 2) pC3P3-G2, pN(pro)/BVDV, pE3L-Zα/ | 6 649 423 ± 208 654; 5.99 (SA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, pK3L/VV, pK1E-Luciferase-4xλBoxBL | 1 976 221 ± 66 730; 1.78 |
| 1 + 2) pC3P3-G2, pK3L/VV, pE3L-Zα/ | 2 885 041 ± 97 417; 2.60 (IA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, pK1Ep-5′UTR/SINV-Luciferase-4xλBoxBL | 3 257 540 ± 91 760; 2.94 |
| 1 + 2) pC3P3-G2, pE3L-Zα/NS1-dsDNA/(G4S)2/LZ, | 9 017 711 ± 254 016; 8.13 (SA) |
| pK1Ep-5′UTR/SINV-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, pICP34.5/HVS1, pK1E-Luciferase-4xλBoxBL | 2 568 071 ± 104 161; 2.31 |
| 1 + 2) pC3P3-G2, pICP34.5/HVS1, pE3L-Zα/ | 6 875 963 ± 278 889; 6.20 (SA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, pDP71L(s)/ASFV, pK1E-Luciferase-4xλBoxBL | 1 438 249 ± 60 141; 1.30 |
| 1 + 2) pC3P3-G2, pDP71L(s)/ASFV, pE3L-Zα/ | 4 253 506 ± 177 863; 3.83 (IA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, pB18R/VV, pK1E-Luciferase-4xλBoxBL | 1 600 590 ± 35 496; 1.44 |
| 1 + 2) pC3P3-G2, pB18R/VV, pE3L-Zα/ | 4 245 040 ± 94 141; 3.82 (IA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, NS1/RSV, pK1E-Luciferase-4xλBoxBL | 1 531 635 ± 55 469; 1.38 |
| 1 + 2) pC3P3-G2, NS1/RSV, pE3L-Zα/ | 4 142 101 ± 150 009; 3.73 (IA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, LMP-1/EBV, pK1E-Luciferase-4xλBoxBL | 1 818 683 ± 59 488; 1.64 |
| 1 + 2) pC3P3-G2, LMP-1/EBV, pE3L-Zα/ | 4 209 742 ± 137 698; 3.79 (IA) |
| NS1-dsDNA/(G4S)2/LZ, pK1E-Luciferase-4xλBoxBL | |
| 1) pC3P3-G2, pE3L-Zα/NS1-dsDNA/(G4S)2/sZIP, | 4 085 116 ± 149 701; 3.68 |
| pK1E-Luciferase-4xλBoxBL | |
| 2) pC3P3-G2, μ2/REOV, pK1E-Luciferase-4xλBoxBL | 1 669 720 ± 58 025; 1.50 |
| 1 + 2) pC3P3-G2, μ2/REOV, pE3L-Zα/ | 3 317 346 ± 115 282; 2.99 (IA) |
| NS1-dsDNA/(G4S)2/LZ, pK1E-Luciferase-4xλBoxBL | |
| pC3P3-G2, pK1E-Luciferase-4xλBoxBL | 1 109 854 ± 8 750; 1.00 |
| pC3P3-G2, empty plasmid | 13 499 ± 228; 0.01 |

Conclusions

These results show synergistic effects between the E3L-Zα/NS1-dsDNA/(G4S)2/sZIP protein and the Nss and N(pro) proteins, as well as with the sequence 5′UTR of the Sindbis virus genome, on the expression of the Firefly Luciferase reporter gene expressed with the C3P3-G2 system.

### Example 6: Assembly of the Artificial E3L-Zα/NS1-dsDNA/(G4S)2/sZIP within the C3P3 Enzyme Results in Active Polyproteins

Objectives

The objective of the following experiment was to assemble in frame the artificial E3L-Zα/NS1-dsDNA/(G4S)2/sZIP within the open-reading frame of the C3P3-G2 enzyme.

Methods

Plasmids

The assemblies tested hereinafter are designed according to two protein scaffolds:
either Nλ-mPAPOLA-[X1]-E3L-Zα/NS1-dsDNA/(G4S)2/sZIP-[X2]-NP868R-(G4S)2-K1ERNAP,
or E3L-Zα/NS1-dsDNA/(G4S)2/sZIP-[X3]-Nλ-mPAPOLA-F2A-NP868R-(G4S)2-K1ERNAP,
   where [X1], [X2] and [X3] are variable. Each of the [X] positions correspond either to a (G4S)2 flexible linker or to an F2A ribosomal skipping motif.

The resulting proteins were named C3P3-G3x, where is the numbering of the construction:
C3P3-G3a, where [X1]=F2A and [X2]=F2A (SEQ ID NO. 19 and SEQ ID NO. 20; FIG. 8C), i.e. Nλ-mPAPOLA-F2A-E3L-Zα/NS1-dsDNA/(G4S)2/sZIP-F2A-NP868R-(G4S)2-K1ERNAP,
C3P3-G3b, where [X1]=(G4S)2 and [X2]=F2A (SEQ ID NO. 21 and SEQ ID NO. 22; FIG. 8D), i.e. Nλ-mPAPOLA-(G4S)2-E3L-Zα/NS1-dsDNA/(G4S)2/sZIP-F2A-NP868R-(G4S)2-K1ERNAP,
C3P3-G3c, where [X1]=F2A and [X2]=(G4S)2 (SEQ ID NO. 23 and SEQ ID NO. 24; FIG. 8E), i.e. Nλ-mPAPOLA-F2A-E3L-Zα/NS1-dsDNA/(G4S)2/sZIP-(G4S)2-NP868R-(G4S)2-K1ERNAP
C3P3-G3d, where [X3]=F2A (SEQ ID NO. 25 and SEQ ID NO. 26; FIG. 8F), i.e. E3L-Zα/NS1-dsDNA/(G4S)2/sZIP-F2A-Nλ-mPAPOLA-F2A-NP868R-(G4S)2-K1ERNAP),
C3P3-G3e, where [X3]=(G4S)2 (SEQ ID NO. 27 and SEQ ID NO. 28; FIG. 8G), i.e. E3L-Zα/NS1-dsDNA/(G4S)2/sZIP-(G4S)2-Nλ-mPAPOLA-F2A-NP868R-(G4S)2-K1ERNAP.

Cell Culture and Transfection

Human cells were cultured and transfected as previously described.

Firefly Luciferase Luminescence and SEAP Colorimetric Assays

Firefly luciferase luminescence was assayed from cell lysate as previously described.

Statistical Analysis

Statistical analyses were performed as previously described.

Results

The E3L-Zα/NS1-dsDNA/(G4S)2/SZIP coding sequence was inserted in-frame into the open-reading frame of the C3P3-G2 enzyme. The E3L-Zα/NS1-dsDNA/(G4S)2/SZIP coding sequence could be readily inserted at only two positions: either within the coding sequence of C3P3 between the Nλ-mPAPOLA and NP868R-(G4S)2-K1ERNAP blocks (C3P3-G3a, C3P3-G3b, and C3P3-G3c), or at the start of the ORF immediately before the Nλ-mPAPOLA block (C3P3-G3d and C3P3-G3e). In contrast, in-frame insertions of E3L-Zα/NS1-dsDNA/(G4S)2/SZIP at the end of the coding sequence of the C3P3 enzyme was not tested because phage RNA polymerases such as K1ERNAP do not tolerate carboxyl-terminal extensions (Mookhtiar, Peluso et al. 1991, Gardner, Mookhtiar et al. 1997).

Two types of intervening sequences have been used, i.e. (G4S)2 and F2A. The (Gly4Ser)n linkers (where n indicates the number of repeats) are prototypes of flexible protein linkers for appropriate separation of the functional domains (Huston, Levinson et al. 1988). The 2A are protein sequence of viral origin, which causes ribosomal skipping during translation, which thereby result in apparent co-translational cleavage of the protein (Donnelly, Luke et al. 2001). The F2A sequence, which was used for present constructions, is from the Foot-and-mouth disease aphtovirus (UniProtKB/Uniprot accession number AAT01756, residues 934-955).

The greatest effect was observed with the CP3P-G3a plasmid (Nλ-mPAPOLA-F2A-E3L-Zα/NS1-dsDNA/2A/sZIP-F2A-NP868R-(G4S)2-K1ERNAP). This construction is characterized by an in-frame insertion of E3L-Zα/NS1-dsDNA coding sequence in the open-reading frame of C3P3-G2, flanked by two F2A motifs, thereby producing a polyprotein consisting of three distinct subunits. The expression levels observed in cells transfected with pC3P3a were statistically significantly higher than those obtained by co-transfection of pC3P3-G2 and pE3L-Zα/NS1-dsDNA (relative ratio of 3.73-fold vs. 3.50-fold; P<0.0001).

The C3P3-G3d construction (E3L-Zα/NS1-dsDNA/(G4S)2/sZIP-F2A-Nλ-mPAPOLA-F2A-NP868R-(G4S)2-K1ERNAP), where the coding sequence of E3L-Zα/NS1-dsDNA/(G4S)2/sZIP is inserted at the start of the C3P3 ORF and flanked by a F2A motif gave similar expression level to that of cells co-transfection of pC3P3-G2 and pE3L-Zα/NS1-dsDNA (relative ratio of 3.51-fold vs. 3.49-fold; P=NS). Other constructions were still functional but to a lesser extent than the E3L-Zα/NS1-dsDNA/(G4S)2/sZIP coding sequence not inserted in the C3P3a open-reading frame. Finally, the performances of these constructions ranged in the following order: C3P3-G3a>C3P3-G3d>C3P3-G3c≈C3P3-G3b>C3P3-G3e.

| | Firefly Luciferase |
|---|---|
| | (mean ± SD); |
| Plasmids | relative ratio |
| pC3P3-G2, pE3L-Zα/NS1-dsDNA/(G4S)2/sZIP, | 5 058 433 ± 180 056; 3.49 |
| pK1E-Luciferase-4xλBoxBL | |
| pC3P3-G3a, pK1E-Luciferase-4xλBoxBL | 5 399 055 ± 142 014; 3.73 |
| pC3P3-G3b, pK1E-Luciferase-4xλBoxBL | 4 391 463 ± 176 880; 3.03 |
| pC3P3-G3c, pK1E-Luciferase-4xλBoxBL | 4 398 126 ± 156 526; 3.04 |
| pC3P3-G3d, pK1E-Luciferase-4xλBoxBL | 5 074 674 ± 133 124; 3.51 |
| pC3P3-G3e, pK1E-Luciferase-4xλBoxBL | 4 192 816 ± 61 479; 2.90 |
| pC3P3-G2, pK1E-Luciferase-4xλBoxBL | 1 447 470 ± 32 976; 1.00 |
| pC3P3-G2, empty plasmid | 28 820 ± 1 143; 0.02 |
| Transfection reagent only | 17 050 ± 171; 0.01 |

Conclusions

The E3L-Zα/NS1-dsDNA/(G4S)2/SZIP coding sequence could be efficiently inserted in-frame into the scaffold of the C3P3-G2 enzyme, the C3P3-G3a construction having the best performance.

### Example 7: The dsRNA-Binding Domain of EIF2AK2 from Different Species can Increase Expression Levels by the C3P3-G2 and C3P3-G1 Systems

Objectives

This experiment has a goal of developing new artificial protein inhibitors of EIF2αphosphorylation. We thought that the dsRNA binding domain of EIF2AK2 deleted from its carboxy-terminal kinase domain could act as an inhibitor by dimerizing with the full-length wild-type EIF2AK2 protein. The resulting dimer is therefore possibly capable of trapping dsRNA, which in turn activates wild-type EIF2AK2. Moreover, due to the absence of a kinase domain, this dimer is likely to have reduced or no phosphorylation activity of its molecular target eIF2α.

Methods

Plasmids

The pC3P3-G1, pC3P3-G2 and pK1Ep-Luciferase-4xλBoxBr plasmids were described previously. EIF2AK2 (eukaryotic translation initiation factor 2-alpha kinase 2 also known as Protein kinase RNA-activated (PKR); human protein UniProtKB/Uniprot accession number P19525) has two functional domains which are separated by a region with no distinct structure or function:
a. N-terminal dsRNA binding domain (dsRBD), which consists of two tandem copies of a conserved double stranded RNA binding motif, dsRBM1 and dsRBM2 (residues 100-167). This domain is capable of homodimerizing and binding to dsRNA (Zhang, Romano et al. 2001),
b. C-terminal serine/threonine kinase domain, capable of autophosphorylating, then phosphorylating eIF2α, after its homodimerization (Zhang, Romano et al. 2001, Dey, Mann et al. 2014).

We hypothesized that the isolated dsRNA domain of EIF2AK2 without a kinase domain can act as an efficient competitive inhibitor. Also note that the relatively small size of this domain is well suited for the construction of C3P3 enzymes as shown in Example 9

The following mutant EIF2AK2 proteins consisting only of the dsRNA-binding domain were tested:
Human EIF2AK2 dsRNA-binding domain (UniProtKB/Uniprot accession number P19525, residues 2-167; phEIF2AK2:DRB),
Mouse EIF2AK2 dsRNA-binding domain (UniProtKB/Uniprot accession number Q03963, residues 2-162; pmEIF2AK2:DRB),
Bovine EIF2AK2 dsRNA-binding domain (UniProtKB/Uniprot accession number A0A4W2CP11 residues 2-167; pbEIF2AK2:DRB).

Firefly Luciferase Luminescence and SEAP Colorimetric Assays

Firefly luciferase luminescence was assayed from cell lysate as previously described.

Statistical Analysis

Statistical analyses were performed as previously described.

Results

The dsRNA binding domains of EIF2AK2 from different species, deleted of their carboxyl-terminal domains, were tested. Coexpression of all these proteins increased the level of expression of the Firefly Luciferase reporter gene expressed with the C3P3-G1 system. The effects observed were in the following order: human>mouse≈bovine.

| | Firefly Luciferase | |
|---|---|---|
| | (mean ± SD); | |
| Plasmids | relative ratio | P-value |
| pC3P3-G1, K1Ep(G)-Luciferase, phEIF2AK2:DRB | 1 575 067 ± 41 527; 1.37 | <0.0001 |
| pC3P3-G1, K1Ep(G)-Luciferase, pmEIF2AK2:DRB | 1 373 402 ± 43 643; 1.20 | <0.0001 |
| pC3P3-G1, K1Ep(G)-Luciferase, pbEIF2AK2:DRB | 1 363 178 ± 78 703; 1.19 | <0.0001 |
| pC3P3-G1, pK1E-Luciferase-4xλBoxBL | 1 147 342 ± 53 282; 1.00 | NA |
| pC3P3-G1, empty plasmid | 9 247 ± 239; 0.01 | |
| Transfection reagent only | 5 581 ± 188; 0.01 | |

A similar effect was observed by the coexpression of these dsRNA binding domains of EIF2AK2 from several species, but more marked with the second generation of the artificial expression system. The dsRNA binding domains from the human EIF2AK2 has the greatest effects.

| | Firefly Luciferase | |
|---|---|---|
| | (mean ± SD); | |
| Plasmids | relative ratio | P-value |
| pC3P3-G2, K1Ep(G)-Luciferase, phEIF2AK2:DRB | 3 788 243 ± 99 879; 1.78 | <0.0001 |
| pC3P3-G2, K1Ep(G)-Luciferase, pmEIF2AK2:DRB | 3 188 517 ± 101 322; 1.50 | <0.0001 |
| pC3P3-G2, K1Ep(G)-Luciferase, pbEIF2AK2:DRB | 3 164 781 ± 182 719; 1.49 | <0.0001 |
| pC3P3-G2, pK1E-Luciferase-4xλBoxBL | 2 130 953 ± 98 961; 1.00 | NA |
| pC3P3-G2, empty plasmid | 12 245 ± 239; 0.01 | |
| Transfection reagent only | 8 721 ± 188; 0.01 | |

Conclusions

dsRNA binding domains of EIF2AK2 can increase the expression of the reporter gene under the control of both the first and the second generation of the artificial expression system C3P3, which supports a dominant negative effect by competitive inhibition. The strongest effect was observed with human protein which has been selected for the development of a new artificial protein capable of inhibiting the phosphorylation of eIF2α.

Such dsRNA binding domains of EIF2AK2 can also be used to drive specifically its ubiquitination by E3 ligases and thereby their degradation by the 28S proteasome. To implement this mechanism of action, the present inventor has designed chimeric proteins resulting of the fusion of wild-type and mutant dsRNA binding domains of EIF2AK2 with specific subunit domains of multimeric E3 ligases, especially Skp1-interacting domains from F-box proteins (e.g. BTRCP, FBW7 or SPK2), elongin BC-interacting domains (e.g. VHL or SOCS2), Cullin3-interacting domains from SPOP, DDB1-interacting domains from CRBN, dimerization domain from STUB1 (also named CHIP) or CUL1-interacting domain from Skp1.

### Example 8: The Co-Expression of the dsRNA-Binding Domain of EIF2AK2 with Proteins Involved in eIF2α Dephosphorylation, can Even Increase Expression Levels by the C3P3-G1 and C3P3-G2 Expression Systems

Objectives

The aim of this experiment is to test whether a potentiation of the effect of the dsRNA-binding domain of EIF2AK2 with other protein factors. Due to the mechanism of action of dsRNA-binding domain of EIF2AK2 which makes it possible to repress the phosphorylation of eIF2α, we were particularly interested to the reverse modification pathway, namely proteins involved in dephosphorylation of eIF2α.

Methods

Plasmids

The pC3P3-G1, pC3P3-G2 and pK1Ep-Luciferase-4xλBoxBr plasmids were described previously. Viral and host-cell gene involved in eIF2α dephosphorylation were subcloned in the pCMVScript plasmid backbone (Stratagene, La Jolla, Calif.), as previously described, most of which were previously described:
pPPP1CA plasmid, which encodes the human serine/threonine-protein phosphatase PP1-alpha catalytic subunit (UniProtKB/Uniprot accession number P62136) that dephosphorylates eIF2α,
pPPP1R15A plasmid, which encodes a regulatory subunit that recruits the serine/threonine-protein phosphatase PPP1CA to dephosphorylate eIF2α (UniProtKB/Uniprot accession number O75807),
pICP34.5/HVS1 plasmid, which encodes the Herpes Simplex Virus ICP34.5 protein that serves as a regulatory subunit of protein phosphatase PPP1CA (UniProtKB/Uniprot accession number P03496),
Plasmids pDP71L(s)/ASFV and pDP71L(I)/ASFV, which respectively encode the short (UniProtKB/Uniprot accession number Q65212) and long (UniProtKB/Uniprot accession number P0C755) isoforms of African swine fever virus DP71L protein, both of which regulatory subunit of protein phosphatase PPP1CA.

Cell Culture and Transfection

Human cells were cultured and transfected as previously described.

Firefly Luciferase Luminescence and SEAP Colorimetric Assays

Firefly luciferase luminescence was assayed from cell lysate as previously described.

Statistical Analysis

Statistical analyses were performed as previously described.

Results

A possible additive effect on the expression of the reporter gene Firefly Luciferase expressed by the

C3P3 system was tested by co-transfection of the previous test plasmids, together with the plasmid phEIF2AK2:DRB.

The results of the table below show successively the effect of each of the two plasmids transfected separately, then of the two plasmids co-transfected simultaneously. Supra-additive effect (SA), infra-additive (IA) or purely additive (PA) effects were statistically defined as previously described in Example 5.

Surprisingly, we discovered supra-additive effect with all the plasmids coding for proteins involved in the eIF2α dephosphorylation pathway listed above. The efficacy on the expression of the reporter gene Firefly Luciferase was observed in the following order: pDP71L(I)/ASFV (ratio 4.54; P<0.001)>pPPP1CA (ratio 4.46; P<0.001)>pPPP1R15A (ratio 4.39; P<0.001) pICP34.5/HVS1 (ratio 4.36; P<0.05)>>pDP71L(s)/ASFV (ratio 3.51; P<0.001).

DP71L(I) encoding the long isoform of African swine fever virus DP71L protein was therefore used for construction of the new generation of C3P3-G3 enzymes shown in Example 9.

| | Firefly Luciferase |
|---|---|
| | (mean ± SD); |
| | relative ratio |
| Plasmids | (additivity, P-value) |
| 1) pC3P3-G2, phEIF2AK2:DRB, pK1E-Luciferase-4xλBoxBL | 3 831 026 ± 102 718; 1.75 |
| 2) pC3P3-G2, pPPP1CA, pK1E-Luciferase-4xλBoxBL | 5 040 224 ± 137 764; 2.30 |
| 1 + 2) pC3P3-G2, phEIF2AK2:DRB, pPPPICA, | 9 796 322 ± 241 884; 4.46 |
| phEIF2AK2:DRB, pK1E-Luciferase-4xλBoxBL | (SA, P < 0.001) |
| 1) pC3P3-G2, phEIF2AK2:DRB, pK1E-Luciferase-4xλBoxBL | 3 831 026 ± 102 718; 1.75 |
| 2) pC3P3-G2, pPPP1R15A, pK1E-Luciferase-4xλBoxBL | 4 990 197 ± 94 700; 2.27 |
| 1 + 2) pC3P3-G2, phEIF2AK2:DRB, pPPP1R15A, | 9 627 003 ± 330 477; 4.39 |
| phEIF2AK2:DRB, pK1E-Luciferase-4xλBoxBL | (SA; P < 0.001) |
| 1) pC3P3-G2, phEIF2AK2:DRB, pK1E-Luciferase-4xλBoxBL | 3 831 026 ± 102 718; 1.75 |
| 2) pC3P3-G2, pICP34.5/HVS1, pK1E-Luciferase-4xλBoxBL | 4 893 675 ± 172 443; 2.23 |
| 1 + 2) pC3P3-G2, phEIF2AK2:DRB, pICP34.5/HVS1, | 9 573 417 ± 413 280; 4.36 |
| phEIF2AK2:DRB, pK1E-Luciferase-4xλBoxBL | (SA; P < 0.05) |
| 1) pC3P3-G2, phEIF2AK2:DRB, pK1E-Luciferase-4xλBoxBL | 3 831 026 ± 102 718; 1.75 |
| 2) pC3P3-G2, pDP71L(s)/ASFV, pK1E-Luciferase-4xλBoxBL | 2 719 144 ± 68 774; 1.24 |
| 1 + 2) pC3P3-G2, phEIF2AK2:DRB, pDP71L(s)/ASFV, | 7 694 041 ± 281 447; 3.51 |
| phEIF2AK2:DRB, pK1E-Luciferase-4xλBoxBL | (SA; P < 0.001) |
| 1) pC3P3-G2, phEIF2AK2:DRB, pK1E-Luciferase-4xλBoxBL | 3 831 026 ± 102 718; 1.75 |
| 2) pC3P3-G2, pDP71L(I)/ASFV, pK1E-Luciferase-4xλBoxBL | 4 837 262 ± 279 279; 2.20 |
| 1 + 2) pC3P3-G2, phEIF2AK2:DRB, pDP71L(I)/ASFV, | 9 954 686 ± 292 508; 4.54 |
| phEIF2AK2:DRB, pK1E-Luciferase-4xλBoxBL | (SA; P < 0.0001) |
| pC3P3-G2, pK1E-Luciferase-4xλBoxBL | 2 194 881 ± 52 197 |
| pC3P3-G2, empty plasmid | 17 431 ± 403 |
| Transfection reagent only | 21 096 ± 870 |

Conclusions

These results therefore demonstrate the supra-additivity effect between the dsRNA-binding domain from hEIF2AK2 and all genes involved the eIF2α dephosphorylation pathway.

### Example 9: Assembly of New Generation C3P3 Enzymes

Objectives

The objective of the following experiment was to assemble in frame the genes identified from Example 8 within the open-reading frame of the C3P3-G2 enzyme. The resulting C3P3 genes are numbered C3P3-G3f to C3P3-G3i.

Methods

Plasmids

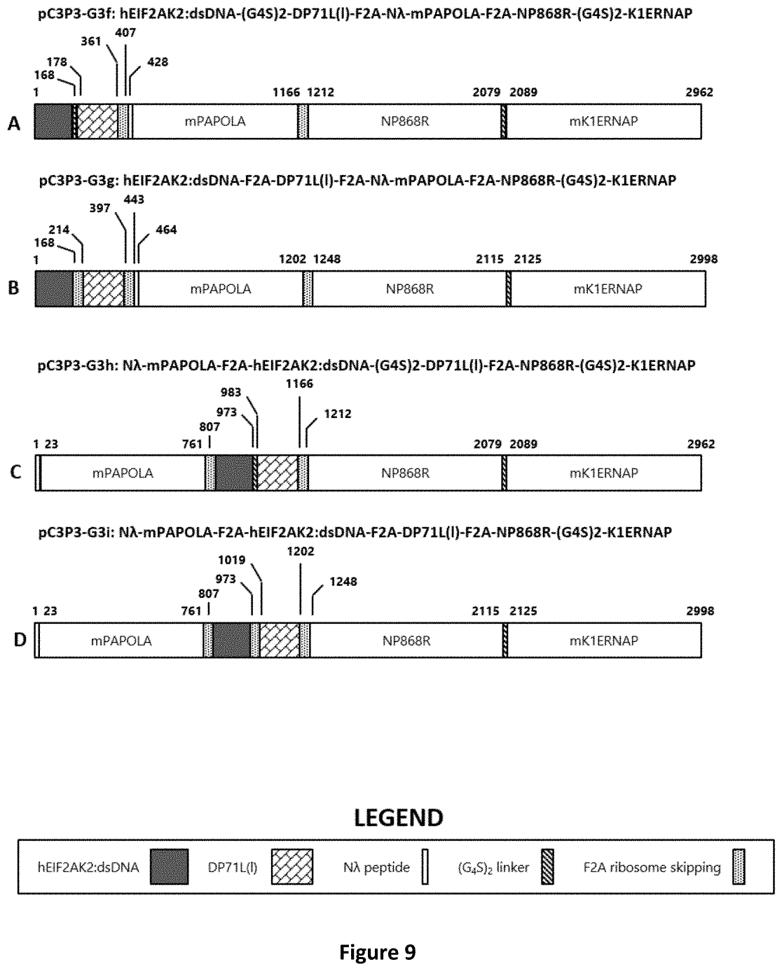

The assemblies tested hereinafter are designed according to two scaffolds:
hEIF2AK2:DRB-[X1]DP71L(I)-F2A-Nλ-mPAPOLA-F2A-NP868R-(G4S)2-K1ERNAP, where [X1] is either a (G4S)2 flexible linker (C3P3-G3f, SEQ ID NO. 35 and SEQ ID NO. 36; FIG. 9A) or an F2A ribosomal skipping motif (C3P3-G3g, SEQ ID NO. 37 and SEQ ID NO. 38; FIG. 9B),
Nλ-mPAPOLA-F2A-hEIF2AK2:DRB-[X2]-DP71L(I)]-F2A-NP868R-(G4S)2-K1ERNAP, where [X2] is either a (G4S)2 flexible linker (C3P3-G3h, SEQ ID NO. 39 and SEQ ID NO. 40; FIG. 9C) or an F2A ribosomal skipping motif (C3P3-G3i, SEQ ID NO. 41 and SEQ ID NO. 42; FIG. 9D).

Firefly Luciferase Luminescence and SEAP Colorimetric Assays

Results

The coding sequence hEIF2AK2:DRB-X-DP71L(I) was inserted in-frame into the open-reading frame of the C3P3-G2 enzyme, where X is an intervening sequence (i.e. either the flexible (Gly4Ser)2 linker, or the 2A are protein sequence, which causes ribosomal skipping). The hEIF2AK2:DRB-X-DP71L(I) coding sequence could be readily inserted at only two positions: either within the coding sequence of C3P3 either at the start of the ORF immediately before the Nλ-mPAPOLA block (C3P3-G3f and C3P3-G3g), or between the Nλ-mPAPOLA and NP868R-(G4S)2-K1ERNAP blocks (C3P3-G3h, C3P3-G3i). As previously stated in Example 6, it was not possible to position the hEIF2AK2:DRB-X-DP71L(I) block at the carboxyl-terminal end of the C3P3 protein because phage RNA polymerases such as K1ERNAP do not tolerate carboxyl-terminal extensions (Mookhtiar, Peluso et al. 1991, Gardner, Mookhtiar et al. 1997). As shown in the table below, all constructions were functional. The efficacy of the C3P3 enzymes on the expression of the reporter gene Firefly Luciferase was observed in the following order: C3P3-G2f (ratio 5.47 vs. C3P3-G2 expression system)>C3P3-G2g (ratio 5.22)>C3P3-G2h (ratio 4.77)>C3P3-G2i (ratio 4.52).

The best results were therefore obtained when the hEIF2AK2:DRB-X-DP71L(I) block was inserted at the start of the protein before the Nλ-mPAPOLA block (C3P3-G3f and C3P3-G3g), rather than between the Nλ-mPAPOLA and NP868R-(G4S)2-K1ERNAP blocks (C3P3-G3h and C3P3-G3i). In addition, better results were apparently obtained when flexible (Gly4Ser)2 linker used as intervening sequences between hEIF2AK2:DRB and DP71L(I) sequences rather than the 2A ribosome skipping sequences. Finally, the C3P3-G3f construction which gave the best performance was selected that has the following design: EIF2AK2:dsDNA-(G4S)2-DP71L(I)-F2A-Nλ-mPAPOLA-F2A-NP868R-(G4S)2-K1ERNAP.

| | Firefly Luciferase | |
|---|---|---|
| | (mean ± SD); | |
| Plasmids | relative ratio | P-value |
| pC3P3-G2, phEIF2AK2:DRB, pDP71L(I)/ASFV, | 9 229 953 ± 291 877; 4.64 | <0.0001 |
| phEIF2AK2:DRB, pK1E-Luciferase-4xλBoxBL | | |
| C3P3-G3f, pK1E-Luciferase-4xλBoxBL | 10 885 745 ± 352 233; 5.47 | <0.0001 |
| C3P3-G3g, pK1E-Luciferase-4xλBoxBL | 10 389 482 ± 318 248; 5.22 | <0.0001 |
| C3P3-G3h, pK1E-Luciferase-4xλBoxBL | 9 491 167 ± 307 109; 4.77 | <0.0001 |
| C3P3-G3i, pK1E-Luciferase-4xλBoxBL | 8 996 018 ± 275 564; 4.52 | <0.0001 |
| pC3P3-G2, pK1E-Luciferase-4xλBoxBL | 1 988 881 ± 47 298; 1.00 | NA |
| pC3P3-G2, empty plasmid | 19 116 ± 788 | |
| Transfection reagent only | 15 795 ± 365 | |

Conclusions

The hEIF2AK2:DRB-X-DP71L(I) coding sequence could be efficiently inserted in-frame into the scaffold of the C3P3-G2 enzyme, the C3P3-G3f construction having the best performance.

### BIBLIOGRAPHY

Abbas, Y. M., B. T. Laudenbach, S. Martinez-Montero, R. Cencic, M. Habjan, A. Pichlmair, M. J. Damha, J. Pelletier and B. Nagar (2017). “Structure of human IFIT1 with capped RNA reveals adaptable mRNA binding and mechanisms for sensing N1 and N2 ribose 2′-O methylations.” Proc Natl Acad Sci USA 114(11): E2106-E2115.
Afonso, C. L., L. Zsak, C. Carrillo, M. V. Borca and D. L. Rock (1998). “African swine fever virus NL gene is not required for virus virulence.” J Gen Virol 79 (Pt 10): 2543-2547.
Alcami, A., J. A. Symons and G. L. Smith (2000). “The vaccinia virus soluble alpha/beta interferon (IFN) receptor binds to the cell surface and protects cells from the antiviral effects of IFN.” J Virol 74(23): 11230-11239.
Alff, P. J., N. Sen, E. Gorbunova, I. N. Gavrilovskaya and E. R. Mackow (2008). “The NY-1 hantavirus Gn cytoplasmic tail coprecipitates TRAF3 and inhibits cellular interferon responses by disrupting TBK1-TRAF3 complex formation.” J Virol 82(18): 9115-9122.
Altschul, S. F., T. L. Madden, A. A. Schaffer, J. Zhang, Z. Zhang, W. Miller and D. J. Lipman (1997). “Gapped BLAST and PSI-BLAST: a new generation of protein database search programs.” Nucleic Acids Res 25(17): 3389-3402.
Anderson, E. M., A. Birmingham, S. Baskerville, A. Reynolds, E. Maksimova, D. Leake, Y. Fedorov, J. Karpilow and A. Khvorova (2008). “Experimental validation of the importance of seed complement frequency to siRNA specificity.” RNA 14(5): 853-861.
Andreev, D. E., P. B. O'Connor, C. Fahey, E. M. Kenny, I. M. Terenin, S. E. Dmitriev, P. Cormican, D. W. Morris, I. N. Shatsky and P. V. Baranov (2015). “Translation of 5′ leaders is pervasive in genes resistant to eIF2 repression.” Elife 4: e03971.
Ashour, J., M. Laurent-Rolle, P. Y. Shi and A. Garcia-Sastre (2009). “N55 of dengue virus mediates STAT2 binding and degradation.” J Virol 83(11): 5408-5418.
Au-Yeung, N., R. Mandhana and C. M. Horvath (2013). “Transcriptional regulation by STAT1 and STAT2 in the interferon JAK-STAT pathway.” JAKSTAT 2(3): e23931.
Baird, T. D., L. R. Palam, M. E. Fusakio, J. A. Willy, C. M. Davis, J. N. McClintick, T. G. Anthony and R. C. Wek (2014). “Selective mRNA translation during eIF2 phosphorylation induces expression of IBTKalpha.” Mol Biol Cell 25(10): 1686-1697.
Barber, C., C. Netherton, L. Goatley, A. Moon, S. Goodbourn and L. Dixon (2017). “Identification of residues within the African swine fever virus DP71L protein required for dephosphorylation of translation initiation factor eIF2alpha and inhibiting activation of pro-apoptotic CHOP.” Virology 504: 107-113.
Barnard, P. and N. A. McMillan (1999). “The human papillomavirus E7 oncoprotein abrogates signaling mediated by interferon-alpha.” Virology 259(2): 305-313.
Barro, M. and J. T. Patton (2005). “Rotavirus nonstructural protein 1 subverts innate immune response by inducing degradation of IFN regulatory factor 3.” Proc Natl Acad Sci USA 102(11): 4114-4119.
Barro, M. and J. T. Patton (2007). “Rotavirus NSP1 inhibits expression of type I interferon by antagonizing the function of interferon regulatory factors IRF3, IRF5, and IRF7.” J Virol 81(9): 4473-4481.
Bergmann, M., A. Garcia-Sastre, E. Carnero, H. Pehamberger, K. Wolff, P. Palese and T. Muster (2000). “Influenza virus NS1 protein counteracts PKR-mediated inhibition of replication.” J Virol 74(13): 6203-6206.
Berlanga, J. J., I. Ventoso, H. P. Harding, J. Deng, D. Ron, N. Sonenberg, L. Carrasco and C. de Haro (2006). “Antiviral effect of the mammalian translation initiation factor 2alpha kinase GCN2 against RNA viruses.” EMBO J 25(8): 1730-1740.
Bertolotti, A., Y. Zhang, L. M. Hendershot, H. P. Harding and D. Ron (2000). “Dynamic interaction of BiP and ER stress transducers in the unfolded-protein response.” Nat Cell Biol 2(6): 326-332.
Birmingham, A., E. M. Anderson, A. Reynolds, D. Ilsley-Tyree, D. Leake, Y. Fedorov, S. Baskerville, E. Maksimova, K. Robinson, J. Karpilow, W. S. Marshall and A. Khvorova (2006). “3′ UTR seed matches, but not overall identity, are associated with RNAi off-targets.” Nat Methods 3(3): 199-204.
Brzozka, K., S. Finke and K. K. Conzelmann (2006). “Inhibition of interferon signaling by rabies virus phosphoprotein P: activation-dependent binding of STAT1 and STAT2.” J Virol 80(6): 2675-2683.
Buettner, N., C. Vogt, L. Martinez-Sobrido, F. Weber, Z. Waibler and G. Kochs (2010). “Thogoto virus ML protein is a potent inhibitor of the interferon regulatory factor-7 transcription factor.” J Gen Virol 91(Pt 1): 220-227.
Caignard, G., M. Bourai, Y. Jacob, M. p. I. M. A. P. Infection, F. Tangy and P. O. Vidalain (2009). “Inhibition of IFN-alpha/beta signaling by two discrete peptides within measles virus V protein that specifically bind STAT1 and STAT2.” Virology 383(1): 112-120.
Cardenas, W. B., Y. M. Loo, M. Gale, Jr., A. L. Hartman, C. R. Kimberlin, L. Martinez-Sobrido, E. O. Saphire and C. F. Basler (2006). “Ebola virus VP35 protein binds double-stranded RNA and inhibits alpha/beta interferon production induced by RIG-I signaling.” J Virol 80(11): 5168-5178.
Chang, T. H., T. Kubota, M. Matsuoka, S. Jones, S. B. Bradfute, M. Bray and K. Ozato (2009). “Ebola Zaire virus blocks type I interferon production by exploiting the host SUMO modification machinery.” PLoS Pathog 5(6): e1000493.
Chasse, H., S. Boulben, V. Costache, P. Cormier and J. Morales (2017). “Analysis of translation using polysome profiling.” Nucleic Acids Res 45(3): e15.
Chelbi-Alix, M. K., A. Vidy, J. E1 Bougrini and D. Blondel (2006). “Rabies viral mechanisms to escape the IFN system: the viral protein P interferes with IRF-3, Stat1, and PML nuclear bodies.” J Interferon Cytokine Res 26(5): 271-280.
Chen, J. J., M. S. Throop, L. Gehrke, I. Kuo, J. K. Pal, M. Brodsky and I. M. London (1991). “Cloning of the cDNA of the heme-regulated eukaryotic initiation factor 2 alpha (eIF-2 alpha) kinase of rabbit reticulocytes: homology to yeast GCN2 protein kinase and human double-stranded-RNA-dependent eIF-2 alpha kinase.” Proc Natl Acad Sci USA 88(17): 7729-7733.
Chen, Z. and T. D. Schneider (2005). “Information theory based T7-like promoter models: classification of bacteriophages and differential evolution of promoters and their polymerases.” Nucleic Acids Res 33(19): 6172-6187.
Child, S. J. and A. P. Geballe (2009). “Binding and relocalization of protein kinase R by murine cytomegalovirus.” J Virol 83(4): 1790-1799.
Childs, K. S., J. Andrejeva, R. E. Randall and S. Goodbourn (2009). “Mechanism of mda-5 Inhibition by paramyxovirus V proteins.” J Virol 83(3): 1465-1473.
Ciancanelli, M. J., V. A. Volchkova, M. L. Shaw, V. E. Volchkov and C. F. Basler (2009). “Nipah virus sequesters inactive STAT1 in the nucleus via a P gene-encoded mechanism.” J Virol 83(16): 7828-7841.
Cloutier, N. and L. Flamand (2010). “Kaposi sarcoma-associated herpesvirus latency-associated nuclear antigen inhibits interferon (IFN) beta expression by competing with IFN regulatory factor-3 for binding to IFNB promoter.” J Biol Chem 285(10): 7208-7221.
Das, A. (1993). “Control of transcription termination by RNA-binding proteins.” Annu Rev Biochem 62(1): 893-930.
Dauber, B., J. Schneider and T. Wolff (2006). “Double-stranded RNA binding of influenza B virus nonstructural NS1 protein inhibits protein kinase R but is not essential to antagonize production of alpha/beta interferon.” J Virol 80(23): 11667-11677.
Davies, M. V., H. W. Chang, B. L. Jacobs and R. J. Kaufman (1993). “The E3L and K3L vaccinia virus gene products stimulate translation through inhibition of the double-stranded RNA-dependent protein kinase by different mechanisms.” J Virol 67(3): 1688-1692.
Devaraj, S. G., N. Wang, Z. Chen, Z. Chen, M. Tseng, N. Barretto, R. Lin, C. J. Peters, C. T. Tseng, S. C. Baker and K. Li (2007). “Regulation of IRF-3-dependent innate immunity by the papain-like protease domain of the severe acute respiratory syndrome coronavirus.” J Biol Chem 282(44): 32208-32221.
Dever, T. E., L. Feng, R. C. Wek, A. M. Cigan, T. F. Donahue and A. G. Hinnebusch (1992). “Phosphorylation of initiation factor 2 alpha by protein kinase GCN2 mediates gene-specific translational control of GCN4 in yeast.” Cell 68(3): 585-596.
Dey, M., B. R. Mann, A. Anshu and M. A. Mannan (2014). “Activation of protein kinase PKR requires dimerization-induced cis-phosphorylation within the activation loop.” J Biol Chem 289(9): 5747-5757.
Dey, S., T. D. Baird, D. Zhou, L. R. Palam, D. F. Spandau and R. C. Wek (2010). “Both transcriptional regulation and translational control of ATF4 are central to the integrated stress response.” J Biol Chem 285(43): 33165-33174.
Didcock, L., D. F. Young, S. Goodbourn and R. E. Randall (1999). “The V protein of simian virus 5 inhibits interferon signalling by targeting STAT1 for proteasome-mediated degradation.” J Virol 73(12): 9928-9933.
Dixon, L. K., D. A. Chapman, C. L. Netherton and C. Upton (2013). “African swine fever virus replication and genomics.” Virus Res 173(1): 3-14.
Donnelly, M. L., G. Luke, A. Mehrotra, X. Li, L. E. Hughes, D. Gani and M. D. Ryan (2001). “Analysis of the aphthovirus 2A/2B polyprotein ‘cleavage’ mechanism indicates not a proteolytic reaction, but a novel translational effect: a putative ribosomal ‘skip’.” J Gen Virol 82(Pt 5): 1013-1025.
Doyle, S. E., H. Schreckhise, K. Khuu-Duong, K. Henderson, R. Rosier, H. Storey, L. Yao, H. Liu, F. Barahmand-pour, P. Sivakumar, C. Chan, C. Birks, D. Foster, C. H. Clegg, P. Wietzke-Braun, S. Mihm and K. M. Klucher (2006). “Interleukin-29 uses a type 1 interferon-like program to promote antiviral responses in human hepatocytes.” Hepatology 44(4): 896-906.
Eglen, R. M., T. Reisine, P. Roby, N. Rouleau, C. Illy, R. Bosse and M. Bielefeld (2008). “The use of AlphaScreen technology in HTS: current status.” Curr Chem Genomics 1: 2-10.
Elliott, J., O. T. Lynch, Y. Suessmuth, P. Qian, C. R. Boyd, J. F. Burrows, R. Buick, N. J. Stevenson, O. Touzelet, M. Gadina, U. F. Power and J. A. Johnston (2007). “Respiratory syncytial virus NS1 protein degrades STAT2 by using the Elongin-Cullin E3 ligase.” J Virol 81(7): 3428-3436.
Essbauer, S., M. Bremont and W. Ahne (2001). “Comparison of the eIF-2alpha homologous proteins of seven ranaviruses (Iridoviridae).” Virus Genes 23(3): 347-359.
Fan, L., T. Briese and W. I. Lipkin (2010). “Z proteins of New World arenaviruses bind RIG-I and interfere with type I interferon induction.” J Virol 84(4): 1785-1791.
Friedman, D. I. and D. L. Court (1995). “Transcription antitermination: the lambda paradigm updated.” Mol Microbiol 18(2): 191-200.
Gack, M. U., R. A. Albrecht, T. Urano, K.-S. Inn, I. C. Huang, E. Carnero, M. Farzan, S. Inoue, J. U. Jung and A. Garcia-Sastre (2009). “Influenza A virus NS1 targets the ubiquitin ligase TRI M25 to evade recognition by the host viral RNA sensor RIG-I.” Cell host & microbe 5(5): 439-449.
Garcin, D., J. B. Marq, L. Strahle, P. le Mercier and D. Kolakofsky (2002). “All four Sendai Virus C proteins bind Stat1, but only the larger forms also induce its mono-ubiquitination and degradation.” Virology 295(2): 256-265.
Gardner, L. P., K. A. Mookhtiar and J. E. Coleman (1997). “Initiation, elongation, and processivity of carboxyl-terminal mutants of T7 RNA polymerase.” Biochemistry 36(10): 2908-2918.
Geiger, T. R. and J. M. Martin (2006). “The Epstein-Barr virus-encoded LMP-1 oncoprotein negatively affects Tyk2 phosphorylation and interferon signaling in human B cells.” J Virol 80(23): 11638-11650.
Goatley, L. C., M. B. Marron, S. C. Jacobs, J. M. Hammond, J. E. Miskin, C. C. Abrams, G. L. Smith and L. K. Dixon (1999). “Nuclear and nucleolar localization of an African swine fever virus protein, I14L, that is similar to the herpes simplex virus-encoded virulence factor ICP34.5.” J Gen Virol 80 (Pt 3)(3): 525-535.
Gosink, M. M. and R. D. Vierstra (1995). “Redirecting the specificity of ubiquitination by modifying ubiquitin-conjugating enzymes.” Proc Natl Acad Sci USA 92(20): 9117-9121.
Greenblatt, J., J. R. Nodwell and S. W. Mason (1993). “Transcriptional antitermination.” Nature 364(6436): 401-406.
Groskreutz, D. J., E. C. Babor, M. M. Monick, S. M. Varga and G. W. Hunninghake (2010). “Respiratory syncytial virus limits alpha subunit of eukaryotic translation initiation factor 2 (eIF2alpha) phosphorylation to maintain translation and viral replication.” J Biol Chem 285(31): 24023-24031.
Guasparri, I., H. Wu and E. Cesarman (2006). “The KSHV oncoprotein vFLIP contains a TRAF-interacting motif and requires TRAF2 and TRAF3 for signalling.” EMBO Rep 7(1): 114-119.
Habjan, M., A. Pichlmair, R. M. Elliott, A. K. Overby, T. Glatter, M. Gstaiger, G. Superti-Furga, H. Unger and F. Weber (2009). “NSs protein of rift valley fever virus induces the specific degradation of the double-stranded RNA-dependent protein kinase.” J Virol 83(9): 4365-4375.
Hahn, A. M., L. E. Huye, S. Ning, J. Webster-Cyriaque and J. S. Pagano (2005). “Interferon regulatory factor 7 is negatively regulated by the Epstein-Barr virus immediate-early gene, BZLF-1.” J Virol 79(15): 10040-10052.
Hakki, M., E. E. Marshall, K. L. De Niro and A. P. Geballe (2011). “Binding and Nuclear Relocalization of Protein Kinase R by Human Cytomegalovirus TRS1.” Journal of Virology 85(23): 12837-12837.
Harbury, P. B., P. S. Kim and T. Alber (1994). “Crystal structure of an isoleucine-zipper trimer.” Nature 371(6492): 80-83.
Harbury, P. B., T. Zhang, P. S. Kim and T. Alber (1993). “A switch between two-, three-, and four-stranded coiled coils in GCN4 leucine zipper mutants.” Science 262(5138): 1401-1407.
Harding, H. P., Y. Zhang and D. Ron (1999). “Protein translation and folding are coupled by an endoplasmic-reticulum-resident kinase.” Nature 397(6716): 271-274.
Hebner, C. M., R. Wilson, J. Rader, M. Bidder and L. A. Laimins (2006). “Human papillomaviruses target the double-stranded RNA protein kinase pathway.” J Gen Virol 87(Pt 11): 3183-3193.
Hetz, C. and F. R. Papa (2018). “The Unfolded Protein Response and Cell Fate Control.” Mol Cell 69(2): 169-181. Ho, C. K. and S. Shuman (1996). “Physical and functional characterization of the double-stranded RNA binding protein encoded by the vaccinia virus E3 gene.” Virology 217(1): 272-284.
Honda, K., A. Takaoka and T. Taniguchi (2006). “Type I interferon [corrected] gene induction by the interferon regulatory factor family of transcription factors.” Immunity 25(3): 349-360.
Hong, M. N., K. Y. Nam, K. K. Kim, S. Y. Kim and I. Kim (2016). “The small molecule ‘1-(4-biphenylylcarbonyl)-4-(5-bromo-2-methoxybenzyl) piperazine oxalate’ and its derivatives regulate global protein synthesis by inactivating eukaryotic translation initiation factor 2-alpha.” Cell Stress Chaperones 21(3): 485-497.
Huston, J. S., D. Levinson, M. Mudgett-Hunter, M. S. Tai, J. Novotny, M. N. Margolies, R. J. Ridge, R. E. Bruccoleri, E. Haber, R. Crea and et al. (1988). “Protein engineering of antibody binding sites: recovery of specific activity in an anti-digoxin single-chain Fv analogue produced in Escherichia coli.” Proc Natl Acad Sci USA 85(16): 5879-5883.
Hyde, J. L., C. L. Gardner, T. Kimura, J. P. White, G. Liu, D. W. Trobaugh, C. Huang, M. Tonelli, S. Paessler, K. Takeda, W. B. Klimstra, G. K. Amarasinghe and M. S. Diamond (2014). “A viral RNA structural element alters host recognition of nonself RNA.” Science 343(6172): 783-787.
Imani, F. and B. L. Jacobs (1988). “Inhibitory activity for the interferon-induced protein kinase is associated with the reovirus serotype 1 sigma 3 protein.” Proc Natl Acad Sci USA 85(21): 7887-7891.
Jackson, A. L., J. Burchard, D. Leake, A. Reynolds, J. Schelter, J. Guo, J. M. Johnson, L. Lim, J. Karpilow, K. Nichols, W. Marshall, A. Khvorova and P. S. Linsley (2006). “Position-specific chemical modification of siRNAs reduces “off-target” transcript silencing.” RNA 12(7): 1197-1205.
Jais, P. H. (2011). Capping-prone RNA polymerase enzymes and their applications, Eukarÿs, France. PCT/EP2011/056051.
Jais, P. H. (2011). Capping-prone RNA polymerase enzymes and their applications, Eukarÿs, France. U.S. Pat. No. 9,540,671.B2, continuation patent.
Jais, P. H. (2017). New chimeric enzymes and their applications, Eukarÿs, France. PCT EP2018/070479.
Jais, P. H., E. Decroly, E. Jacquet, M. Le Boulch, A. Jais, O. Jean-Jean, H. Eaton, P. Ponien, F. Verdier, B. Canard, S. Goncalves, S. Chiron, M. Le Gall, P. Mayeux and M. Shmulevitz (2019). “C3P3-G1: first generation of a eukaryotic artificial cytoplasmic expression system.” Nucleic Acids Res 47(5): 2681-2698.
Jammi, N. V., L. R. Whitby and P. A. Beal (2003). “Small molecule inhibitors of the RNA-dependent protein kinase.” Biochem Biophys Res Commun 308(1): 50-57.
Jiang, F., A. Ramanathan, M. T. Miller, G. Q. Tang, M. Gale, Jr., S. S. Patel and J. Marcotrigiano (2011). “Structural basis of RNA recognition and activation by innate immune receptor RIG-I.” Nature 479(7373): 423-427.
Jousse, C., A. Bruhat, V. Carraro, F. Urano, M. Ferrara, D. Ron and P. Fafournoux (2001). “Inhibition of CHOP translation by a peptide encoded by an open reading frame localized in the chop 5′UTR.” Nucleic Acids Res 29(21): 4341-4351.
Kang, H. R., W. C. Cheong, J. E. Park, S. Ryu, H. J. Cho, H. Youn, J. H. Ahn and M. J. Song (2014). “Murine gammaherpesvirus 68 encoding open reading frame 11 targets TANK binding kinase 1 to negatively regulate the host type I interferon response.” J Virol 88(12): 6832-6846.
Kashiwabara, S. I., S. Tsuruta, K. Okada, Y. Yamaoka and T. Baba (2016). “Adenylation by testis-specific cytoplasmic poly(A) polymerase, PAPOLB/TPAP, is essential for spermatogenesis.” J Reprod Dev 62(6): 607-614.
Kato, H., O. Takeuchi, S. Sato, M. Yoneyama, M. Yamamoto, K. Matsui, S. Uematsu, A. Jung, T. Kawai, K. J. Ishii, O. Yamaguchi, K. Otsu, T. Tsujimura, C. S. Koh, C. Reis e Sousa, Y. Matsuura, T. Fujita and S. Akira (2006). “Differential roles of MDA5 and RIG-I helicases in the recognition of RNA viruses.” Nature 441(7089): 101-105.
Katze, M. G., M. Wambach, M. L. Wong, M. Garfinkel, E. Meurs, K. Chong, B. R. Williams, A. G. Hovanessian and G. N. Barber (1991). “Functional expression and RNA binding analysis of the interferon-induced, double-stranded RNA-activated, 68,000-Mr protein kinase in a cell-free system.” Mol Cell Biol 11(11): 5497-5505.
Kawagishi-Kobayashi, M., C. Cao, J. Lu, K. Ozato and T. E. Dever (2000). “Pseudosubstrate inhibition of protein kinase PKR by swine pox virus C8L gene product.” Virology 276(2): 424-434.
Kawai, T. and S. Akira (2006). “TLR signaling.” Cell Death Differ 13(5): 816-825.
Khan, A., M. Tahir Khan, S. Saleem, M. Junaid, A. Ali, S. Shujait Ali, M. Khan and D. Q. Wei (2020). “Structural insights into the mechanism of RNA recognition by the N-terminal RNA-binding domain of the SARS-CoV-2 nucleocapsid phosphoprotein.” Comput Struct Biotechnol J 18: 2174-2184.
Khoo, D., C. Perez and I. Mohr (2002). “Characterization of RNA determinants recognized by the arginine- and proline-rich region of Us11, a herpes simplex virus type 1-encoded double-stranded RNA binding protein that prevents PKR activation.” J Virol 76(23): 11971-11981.
Kim, Y. G., M. Muralinath, T. Brandt, M. Pearcy, K. Hauns, K. Lowenhaupt, B. L. Jacobs and A. Rich (2003). “A role for Z-DNA binding in vaccinia virus pathogenesis.” Proc Natl Acad Sci USA 100(12): 6974-6979.
Kimberlin, C. R., Z. A. Bornholdt, S. Li, V. L. Woods, Jr., I. J. MacRae and E. O. Saphire (2010). “Ebolavirus VP35 uses a bimodal strategy to bind dsRNA for innate immune suppression.” Proc Natl Acad Sci USA 107(1): 314-319.
Knutsen, J. H. J., G. E. Rødland, C. A. Bøe, T. W. H5land, P. Sunnerhagen, B. Grallert and E. Boye (2015). “Stress-induced inhibition of translation independently of eIF2α phosphorylation.” Journal of Cell Science 128(23): 4420-4427.
Kowalinski, E., T. Lunardi, A. A. McCarthy, J. Louber, J. Brunel, B. Grigorov, D. Gerlier and S. Cusack (2011). “Structural basis for the activation of innate immune pattern-recognition receptor RIG-I by viral RNA.” Cell 147(2): 423-435.
Kubota, T., N. Yokosawa, S. Yokota and N. Fujii (2002). “Association of mumps virus V protein with RACK1 results in dissociation of STAT-1 from the alpha interferon receptor complex.” J Virol 76(24): 12676-12682.
Kyriakopoulou, C. B., H. Nordvarg and A. Virtanen (2001). “A novel nuclear human poly(A) polymerase (PAP), PAP gamma.” J Biol Chem 276(36): 33504-33511.
LaMonica, R., S. S. Kocer, J. Nazarova, W. Dowling, E. Geimonen, R. D. Shaw and E. R. Mackow (2001). “VP4 differentially regulates TRAF2 signaling, disengaging JNK activation while directing NF-kappa B to effect rotavirus-specific cellular responses.” J Biol Chem 276(23): 19889-19896.
Langland, J. O. and B. L. Jacobs (2002). “The role of the PKR-inhibitory genes, E3L and K3L, in determining vaccinia virus host range.” Virology 299(1): 133-141.
Langland, J. O., S. Pettiford, B. Jiang and B. L. Jacobs (1994). “Products of the porcine group C rotavirus NSP3 gene bind specifically to double-stranded RNA and inhibit activation of the interferon-induced protein kinase PKR.” J Virol 68(6): 3821-3829.
Lee, Y. Y., R. C. Cevallos and E. Jan (2009). “An upstream open reading frame regulates translation of GADD34 during cellular stresses that induce eIF2alpha phosphorylation.” J Biol Chem 284(11): 6661-6673.
Leung, D. W., K. C. Prins, D. M. Borek, M. Farahbakhsh, J. M. Tufariello, P. Ramanan, J. C. Nix, L. A. Helgeson, Z. Otwinowski, R. B. Honzatko, C. F. Basler and G. K. Amarasinghe (2010). “Structural basis for dsRNA recognition and interferon antagonism by Ebola VP35.” Nat Struct Mol Biol 17(2): 165-172.
Levin, D. H., D. Kyner and G. Acs (1973). “Protein initiation in eukaryotes: formation and function of a ternary complex composed of a partially purified ribosomal factor, methionyl transfer RNA, and guanosine triphosphate.” Proc Natl Acad Sci USA 70(1): 41-45.
Levin, D. H., R. Petryshyn and I. M. London (1980). “Characterization of double-stranded-RNA-activated kinase that phosphorylates alpha subunit of eukaryotic initiation factor 2 (eIF-2 alpha) in reticulocyte lysates.” Proc Natl Acad Sci USA 77(2): 832-836.
Li, Q., R. Means, S. Lang and J. U. Jung (2007). “Downregulation of gamma interferon receptor 1 by Kaposi's sarcoma-associated herpesvirus K3 and K5.” J Virol 81(5): 2117-2127.
Li, S., S. Labrecque, M. C. Gauzzi, A. R. Cuddihy, A. H. Wong, S. Pellegrini, G. J. Matlashewski and A. E. Koromilas (1999). “The human papilloma virus (HPV)-18 E6 oncoprotein physically associates with Tyk2 and impairs Jak-STAT activation by interferon-alpha.” Oncogene 18(42): 5727-5737.
Li, X. D., L. Sun, R. B. Seth, G. Pineda and Z. J. Chen (2005). “Hepatitis C virus protease NS3/4A cleaves mitochondrial antiviral signaling protein off the mitochondria to evade innate immunity.” Proc Natl Acad Sci USA 102(49): 17717-17722.
Li, X. D., J. Wu, D. Gao, H. Wang, L. Sun and Z. J. Chen (2013). “Pivotal roles of cGAS-cGAMP signaling in antiviral defense and immune adjuvant effects.” Science 341(6152): 1390-1394.
Lim, S., R. Khoo, K. M. Peh, J. Teo, S. C. Chang, S. Ng, G. L. Beilhartz, R. A. Melnyk, C. W. Johannes, C. J. Brown, D. P. Lane, B. Henry and A. W. Partridge (2020). “bioPROTACs as versatile modulators of intracellular therapeutic targets including proliferating cell nuclear antigen (PCNA).” Proc Natl Acad Sci USA 117(11): 5791-5800.
Lin, R. J., B. L. Chang, H. P. Yu, C. L. Liao and Y. L. Lin (2006). “Blocking of interferon-induced Jak-Stat signaling by Japanese encephalitis virus NS5 through a protein tyrosine phosphatase-mediated mechanism.” J Virol 80(12): 5908-5918.
Ling, Z., K. C. Tran and M. N. Teng (2009). “Human respiratory syncytial virus nonstructural protein NS2 antagonizes the activation of beta interferon transcription by interacting with RIG-I.” J Virol 83(8): 3734-3742.
Lingel, A., B. Simon, E. Izaurralde and M. Sattler (2005). “The structure of the flock house virus B2 protein, a viral suppressor of RNA interference, shows a novel mode of double-stranded RNA recognition.” EMBO Rep 6(12): 1149-1155.
Look, D. C., W. T. Roswit, A. G. Frick, Y. Gris-Alevy, D. M. Dickhaus, M. J. Walter and M. J. Holtzman (1998). “Direct suppression of Stat1 function during adenoviral infection.” Immunity 9(6): 871-880.
Lundstrom, K. (2016). “Replicon RNA Viral Vectors as Vaccines.” Vaccines (Basel) 4(4): 39.
Lundstrom, K. (2019). “Plasmid DNA-based Alphavirus Vaccines.” Vaccines (Basel) 7(1): 29.
Ma, Y., H. Jin, T. Valyi-Nagy, Y. Cao, Z. Yan and B. He (2012). “Inhibition of TANK binding kinase 1 by herpes simplex virus 1 facilitates productive infection.” J Virol 86(4): 2188-2196.
Makarova, O. V., E. M. Makarov, R. Sousa and M. Dreyfus (1995). “Transcribing of Escherichia coli genes with mutant T7 RNA polymerases: stability of lacZ mRNA inversely correlates with polymerase speed.” Proc Natl Acad Sci USA 92(26): 12250-12254.
Marcello, T., A. Grakoui, G. Barba-Spaeth, E. S. Machlin, S. V. Kotenko, M. R. MacDonald and C. M. Rice (2006). “Interferons alpha and lambda inhibit hepatitis C virus replication with distinct signal transduction and gene regulation kinetics.” Gastroenterology 131(6): 1887-1898.
Masatani, T., N. Ito, K. Shimizu, Y. Ito, K. Nakagawa, Y. Sawaki, H. Koyama and M. Sugiyama (2010). “Rabies virus nucleoprotein functions to evade activation of the RIG-I-mediated antiviral response.” J Virol 84(8): 4002-4012.
Meurs, E., K. Chong, J. Galabru, N. S. Thomas, I. M. Kerr, B. R. Williams and A. G. Hovanessian (1990). “Molecular cloning and characterization of the human double-stranded RNA-activated protein kinase induced by interferon.” Cell 62(2): 379-390.
Minakshi, R., K. Padhan, M. Rani, N. Khan, F. Ahmad and S. Jameel (2009). “The SARS Coronavirus 3a protein causes endoplasmic reticulum stress and induces ligand-independent downregulation of the type 1 interferon receptor.” PLoS One 4(12): e8342.
Mookhtiar, K. A., P. S. Peluso, D. K. Muller, J. J. Dunn and J. E. Coleman (1991). “Processivity of T7 RNA polymerase requires the C-terminal Phe882-Ala883-COO— or “foot”.” Biochemistry 30(25): 6305-6313.
Mossman, K. L. and J. R. Smiley (2002). “Herpes simplex virus ICP0 and ICP34.5 counteract distinct interferon-induced barriers to virus replication.” J Virol 76(4): 1995-1998.
Novoa, I., H. Zeng, H. P. Harding and D. Ron (2001). “Feedback inhibition of the unfolded protein response by GADD34-mediated dephosphorylation of eIF2alpha.” J Cell Biol 153(5): 1011-1022.
O'Shea, E. K., J. D. Klemm, P. S. Kim and T. Alber (1991). “X-ray structure of the GCN4 leucine zipper, a two-stranded, parallel coiled coil.” Science 254(5031): 539-544.
Olland, A. M., J. Jane-Valbuena, L. A. Schiff, M. L. Nibert and S. C. Harrison (2001). “Structure of the reovirus outer capsid and dsRNA-binding protein sigma3 at 1.8 A resolution.” EMBO J 20(5): 979-989.
Osumi-Davis, P. A., M. C. de Aguilera, R. W. Woody and A. Y. Woody (1992). “Asp537, Asp812 are essential and Lys631, His811 are catalytically significant in bacteriophage T7 RNA polymerase activity.” J Mol Biol 226(1): 37-45.
Osumi-Davis, P. A., N. Sreerama, D. B. Volkin, C. R. Middaugh, R. W. Woody and A. Y. Woody (1994). “Bacteriophage T7 RNA polymerase and its active-site mutants. Kinetic, spectroscopic and calorimetric characterization.” J Mol Biol 237(1): 5-19.
Pack, P., M. Kujau, V. Schroeckh, U. Knupfer, R. Wenderoth, D. Riesenberg and A. Pluckthun (1993). “Improved bivalent miniantibodies, with identical avidity as whole antibodies, produced by high cell density fermentation of Escherichia coli.” Biotechnology (N Y) 11(11): 1271-1277.
Parisien, J. P., J. F. Lau, J. J. Rodriguez, B. M. Sullivan, A. Moscona, G. D. Parks, R. A. Lamb and C. M. Horvath (2001). “The V protein of human parainfluenza virus 2 antagonizes type I interferon responses by destabilizing signal transducer and activator of transcription 2.” Virology 283(2): 230-239.
Park, C., C. Peng, M. J. Rahman, S. L. Haller, L. Tazi, G. Brennan and S. Rothenburg (2020). “Orthopoxvirus K3 orthologs show virus- and host-specific inhibition of the antiviral protein kinase PKR.” bioRxiv: 2020.2002.2020.958645.
Park, C. Y., S. H. Oh, S. M. Kang, Y. S. Lim and S. B. Hwang (2009). “Hepatitis delta virus large antigen sensitizes to TNF-alpha-induced NF-kappaB signaling.” Mol Cells 28(1): 49-55.
Park, K. J., S. H. Choi, D. H. Choi, J. M. Park, S. W. Yie, S. Y. Lee and S. B. Hwang (2003). “1Hepatitis C virus NS5A protein modulates c-Jun N-terminal kinase through interaction with tumor necrosis factor receptor-associated factor 2.” J Biol Chem 278(33): 30711-30718.
Patel, R. C. and G. C. Sen (1992). “Identification of the double-stranded RNA-binding domain of the human interferon-inducible protein kinase.” J Biol Chem 267(11): 7671-7676.
Patel, R. C. and G. C. Sen (1998). “Requirement of PKR dimerization mediated by specific hydrophobic residues for its activation by double-stranded RNA and its antigrowth effects in yeast.” Mol Cell Biol 18(12): 7009-7019.
Patel, R. C., P. Stanton and G. C. Sen (1996). “Specific mutations near the amino terminus of double-stranded RNA-dependent protein kinase (PKR) differentially affect its double-stranded RNA binding and dimerization properties.” J Biol Chem 271(41): 25657-25663.
Pavitt, G. D., K. V. Ramaiah, S. R. Kimball and A. G. Hinnebusch (1998). “eIF2 independently binds two distinct eIF2B subcomplexes that catalyze and regulate guanine-nucleotide exchange.” Genes Dev 12(4): 514-526.
Pena, L., R. J. Yanez, Y. Revilla, E. Vinuela and M. L. Salas (1993). “African swine fever virus guanylyltransferase.” Virology 193(1): 319-328.
Peterhans, E. and M. Schweizer (2013). “BVDV: a pestivirus inducing tolerance of the innate immune response.” Biologicals 41(1): 39-51.
Pichlmair, A., O. Schulz, C. P. Tan, T. I. Naslund, P. Liljestrom, F. Weber and C. Reis e Sousa (2006). “RIG-I-mediated antiviral responses to single-stranded RNA bearing 5′-phosphates.” Science 314(5801): 997-1001.
Piehler, J., C. Thomas, K. C. Garcia and G. Schreiber (2012). “Structural and dynamic determinants of type I interferon receptor assembly and their functional interpretation.” Immunol Rev 250(1): 317-334.
Platanitis, E., D. Demiroz, A. Schneller, K. Fischer, C. Capelle, M. Hartl, T. Gossenreiter, M. Muller, M. Novatchkova and T. Decker (2019). “A molecular switch from STAT2-IRF9 to ISGF3 underlies interferon-induced gene transcription.” Nat Commun 10(1): 2921.
Pluckthun, A. and P. Pack (1997). “New protein engineering approaches to multivalent and bispecific antibody fragments.” Immunotechnology 3(2): 83-105.
Portnoff, A. D., E. A. Stephens, J. D. Varner and M. P. DeLisa (2014). “Ubiquibodies, synthetic E3 ubiquitin ligases endowed with unnatural substrate specificity for targeted protein silencing.” J Biol Chem 289(11): 7844-7855.
Precious, B. L., T. S. Carlos, S. Goodbourn and R. E. Randall (2007). “Catalytic turnover of STAT1 allows PIV5 to dismantle the interferon-induced anti-viral state of cells.” Virology 368(1): 114-121.
Pytel, D., K. Seyb, M. Liu, S. S. Ray, J. Concannon, M. Huang, G. D. Cuny, J. A. Diehl and M. A. Glicksman (2014).
“Enzymatic Characterization of ER Stress-Dependent Kinase, PERK, and Development of a High-Throughput Assay for Identification of PERK Inhibitors.” J Biomol Screen 19(7): 1024-1034.
Raab, D., M. Graf, F. Notka, T. Schodl and R. Wagner (2010). “The GeneOptimizer Algorithm: using a sliding window approach to cope with the vast sequence space in multiparameter DNA sequence optimization.” Syst Synth Biol 4(3): 215-225.
Raabe, T., F. J. Bollum and J. L. Manley (1991). “Primary structure and expression of bovine poly(A) polymerase.” Nature 353(6341): 229-234.
Raabe, T., K. G. Murthy and J. L. Manley (1994). “Poly(A) polymerase contains multiple functional domains.” Mol Cell Biol 14(5): 2946-2957.
Ramachandran, A., J. P. Parisien and C. M. Horvath (2008). “STAT2 is a primary target for measles virus V protein-mediated alpha/beta interferon signaling inhibition.” J Virol 82(17): 8330-8338.
Reynaud, J. M., D. Y. Kim, S. Atasheva, A. Rasalouskaya, J. P. White, M. S. Diamond, S. C. Weaver, E. I. Frolova and I. Frolov (2015). “IFIT1 Differentially Interferes with Translation and Replication of Alphavirus Genomes and Promotes Induction of Type I Interferon.” PLoS Pathog 11(4): e1004863.
Rodriguez, J. J., J. P. Parisien and C. M. Horvath (2002). “Nipah virus V protein evades alpha and gamma interferons by preventing STAT1 and STAT2 activation and nuclear accumulation.” J Virol 76(22): 11476-11483.
Ronald, J. A., L. Cusso, H. Y. Chuang, X. Yan, A. Dragulescu-Andrasi and S. S. Gambhir (2013). “Development and validation of non-integrative, self-limited, and replicating minicircles for safe reporter gene imaging of cell-based therapies.” PLoS One 8(8): e73138.
Ronco, L. V., A. Y. Karpova, M. Vidal and P. M. Howley (1998). “Human papillomavirus 16 E6 oncoprotein binds to interferon regulatory factor-3 and inhibits its transcriptional activity.” Genes Dev 12(13): 2061-2072.
Saira, K., Y. Zhou and C. Jones (2007). “The infected cell protein 0 encoded by bovine herpesvirus 1 (bICP0) induces degradation of interferon response factor 3 and, consequently, inhibits beta interferon promoter activity.” J Virol 81(7): 3077-3086.
Saito, T. and M. Gale, Jr. (2008). “Differential recognition of double-stranded RNA by RIG-I-like receptors in antiviral immunity.” J Exp Med 205(7): 1523-1527.
Sakamoto, K. M., K. B. Kim, A. Kumagai, F. Mercurio, C. M. Crews and R. J. Deshaies (2001). “Protacs: chimeric molecules that target proteins to the Skp1-Cullin-F box complex for ubiquitination and degradation.” Proc Natl Acad Sci USA 98(15): 8554-8559.
Schakowski, F., M. Gorschluter, C. Junghans, M. Schroff, P. Buttgereit, C. Ziske, B. Schottker, S. A. Konig-Merediz, T. Sauerbruch, B. Wittig and I. G. Schmidt-Wolf (2001). “A novel minimal-size vector (MIDGE) improves transgene expression in colon carcinoma cells and avoids transfection of undesired DNA.” Mol Ther 3(5 Pt 1): 793-800.
Schwartz, T., M. A. Rould, K. Lowenhaupt, A. Herbert and A. Rich (1999). “Crystal structure of the Zalpha domain of the human editing enzyme ADAR1 bound to left-handed Z-DNA.” Science 284(5421): 1841-1845.
Seago, J., L. Hilton, E. Reid, V. Doceul, J. Jeyatheesan, K. Moganeradj, J. McCauley, B. Charleston and S. Goodbourn (2007). “The Npro product of classical swine fever virus and bovine viral diarrhea virus uses a conserved mechanism to target interferon regulatory factor-3.” J Gen Virol 88(Pt 11): 3002-3006.
Siu, K. L., K. H. Kok, M. J. Ng, V. K. M. Poon, K. Y. Yuen, B. J. Zheng and D. Y. Jin (2009). “Severe acute respiratory syndrome coronavirus M protein inhibits type I interferon production by impeding the formation of TRAF3.TANK.TBK1/IKKepsilon complex.” J Biol Chem 284(24): 16202-16209.
Spiegel, M., A. Pichlmair, L. Martinez-Sobrido, J. Cros, A. Garcia-Sastre, O. Haller and F. Weber (2005). “Inhibition of Beta interferon induction by severe acute respiratory syndrome coronavirus suggests a two-step model for activation of interferon regulatory factor 3.” J Virol 79(4): 2079-2086.
Spurgeon, M. E. and D. A. Ornelles (2009). “The adenovirus E1B 55-kilodalton and E4 open reading frame 6 proteins limit phosphorylation of eIF2alpha during the late phase of infection.” J Virol 83(19): 9970-9982.
Su, Y., S. Ishikawa, M. Kojima and B. Liu (2003). “Eradication of pathogenic beta-catenin by Skp1/Cullin/F box ubiquitination machinery.” Proc Natl Acad Sci USA 100(22): 12729-12734.
Teske, B. F., T. D. Baird and R. C. Wek (2011). “Methods for analyzing eIF2 kinases and translational control in the unfolded protein response.” Methods Enzymol 490: 333-356.
Unterholzner, L., R. P. Sumner, M. Baran, H. Ren, D. S. Mansur, N. M. Bourke, F. Randow, G. L. Smith and A. G. Bowie (2011). “Vaccinia virus protein C6 is a virulence factor that binds TBK-1 adaptor proteins and inhibits activation of IRF3 and IRF7.” PLoS Pathog 7(9): e1002247.
Valegard, K., J. B. Murray, N. J. Stonehouse, S. van den Worm, P. G. Stockley and L. Liljas (1997). “The three-dimensional structures of two complexes between recombinant MS2 capsids and RNA operator fragments reveal sequence-specific protein-RNA interactions.” J Mol Biol 270(5): 724-738.
Valmas, C. and C. F. Basler (2011). “Marburg virus VP40 antagonizes interferon signaling in a species-specific manner.” J Virol 85(9): 4309-4317.
Vander Veen, R. L., D. L. Harris and K. I. Kamrud (2012). “Alphavirus replicon vaccines.” Anim Health Res Rev 13(1): 1-9.
Varga, Z. T., A. Grant, B. Manicassamy and P. Palese (2012). “Influenza virus protein PB1-F2 inhibits the induction of type I interferon by binding to MAVS and decreasing mitochondrial membrane potential.” J Virol 86(16): 8359-8366.
Vattern, K. M. and R. C. Wek (2004). “Reinitiation involving upstream ORFS regulates ATF4 mRNA translation in mammalian cells.” Proc Natl Acad Sci USA 101(31): 11269-11274.
Venkataraman, T., M. Valdes, R. Elsby, S. Kakuta, G. Caceres, S. Saijo, Y. Iwakura and G. N. Barber (2007). “Loss of DExD/H box RNA helicase LGP2 manifests disparate antiviral responses.” J Immunol 178(10): 6444-6455.
Verrier, S. B. and O. Jean-Jean (2000). “Complementarity between the mRNA 5′ untranslated region and 18S ribosomal RNA can inhibit translation.” RNA 6(4): 584-597.
Vethantham, V., N. Rao and J. L. Manley (2008). “Sumoylation regulates multiple aspects of mammalian poly(A) polymerase function.” Genes Dev 22(4): 499-511.
Wang, J. T., S. L. Doong, S. C. Teng, C. P. Lee, C. H. Tsai and M. R. Chen (2009). “Epstein-Barr virus BGLF4 kinase suppresses the interferon regulatory factor 3 signaling pathway.” J Virol 83(4): 1856-1869.
Wei, C., C. Ni, T. Song, Y. Liu, X. Yang, Z. Zheng, Y. Jia, Y. Yuan, K. Guan, Y. Xu, X. Cheng, Y. Zhang, X. Yang, Y. Wang, C. Wen, Q. Wu, W. Shi and H. Zhong (2010). “The hepatitis B virus X protein disrupts innate immunity by downregulating mitochondrial antiviral signaling protein.” J Immunol 185(2): 1158-1168.
Weihua, X., S. Ramanujam, D. J. Lindner, R. D. Kudaravalli, R. Freund and D. V. Kalvakolanu (1998). “The polyoma virus T antigen interferes with interferon-inducible gene expression.” Proc Natl Acad Sci USA 95(3): 1085-1090.
White, S. D. and B. L. Jacobs (2012). “The amino terminus of the vaccinia virus E3 protein is necessary to inhibit the interferon response.” J Virol 86(10): 5895-5904.
Wu, S., P. Xie, K. Welsh, C. Li, C. Z. Ni, X. Zhu, J. C. Reed, A. C. Satterthwait, G. A. Bishop and K. R. Ely (2005). “LMP1 protein from the Epstein-Barr virus is a structural CD40 decoy in B lymphocytes for binding to TRAF3.” J Biol Chem 280(39): 33620-33626.
Yang, W. and A. G. Hinnebusch (1996). “Identification of a regulatory subcomplex in the guanine nucleotide exchange factor eIF2B that mediates inhibition by phosphorylated eIF2.” Mol Cell Biol 16(11): 6603-6616.
Yang, Y., Y. Liang, L. Qu, Z. Chen, M. Yi, K. Li and S. M. Lemon (2007). “Disruption of innate immunity due to mitochondrial targeting of a picornaviral protease precursor.” Proc Natl Acad Sci USA 104(17): 7253-7258.
Zhang, F., A. Moon, K. Childs, S. Goodbourn and L. K. Dixon (2010). “The African swine fever virus DP71L protein recruits the protein phosphatase 1 catalytic subunit to dephosphorylate eIF2alpha and inhibits CHOP induction but is dispensable for these activities during virus infection.” J Virol 84(20): 10681-10689.
Zhang, F., P. R. Romano, T. Nagamura-Inoue, B. Tian, T. E. Dever, M. B. Mathews, K. Ozato and A. G. Hinnebusch (2001). “Binding of double-stranded RNA to protein kinase PKR is required for dimerization and promotes critical autophosphorylation events in the activation loop.” J Biol Chem 276(27): 24946-24958.
Zhang, P., B. C. McGrath, J. Reinert, D. S. Olsen, L. Lei, S. Gill, S. A. Wek, K. M. Vattern, R. C. Wek, S. R. Kimball, L. S. Jefferson and D. R. Cavener (2002). “The GCN2 eIF2alpha kinase is required for adaptation to amino acid deprivation in mice.” Mol Cell Biol 22(19): 6681-6688.
Zhou, P., R. Bogacki, L. McReynolds and P. M. Howley (2000). “Harnessing the ubiquitination machinery to target the degradation of specific cellular proteins.” Mol Cell 6(3): 751-756.
Zhu, F. X., S. M. King, E. J. Smith, D. E. Levy and Y. Yuan (2002). “A Kaposi's sarcoma-associated herpesviral protein inhibits virus-mediated induction of type I interferon by blocking IRF-7 phosphorylation and nuclear accumulation.” Proc Natl Acad Sci USA 99(8): 5573-5578.
Zurney, J., T. Kobayashi, G. H. Holm, T. S. Dermody and B. Sherry (2009). “Reovirus mug protein inhibits interferon signaling through a novel mechanism involving nuclear accumulation of interferon regulatory factor 9.” J Virol 83(5): 2178-2187.

1. An ex vivo, in vitro or in cellulo method for expressing a recombinant DNA molecule in a eukaryotic host cell, comprising the steps of:
(a) expressing or introducing at least one chimeric protein, in said host cell, wherein said chimeric protein comprises:
at least one catalytic domain of a capping enzyme, in particular selected in the group consisting of cap-0 canonical capping enzymes, cap-0 non-canonical capping enzymes, cap-1 capping enzymes and cap-2 capping enzymes; and
at least one catalytic domain of a DNA-dependent RNA polymerase, in particular a bacteriophage DNA-dependent RNA polymerase,
(b) constitutively or transiently downregulating the phosphorylation level of subunit a of translation initiation factor eIF2 (eIF2α) in said host cell.

2. The method according to claim 1, wherein step (b) comprises introducing, into said host cell, at least one polypeptide or a nucleic acid molecule encoding said polypeptide, wherein the polypeptide modulates the activity or the expression of a target host cell protein involved in the regulation of the phosphorylation level of eIF2α, preferably of a target host cell protein selected from EIF2AK2, EIF2AK3, DDX58, IFIH1, MAVS, IFNAR1, IFNAR2, IRF3, IRF7, IFNB1, TBK1, TRAF2, TRAF3, IFIT1, JAK1, TYK2, STAT1, STAT2, IRF9, or protein phosphatase 1 PP1 or a subunit thereof, in particular PPP1CA or PPP1R15.

3. The method according to claim 2, wherein said polypeptide modulating the activity or the expression of a target host cell protein involved in the regulation of the phosphorylation level of eIF2α is selected from:
(a) a viral protein selected from E3L of vaccinia virus, NSs from Rift Valley fever virus, NPRO from Bovine Viral Diarrhea Virus, V protein from parainfluenza virus type 5, ICP34.5 from human Herpes-simplex virus-1, NS1 from Influenza A virus, NS1 protein from human respiratory syncytial virus, K3L of vaccinia virus, DP71L from African swine fever virus, in particular the DP71(s) and DP71L(1) isoforms, VP35 from Zaire Ebolavirus, VP40 from the Marburg virus, LMP-1 from Epstein-Barr virus, μ2 from reovirus, B18R of vaccinia virus, and ORF4a from Middle East respiratory syndrome coronavirus, a protein with at least 40% amino acid sequence identity with one of E3L of vaccinia virus, NSs from Rift Valley fever virus, NPRO from Bovine Viral Diarrhea Virus, V protein from parainfluenza virus type 5, ICP34.5 from human Herpes-simplex virus-1, NS1 from Influenza A virus, NS1 protein from human respiratory syncytial virus, K3L of vaccinia virus, DP71L from African swine fever virus, in particular the DP71(s) and DP71L(1) isoforms, VP35 from Zaire Ebolavirus, VP40 from the Marburg virus from the Marburg virus, LMP-1 from Epstein-Barr virus, μ2 from reovirus, B18R of vaccinia virus and ORF4a from Middle East respiratory syndrome coronavirus, or a biologically active fragment thereof;
(b) PPP1CA catalytic subunit and its regulatory proteins, in particular its host-cell regulatory proteins such as the eukaryotic protein PPP1R15, or a protein with at least 40% amino acid sequence identity with PPP1CA or PPP1R15, or a biologically active fragment thereof;
(c) an inactive mutant of a host cell protein involved in the regulation of the phosphorylation level of eIF2α, in particular selected from EIF2AK2 or EIF2AK3, or a biologically active fragment thereof, in particular the K296R mutant of the human EIF2AK2, or the dsRNA binding domain from EIF2AK2 deleted of its carboxy-terminal kinase domain, or a biologically active fragment thereof.

4. The method according to claim 2, wherein said polypeptide modulating the activity or the expression of a target host cell protein involved in the regulation of the phosphorylation level of eIF2α is an eIF2AK2 inhibitor comprising at least one Zα domain, in particular a Zα domain from E3L of vaccinia virus or mammalian ADAR1 operably linked to at least one dsRNA-binding domain, in particular a dsRNA-binding domain from Influenza A virus NS1 protein, mammalian EIF2AK2, Flock House virus B2 protein, orthoreovirus σ3 protein, preferably selected from Influenza A virus NS1 and mammalian EIF2AK2 proteins.

5. The method according to claim 2, wherein said polypeptide modulating the activity or the expression of a target host cell protein involved in the regulation of the phosphorylation level of eIF2α is an eIF2AK2 inhibitor comprising:
(a) the amino acid sequence set forth in SEQ ID NO. 16; or
(b) an amino acid sequence with at least 40% amino acid sequence identity with SEQ ID NO. 16; or
(c) a biologically active fragment of (a) or (b).

6. The method according to claim 2, wherein said polypeptide modulating the activity or the expression of a target host cell protein involved in the regulation of the phosphorylation level of eIF2α is a chimeric protein comprising:
a. a polypeptide capable of selectively binding to EIF2AK2, preferably selected from
dsRNA-binding region from EIF2AK2 protein deleted of its carboxyl-terminal kinase domain; or
orthologous dsRNA binding domains such as the dsRNA-binding domain of E3L protein from vaccinia virus; or
single-chain antibodies, such as nanobodies or ScFv, raised against EIF2AK2; and
b. a specific domain from multimeric E3 ligases, preferably selected from:
Skp1-interacting domains from BTRCP, FBW7, SPK2; or
Elongin BC-interacting domains from VHL; or
Cullin3-interacting domains from SPOP; or
DDB1-interacting domains from CRBN or DDB2; or
Elongin BC-interacting domains from SOCS2; or
U-box interacting domain and coiled-coil dimerization domain from STUB1; or
CUL1-interacting domain from Skp1.

7. The method according to claim 1, wherein step (b) comprises introducing, into said host cell, at least two polypeptides, or one or more nucleic acid molecules encoding said polypeptides, wherein said polypeptides modulate the activity or the expression of at least two different target host cell proteins involved in the regulation of the phosphorylation level of eIF2α, preferably wherein the modulation by said polypeptides has a supra-additive effect on the expression of said recombinant DNA by said host cell.

8. The method according to claim 7, wherein one of said at least two polypeptides inhibits the phosphorylation of eIF2α, preferably is an EIF2AK2 inhibitor such as the dsRNA binding domain from EIF2AK2 deleted of its carboxy-terminal kinase domain, or a biologically active fragment thereof, and wherein another of said at least two polypeptides activates the dephosphorylation of eIF2α, preferably is selected from PPP1CA or its viral and host-cell regulatory proteins, in particular PPP1R15, DP71L from African swine fever virus, such as its isoforms DP71L(s) or DP71L(1) and ICP34.5 from human Herpes-simplex virus-1 or a biologically active fragment thereof.

9. The method according to claim 1, wherein step (b) comprises introducing, into said host cell, a polypeptide comprising, the sequence of SEQ ID NO. 20 or SEQ ID NO. 36 or a sequence with at least 40% identity to SEQ ID NO. 20 or SEQ ID NO. 36, or a nucleic acid sequence encoding said polypeptide, wherein said polypeptide is capable of downregulating the phosphorylation level of eIF2α.

10. The method according to claim 1, wherein step (a) further comprises expressing at least one catalytic domain of a poly(A) polymerase, potentially tethered through a lambdoid N-peptide, in said host cell.

11. A eukaryotic host cell for the expression of a recombinant protein, characterized in that the phosphorylation level of eIF2α is constitutively or transiently downregulated in said cell, and wherein said cell comprises at least one nucleic acid molecule encoding at least one chimeric protein comprising:
(i) at least one catalytic domain of a capping enzyme, in particular selected in the group consisting of cap-0 canonical capping enzymes, cap-0 non-canonical capping enzymes, cap-1 capping enzymes and cap-2 capping enzymes; and
(ii) at least one catalytic domain of a DNA-dependent RNA polymerase, in particular a bacteriophage DNA-dependent RNA polymerase.

12. A eukaryotic host cell according to claim 11, further comprising a heterologous nucleic acid sequence encoding at least one polypeptide modulating the activity or the expression of a target host cell protein involved in the regulation of the phosphorylation level of eIF2α by introducing, into said host cell, at least one polypeptide or a nucleic acid molecule encoding said polypeptide, wherein the polypeptide modulates the activity or the expression of a target host cell protein involved in the regulation of the phosphorylation level of eIF2α, preferably of a target host cell protein selected from EIF2AK2, EIF2AK3, DDX58, IFIH1, MAVS, IFNAR1, IFNAR2, IRF3, IRF7, IFNB1, TBK1, TRAF2, TRAF3, IFIT1, JAK1, TYK2, STAT1, STAT2, IRF9, or protein phosphatase 1 PP1 or a subunit thereof, in particular PPP1CA or PPP1R15.

13. The eukaryotic host cell according to claim 12, further comprising a nucleic acid molecule comprising:
at least one nucleic acid sequence encoding a chimeric protein comprising:
(i) at least one catalytic domain of a capping enzyme; and
(ii) at least one catalytic domain of a DNA-dependent RNA polymerase;
at least one nucleic acid sequence encoding at least one polypeptide modulating the activity or the expression of a target host cell protein involved in the regulation of the phosphorylation level of eIF2α by introducing, into said host cell, at least one polypeptide or a nucleic acid molecule encoding said polypeptide, wherein the polypeptide modulates the activity or the expression of a target host cell protein involved in the regulation of the phosphorylation level of eIF2α, preferably of a target host cell protein selected from EIF2AK2, EIF2AK3, DDX58, IFIH1, MAVS, IFNAR1, IFNAR2, IRF3, IRF7, IFNB1, TBK1, TRAF2, TRAF3, IFIT1, JAK1, TYK2, STAT1, STAT2, IRF9, or protein phosphatase 1 PP1 or a subunit thereof, in particular PPP1CA or PPP1R15; and optionally, at least one nucleic acid sequence encoding a poly(A) polymerase, potentially tethered through a lambdoid N-peptide.

14. An isolated nucleic acid molecule or a set of nucleic acid molecules, comprising:
(a) at least one nucleic acid sequence encoding a chimeric protein comprising:
(i) at least one catalytic domain of a capping enzyme, in particular selected in the group consisting of cap-0 canonical capping enzymes, cap-0 non-canonical capping enzymes, cap-1 capping enzymes and cap-2 capping enzymes; and
(ii) at least one catalytic domain of a DNA-dependent RNA polymerase; and
(b) at least one nucleic acid sequence downregulating the phosphorylation level of eIF2α in a eukaryotic host cell, or encoding a polypeptide downregulating said phosphorylation level.

15. The isolated nucleic acid molecule or set of nucleic acid molecules according to claim 14, wherein said nucleic acid sequence downregulating the phosphorylation level of eIF2α encodes at least one polypeptide modulating the activity or the expression of a target host cell protein involved in the regulation of the phosphorylation level of eIF2α in a eukaryotic host cell, preferably modulating the activity or the expression of a target host cell protein selected from EIF2AK2, EIF2AK3, DDX58, IFIH1, MAVS, IFNAR1, IFNAR2, IRF3, IRF7, IFNB1, TBK1, TRAF2, TRAF3, IFIT1, a type-I interferon protein, JAK1, TYK2, STAT1, STAT2, IRF9, or protein phosphatase 1 PP1 or a subunit thereof, in particular PPP1CA or PPP1R15.

16. The isolated nucleic acid molecule or set of nucleic acid molecules according to claim 14, wherein said polypeptide modulating the activity or the expression of a target host cell protein involved in the regulation of the phosphorylation level of eIF2α is selected from:
(a) a viral protein selected from E3L from vaccinia virus, NSs from Rift Valley fever virus, NPRO from Bovine Viral Diarrhea Virus, V protein from parainfluenza virus type 5, ICP34.5 from human Herpes-simplex virus-1, NS1 from influenza A virus, K3L from vaccinia virus, DP71L from African swine fever virus, in particular the DP71(s) and DP71L(1) isoforms, VP35 from Zaire Ebolavirus, VP40 from Marburg virus, LMP-1 from Epstein-Barr virus, μ2 from reovirus, B18R from vaccinia virus and ORF4a from Middle East respiratory syndrome coronavirus, a protein with at least 40% amino acid sequence identity with one of E3L from vaccinia virus, NSs from Rift Valley fever virus, NPRO from Bovine Viral Diarrhea Virus, V protein from parainfluenza virus type 5, ICP34.5 from human Herpes-simplex virus-1, NS1 from influenza A virus, K3L from vaccinia virus, DP71L from African swine fever virus, in particular the DP71(s) and DP71L(1) isoforms, VP35 from Zaire Ebolavirus, VP40 from Marburg virus, LMP-1 from Epstein-Barr virus, μ2 from reovirus, B18R from vaccinia virus and ORF4a from Middle East respiratory syndrome coronavirus, or a biologically active fragment thereof;
(b) PPP1CA catalytic subunit and its regulatory proteins, in particular host-cell regulatory proteins such as the eukaryotic protein PPP1R15, or a protein with at least 40% amino acid sequence identity with PPP1CA or PPP1R15, or a biologically active fragment thereof;
(c) an inactive mutant of a host cell protein involved in the regulation of the phosphorylation level of eIF2α, in particular selected from EIF2AK2 or EIF2AK3 or a biologically active fragment thereof, in particular the K296R mutant of the human EIF2AK2 or a biologically active fragment thereof.

17. The isolated nucleic acid molecule or set of nucleic acid molecules according to claim 15, wherein said polypeptide modulating the activity or the expression of a target host cell protein involved in the regulation of the phosphorylation level of eIF2α is an eIF2AK2 inhibitor comprising at least one Zα domain, in particular a Zα domain from E3L of vaccinia virus or mammalian ADAR1 operably linked to at least one dsRNA-binding domain, in particular a dsRNA-binding domain from Influenza A virus NS1 protein, mammalian EIF2AK2, Flock House virus B2 protein, orthoreovirus σ3 protein, preferably selected from Influenza A virus NS1 and mammalian EIF2AK2 proteins.

18. The isolated nucleic acid molecule or set of nucleic acid molecules according to claim 14, further comprising at least one nucleic acid sequence encoding a poly(A) polymerase, potentially tethered through a lambdoid N-peptide and, from the 5′-terminus to the 3′-terminus:
said at least one nucleic acid sequence encoding a catalytic domain of a poly(A) polymerase potentially tethered through a lambdoid N-peptide;
said at least one nucleic acid sequence encoding said polypeptide downregulating the phosphorylation level of eIF2α; and
said at least one nucleic acid sequence encoding a chimeric protein comprising:
(i) at least one catalytic domain of a capping enzyme; and
(ii) at least one catalytic domain of a DNA-dependent RNA polymerase.

19. The isolated nucleic acid molecule or set of nucleic acid molecules according to any one of claim 14, further comprising at least one nucleic acid sequence encoding a poly(A) polymerase, potentially tethered through a lambdoid N-peptide and, from the 5′-terminus to the 3′-terminus:
said at least one nucleic acid sequence encoding said polypeptide downregulating the phosphorylation level of eIF2α;
said at least one nucleic acid sequence encoding a catalytic domain of a poly(A) polymerase potentially tethered through a lambdoid N-peptide; and
said at least one nucleic acid sequence encoding a chimeric protein comprising:
(iii) at least one catalytic domain of a capping enzyme; and
(iv) at least one catalytic domain of a DNA-dependent RNA polymerase.

20.-24. (canceled)


SEQUENCE LISTING (42 sequences)

SEQ ID NO: 1 - DNA, 5259 bp, Artificial Sequence
  Recombinant DNA open-reading frame from pC3P3- G1 plasmid
atggccagcctggacaacctggtggccagataccagcggtgcttcaacgaccagagcctgaagaacagcaccatcgagctggaaatccggttccagcagatcaacttcctgctgttcaagaccgtgtacgaggccctggtcgcccaggaaatccccagcaccatcagccacagcatccggtgcatcaagaaggtgcaccacgagaaccactgccgggagaagatcctgcccagcgagaacctgtacttcaagaaacagcccctgatgttcttcaagttcagcgagcccgccagcctgggctgtaaagtgtccctggccatcgagcagcccatccggaagttcatcctggacagcagcgtgctggtccggctgaagaaccggaccaccttccgggtgtccgagctgtggaagatcgagctgaccatcgtgaagcagctgatgggcagcgaggtgtcagccaagctggccgccttcaagaccctgctgttcgacacccccgagcagcagaccaccaagaacatgatgaccctgatcaaccccgacgacgagtacctgtacgagatcgagatcgagtacaccggcaagcctgagagcctgacagccgccgacgtgatcaagatcaagaacaccgtgctgacactgatcagccccaaccacctgatgctgaccgcctaccaccaggccatcgagtttatcgccagccacatcctgagcagcgagatcctgctggcccggatcaagagcggcaagtggggcctgaagagactgctgccccaggtcaagtccatgaccaaggccgactacatgaagttctacccccccgtgggctactacgtgaccgacaaggccgacggcatccggggcattgccgtgatccaggacacccagatctacgtggtggccgaccagctgtacagcctgggcaccaccggcatcgagcccctgaagcccaccatcctggacggcgagttcatgcccgagaagaaagagttctacggctttgacgtgatcatgtacgagggcaacctgctgacccagcagggcttcgagacacggatcgagagcctgagcaagggcatcaaggtgctgcaggccttcaacatcaaggccgagatgaagcccttcatcagcctgacctccgccgaccccaacgtgctgctgaagaatttcgagagcatcttcaagaagaaaacccggccctacagcatcgacggcatcatcctggtggagcccggcaacagctacctgaacaccaacaccttcaagtggaagcccacctgggacaacaccctggactttctggtccggaagtgccccgagtccctgaacgtgcccgagtacgcccccaagaagggcttcagcctgcatctgctgttcgtgggcatcagcggcgagctgtttaagaagctggccctgaactggtgccccggctacaccaagctgttccccgtgacccagcggaaccagaactacttccccgtgcagttccagcccagcgacttccccctggccttcctgtactaccaccccgacaccagcagcttcagcaacatcgatggcaaggtgctggaaatgcggtgcctgaagcgggagatcaactacgtgcgctgggagatcgtgaagatccgggaggaccggcagcaggatctgaaaaccggcggctacttcggcaacgacttcaagaccgccgagctgacctggctgaactacatggaccccttcagcttcgaggaactggccaagggacccagcggcatgtacttcgctggcgccaagaccggcatctacagagcccagaccgccctgatcagcttcatcaagcaggaaatcatccagaagatcagccaccagagctgggtgatcgacctgggcatcggcaagggccaggacctgggcagatacctggacgccggcgtgagacacctggtcggcatcgataaggaccagacagccctggccgagctggtgtaccggaagttctcccacgccaccaccagacagcacaagcacgccaccaacatctacgtgctgcaccaggatctggccgagcctgccaaagaaatcagcgagaaagtgcaccagatctatggcttccccaaagagggcgccagcagcatcgtgtccaacctgttcatccactacctgatgaagaacacccagcaggtcgagaacctggctgtgctgtgccacaagctgctgcagcctggcggcatggtctggttcaccaccatgctgggcgaacaggtgctggaactgctgcacgagaaccggatcgaactgaacgaagtgtgggaggcccgggagaacgaggtggtcaagttcgccatcaagcggctgttcaaagaggacatcctgcaggaaaccggccaggaaatcggcgtcctgctgcccttcagcaacggcgacttctacaatgagtacctggtcaacaccgcctttctgatcaagattttcaagcaccatggctttagcctcgtgcagaagcagagcttcaaggactggatccccgagttccagaacttcagcaagagcctgtacaagatcctgaccgaggccgacaagacctggaccagcctgttcggcttcatctgcctgcggaagaacggaggcgggggaagtggagggggcggcagtcaggacctgcacgccatccagctgcagctcgaagaggaaatgttcaacggcggcatcagaagattcgaggccgaccagcagagacagatcgcctctggcaacgagagcgacaccgcctggaatagaaggctgctgtctgagctgatcgcccctatggccgaaggcatccaggcctacaaagaggaatacgagggcaagagaggcagagcccctagagccctggccttcatcaactgtgtgggcaatgaggtggccgcctacatcaccatgaagatcgtgatggacatgctgaacaccgacgtgaccctgcaggccattgccatgaacgtggccgacagaatcgaggaccaggtccgattcagcaagctggaaggacacgccgccaagtacttcgagaaagtgaagaagtccctgaaggccagcaagaccaagagctacagacacgcccacaacgtggccgtggtggccgaaaaatctgtggccgatagggacgccgacttctctagatgggaggcctggcctaaggacaccctgctgcagatcggcatgaccctgctggaaatcctggaaaacagcgtgttcttcaacggccagcccgtgttcctgagaaccctgaggacaaatggcggcaagcacggcgtgtactacctgcagacatctgagcacgtgggcgagtggatcaccgccttcaaagaacatgtggcccagctgagccctgcctatgccccttgtgtgatccctcctagaccctgggtgtcccctttcaatggcggctttcacaccgagaaggtggccagcagaatcagactggtcaagggcaaccgggaacacgtgcggaagctgaccaagaaacagatgcccgccgtgtacaaggccgtgaatgctctgcaggccaccaagtggcaggtcaacaaagaggtgctgcaggtcgtcgaggacgtgatcagactggatctgggctacggcgtgccaagctttaagcccctgatcgacagagagaacaagcccgccaaccctgtgcccctggaatttcagcacctgagaggccgcgagctgaaagagatgctgacacctgaacagtggcaggcctttatcaattggaagggcgagtgcaccaagctgtacaccgccgagacaaagaggggctctaagtctgccgccacagtgcgaatggtcggacaggccagaaagtacagccagttcgacgccatctacttcgtgtacgccctggacagccggtctagagtgtatgcccagagcagcacactgagcccccagtctaacgatctgggaaaggccctgctgagattcaccgagggccagagactggattctgccgaagccctgaagtggttcctggtcaacggcgccaacaactggggctgggacaagaaaaccttcgatgtgcggaccgccaacgtgctggatagcgagttccaggacatgtgcagagatatcgccgccgaccctctgacctttacccagtgggtcaacgccgatagcccctatggattcctggcctggtgcttcgagtacgccagatacctggacgccctggatgagggaacccaggatcagttcatgacccatctgcccgtgcaccaggatggctcttgttctggcatccagcactacagcgccatgctgagcgatgccgtgggagccaaagccgtgaacctgaagcctagcgacagcccccaggatatctatggcgctgtggcccaggtggtcatccagaaaaactacgcctacatgaacgccgaggacgccgagacattcacaagcggaagcgtgacactgacaggcgccgagctgagatctatggcctctgcctgggacatgatcggcatcacacggggcctgaccaaaaagcctgtgatgacactgccctacggcagcaccagactgacctgtagagaaagcgtgatcgactacatcgtggacctggaagagaaagaggcccagagagccattgccgagggcagaacagccaatcctgtgcaccccttcgacaacgaccggaaggatagcctgacacctagcgccgcctacaactacatgaccgccctgatctggcccagcatctctgaagtggtcaaggcccctatcgtggccatgaagatgatcagacagctggccagattcgccgccaagagaaatgagggcctggaataccctctgcccaccggctttatcctgcagcagaaaatcatggccaccgacatgctgcgggtgtccacatgtctgatgggcgagatcaagatgagcctgcagatcgagacagacgtggtggacgagacagccatgatgggagccgccgctcctaattttgtgcacggacacgatgccagccacctgatcctgaccgtgtgcgatctggtggacaagggcatcactagcgtggccgtgatccacgatagctttggaacacacgccggcagaaccgccgacctgagagattctctgcgggaagagatggtcaagatgtaccagaaccacaacgccctgcagaacctgctggacgtgcacgaagaaagatggctggtggacaccggcatccaggtgccagaacagggagagttcgacctgaacgagatcctggtgtccgactactgcttcgcctga

SEQ ID NO: 2 - protein, 1751 aa, Artificial Sequence
  Recombinant protein encoded by pC3P3-G1 plasmid
ASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSQDLHAIQLQLEEEMFNGGIRRFEADQQRQIASGNESDTAWNRRLLSELIAPMAEGIQAYKEEYEGKRGRAPRALAFINCVGNEVAAYITMKIVMDMLNTDVTLQAIAMNVADRIEDQVRFSKLEGHAAKYFEKVKKSLKASKTKSYRHAHNVAVVAEKSVADRDADFSRWEAWPKDTLLQIGMTLLEILENSVFFNGQPVFLRTLRTNGGKHGVYYLQTSEHVGEWITAFKEHVAQLSPAYAPCVIPPRPWVSPFNGGFHTEKVASRIRLVKGNREHVRKLTKKQMPAVYKAVNALQATKWQVNKEVLQVVEDVIRLDLGYGVPSFKPLIDRENKPANPVPLEFQHLRGRELKEMLTPEQWQAFINWKGECTKLYTAETKRGSKSAATVRMVGQARKYSQFDAIYFVYALDSRSRVYAQSSTLSPQSNDLGKALLRFTEGQRLDSAEALKWFLVNGANNWGWDKKTFDVRTANVLDSEFQDMCRDIAADPLTFTQWVNADSPYGFLAWCFEYARYLDALDEGTQDQFMTHLPVHQDGSCSGIQHYSAMLSDAVGAKAVNLKPSDSPQDIYGAVAQVVIQKNYAYMNAEDAETFTSGSVTLTGAELRSMASAWDMIGITRGLTKKPVMTLPYGSTRLTCRESVIDYIVDLEEKEAQRAIAEGRTANPVHPFDNDRKDSLTPSAAYNYMTALIWPSISEVVKAPIVAMKMIRQLARFAAKRNEGLEYPLPTGFILQQKIMATDMLRVSTCLMGEIKMSLQIETDVVDETAMMGAAAPNFVHGHDASHLILTVCDLVDKGITSVAVIHDSFGTHAGRTADLRDSLREEMVKMYQNHNALQNLLDVHEERWLVDTGIQVPEQGEFDLNEILVSDYCFA

SEQ ID NO: 3 - DNA, 7674 bp, Artificial Sequence
  Recombinant DNA open-reading frame from pC3P3- G2 plasmid
atggatgctcagaccagacgcagagaacggcgggcagagaagcaggcacagtggaaggccgctaatccgtttccagttacaacgcagggatcacaacaaacgcagccaccacagaggcactatggcattacctctcctatcagcttagcggcccccaaggagactgactgcctactcacacagaagctcatcgagacgctgaagccctttggggtttttgaagaagaagaggaactgcagcgcaggattttaattttgggaaaattaaataacctggtgaaagaatggattcgagaaatcagtgaaagcaagaatctcccacaatctgtaattgaaaatgttggagggaagatttttacatttggatcttacagactaggagtccacacgaaaggtgctgatattgatgcgttgtgtgttgcaccaagacatgttgatcgaagtgactttttcacctcattctatgataaattgaaattacaagaagaagtgaaagatttaagagctgttgaagaggcatttgtaccagttatcaaactctgttttgatggaatagagattgatattttgtttgcaagattagcactgcagactattccagaagatttggacctacgagatgacagtctgcttaaaaacctagatataagatgcataagaagccttaatggttgcagggtaaccgatgaaattttacatctagtaccaaacattgacaacttcaggttaactctgagagccatcaaactgtgggccaaacggcacaacatctattccaatatattaggtttcctcggtggtgtttcctgggctatgctagtagcaagaacttgccagctttatccaaatgcaatagcatcaactcttgtacataaatttttcttggtattttctaaatgggaatggccaaatccagtgctattgaaacagcctgaagaatgcaatcttaatttgcctgtgtgggacccaagggtaaaccccagtgataggtaccatcttatgcctataattacaccagcatacccacagcagaactccacgtacaatgtgtccgtttcaacacggatggtcatggttgaggagtttaaacaaggtcttgctatcacagatgaaattttgctgagtaaggcagagtggtccaaactttttgaagctccaaacttctttcagaagtacaagcattatattgtacttctagcaagtgcgcccacggaaaagcagcgtctggaatgggtgggcttggtggaatcaaaaatccgcatcctggttggaagcttggagaagaatgagtttattacactggctcatgtgaatccccagtcatttccagcccccaaagaaagtcctgacagggaagaatttcgcacaatgtgggtgattgggttagtgtttaaaaaaactgaaaactctgaaaatctcagtgtcgacctcacctatgatatccagtctttcacagacacagtttataggcaagcaataaacagcaaaatgtttgagttggatatgaagattgcagcaatgcatgtgaagagaaagcaactccatcagctgctgcctagtcacgtgcttcagaagaggaagaagcattcaacagaaggagtcaagttaacagctctgaatgacagcagccttgacttgtctatggacagtgataacagcatgtctgtgccttcacccaccagtgctatgaagaccagtccattgaatagttctggcagctcccagggcagaaacagtcctgctccagctgtgaccgcagcatctgtgaccagcatccaggcttctgaggtttctgtaccgcaagcaaattccagtgaaagcccagggggtccatcgagcgaaagcattcctcaaactgccacacagccagccattgccccaccaccaaagcctacagtctccagagttgtctcctcaacacgactggtaaacccatcgcctagaccttcaggaaacacagcaacaaaagtccctaatcctatagtaggagtcaagagaacgtccgcccccaataaagaagaagcccctagaaggaccaaaacagaagaggatgaaacaagtgaagatgctaactgtcttgctttgagtggacatgataaaacagagacaaaggaacaagttgatctggagacaagtgcggttcaatcagaaactgttccggcatcggcttctctgttggcctctcagaaaacatccagtacagacctttctgatatccctgctctccctgcaaatcctattcctgttatcaagaactcaataaaactgagactgaatcggggatcaggccctcgcccactgctcgcaatccaccctactgaagcaaggcacaagcaaaaaattgtggctcctgtgaagcagacgctgaactttgatctgctgaagctcgctggcgacgtggagtcgaaccctggccctgccagcctggacaacctggtggccagataccagcggtgcttcaacgaccagagcctgaagaacagcaccatcgagctggaaatccggttccagcagatcaacttcctgctgttcaagaccgtgtacgaggccctggtcgcccaggaaatccccagcaccatcagccacagcatccggtgcatcaagaaggtgcaccacgagaaccactgccgggagaagatcctgcccagcgagaacctgtacttcaagaaacagcccctgatgttcttcaagttcagcgagcccgccagcctgggctgtaaagtgtccctggccatcgagcagcccatccggaagttcatcctggacagcagcgtgctggtccggctgaagaaccggaccaccttccgggtgtccgagctgtggaagatcgagctgaccatcgtgaagcagctgatgggcagcgaggtgtcagccaagctggccgccttcaagaccctgctgttcgacacccccgagcagcagaccaccaagaacatgatgaccctgatcaaccccgacgacgagtacctgtacgagatcgagatcgagtacaccggcaagcctgagagcctgacagccgccgacgtgatcaagatcaagaacaccgtgctgacactgatcagccccaaccacctgatgctgaccgcctaccaccaggccatcgagtttatcgccagccacatcctgagcagcgagatcctgctggcccggatcaagagcggcaagtggggcctgaagagactgctgccccaggtcaagtccatgaccaaggccgactacatgaagttctacccccccgtgggctactacgtgaccgacaaggccgacggcatccggggcattgccgtgatccaggacacccagatctacgtggtggccgaccagctgtacagcctgggcaccaccggcatcgagcccctgaagcccaccatcctggacggcgagttcatgcccgagaagaaagagttctacggctttgacgtgatcatgtacgagggcaacctgctgacccagcagggcttcgagacacggatcgagagcctgagcaagggcatcaaggtgctgcaggccttcaacatcaaggccgagatgaagcccttcatcagcctgacctccgccgaccccaacgtgctgctgaagaatttcgagagcatcttcaagaagaaaacccggccctacagcatcgacggcatcatcctggtggagcccggcaacagctacctgaacaccaacaccttcaagtggaagcccacctgggacaacaccctggactttctggtccggaagtgccccgagtccctgaacgtgcccgagtacgcccccaagaagggcttcagcctgcatctgctgttcgtgggcatcagcggcgagctgtttaagaagctggccctgaactggtgccccggctacaccaagctgttccccgtgacccagcggaaccagaactacttccccgtgcagttccagcccagcgacttccccctggccttcctgtactaccaccccgacaccagcagcttcagcaacatcgatggcaaggtgctggaaatgcggtgcctgaagcgggagatcaactacgtgcgctgggagatcgtgaagatccgggaggaccggcagcaggatctgaaaaccggcggctacttcggcaacgacttcaagaccgccgagctgacctggctgaactacatggaccccttcagcttcgaggaactggccaagggacccagcggcatgtacttcgctggcgccaagaccggcatctacagagcccagaccgccctgatcagcttcatcaagcaggaaatcatccagaagatcagccaccagagctgggtgatcgacctgggcatcggcaagggccaggacctgggcagatacctggacgccggcgtgagacacctggtcggcatcgataaggaccagacagccctggccgagctggtgtaccggaagttctcccacgccaccaccagacagcacaagcacgccaccaacatctacgtgctgcaccaggatctggccgagcctgccaaagaaatcagcgagaaagtgcaccagatctatggcttccccaaagagggcgccagcagcatcgtgtccaacctgttcatccactacctgatgaagaacacccagcaggtcgagaacctggctgtgctgtgccacaagctgctgcagcctggcggcatggtctggttcaccaccatgctgggcgaacaggtgctggaactgctgcacgagaaccggatcgaactgaacgaagtgtgggaggcccgggagaacgaggtggtcaagttcgccatcaagcggctgttcaaagaggacatcctgcaggaaaccggccaggaaatcggcgtcctgctgcccttcagcaacggcgacttctacaatgagtacctggtcaacaccgcctttctgatcaagattttcaagcaccatggctttagcctcgtgcagaagcagagcttcaaggactggatccccgagttccagaacttcagcaagagcctgtacaagatcctgaccgaggccgacaagacctggaccagcctgttcggcttcatctgcctgcggaagaacggaggcgggggaagtggagggggcggcagtcaggacctgcacgccatccagctgcagctcgaagaggaaatgttcaacggcggcatcagaagattcgaggccgaccagcagagacagatcgcctctggcaacgagagcgacaccgcctggaatagaaggctgctgtctgagctgatcgcccctatggccgaaggcatccaggcctacaaagaggaatacgagggcaagagaggcagagcccctagagccctggccttcatcaactgtgtgggcaatgaggtggccgcctacatcaccatgaagatcgtgatggacatgctgaacaccgacgtgaccctgcaggccattgccatgaacgtggccgacagaatcgaggaccaggtccgattcagcaagctggaaggacacgccgccaagtacttcgagaaagtgaagaagtccctgaaggccagcaagaccaagagctacagacacgcccacaacgtggccgtggtggccgaaaaatctgtggccgatagggacgccgacttctctagatgggaggcctggcctaaggacaccctgctgcagatcggcatgaccctgctggaaatcctggaaaacagcgtgttcttcaacggccagcccgtgttcctgagaaccctgaggacaaatggcggcaagcacggcgtgtactacctgcagacatctgagcacgtgggcgagtggatcaccgccttcaaagaacatgtggcccagctgagccctgcctatgccccttgtgtgatccctcctagaccctgggtgtcccctttcaatggcggctttcacaccgagaaggtggccagcagaatcagactggtcaagggcaaccgggaacacgtgcggaagctgaccaagaaacagatgcccgccgtgtacaaggccgtgaatgctctgcaggccaccaagtggcaggtcaacaaagaggtgctgcaggtcgtcgaggacgtgatcagactggatctgggctacggcgtgccaagctttaagcccctgatcgacagagagaacaagcccgccaaccctgtgcccctggaatttcagcacctgagaggccgcgagctgaaagagatgctgacacctgaacagtggcaggcctttatcaattggaagggcgagtgcaccaagctgtacaccgccgagacaaagaggggctctaagtctgccgccacagtgcgaatggtcggacaggccagaaagtacagccagttcgacgccatctacttcgtgtacgccctggacagccggtctagagtgtatgcccagagcagcacactgagcccccagtctaacgatctgggaaaggccctgctgagattcaccgagggccagagactggattctgccgaagccctgaagtggttcctggtcaacggcgccaacaactggggctgggacaagaaaaccttcgatgtgcggaccgccaacgtgctggatagcgagttccaggacatgtgcagagatatcgccgccgaccctctgacctttacccagtgggtcaacgccgatagcccctatggattcctggcctggtgcttcgagtacgccagatacctggacgccctggatgagggaacccaggatcagttcatgacccatctgcccgtgcaccaggatggctcttgttctggcatccagcactacagcgccatgctgagcgatgccgtgggagccaaagccgtgaacctgaagcctagcgacagcccccaggatatctatggcgctgtggcccaggtggtcatccagaaaaactacgcctacatgaacgccgaggacgccgagacattcacaagcggaagcgtgacactgacaggcgccgagctgagatctatggcctctgcctgggacatgatcggcatcacacggggcctgaccaaaaagcctgtgatgacactgccctacggcagcaccagactgacctgtagagaaagcgtgatcgactacatcgtggacctggaagagaaagaggcccagagagccattgccgagggcagaacagccaatcctgtgcaccccttcgacaacgaccggaaggatagcctgacacctagcgccgcctacaactacatgaccgccctgatctggcccagcatctctgaagtggtcaaggcccctatcgtggccatgaagatgatcagacagctggccagattcgccgccaagagaaatgagggcctggaataccctctgcccaccggctttatcctgcagcagaaaatcatggccaccgacatgctgcgggtgtccacatgtctgatgggcgagatcaagatgagcctgcagatcgagacagacgtggtggacgagacagccatgatgggagccgccgctcctaattttgtgcacggacacgatgccagccacctgatcctgaccgtgtgcgatctggtggacaagggcatcactagcgtggccgtgatccacgatagctttggaacacacgccggcagaaccgccgacctgagagattctctgcgggaagagatggtcaagatgtaccagaaccacaacgccctgcagaacctgctggacgtgcacgaagaaagatggctggtggacaccggcatccaggtgccagaacagggagagttcgacctgaacgagatcctggtgtccgactactgcttcgcctga

SEQ ID NO: 4 - protein, 2557 aa, Artificial Sequence
  Recombinant protein encoded by pC3P3-G2 plasmid
MDAQTRRRERRAEKQAQWKAANPFPVTTQGSQQTQPPQRHYGITSPISLAAPKETDCLLTQKLIETLKPFGVFEEEEELQRRILILGKLNNLVKEWIREISESKNLPQSVIENVGGKIFTFGSYRLGVHTKGADIDALCVAPRHVDRSDFFTSFYDKLKLQEEVKDLRAVEEAFVPVIKLCFDGIEIDILFARLALQTIPEDLDLRDDSLLKNLDIRCIRSLNGCRVTDEILHLVPNIDNFRLTLRAIKLWAKRHNIYSNILGFLGGVSWAMLVARTCQLYPNAIASTLVHKFFLVFSKWEWPNPVLLKQPEECNLNLPVWDPRVNPSDRYHLMPIITPAYPQQNSTYNVSVSTRMVMVEEFKQGLAITDEILLSKAEWSKLFEAPNFFQKYKHYIVLLASAPTEKQRLEWVGLVESKIRILVGSLEKNEFITLAHVNPQSFPAPKESPDREEFRTMWVIGLVFKKTENSENLSVDLTYDIQSFTDTVYRQAINSKMFELDMKIAAMHVKRKQLHQLLPSHVLQKRKKHSTEGVKLTALNDSSLDLSMDSDNSMSVPSPTSAMKTSPLNSSGSSQGRNSPAPAVTAASVTSIQASEVSVPQANSSESPGGPSSESIPQTATQPAIAPPPKPTVSRVVSSTRLVNPSPRPSGNTATKVPNPIVGVKRTSAPNKEEAPRRTKTEEDETSEDANCLALSGHDKTETKEQVDLETSAVQSETVPASASLLASQKTSSTDLSDIPALPANPIPVIKNSIKLRLNRGSGPRPLLAIHPTEARHKQKIVAPVKQTLNFDLLKLAGDVESNPGPASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSQDLHAIQLQLEEEMFNGGIRRFEADQQRQIASGNESDTAWNRRLLSELIAPMAEGIQAYKEEYEGKRGRAPRALAFINCVGNEVAAYITMKIVMDMLNTDVTLQAIAMNVADRIEDQVRFSKLEGHAAKYFEKVKKSLKASKTKSYRHAHNVAVVAEKSVADRDADFSRWEAWPKDTLLQIGMTLLEILENSVFFNGQPVFLRTLRTNGGKHGVYYLQTSEHVGEWITAFKEHVAQLSPAYAPCVIPPRPWVSPFNGGFHTEKVASRIRLVKGNREHVRKLTKKQMPAVYKAVNALQATKWQVNKEVLQVVEDVIRLDLGYGVPSFKPLIDRENKPANPVPLEFQHLRGRELKEMLTPEQWQAFINWKGECTKLYTAETKRGSKSAATVRMVGQARKYSQFDAIYFVYALDSRSRVYAQSSTLSPQSNDLGKALLRFTEGQRLDSAEALKWFLVNGANNWGWDKKTFDVRTANVLDSEFQDMCRDIAADPLTFTQWVNADSPYGFLAWCFEYARYLDALDEGTQDQFMTHLPVHQDGSCSGIQHYSAMLSDAVGAKAVNLKPSDSPQDIYGAVAQVVIQKNYAYMNAEDAETFTSGSVTLTGAELRSMASAWDMIGITRGLTKKPVMTLPYGSTRLTCRESVIDYIVDLEEKEAQRAIAEGRTANPVHPFDNDRKDSLTPSAAYNYMTALIWPSISEVVKAPIVAMKMIRQLARFAAKRNEGLEYPLPTGFILQQKIMATDMLRVSTCLMGEIKMSLQIETDVVDETAMMGAAAPNFVHGHDASHLILTVCDLVDKGITSVAVIHDSFGTHAGRTADLRDSLREEMVKMYQNHNALQNLLDVHEERWLVDTGIQVPEQGEFDLNEILVSDYCFA

SEQ ID NO: 5 - DNA, 933 bp, Artificial Sequence
  Recombinant DNA open-reading frame from pADAR1-Zalpha/(G4S)2/E3L-dsDNA plasmid
atgctgtccatctaccaagaccaagaacagaggatactcaaatttttggaggaattgggtgagggaaaggccaccactgcacacgatttgtctgggaaactcgggaccccaaaaaaggagattaacagggttctgtactccctggctaagaaaggcaaattgcagaaagaagccggaactccccctctgtggaagatcgcagtgagcacacaagcttggaatcaacatagcggagtcgtgcgacccgatggtcattcacagggcgccccgaactccgacccctccctagaaccagaggacaggaattcaacgagcgtgagtgaagacctgcttgaaccgttcatagctgtctctgcacaggcatggaatcagcactccggtgtggtgagacccgactctcatagccagggaagccccaacagcgacccgggtctcgagcctgaagactctaattcgactagtgctcttgaggatccattggaatttctcgatatggcagaaatcaaggaaaaaatttgcgattacctttttaacgtctctgatagcagcgctctgaacctggctaaaaatataggtcttaccaaagctcgtgatatcaatgccgtgctgatcgatatggagcggcagggagacgtatatcgccagggcactactcccccaatctggcacctcacggatggaggaggaggctctggtggtggcgggagcaaccccgtgaccatcatcaacgagtactgccagatcaccaagagagactggagcttcagaatcgagagcgtgggccccagcaacagccccaccttctacgcctgcgtggacatcgacggcagagtgttcgacaaggccgacggcaagagcaagagagacgccaagaacaacgccgccaagctggccgtggacaagctgctgggctacgtgatcatcagattctga

SEQ ID NO: 6 - protein, 310 aa, Artificial Sequence
  recombinant protein encoded by pADAR1-Zalpha/ (G4S)2/E3L-dsDNA plasmid
MLSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDGGGGSGGGGSNPVTIINEYCQITKRDWSFRIESVGPSNSPTFYACVDIDGRVFDKADGKSKRDAKNNAAKLAVDKLLGYVIIRF

SEQ ID NO: 7 - DNA, 567 bp, Artificial Sequence
  recombinant DNA open-reading frame from pE3L- Zalpha/NS1-dsDNA plasmid
atgagcaagatctacatcgacgagagaagcgacgccgagatcgtgtgcgccgccatcaagaacatcggcatcgagggcgccaccgccgcccagctgaccagacagctgaacatggagaagagagaggtgaacaaggccctgtacgacctgcagagaagcgccatggtgtacagcagcgacgacatcccccccagatggttcatgaccaccgaggccgacaagcccgacgccgacgccatggccgacgtgatcatcgacgacgtgagcagagagaagagcatgagagaggaccacaagagcttcgacgacgtgatccccgccaagaagatcatcgactggaaggacgccgaccccaatacagtctcctcttttcaagtggactgttttttgtggcatgttcgaaagcgggttgctgaccaagagctgggggatgccccgtttctagaccggctacggagagaccaaaagagcttgcgtgggagaggcagtaccctgggcctcgacatcgagaccgctacacgcgccggcaagcagatcgtggaacgcatcttaaaagaggagtcctaa

SEQ ID NO: 8 - protein, 188 aa, Artificial Sequence
  recombinant protein encoded by pE3L-Zalpha/NS1- dsDNA plasmid
MSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDADPNTVSSFQVDCFLWHVRKRVADQELGDAPFLDRLRRDQKSLRGRGSTLGLDIETATRAGKQIVERILKEES

SEQ ID NO: 9 - DNA, 567 bp, Artificial Sequence
  recombinant DNA open-reading frame from pE3L- Zalpha/B2-dsDNA plasmid
atgagcaagatctacatcgacgagagaagcgacgccgagatcgtgtgcgccgccatcaagaacatcggcatcgagggcgccaccgccgcccagctgaccagacagctgaacatggagaagagagaggtgaacaaggccctgtacgacctgcagagaagcgccatggtgtacagcagcgacgacatcccccccagatggttcatgaccaccgaggccgacaagcccgacgccgacgccatggccgacgtgatcatcgacgacgtgagcagagagaagagcatgagagaggaccacaagagcttcgacgacgtgatccccgccaagaagatcatcgactggaaggacgccccctctaaattggcattgatccaggagctgcccgataggattcaaacagccgtggaagcagccatgggaatgtcataccaggatgcaccaaacaatgttagaagagacctggacaatttgcatgcctgccttaacaaagcaaagctcactgtgtccagaatggtgactagcctgttggaaaaaccatccgtcgtcgcgtacctggagggaaaggcataa

SEQ ID NO: 10 - protein, 188 aa, Artificial Sequence
  recombinant protein encoded by pE3L-Zalpha/B2- dsDNA plasmid
MSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDAPSKLALIQELPDRIQTAVEAAMGMSYQDAPNNVRRDLDNLHACLNKAKLTVSRMVTSLLEKPSVVAYLEGKA

SEQ ID NO: 11 - DNA, 828 bp, Artificial Sequence
  recombinant DNA open-reading frame from pE3L-Zalpha/hEIF2AK2-dsDNA plasmid
atgagcaagatctacatcgacgagagaagcgacgccgagatcgtgtgcgccgccatcaagaacatcggcatcgagggcgccaccgccgcccagctgaccagacagctgaacatggagaagagagaggtgaacaaggccctgtacgacctgcagagaagcgccatggtgtacagcagcgacgacatcccccccagatggttcatgaccaccgaggccgacaagcccgacgccgacgccatggccgacgtgatcatcgacgacgtgagcagagagaagagcatgagagaggaccacaagagcttcgacgacgtgatccccgccaagaagatcatcgactggaaggacgccttcttcatggaggaactgaacacataccggcagaagcagggcgttgtgctgaagtaccaggaactgccgaattcaggtcctccccatgatcgccggttcacgtttcaggtaataatcgacggcagggaattccccgaaggcgagggcagatctaagaaggaagcgaagaatgccgcggccaagttggctgtcgaaatacttaataaagaaaagaaggctgtcagtccactgcttctgacaacaactaatagtagcgaaggactaagcatgggaaattacatcggtctaatcaatcggattgctcagaaaaaacgccttactgtgaattatgagcagtgcgcctccggagttcacggacctgagggattccactacaagtgcaagatgggccaaaaggagtatagcatcggcactgggtctactaaacaagaggctaagcagctggccgcaaagctggcgtatctacagatcctgagttaa

SEQ ID NO: 12 - protein, 275 aa, Artificial Sequence
  recombinant protein encoded by pE3L-Zalpha/ hEIF2AK2-dsDNA plasmid
MSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDAFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILS

SEQ ID NO: 13 - DNA, 603 bp, Artificial Sequence
  recombinant DNA open-reading frame from pE3L- Zalpha/sigma3-dsDNA plasmid
atgagcaagatctacatcgacgagagaagcgacgccgagatcgtgtgcgccgccatcaagaacatcggcatcgagggcgccaccgccgcccagctgaccagacagctgaacatggagaagagagaggtgaacaaggccctgtacgacctgcagagaagcgccatggtgtacagcagcgacgacatcccccccagatggttcatgaccaccgaggccgacaagcccgacgccgacgccatggccgacgtgatcatcgacgacgtgagcagagagaagagcatgagagaggaccacaagagcttcgacgacgtgatccccgccaagaagatcatcgactggaaggacgccgtgtatgactattctgagctagagcatgatcctagtaaggggcgggcctatcggaaggaattagttacccctgctagagacttcgggcattttggccttagtcactactctagagcgactacccctatccttgggaagatgccggctgtctttagtggaatgttgaccggcaactgtaagatgtatccgtttataaaagggacggccaaactgaagacagtgcgcaaactcgtagactccgtgaatcacgcctga

SEQ ID NO: 14 - protein, 200 aa, Artificial Sequence
  recombinant protein encoded by pE3L-Zalpha/ sigma3-dsDNA plasmid
MSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDAVYDYSELEHDPSKGRAYRKELVTPARDFGHFGLSHYSRATTPILGKMPAVFSGMLTGNCKMYPFIKGTAKLKTVRKLVDSVNHA

SEQ ID NO: 15 - DNA, 696 bp, Artificial Sequence
  recombinant DNA open-reading frame from pE3L-Zalpha/NS1-dsDNA/(G4S)2/SZIP plasmid
atgagcaagatctacatcgacgagagaagcgacgccgagatcgtgtgcgccgccatcaagaacatcggcatcgagggcgccaccgccgcccagctgaccagacagctgaacatggagaagagagaggtgaacaaggccctgtacgacctgcagagaagcgccatggtgtacagcagcgacgacatcccccccagatggttcatgaccaccgaggccgacaagcccgacgccgacgccatggccgacgtgatcatcgacgacgtgagcagagagaagagcatgagagaggaccacaagagcttcgacgacgtgatccccgccaagaagatcatcgactggaaggacgccgaccccaatacagtctcctcttttcaagtggactgttttttgtggcatgttcgaaagcgggttgctgaccaagagctgggggatgccccgtttctagaccggctacggagagaccaaaagagcttgcgtgggagaggcagtaccctgggcctcgacatcgagaccgctacacgcgccggcaagcagatcgtggaacgcatcttaaaagaggagtccggaggaggaggctctggtggtggcgggagcaggatggctcagttggaggctaaggtagaggaactattgtccaaaaattggaatctcgagaacgaagtagctcgcctcaagaaactggtcggcgagcgctaa

SEQ ID NO: 16 - protein, 231 aa, Artificial Sequence
  recombinant protein encoded by pE3L-Zaalpha/NS1 -dsDNA/(G4S)2/SZIP plasmid
MSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDADPNTVSSFQVDCFLWHVRKRVADQELGDAPFLDRLRRDQKSLRGRGSTLGLDIETATRAGKQIVERILKEESGGGGSGGGGSRMAQLEAKVEELLSKNWNLENEVARLKKLVGER

SEQ ID NO: 17 - DNA, 699 bp, Artificial Sequence
  recombinant DNA open-reading frame from pE3L-Zalpha/NS1-dsDNA/(G4S)2/GCN4 plasmid
atgagcaagatctacatcgacgagagaagcgacgccgagatcgtgtgcgccgccatcaagaacatcggcatcgagggcgccaccgccgcccagctgaccagacagctgaacatggagaagagagaggtgaacaaggccctgtacgacctgcagagaagcgccatggtgtacagcagcgacgacatcccccccagatggttcatgaccaccgaggccgacaagcccgacgccgacgccatggccgacgtgatcatcgacgacgtgagcagagagaagagcatgagagaggaccacaagagcttcgacgacgtgatccccgccaagaagatcatcgactggaaggacgccgaccccaatacagtctcctcttttcaagtggactgttttttgtggcatgttcgaaagcgggttgctgaccaagagctgggggatgccccgtttctagaccggctacggagagaccaaaagagcttgcgtgggagaggcagtaccctgggcctcgacatcgagaccgctacacgcgccggcaagcagatcgtggaacgcatcttaaaagaggagtccggaggaggaggctctggtggtggcgggagcatgaaggtcaagcaactggaagacgttgtggaggagttactctccgtaaactatcacctggagaatgtagttgcccgcctgaagaaactggtcggggagagatga

SEQ ID NO: 18 - protein, 232 aa, Artificial Sequence
  recombinant protein encoded by pE3L-Zalpha/NS1- dsDNA/(G4S)2/GCN4 plasmid
MSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDADPNTVSSFQVDCFLWHVRKRVADQELGDAPFLDRLRRDQKSLRGRGSTLGLDIETATRAGKQIVERILKEESGGGGSGGGGSMKVKQLEDVVEELLSVNYHLENVVARLKKLVGER

SEQ ID NO: 19 - DNA, 8502 bp, Artificial Sequence
  recombinant DNA open-reading frame from pC3P3- G3a plasmid
atggatgctcagaccagacgcagagaacggcgggcagagaagcaggcacagtggaaggccgctaatccgtttccagttacaacgcagggatcacaacaaacgcagccaccacagaggcactatggcattacctctcctatcagcttagcggcccccaaggagactgactgcctactcacacagaagctcatcgagacgctgaagccctttggggtttttgaagaagaagaggaactgcagcgcaggattttaattttgggaaaattaaataacctggtgaaagaatggattcgagaaatcagtgaaagcaagaatctcccacaatctgtaattgaaaatgttggagggaagatttttacatttggatcttacagactaggagtccacacgaaaggtgctgatattgatgcgttgtgtgttgcaccaagacatgttgatcgaagtgactttttcacctcattctatgataaattgaaattacaagaagaagtgaaagatttaagagctgttgaagaggcatttgtaccagttatcaaactctgttttgatggaatagagattgatattttgtttgcaagattagcactgcagactattccagaagatttggacctacgagatgacagtctgcttaaaaacctagatataagatgcataagaagccttaatggttgcagggtaaccgatgaaattttacatctagtaccaaacattgacaacttcaggttaactctgagagccatcaaactgtgggccaaacggcacaacatctattccaatatattaggtttcctcggtggtgtttcctgggctatgctagtagcaagaacttgccagctttatccaaatgcaatagcatcaactcttgtacataaatttttcttggtattttctaaatgggaatggccaaatccagtgctattgaaacagcctgaagaatgcaatcttaatttgcctgtgtgggacccaagggtaaaccccagtgataggtaccatcttatgcctataattacaccagcatacccacagcagaactccacgtacaatgtgtccgtttcaacacggatggtcatggttgaggagtttaaacaaggtcttgctatcacagatgaaattttgctgagtaaggcagagtggtccaaactttttgaagctccaaacttctttcagaagtacaagcattatattgtacttctagcaagtgcgcccacggaaaagcagcgtctggaatgggtgggcttggtggaatcaaaaatccgcatcctggttggaagcttggagaagaatgagtttattacactggctcatgtgaatccccagtcatttccagcccccaaagaaagtcctgacagggaagaatttcgcacaatgtgggtgattgggttagtgtttaaaaaaactgaaaactctgaaaatctcagtgtcgacctcacctatgatatccagtctttcacagacacagtttataggcaagcaataaacagcaaaatgtttgagttggatatgaagattgcagcaatgcatgtgaagagaaagcaactccatcagctgctgcctagtcacgtgcttcagaagaggaagaagcattcaacagaaggagtcaagttaacagctctgaatgacagcagccttgacttgtctatggacagtgataacagcatgtctgtgccttcacccaccagtgctatgaagaccagtccattgaatagttctggcagctcccagggcagaaacagtcctgctccagctgtgaccgcagcatctgtgaccagcatccaggcttctgaggtttctgtaccgcaagcaaattccagtgaaagcccagggggtccatcgagcgaaagcattcctcaaactgccacacagccagccattgccccaccaccaaagcctacagtctccagagttgtctcctcaacacgactggtaaacccatcgcctagaccttcaggaaacacagcaacaaaagtccctaatcctatagtaggagtcaagagaacgtccgcccccaataaagaagaagcccctagaaggaccaaaacagaagaggatgaaacaagtgaagatgctaactgtcttgctttgagtggacatgataaaacagagacaaaggaacaagttgatctggagacaagtgcggttcaatcagaaactgttccggcatcggcttctctgttggcctctcagaaaacatccagtacagacctttctgatatccctgctctccctgcaaatcctattcctgttatcaagaactcaataaaactgagactgaatcggggatcaggccctcgcccactgctcgcaatccaccctactgaagcaaggcacaagcaaaaaattgtggctcctgtgaagcagacgctgaactttgatctgctgaagctcgctggcgacgtggagtcgaaccctggccctagcaagatctacatcgacgagagaagcgacgccgagatcgtgtgcgccgccatcaagaacatcggcatcgagggcgccaccgccgcccagctgaccagacagctgaacatggagaagagagaggtgaacaaggccctgtacgacctgcagagaagcgccatggtgtacagcagcgacgacatcccccccagatggttcatgaccaccgaggccgacaagcccgacgccgacgccatggccgacgtgatcatcgacgacgtgagcagagagaagagcatgagagaggaccacaagagcttcgacgacgtgatccccgccaagaagatcatcgactggaaggacgccgaccccaatacagtctcctcttttcaagtggactgttttttgtggcatgttcgaaagcgggttgctgaccaagagctgggggatgccccgtttctagaccggctacggagagaccaaaagagcttgcgtgggagaggcagtaccctgggcctcgacatcgagaccgctacacgcgccggcaagcagatcgtggaacgcatcttaaaagaggagtccggaggcgggggaagtggagggggcggcagtaggatggctcagttggaggctaaggtagaggaactattgtccaaaaattggaatctcgagaacgaagtagctcgcctcaagaaactggtcggcgagcgcggatcaggcccgcgacccctccttgccatccacccaaccgaggcacggcacaagcagaaaatagtggcacccgttaagcagactctcaactttgatctcctgaagcttgccggcgacgtagagtccaaccccggccccgccagcctggacaacctggtggccagataccagcggtgcttcaacgaccagagcctgaagaacagcaccatcgagctggaaatccggttccagcagatcaacttcctgctgttcaagaccgtgtacgaggccctggtcgcccaggaaatccccagcaccatcagccacagcatccggtgcatcaagaaggtgcaccacgagaaccactgccgggagaagatcctgcccagcgagaacctgtacttcaagaaacagcccctgatgttcttcaagttcagcgagcccgccagcctgggctgtaaagtgtccctggccatcgagcagcccatccggaagttcatcctggacagcagcgtgctggtccggctgaagaaccggaccaccttccgggtgtccgagctgtggaagatcgagctgaccatcgtgaagcagctgatgggcagcgaggtgtcagccaagctggccgccttcaagaccctgctgttcgacacccccgagcagcagaccaccaagaacatgatgaccctgatcaaccccgacgacgagtacctgtacgagatcgagatcgagtacaccggcaagcctgagagcctgacagccgccgacgtgatcaagatcaagaacaccgtgctgacactgatcagccccaaccacctgatgctgaccgcctaccaccaggccatcgagtttatcgccagccacatcctgagcagcgagatcctgctggcccggatcaagagcggcaagtggggcctgaagagactgctgccccaggtcaagtccatgaccaaggccgactacatgaagttctacccccccgtgggctactacgtgaccgacaaggccgacggcatccggggcattgccgtgatccaggacacccagatctacgtggtggccgaccagctgtacagcctgggcaccaccggcatcgagcccctgaagcccaccatcctggacggcgagttcatgcccgagaagaaagagttctacggctttgacgtgatcatgtacgagggcaacctgctgacccagcagggcttcgagacacggatcgagagcctgagcaagggcatcaaggtgctgcaggccttcaacatcaaggccgagatgaagcccttcatcagcctgacctccgccgaccccaacgtgctgctgaagaatttcgagagcatcttcaagaagaaaacccggccctacagcatcgacggcatcatcctggtggagcccggcaacagctacctgaacaccaacaccttcaagtggaagcccacctgggacaacaccctggactttctggtccggaagtgccccgagtccctgaacgtgcccgagtacgcccccaagaagggcttcagcctgcatctgctgttcgtgggcatcagcggcgagctgtttaagaagctggccctgaactggtgccccggctacaccaagctgttccccgtgacccagcggaaccagaactacttccccgtgcagttccagcccagcgacttccccctggccttcctgtactaccaccccgacaccagcagcttcagcaacatcgatggcaaggtgctggaaatgcggtgcctgaagcgggagatcaactacgtgcgctgggagatcgtgaagatccgggaggaccggcagcaggatctgaaaaccggcggctacttcggcaacgacttcaagaccgccgagctgacctggctgaactacatggaccccttcagcttcgaggaactggccaagggacccagcggcatgtacttcgctggcgccaagaccggcatctacagagcccagaccgccctgatcagcttcatcaagcaggaaatcatccagaagatcagccaccagagctgggtgatcgacctgggcatcggcaagggccaggacctgggcagatacctggacgccggcgtgagacacctggtcggcatcgataaggaccagacagccctggccgagctggtgtaccggaagttctcccacgccaccaccagacagcacaagcacgccaccaacatctacgtgctgcaccaggatctggccgagcctgccaaagaaatcagcgagaaagtgcaccagatctatggcttccccaaagagggcgccagcagcatcgtgtccaacctgttcatccactacctgatgaagaacacccagcaggtcgagaacctggctgtgctgtgccacaagctgctgcagcctggcggcatggtctggttcaccaccatgctgggcgaacaggtgctggaactgctgcacgagaaccggatcgaactgaacgaagtgtgggaggcccgggagaacgaggtggtcaagttcgccatcaagcggctgttcaaagaggacatcctgcaggaaaccggccaggaaatcggcgtcctgctgcccttcagcaacggcgacttctacaatgagtacctggtcaacaccgcctttctgatcaagattttcaagcaccatggctttagcctcgtgcagaagcagagcttcaaggactggatccccgagttccagaacttcagcaagagcctgtacaagatcctgaccgaggccgacaagacctggaccagcctgttcggcttcatctgcctgcggaagaacggaggcgggggaagtggagggggcggcagtcaggacctgcacgccatccagctgcagctcgaagaggaaatgttcaacggcggcatcagaagattcgaggccgaccagcagagacagatcgcctctggcaacgagagcgacaccgcctggaatagaaggctgctgtctgagctgatcgcccctatggccgaaggcatccaggcctacaaagaggaatacgagggcaagagaggcagagcccctagagccctggccttcatcaactgtgtgggcaatgaggtggccgcctacatcaccatgaagatcgtgatggacatgctgaacaccgacgtgaccctgcaggccattgccatgaacgtggccgacagaatcgaggaccaggtccgattcagcaagctggaaggacacgccgccaagtacttcgagaaagtgaagaagtccctgaaggccagcaagaccaagagctacagacacgcccacaacgtggccgtggtggccgaaaaatctgtggccgatagggacgccgacttctctagatgggaggcctggcctaaggacaccctgctgcagatcggcatgaccctgctggaaatcctggaaaacagcgtgttcttcaacggccagcccgtgttcctgagaaccctgaggacaaatggcggcaagcacggcgtgtactacctgcagacatctgagcacgtgggcgagtggatcaccgccttcaaagaacatgtggcccagctgagccctgcctatgccccttgtgtgatccctcctagaccctgggtgtcccctttcaatggcggctttcacaccgagaaggtggccagcagaatcagactggtcaagggcaaccgggaacacgtgcggaagctgaccaagaaacagatgcccgccgtgtacaaggccgtgaatgctctgcaggccaccaagtggcaggtcaacaaagaggtgctgcaggtcgtcgaggacgtgatcagactggatctgggctacggcgtgccaagctttaagcccctgatcgacagagagaacaagcccgccaaccctgtgcccctggaatttcagcacctgagaggccgcgagctgaaagagatgctgacacctgaacagtggcaggcctttatcaattggaagggcgagtgcaccaagctgtacaccgccgagacaaagaggggctctaagtctgccgccacagtgcgaatggtcggacaggccagaaagtacagccagttcgacgccatctacttcgtgtacgccctggacagccggtctagagtgtatgcccagagcagcacactgagcccccagtctaacgatctgggaaaggccctgctgagattcaccgagggccagagactggattctgccgaagccctgaagtggttcctggtcaacggcgccaacaactggggctgggacaagaaaaccttcgatgtgcggaccgccaacgtgctggatagcgagttccaggacatgtgcagagatatcgccgccgaccctctgacctttacccagtgggtcaacgccgatagcccctatggattcctggcctggtgcttcgagtacgccagatacctggacgccctggatgagggaacccaggatcagttcatgacccatctgcccgtgcaccaggatggctcttgttctggcatccagcactacagcgccatgctgagcgatgccgtgggagccaaagccgtgaacctgaagcctagcgacagcccccaggatatctatggcgctgtggcccaggtggtcatccagaaaaactacgcctacatgaacgccgaggacgccgagacattcacaagcggaagcgtgacactgacaggcgccgagctgagatctatggcctctgcctgggacatgatcggcatcacacggggcctgaccaaaaagcctgtgatgacactgccctacggcagcaccagactgacctgtagagaaagcgtgatcgactacatcgtggacctggaagagaaagaggcccagagagccattgccgagggcagaacagccaatcctgtgcaccccttcgacaacgaccggaaggatagcctgacacctagcgccgcctacaactacatgaccgccctgatctggcccagcatctctgaagtggtcaaggcccctatcgtggccatgaagatgatcagacagctggccagattcgccgccaagagaaatgagggcctggaataccctctgcccaccggctttatcctgcagcagaaaatcatggccaccgacatgctgcgggtgtccacatgtctgatgggcgagatcaagatgagcctgcagatcgagacagacgtggtggacgagacagccatgatgggagccgccgctcctaattttgtgcacggacacgatgccagccacctgatcctgaccgtgtgcgatctggtggacaagggcatcactagcgtggccgtgatccacgatagctttggaacacacgccggcagaaccgccgacctgagagattctctgcgggaagagatggtcaagatgtaccagaaccacaacgccctgcagaacctgctggacgtgcacgaagaaagatggctggtggacaccggcatccaggtgccagaacagggagagttcgacctgaacgagatcctggtgtccgactactgcttcgcctga

SEQ ID NO: 20 - protein, 2833 aa, Artificial Sequence
  recombinant protein encoded by pC3P3-G3a plasmid
MDAQTRRRERRAEKQAQWKAANPFPVTTQGSQQTQPPQRHYGITSPISLAAPKETDCLLTQKLIETLKPFGVFEEEEELQRRILILGKLNNLVKEWIREISESKNLPQSVIENVGGKIFTFGSYRLGVHTKGADIDALCVAPRHVDRSDFFTSFYDKLKLQEEVKDLRAVEEAFVPVIKLCFDGIEIDILFARLALQTIPEDLDLRDDSLLKNLDIRCIRSLNGCRVTDEILHLVPNIDNFRLTLRAIKLWAKRHNIYSNILGFLGGVSWAMLVARTCQLYPNAIASTLVHKFFLVFSKWEWPNPVLLKQPEECNLNLPVWDPRVNPSDRYHLMPIITPAYPQQNSTYNVSVSTRMVMVEEFKQGLAITDEILLSKAEWSKLFEAPNFFQKYKHYIVLLASAPTEKQRLEWVGLVESKIRILVGSLEKNEFITLAHVNPQSFPAPKESPDREEFRTMWVIGLVFKKTENSENLSVDLTYDIQSFTDTVYRQAINSKMFELDMKIAAMHVKRKQLHQLLPSHVLQKRKKHSTEGVKLTALNDSSLDLSMDSDNSMSVPSPTSAMKTSPLNSSGSSQGRNSPAPAVTAASVTSIQASEVSVPQANSSESPGGPSSESIPQTATQPAIAPPPKPTVSRVVSSTRLVNPSPRPSGNTATKVPNPIVGVKRTSAPNKEEAPRRTKTEEDETSEDANCLALSGHDKTETKEQVDLETSAVQSETVPASASLLASQKTSSTDLSDIPALPANPIPVIKNSIKLRLNRGSGPRPLLAIHPTEARHKQKIVAPVKQTLNFDLLKLAGDVESNPGPSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDADPNTVSSFQVDCFLWHVRKRVADQELGDAPFLDRLRRDQKSLRGRGSTLGLDIETATRAGKQIVERILKEESGGGGSGGGGSRMAQLEAKVEELLSKNWNLENEVARLKKLVGERGSGPRPLLAIHPTEARHKQKIVAPVKQTLNFDLLKLAGDVESNPGPASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSQDLHAIQLQLEEEMFNGGIRRFEADQQRQIASGNESDTAWNRRLLSELIAPMAEGIQAYKEEYEGKRGRAPRALAFINCVGNEVAAYITMKIVMDMLNTDVTLQAIAMNVADRIEDQVRFSKLEGHAAKYFEKVKKSLKASKTKSYRHAHNVAVVAEKSVADRDADFSRWEAWPKDTLLQIGMTLLEILENSVFFNGQPVFLRTLRTNGGKHGVYYLQTSEHVGEWITAFKEHVAQLSPAYAPCVIPPRPWVSPFNGGFHTEKVASRIRLVKGNREHVRKLTKKQMPAVYKAVNALQATKWQVNKEVLQVVEDVIRLDLGYGVPSFKPLIDRENKPANPVPLEFQHLRGRELKEMLTPEQWQAFINWKGECTKLYTAETKRGSKSAATVRMVGQARKYSQFDAIYFVYALDSRSRVYAQSSTLSPQSNDLGKALLRFTEGQRLDSAEALKWFLVNGANNWGWDKKTFDVRTANVLDSEFQDMCRDIAADPLTFTQWVNADSPYGFLAWCFEYARYLDALDEGTQDQFMTHLPVHQDGSCSGIQHYSAMLSDAVGAKAVNLKPSDSPQDIYGAVAQVVIQKNYAYMNAEDAETFTSGSVTLTGAELRSMASAWDMIGITRGLTKKPVMTLPYGSTRLTCRESVIDYIVDLEEKEAQRAIAEGRTANPVHPFDNDRKDSLTPSAAYNYMTALIWPSISEVVKAPIVAMKMIRQLARFAAKRNEGLEYPLPTGFILQQKIMATDMLRVSTCLMGEIKMSLQIETDVVDETAMMGAAAPNFVHGHDASHLILTVCDLVDKGITSVAVIHDSFGTHAGRTADLRDSLREEMVKMYQNHNALQNLLDVHEERWLVDTGIQVPEQGEFDLNEILVSDYCFA

SEQ ID NO: 21 - DNA, 8394 bp, Artificial Sequence
  recombinant DNA open-reading frame from pC3P3- G3b plasmid
atggatgctcagaccagacgcagagaacggcgggcagagaagcaggcacagtggaaggccgctaatccgtttccagttacaacgcagggatcacaacaaacgcagccaccacagaggcactatggcattacctctcctatcagcttagcggcccccaaggagactgactgcctactcacacagaagctcatcgagacgctgaagccctttggggtttttgaagaagaagaggaactgcagcgcaggattttaattttgggaaaattaaataacctggtgaaagaatggattcgagaaatcagtgaaagcaagaatctcccacaatctgtaattgaaaatgttggagggaagatttttacatttggatcttacagactaggagtccacacgaaaggtgctgatattgatgcgttgtgtgttgcaccaagacatgttgatcgaagtgactttttcacctcattctatgataaattgaaattacaagaagaagtgaaagatttaagagctgttgaagaggcatttgtaccagttatcaaactctgttttgatggaatagagattgatattttgtttgcaagattagcactgcagactattccagaagatttggacctacgagatgacagtctgcttaaaaacctagatataagatgcataagaagccttaatggttgcagggtaaccgatgaaattttacatctagtaccaaacattgacaacttcaggttaactctgagagccatcaaactgtgggccaaacggcacaacatctattccaatatattaggtttcctcggtggtgtttcctgggctatgctagtagcaagaacttgccagctttatccaaatgcaatagcatcaactcttgtacataaatttttcttggtattttctaaatgggaatggccaaatccagtgctattgaaacagcctgaagaatgcaatcttaatttgcctgtgtgggacccaagggtaaaccccagtgataggtaccatcttatgcctataattacaccagcatacccacagcagaactccacgtacaatgtgtccgtttcaacacggatggtcatggttgaggagtttaaacaaggtcttgctatcacagatgaaattttgctgagtaaggcagagtggtccaaactttttgaagctccaaacttctttcagaagtacaagcattatattgtacttctagcaagtgcgcccacggaaaagcagcgtctggaatgggtgggcttggtggaatcaaaaatccgcatcctggttggaagcttggagaagaatgagtttattacactggctcatgtgaatccccagtcatttccagcccccaaagaaagtcctgacagggaagaatttcgcacaatgtgggtgattgggttagtgtttaaaaaaactgaaaactctgaaaatctcagtgtcgacctcacctatgatatccagtctttcacagacacagtttataggcaagcaataaacagcaaaatgtttgagttggatatgaagattgcagcaatgcatgtgaagagaaagcaactccatcagctgctgcctagtcacgtgcttcagaagaggaagaagcattcaacagaaggagtcaagttaacagctctgaatgacagcagccttgacttgtctatggacagtgataacagcatgtctgtgccttcacccaccagtgctatgaagaccagtccattgaatagttctggcagctcccagggcagaaacagtcctgctccagctgtgaccgcagcatctgtgaccagcatccaggcttctgaggtttctgtaccgcaagcaaattccagtgaaagcccagggggtccatcgagcgaaagcattcctcaaactgccacacagccagccattgccccaccaccaaagcctacagtctccagagttgtctcctcaacacgactggtaaacccatcgcctagaccttcaggaaacacagcaacaaaagtccctaatcctatagtaggagtcaagagaacgtccgcccccaataaagaagaagcccctagaaggaccaaaacagaagaggatgaaacaagtgaagatgctaactgtcttgctttgagtggacatgataaaacagagacaaaggaacaagttgatctggagacaagtgcggttcaatcagaaactgttccggcatcggcttctctgttggcctctcagaaaacatccagtacagacctttctgatatccctgctctccctgcaaatcctattcctgttatcaagaactcaataaaactgagactgaatcggggaggaggaggctctggtggtggcgggagcagcaagatctacatcgacgagagaagcgacgccgagatcgtgtgcgccgccatcaagaacatcggcatcgagggcgccaccgccgcccagctgaccagacagctgaacatggagaagagagaggtgaacaaggccctgtacgacctgcagagaagcgccatggtgtacagcagcgacgacatcccccccagatggttcatgaccaccgaggccgacaagcccgacgccgacgccatggccgacgtgatcatcgacgacgtgagcagagagaagagcatgagagaggaccacaagagcttcgacgacgtgatccccgccaagaagatcatcgactggaaggacgccgaccccaatacagtctcctcttttcaagtggactgttttttgtggcatgttcgaaagcgggttgctgaccaagagctgggggatgccccgtttctagaccggctacggagagaccaaaagagcttgcgtgggagaggcagtaccctgggcctcgacatcgagaccgctacacgcgccggcaagcagatcgtggaacgcatcttaaaagaggagtccggaggcgggggaagtggagggggcggcagtaggatggctcagttggaggctaaggtagaggaactattgtccaaaaattggaatctcgagaacgaagtagctcgcctcaagaaactggtcggcgagcgcggatcaggcccgcgacccctccttgccatccacccaaccgaggcacggcacaagcagaaaatagtggcacccgttaagcagactctcaactttgatctcctgaagcttgccggcgacgtagagtccaaccccggccccgccagcctggacaacctggtggccagataccagcggtgcttcaacgaccagagcctgaagaacagcaccatcgagctggaaatccggttccagcagatcaacttcctgctgttcaagaccgtgtacgaggccctggtcgcccaggaaatccccagcaccatcagccacagcatccggtgcatcaagaaggtgcaccacgagaaccactgccgggagaagatcctgcccagcgagaacctgtacttcaagaaacagcccctgatgttcttcaagttcagcgagcccgccagcctgggctgtaaagtgtccctggccatcgagcagcccatccggaagttcatcctggacagcagcgtgctggtccggctgaagaaccggaccaccttccgggtgtccgagctgtggaagatcgagctgaccatcgtgaagcagctgatgggcagcgaggtgtcagccaagctggccgccttcaagaccctgctgttcgacacccccgagcagcagaccaccaagaacatgatgaccctgatcaaccccgacgacgagtacctgtacgagatcgagatcgagtacaccggcaagcctgagagcctgacagccgccgacgtgatcaagatcaagaacaccgtgctgacactgatcagccccaaccacctgatgctgaccgcctaccaccaggccatcgagtttatcgccagccacatcctgagcagcgagatcctgctggcccggatcaagagcggcaagtggggcctgaagagactgctgccccaggtcaagtccatgaccaaggccgactacatgaagttctacccccccgtgggctactacgtgaccgacaaggccgacggcatccggggcattgccgtgatccaggacacccagatctacgtggtggccgaccagctgtacagcctgggcaccaccggcatcgagcccctgaagcccaccatcctggacggcgagttcatgcccgagaagaaagagttctacggctttgacgtgatcatgtacgagggcaacctgctgacccagcagggcttcgagacacggatcgagagcctgagcaagggcatcaaggtgctgcaggccttcaacatcaaggccgagatgaagcccttcatcagcctgacctccgccgaccccaacgtgctgctgaagaatttcgagagcatcttcaagaagaaaacccggccctacagcatcgacggcatcatcctggtggagcccggcaacagctacctgaacaccaacaccttcaagtggaagcccacctgggacaacaccctggactttctggtccggaagtgccccgagtccctgaacgtgcccgagtacgcccccaagaagggcttcagcctgcatctgctgttcgtgggcatcagcggcgagctgtttaagaagctggccctgaactggtgccccggctacaccaagctgttccccgtgacccagcggaaccagaactacttccccgtgcagttccagcccagcgacttccccctggccttcctgtactaccaccccgacaccagcagcttcagcaacatcgatggcaaggtgctggaaatgcggtgcctgaagcgggagatcaactacgtgcgctgggagatcgtgaagatccgggaggaccggcagcaggatctgaaaaccggcggctacttcggcaacgacttcaagaccgccgagctgacctggctgaactacatggaccccttcagcttcgaggaactggccaagggacccagcggcatgtacttcgctggcgccaagaccggcatctacagagcccagaccgccctgatcagcttcatcaagcaggaaatcatccagaagatcagccaccagagctgggtgatcgacctgggcatcggcaagggccaggacctgggcagatacctggacgccggcgtgagacacctggtcggcatcgataaggaccagacagccctggccgagctggtgtaccggaagttctcccacgccaccaccagacagcacaagcacgccaccaacatctacgtgctgcaccaggatctggccgagcctgccaaagaaatcagcgagaaagtgcaccagatctatggcttccccaaagagggcgccagcagcatcgtgtccaacctgttcatccactacctgatgaagaacacccagcaggtcgagaacctggctgtgctgtgccacaagctgctgcagcctggcggcatggtctggttcaccaccatgctgggcgaacaggtgctggaactgctgcacgagaaccggatcgaactgaacgaagtgtgggaggcccgggagaacgaggtggtcaagttcgccatcaagcggctgttcaaagaggacatcctgcaggaaaccggccaggaaatcggcgtcctgctgcccttcagcaacggcgacttctacaatgagtacctggtcaacaccgcctttctgatcaagattttcaagcaccatggctttagcctcgtgcagaagcagagcttcaaggactggatccccgagttccagaacttcagcaagagcctgtacaagatcctgaccgaggccgacaagacctggaccagcctgttcggcttcatctgcctgcggaagaacggaggcgggggaagtggagggggcggcagtcaggacctgcacgccatccagctgcagctcgaagaggaaatgttcaacggcggcatcagaagattcgaggccgaccagcagagacagatcgcctctggcaacgagagcgacaccgcctggaatagaaggctgctgtctgagctgatcgcccctatggccgaaggcatccaggcctacaaagaggaatacgagggcaagagaggcagagcccctagagccctggccttcatcaactgtgtgggcaatgaggtggccgcctacatcaccatgaagatcgtgatggacatgctgaacaccgacgtgaccctgcaggccattgccatgaacgtggccgacagaatcgaggaccaggtccgattcagcaagctggaaggacacgccgccaagtacttcgagaaagtgaagaagtccctgaaggccagcaagaccaagagctacagacacgcccacaacgtggccgtggtggccgaaaaatctgtggccgatagggacgccgacttctctagatgggaggcctggcctaaggacaccctgctgcagatcggcatgaccctgctggaaatcctggaaaacagcgtgttcttcaacggccagcccgtgttcctgagaaccctgaggacaaatggcggcaagcacggcgtgtactacctgcagacatctgagcacgtgggcgagtggatcaccgccttcaaagaacatgtggcccagctgagccctgcctatgccccttgtgtgatccctcctagaccctgggtgtcccctttcaatggcggctttcacaccgagaaggtggccagcagaatcagactggtcaagggcaaccgggaacacgtgcggaagctgaccaagaaacagatgcccgccgtgtacaaggccgtgaatgctctgcaggccaccaagtggcaggtcaacaaagaggtgctgcaggtcgtcgaggacgtgatcagactggatctgggctacggcgtgccaagctttaagcccctgatcgacagagagaacaagcccgccaaccctgtgcccctggaatttcagcacctgagaggccgcgagctgaaagagatgctgacacctgaacagtggcaggcctttatcaattggaagggcgagtgcaccaagctgtacaccgccgagacaaagaggggctctaagtctgccgccacagtgcgaatggtcggacaggccagaaagtacagccagttcgacgccatctacttcgtgtacgccctggacagccggtctagagtgtatgcccagagcagcacactgagcccccagtctaacgatctgggaaaggccctgctgagattcaccgagggccagagactggattctgccgaagccctgaagtggttcctggtcaacggcgccaacaactggggctgggacaagaaaaccttcgatgtgcggaccgccaacgtgctggatagcgagttccaggacatgtgcagagatatcgccgccgaccctctgacctttacccagtgggtcaacgccgatagcccctatggattcctggcctggtgcttcgagtacgccagatacctggacgccctggatgagggaacccaggatcagttcatgacccatctgcccgtgcaccaggatggctcttgttctggcatccagcactacagcgccatgctgagcgatgccgtgggagccaaagccgtgaacctgaagcctagcgacagcccccaggatatctatggcgctgtggcccaggtggtcatccagaaaaactacgcctacatgaacgccgaggacgccgagacattcacaagcggaagcgtgacactgacaggcgccgagctgagatctatggcctctgcctgggacatgatcggcatcacacggggcctgaccaaaaagcctgtgatgacactgccctacggcagcaccagactgacctgtagagaaagcgtgatcgactacatcgtggacctggaagagaaagaggcccagagagccattgccgagggcagaacagccaatcctgtgcaccccttcgacaacgaccggaaggatagcctgacacctagcgccgcctacaactacatgaccgccctgatctggcccagcatctctgaagtggtcaaggcccctatcgtggccatgaagatgatcagacagctggccagattcgccgccaagagaaatgagggcctggaataccctctgcccaccggctttatcctgcagcagaaaatcatggccaccgacatgctgcgggtgtccacatgtctgatgggcgagatcaagatgagcctgcagatcgagacagacgtggtggacgagacagccatgatgggagccgccgctcctaattttgtgcacggacacgatgccagccacctgatcctgaccgtgtgcgatctggtggacaagggcatcactagcgtggccgtgatccacgatagctttggaacacacgccggcagaaccgccgacctgagagattctctgcgggaagagatggtcaagatgtaccagaaccacaacgccctgcagaacctgctggacgtgcacgaagaaagatggctggtggacaccggcatccaggtgccagaacagggagagttcgacctgaacgagatcctggtgtccgactactgcttcgcctga

SEQ ID NO: 22 - protein, 2797 aa, Artificial Sequence
  recombinant protein encoded by pC3P3-G3b plasmid
MDAQTRRRERRAEKQAQWKAANPFPVTTQGSQQTQPPQRHYGITSPISLAAPKETDCLLTQKLIETLKPFGVFEEEEELQRRILILGKLNNLVKEWIREISESKNLPQSVIENVGGKIFTFGSYRLGVHTKGADIDALCVAPRHVDRSDFFTSFYDKLKLQEEVKDLRAVEEAFVPVIKLCFDGIEIDILFARLALQTIPEDLDLRDDSLLKNLDIRCIRSLNGCRVTDEILHLVPNIDNFRLTLRAIKLWAKRHNIYSNILGFLGGVSWAMLVARTCQLYPNAIASTLVHKFFLVFSKWEWPNPVLLKQPEECNLNLPVWDPRVNPSDRYHLMPIITPAYPQQNSTYNVSVSTRMVMVEEFKQGLAITDEILLSKAEWSKLFEAPNFFQKYKHYIVLLASAPTEKQRLEWVGLVESKIRILVGSLEKNEFITLAHVNPQSFPAPKESPDREEFRTMWVIGLVFKKTENSENLSVDLTYDIQSFTDTVYRQAINSKMFELDMKIAAMHVKRKQLHQLLPSHVLQKRKKHSTEGVKLTALNDSSLDLSMDSDNSMSVPSPTSAMKTSPLNSSGSSQGRNSPAPAVTAASVTSIQASEVSVPQANSSESPGGPSSESIPQTATQPAIAPPPKPTVSRVVSSTRLVNPSPRPSGNTATKVPNPIVGVKRTSAPNKEEAPRRTKTEEDETSEDANCLALSGHDKTETKEQVDLETSAVQSETVPASASLLASQKTSSTDLSDIPALPANPIPVIKNSIKLRLNRGGGGSGGGGSSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDADPNTVSSFQVDCFLWHVRKRVADQELGDAPFLDRLRRDQKSLRGRGSTLGLDIETATRAGKQIVERILKEESGGGGSGGGGSRMAQLEAKVEELLSKNWNLENEVARLKKLVGERGSGPRPLLAIHPTEARHKQKIVAPVKQTLNFDLLKLAGDVESNPGPASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSQDLHAIQLQLEEEMFNGGIRRFEADQQRQIASGNESDTAWNRRLLSELIAPMAEGIQAYKEEYEGKRGRAPRALAFINCVGNEVAAYITMKIVMDMLNTDVTLQAIAMNVADRIEDQVRFSKLEGHAAKYFEKVKKSLKASKTKSYRHAHNVAVVAEKSVADRDADFSRWEAWPKDTLLQIGMTLLEILENSVFFNGQPVFLRTLRTNGGKHGVYYLQTSEHVGEWITAFKEHVAQLSPAYAPCVIPPRPWVSPFNGGFHTEKVASRIRLVKGNREHVRKLTKKQMPAVYKAVNALQATKWQVNKEVLQVVEDVIRLDLGYGVPSFKPLIDRENKPANPVPLEFQHLRGRELKEMLTPEQWQAFINWKGECTKLYTAETKRGSKSAATVRMVGQARKYSQFDAIYFVYALDSRSRVYAQSSTLSPQSNDLGKALLRFTEGQRLDSAEALKWFLVNGANNWGWDKKTFDVRTANVLDSEFQDMCRDIAADPLTFTQWVNADSPYGFLAWCFEYARYLDALDEGTQDQFMTHLPVHQDGSCSGIQHYSAMLSDAVGAKAVNLKPSDSPQDIYGAVAQVVIQKNYAYMNAEDAETFTSGSVTLTGAELRSMASAWDMIGITRGLTKKPVMTLPYGSTRLTCRESVIDYIVDLEEKEAQRAIAEGRTANPVHPFDNDRKDSLTPSAAYNYMTALIWPSISEVVKAPIVAMKMIRQLARFAAKRNEGLEYPLPTGFILQQKIMATDMLRVSTCLMGEIKMSLQIETDVVDETAMMGAAAPNFVHGHDASHLILTVCDLVDKGITSVAVIHDSFGTHAGRTADLRDSLREEMVKMYQNHNALQNLLDVHEERWLVDTGIQVPEQGEFDLNEILVSDYCFA

SEQ ID NO: 23 - DNA, 8394 bp, Artificial Sequence
  recombinant DNA open-reading frame from pC3P3- G3c plasmid
atggatgctcagaccagacgcagagaacggcgggcagagaagcaggcacagtggaaggccgctaatccgtttccagttacaacgcagggatcacaacaaacgcagccaccacagaggcactatggcattacctctcctatcagcttagcggcccccaaggagactgactgcctactcacacagaagctcatcgagacgctgaagccctttggggtttttgaagaagaagaggaactgcagcgcaggattttaattttgggaaaattaaataacctggtgaaagaatggattcgagaaatcagtgaaagcaagaatctcccacaatctgtaattgaaaatgttggagggaagatttttacatttggatcttacagactaggagtccacacgaaaggtgctgatattgatgcgttgtgtgttgcaccaagacatgttgatcgaagtgactttttcacctcattctatgataaattgaaattacaagaagaagtgaaagatttaagagctgttgaagaggcatttgtaccagttatcaaactctgttttgatggaatagagattgatattttgtttgcaagattagcactgcagactattccagaagatttggacctacgagatgacagtctgcttaaaaacctagatataagatgcataagaagccttaatggttgcagggtaaccgatgaaattttacatctagtaccaaacattgacaacttcaggttaactctgagagccatcaaactgtgggccaaacggcacaacatctattccaatatattaggtttcctcggtggtgtttcctgggctatgctagtagcaagaacttgccagctttatccaaatgcaatagcatcaactcttgtacataaatttttcttggtattttctaaatgggaatggccaaatccagtgctattgaaacagcctgaagaatgcaatcttaatttgcctgtgtgggacccaagggtaaaccccagtgataggtaccatcttatgcctataattacaccagcatacccacagcagaactccacgtacaatgtgtccgtttcaacacggatggtcatggttgaggagtttaaacaaggtcttgctatcacagatgaaattttgctgagtaaggcagagtggtccaaactttttgaagctccaaacttctttcagaagtacaagcattatattgtacttctagcaagtgcgcccacggaaaagcagcgtctggaatgggtgggcttggtggaatcaaaaatccgcatcctggttggaagcttggagaagaatgagtttattacactggctcatgtgaatccccagtcatttccagcccccaaagaaagtcctgacagggaagaatttcgcacaatgtgggtgattgggttagtgtttaaaaaaactgaaaactctgaaaatctcagtgtcgacctcacctatgatatccagtctttcacagacacagtttataggcaagcaataaacagcaaaatgtttgagttggatatgaagattgcagcaatgcatgtgaagagaaagcaactccatcagctgctgcctagtcacgtgcttcagaagaggaagaagcattcaacagaaggagtcaagttaacagctctgaatgacagcagccttgacttgtctatggacagtgataacagcatgtctgtgccttcacccaccagtgctatgaagaccagtccattgaatagttctggcagctcccagggcagaaacagtcctgctccagctgtgaccgcagcatctgtgaccagcatccaggcttctgaggtttctgtaccgcaagcaaattccagtgaaagcccagggggtccatcgagcgaaagcattcctcaaactgccacacagccagccattgccccaccaccaaagcctacagtctccagagttgtctcctcaacacgactggtaaacccatcgcctagaccttcaggaaacacagcaacaaaagtccctaatcctatagtaggagtcaagagaacgtccgcccccaataaagaagaagcccctagaaggaccaaaacagaagaggatgaaacaagtgaagatgctaactgtcttgctttgagtggacatgataaaacagagacaaaggaacaagttgatctggagacaagtgcggttcaatcagaaactgttccggcatcggcttctctgttggcctctcagaaaacatccagtacagacctttctgatatccctgctctccctgcaaatcctattcctgttatcaagaactcaataaaactgagactgaatcggggatcaggccctcgcccactgctcgcaatccaccctactgaagcaaggcacaagcaaaaaattgtggctcctgtgaagcagacgctgaactttgatctgctgaagctcgctggcgacgtggagtcgaaccctggccctagcaagatctacatcgacgagagaagcgacgccgagatcgtgtgcgccgccatcaagaacatcggcatcgagggcgccaccgccgcccagctgaccagacagctgaacatggagaagagagaggtgaacaaggccctgtacgacctgcagagaagcgccatggtgtacagcagcgacgacatcccccccagatggttcatgaccaccgaggccgacaagcccgacgccgacgccatggccgacgtgatcatcgacgacgtgagcagagagaagagcatgagagaggaccacaagagcttcgacgacgtgatccccgccaagaagatcatcgactggaaggacgccgaccccaatacagtctcctcttttcaagtggactgttttttgtggcatgttcgaaagcgggttgctgaccaagagctgggggatgccccgtttctagaccggctacggagagaccaaaagagcttgcgtgggagaggcagtaccctgggcctcgacatcgagaccgctacacgcgccggcaagcagatcgtggaacgcatcttaaaagaggagtccggaggcgggggaagtggagggggcggcagtaggatggctcagttggaggctaaggtagaggaactattgtccaaaaattggaatctcgagaacgaagtagctcgcctcaagaaactggtcggcgagcgcggaggaggaggctctggtggtggcgggagcgccagcctggacaacctggtggccagataccagcggtgcttcaacgaccagagcctgaagaacagcaccatcgagctggaaatccggttccagcagatcaacttcctgctgttcaagaccgtgtacgaggccctggtcgcccaggaaatccccagcaccatcagccacagcatccggtgcatcaagaaggtgcaccacgagaaccactgccgggagaagatcctgcccagcgagaacctgtacttcaagaaacagcccctgatgttcttcaagttcagcgagcccgccagcctgggctgtaaagtgtccctggccatcgagcagcccatccggaagttcatcctggacagcagcgtgctggtccggctgaagaaccggaccaccttccgggtgtccgagctgtggaagatcgagctgaccatcgtgaagcagctgatgggcagcgaggtgtcagccaagctggccgccttcaagaccctgctgttcgacacccccgagcagcagaccaccaagaacatgatgaccctgatcaaccccgacgacgagtacctgtacgagatcgagatcgagtacaccggcaagcctgagagcctgacagccgccgacgtgatcaagatcaagaacaccgtgctgacactgatcagccccaaccacctgatgctgaccgcctaccaccaggccatcgagtttatcgccagccacatcctgagcagcgagatcctgctggcccggatcaagagcggcaagtggggcctgaagagactgctgccccaggtcaagtccatgaccaaggccgactacatgaagttctacccccccgtgggctactacgtgaccgacaaggccgacggcatccggggcattgccgtgatccaggacacccagatctacgtggtggccgaccagctgtacagcctgggcaccaccggcatcgagcccctgaagcccaccatcctggacggcgagttcatgcccgagaagaaagagttctacggctttgacgtgatcatgtacgagggcaacctgctgacccagcagggcttcgagacacggatcgagagcctgagcaagggcatcaaggtgctgcaggccttcaacatcaaggccgagatgaagcccttcatcagcctgacctccgccgaccccaacgtgctgctgaagaatttcgagagcatcttcaagaagaaaacccggccctacagcatcgacggcatcatcctggtggagcccggcaacagctacctgaacaccaacaccttcaagtggaagcccacctgggacaacaccctggactttctggtccggaagtgccccgagtccctgaacgtgcccgagtacgcccccaagaagggcttcagcctgcatctgctgttcgtgggcatcagcggcgagctgtttaagaagctggccctgaactggtgccccggctacaccaagctgttccccgtgacccagcggaaccagaactacttccccgtgcagttccagcccagcgacttccccctggccttcctgtactaccaccccgacaccagcagcttcagcaacatcgatggcaaggtgctggaaatgcggtgcctgaagcgggagatcaactacgtgcgctgggagatcgtgaagatccgggaggaccggcagcaggatctgaaaaccggcggctacttcggcaacgacttcaagaccgccgagctgacctggctgaactacatggaccccttcagcttcgaggaactggccaagggacccagcggcatgtacttcgctggcgccaagaccggcatctacagagcccagaccgccctgatcagcttcatcaagcaggaaatcatccagaagatcagccaccagagctgggtgatcgacctgggcatcggcaagggccaggacctgggcagatacctggacgccggcgtgagacacctggtcggcatcgataaggaccagacagccctggccgagctggtgtaccggaagttctcccacgccaccaccagacagcacaagcacgccaccaacatctacgtgctgcaccaggatctggccgagcctgccaaagaaatcagcgagaaagtgcaccagatctatggcttccccaaagagggcgccagcagcatcgtgtccaacctgttcatccactacctgatgaagaacacccagcaggtcgagaacctggctgtgctgtgccacaagctgctgcagcctggcggcatggtctggttcaccaccatgctgggcgaacaggtgctggaactgctgcacgagaaccggatcgaactgaacgaagtgtgggaggcccgggagaacgaggtggtcaagttcgccatcaagcggctgttcaaagaggacatcctgcaggaaaccggccaggaaatcggcgtcctgctgcccttcagcaacggcgacttctacaatgagtacctggtcaacaccgcctttctgatcaagattttcaagcaccatggctttagcctcgtgcagaagcagagcttcaaggactggatccccgagttccagaacttcagcaagagcctgtacaagatcctgaccgaggccgacaagacctggaccagcctgttcggcttcatctgcctgcggaagaacggaggcgggggaagtggagggggcggcagtcaggacctgcacgccatccagctgcagctcgaagaggaaatgttcaacggcggcatcagaagattcgaggccgaccagcagagacagatcgcctctggcaacgagagcgacaccgcctggaatagaaggctgctgtctgagctgatcgcccctatggccgaaggcatccaggcctacaaagaggaatacgagggcaagagaggcagagcccctagagccctggccttcatcaactgtgtgggcaatgaggtggccgcctacatcaccatgaagatcgtgatggacatgctgaacaccgacgtgaccctgcaggccattgccatgaacgtggccgacagaatcgaggaccaggtccgattcagcaagctggaaggacacgccgccaagtacttcgagaaagtgaagaagtccctgaaggccagcaagaccaagagctacagacacgcccacaacgtggccgtggtggccgaaaaatctgtggccgatagggacgccgacttctctagatgggaggcctggcctaaggacaccctgctgcagatcggcatgaccctgctggaaatcctggaaaacagcgtgttcttcaacggccagcccgtgttcctgagaaccctgaggacaaatggcggcaagcacggcgtgtactacctgcagacatctgagcacgtgggcgagtggatcaccgccttcaaagaacatgtggcccagctgagccctgcctatgccccttgtgtgatccctcctagaccctgggtgtcccctttcaatggcggctttcacaccgagaaggtggccagcagaatcagactggtcaagggcaaccgggaacacgtgcggaagctgaccaagaaacagatgcccgccgtgtacaaggccgtgaatgctctgcaggccaccaagtggcaggtcaacaaagaggtgctgcaggtcgtcgaggacgtgatcagactggatctgggctacggcgtgccaagctttaagcccctgatcgacagagagaacaagcccgccaaccctgtgcccctggaatttcagcacctgagaggccgcgagctgaaagagatgctgacacctgaacagtggcaggcctttatcaattggaagggcgagtgcaccaagctgtacaccgccgagacaaagaggggctctaagtctgccgccacagtgcgaatggtcggacaggccagaaagtacagccagttcgacgccatctacttcgtgtacgccctggacagccggtctagagtgtatgcccagagcagcacactgagcccccagtctaacgatctgggaaaggccctgctgagattcaccgagggccagagactggattctgccgaagccctgaagtggttcctggtcaacggcgccaacaactggggctgggacaagaaaaccttcgatgtgcggaccgccaacgtgctggatagcgagttccaggacatgtgcagagatatcgccgccgaccctctgacctttacccagtgggtcaacgccgatagcccctatggattcctggcctggtgcttcgagtacgccagatacctggacgccctggatgagggaacccaggatcagttcatgacccatctgcccgtgcaccaggatggctcttgttctggcatccagcactacagcgccatgctgagcgatgccgtgggagccaaagccgtgaacctgaagcctagcgacagcccccaggatatctatggcgctgtggcccaggtggtcatccagaaaaactacgcctacatgaacgccgaggacgccgagacattcacaagcggaagcgtgacactgacaggcgccgagctgagatctatggcctctgcctgggacatgatcggcatcacacggggcctgaccaaaaagcctgtgatgacactgccctacggcagcaccagactgacctgtagagaaagcgtgatcgactacatcgtggacctggaagagaaagaggcccagagagccattgccgagggcagaacagccaatcctgtgcaccccttcgacaacgaccggaaggatagcctgacacctagcgccgcctacaactacatgaccgccctgatctggcccagcatctctgaagtggtcaaggcccctatcgtggccatgaagatgatcagacagctggccagattcgccgccaagagaaatgagggcctggaataccctctgcccaccggctttatcctgcagcagaaaatcatggccaccgacatgctgcgggtgtccacatgtctgatgggcgagatcaagatgagcctgcagatcgagacagacgtggtggacgagacagccatgatgggagccgccgctcctaattttgtgcacggacacgatgccagccacctgatcctgaccgtgtgcgatctggtggacaagggcatcactagcgtggccgtgatccacgatagctttggaacacacgccggcagaaccgccgacctgagagattctctgcgggaagagatggtcaagatgtaccagaaccacaacgccctgcagaacctgctggacgtgcacgaagaaagatggctggtggacaccggcatccaggtgccagaacagggagagttcgacctgaacgagatcctggtgtccgactactgcttcgcctga

SEQ ID NO: 24 - protein, 2797 aa, Artificial Sequence
  recombinant protein encoded by pC3P3-G3c plasmid
MDAQTRRRERRAEKQAQWKAANPFPVTTQGSQQTQPPQRHYGITSPISLAAPKETDCLLTQKLIETLKPFGVFEEEEELQRRILILGKLNNLVKEWIREISESKNLPQSVIENVGGKIFTFGSYRLGVHTKGADIDALCVAPRHVDRSDFFTSFYDKLKLQEEVKDLRAVEEAFVPVIKLCFDGIEIDILFARLALQTIPEDLDLRDDSLLKNLDIRCIRSLNGCRVTDEILHLVPNIDNFRLTLRAIKLWAKRHNIYSNILGFLGGVSWAMLVARTCQLYPNAIASTLVHKFFLVFSKWEWPNPVLLKQPEECNLNLPVWDPRVNPSDRYHLMPIITPAYPQQNSTYNVSVSTRMVMVEEFKQGLAITDEILLSKAEWSKLFEAPNFFQKYKHYIVLLASAPTEKQRLEWVGLVESKIRILVGSLEKNEFITLAHVNPQSFPAPKESPDREEFRTMWVIGLVFKKTENSENLSVDLTYDIQSFTDTVYRQAINSKMFELDMKIAAMHVKRKQLHQLLPSHVLQKRKKHSTEGVKLTALNDSSLDLSMDSDNSMSVPSPTSAMKTSPLNSSGSSQGRNSPAPAVTAASVTSIQASEVSVPQANSSESPGGPSSESIPQTATQPAIAPPPKPTVSRVVSSTRLVNPSPRPSGNTATKVPNPIVGVKRTSAPNKEEAPRRTKTEEDETSEDANCLALSGHDKTETKEQVDLETSAVQSETVPASASLLASQKTSSTDLSDIPALPANPIPVIKNSIKLRLNRGSGPRPLLAIHPTEARHKQKIVAPVKQTLNFDLLKLAGDVESNPGPSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDADPNTVSSFQVDCFLWHVRKRVADQELGDAPFLDRLRRDQKSLRGRGSTLGLDIETATRAGKQIVERILKEESGGGGSGGGGSRMAQLEAKVEELLSKNWNLENEVARLKKLVGERGGGGSGGGGSASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSQDLHAIQLQLEEEMFNGGIRRFEADQQRQIASGNESDTAWNRRLLSELIAPMAEGIQAYKEEYEGKRGRAPRALAFINCVGNEVAAYITMKIVMDMLNTDVTLQAIAMNVADRIEDQVRFSKLEGHAAKYFEKVKKSLKASKTKSYRHAHNVAVVAEKSVADRDADFSRWEAWPKDTLLQIGMTLLEILENSVFFNGQPVFLRTLRTNGGKHGVYYLQTSEHVGEWITAFKEHVAQLSPAYAPCVIPPRPWVSPFNGGFHTEKVASRIRLVKGNREHVRKLTKKQMPAVYKAVNALQATKWQVNKEVLQVVEDVIRLDLGYGVPSFKPLIDRENKPANPVPLEFQHLRGRELKEMLTPEQWQAFINWKGECTKLYTAETKRGSKSAATVRMVGQARKYSQFDAIYFVYALDSRSRVYAQSSTLSPQSNDLGKALLRFTEGQRLDSAEALKWFLVNGANNWGWDKKTFDVRTANVLDSEFQDMCRDIAADPLTFTQWVNADSPYGFLAWCFEYARYLDALDEGTQDQFMTHLPVHQDGSCSGIQHYSAMLSDAVGAKAVNLKPSDSPQDIYGAVAQVVIQKNYAYMNAEDAETFTSGSVTLTGAELRSMASAWDMIGITRGLTKKPVMTLPYGSTRLTCRESVIDYIVDLEEKEAQRAIAEGRTANPVHPFDNDRKDSLTPSAAYNYMTALIWPSISEVVKAPIVAMKMIRQLARFAAKRNEGLEYPLPTGFILQQKIMATDMLRVSTCLMGEIKMSLQIETDVVDETAMMGAAAPNFVHGHDASHLILTVCDLVDKGITSVAVIHDSFGTHAGRTADLRDSLREEMVKMYQNHNALQNLLDVHEERWLVDTGIQVPEQGEFDLNEILVSDYCFA

SEQ ID NO: 25 - DNA, 8502 bp, Artificial Sequence
  recombinant DNA open-reading frame from pC3P3- G3d plasmid
atgagcaagatctacatcgacgagagaagcgacgccgagatcgtgtgcgccgccatcaagaacatcggcatcgagggcgccaccgccgcccagctgaccagacagctgaacatggagaagagagaggtgaacaaggccctgtacgacctgcagagaagcgccatggtgtacagcagcgacgacatcccccccagatggttcatgaccaccgaggccgacaagcccgacgccgacgccatggccgacgtgatcatcgacgacgtgagcagagagaagagcatgagagaggaccacaagagcttcgacgacgtgatccccgccaagaagatcatcgactggaaggacgccgaccccaatacagtctcctcttttcaagtggactgttttttgtggcatgttcgaaagcgggttgctgaccaagagctgggggatgccccgtttctagaccggctacggagagaccaaaagagcttgcgtgggagaggcagtaccctgggcctcgacatcgagaccgctacacgcgccggcaagcagatcgtggaacgcatcttaaaagaggagtccggaggcgggggaagtggagggggcggcagtaggatggctcagttggaggctaaggtagaggaactattgtccaaaaattggaatctcgagaacgaagtagctcgcctcaagaaactggtcggcgagcgcggatcaggcccgcgacccctccttgccatccacccaaccgaggcacggcacaagcagaaaatagtggcacccgttaagcagactctcaactttgatctcctgaagcttgccggcgacgtagagtccaaccccggccccgatgctcagaccagacgcagagaacggcgggcagagaagcaggcacagtggaaggccgctaatccgtttccagttacaacgcagggatcacaacaaacgcagccaccacagaggcactatggcattacctctcctatcagcttagcggcccccaaggagactgactgcctactcacacagaagctcatcgagacgctgaagccctttggggtttttgaagaagaagaggaactgcagcgcaggattttaattttgggaaaattaaataacctggtgaaagaatggattcgagaaatcagtgaaagcaagaatctcccacaatctgtaattgaaaatgttggagggaagatttttacatttggatcttacagactaggagtccacacgaaaggtgctgatattgatgcgttgtgtgttgcaccaagacatgttgatcgaagtgactttttcacctcattctatgataaattgaaattacaagaagaagtgaaagatttaagagctgttgaagaggcatttgtaccagttatcaaactctgttttgatggaatagagattgatattttgtttgcaagattagcactgcagactattccagaagatttggacctacgagatgacagtctgcttaaaaacctagatataagatgcataagaagccttaatggttgcagggtaaccgatgaaattttacatctagtaccaaacattgacaacttcaggttaactctgagagccatcaaactgtgggccaaacggcacaacatctattccaatatattaggtttcctcggtggtgtttcctgggctatgctagtagcaagaacttgccagctttatccaaatgcaatagcatcaactcttgtacataaatttttcttggtattttctaaatgggaatggccaaatccagtgctattgaaacagcctgaagaatgcaatcttaatttgcctgtgtgggacccaagggtaaaccccagtgataggtaccatcttatgcctataattacaccagcatacccacagcagaactccacgtacaatgtgtccgtttcaacacggatggtcatggttgaggagtttaaacaaggtcttgctatcacagatgaaattttgctgagtaaggcagagtggtccaaactttttgaagctccaaacttctttcagaagtacaagcattatattgtacttctagcaagtgcgcccacggaaaagcagcgtctggaatgggtgggcttggtggaatcaaaaatccgcatcctggttggaagcttggagaagaatgagtttattacactggctcatgtgaatccccagtcatttccagcccccaaagaaagtcctgacagggaagaatttcgcacaatgtgggtgattgggttagtgtttaaaaaaactgaaaactctgaaaatctcagtgtcgacctcacctatgatatccagtctttcacagacacagtttataggcaagcaataaacagcaaaatgtttgagttggatatgaagattgcagcaatgcatgtgaagagaaagcaactccatcagctgctgcctagtcacgtgcttcagaagaggaagaagcattcaacagaaggagtcaagttaacagctctgaatgacagcagccttgacttgtctatggacagtgataacagcatgtctgtgccttcacccaccagtgctatgaagaccagtccattgaatagttctggcagctcccagggcagaaacagtcctgctccagctgtgaccgcagcatctgtgaccagcatccaggcttctgaggtttctgtaccgcaagcaaattccagtgaaagcccagggggtccatcgagcgaaagcattcctcaaactgccacacagccagccattgccccaccaccaaagcctacagtctccagagttgtctcctcaacacgactggtaaacccatcgcctagaccttcaggaaacacagcaacaaaagtccctaatcctatagtaggagtcaagagaacgtccgcccccaataaagaagaagcccctagaaggaccaaaacagaagaggatgaaacaagtgaagatgctaactgtcttgctttgagtggacatgataaaacagagacaaaggaacaagttgatctggagacaagtgcggttcaatcagaaactgttccggcatcggcttctctgttggcctctcagaaaacatccagtacagacctttctgatatccctgctctccctgcaaatcctattcctgttatcaagaactcaataaaactgagactgaatcggggatcaggccctcgcccactgctcgcaatccaccctactgaagcaaggcacaagcaaaaaattgtggctcctgtgaagcagacgctgaactttgatctgctgaagctcgctggcgacgtggagtcgaaccctggccctgccagcctggacaacctggtggccagataccagcggtgcttcaacgaccagagcctgaagaacagcaccatcgagctggaaatccggttccagcagatcaacttcctgctgttcaagaccgtgtacgaggccctggtcgcccaggaaatccccagcaccatcagccacagcatccggtgcatcaagaaggtgcaccacgagaaccactgccgggagaagatcctgcccagcgagaacctgtacttcaagaaacagcccctgatgttcttcaagttcagcgagcccgccagcctgggctgtaaagtgtccctggccatcgagcagcccatccggaagttcatcctggacagcagcgtgctggtccggctgaagaaccggaccaccttccgggtgtccgagctgtggaagatcgagctgaccatcgtgaagcagctgatgggcagcgaggtgtcagccaagctggccgccttcaagaccctgctgttcgacacccccgagcagcagaccaccaagaacatgatgaccctgatcaaccccgacgacgagtacctgtacgagatcgagatcgagtacaccggcaagcctgagagcctgacagccgccgacgtgatcaagatcaagaacaccgtgctgacactgatcagccccaaccacctgatgctgaccgcctaccaccaggccatcgagtttatcgccagccacatcctgagcagcgagatcctgctggcccggatcaagagcggcaagtggggcctgaagagactgctgccccaggtcaagtccatgaccaaggccgactacatgaagttctacccccccgtgggctactacgtgaccgacaaggccgacggcatccggggcattgccgtgatccaggacacccagatctacgtggtggccgaccagctgtacagcctgggcaccaccggcatcgagcccctgaagcccaccatcctggacggcgagttcatgcccgagaagaaagagttctacggctttgacgtgatcatgtacgagggcaacctgctgacccagcagggcttcgagacacggatcgagagcctgagcaagggcatcaaggtgctgcaggccttcaacatcaaggccgagatgaagcccttcatcagcctgacctccgccgaccccaacgtgctgctgaagaatttcgagagcatcttcaagaagaaaacccggccctacagcatcgacggcatcatcctggtggagcccggcaacagctacctgaacaccaacaccttcaagtggaagcccacctgggacaacaccctggactttctggtccggaagtgccccgagtccctgaacgtgcccgagtacgcccccaagaagggcttcagcctgcatctgctgttcgtgggcatcagcggcgagctgtttaagaagctggccctgaactggtgccccggctacaccaagctgttccccgtgacccagcggaaccagaactacttccccgtgcagttccagcccagcgacttccccctggccttcctgtactaccaccccgacaccagcagcttcagcaacatcgatggcaaggtgctggaaatgcggtgcctgaagcgggagatcaactacgtgcgctgggagatcgtgaagatccgggaggaccggcagcaggatctgaaaaccggcggctacttcggcaacgacttcaagaccgccgagctgacctggctgaactacatggaccccttcagcttcgaggaactggccaagggacccagcggcatgtacttcgctggcgccaagaccggcatctacagagcccagaccgccctgatcagcttcatcaagcaggaaatcatccagaagatcagccaccagagctgggtgatcgacctgggcatcggcaagggccaggacctgggcagatacctggacgccggcgtgagacacctggtcggcatcgataaggaccagacagccctggccgagctggtgtaccggaagttctcccacgccaccaccagacagcacaagcacgccaccaacatctacgtgctgcaccaggatctggccgagcctgccaaagaaatcagcgagaaagtgcaccagatctatggcttccccaaagagggcgccagcagcatcgtgtccaacctgttcatccactacctgatgaagaacacccagcaggtcgagaacctggctgtgctgtgccacaagctgctgcagcctggcggcatggtctggttcaccaccatgctgggcgaacaggtgctggaactgctgcacgagaaccggatcgaactgaacgaagtgtgggaggcccgggagaacgaggtggtcaagttcgccatcaagcggctgttcaaagaggacatcctgcaggaaaccggccaggaaatcggcgtcctgctgcccttcagcaacggcgacttctacaatgagtacctggtcaacaccgcctttctgatcaagattttcaagcaccatggctttagcctcgtgcagaagcagagcttcaaggactggatccccgagttccagaacttcagcaagagcctgtacaagatcctgaccgaggccgacaagacctggaccagcctgttcggcttcatctgcctgcggaagaacggaggcgggggaagtggagggggcggcagtcaggacctgcacgccatccagctgcagctcgaagaggaaatgttcaacggcggcatcagaagattcgaggccgaccagcagagacagatcgcctctggcaacgagagcgacaccgcctggaatagaaggctgctgtctgagctgatcgcccctatggccgaaggcatccaggcctacaaagaggaatacgagggcaagagaggcagagcccctagagccctggccttcatcaactgtgtgggcaatgaggtggccgcctacatcaccatgaagatcgtgatggacatgctgaacaccgacgtgaccctgcaggccattgccatgaacgtggccgacagaatcgaggaccaggtccgattcagcaagctggaaggacacgccgccaagtacttcgagaaagtgaagaagtccctgaaggccagcaagaccaagagctacagacacgcccacaacgtggccgtggtggccgaaaaatctgtggccgatagggacgccgacttctctagatgggaggcctggcctaaggacaccctgctgcagatcggcatgaccctgctggaaatcctggaaaacagcgtgttcttcaacggccagcccgtgttcctgagaaccctgaggacaaatggcggcaagcacggcgtgtactacctgcagacatctgagcacgtgggcgagtggatcaccgccttcaaagaacatgtggcccagctgagccctgcctatgccccttgtgtgatccctcctagaccctgggtgtcccctttcaatggcggctttcacaccgagaaggtggccagcagaatcagactggtcaagggcaaccgggaacacgtgcggaagctgaccaagaaacagatgcccgccgtgtacaaggccgtgaatgctctgcaggccaccaagtggcaggtcaacaaagaggtgctgcaggtcgtcgaggacgtgatcagactggatctgggctacggcgtgccaagctttaagcccctgatcgacagagagaacaagcccgccaaccctgtgcccctggaatttcagcacctgagaggccgcgagctgaaagagatgctgacacctgaacagtggcaggcctttatcaattggaagggcgagtgcaccaagctgtacaccgccgagacaaagaggggctctaagtctgccgccacagtgcgaatggtcggacaggccagaaagtacagccagttcgacgccatctacttcgtgtacgccctggacagccggtctagagtgtatgcccagagcagcacactgagcccccagtctaacgatctgggaaaggccctgctgagattcaccgagggccagagactggattctgccgaagccctgaagtggttcctggtcaacggcgccaacaactggggctgggacaagaaaaccttcgatgtgcggaccgccaacgtgctggatagcgagttccaggacatgtgcagagatatcgccgccgaccctctgacctttacccagtgggtcaacgccgatagcccctatggattcctggcctggtgcttcgagtacgccagatacctggacgccctggatgagggaacccaggatcagttcatgacccatctgcccgtgcaccaggatggctcttgttctggcatccagcactacagcgccatgctgagcgatgccgtgggagccaaagccgtgaacctgaagcctagcgacagcccccaggatatctatggcgctgtggcccaggtggtcatccagaaaaactacgcctacatgaacgccgaggacgccgagacattcacaagcggaagcgtgacactgacaggcgccgagctgagatctatggcctctgcctgggacatgatcggcatcacacggggcctgaccaaaaagcctgtgatgacactgccctacggcagcaccagactgacctgtagagaaagcgtgatcgactacatcgtggacctggaagagaaagaggcccagagagccattgccgagggcagaacagccaatcctgtgcaccccttcgacaacgaccggaaggatagcctgacacctagcgccgcctacaactacatgaccgccctgatctggcccagcatctctgaagtggtcaaggcccctatcgtggccatgaagatgatcagacagctggccagattcgccgccaagagaaatgagggcctggaataccctctgcccaccggctttatcctgcagcagaaaatcatggccaccgacatgctgcgggtgtccacatgtctgatgggcgagatcaagatgagcctgcagatcgagacagacgtggtggacgagacagccatgatgggagccgccgctcctaattttgtgcacggacacgatgccagccacctgatcctgaccgtgtgcgatctggtggacaagggcatcactagcgtggccgtgatccacgatagctttggaacacacgccggcagaaccgccgacctgagagattctctgcgggaagagatggtcaagatgtaccagaaccacaacgccctgcagaacctgctggacgtgcacgaagaaagatggctggtggacaccggcatccaggtgccagaacagggagagttcgacctgaacgagatcctggtgtccgactactgcttcgcctga

SEQ ID NO: 26 - protein, 2833 aa, Artificial Sequence
  recombinant protein encoded by pC3P3-G3d plasmid
MSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDADPNTVSSFQVDCFLWHVRKRVADQELGDAPFLDRLRRDQKSLRGRGSTLGLDIETATRAGKQIVERILKEESGGGGSGGGGSRMAQLEAKVEELLSKNWNLENEVARLKKLVGERGSGPRPLLAIHPTEARHKQKIVAPVKQTLNFDLLKLAGDVESNPGPDAQTRRRERRAEKQAQWKAANPFPVTTQGSQQTQPPQRHYGITSPISLAAPKETDCLLTQKLIETLKPFGVFEEEEELQRRILILGKLNNLVKEWIREISESKNLPQSVIENVGGKIFTFGSYRLGVHTKGADIDALCVAPRHVDRSDFFTSFYDKLKLQEEVKDLRAVEEAFVPVIKLCFDGIEIDILFARLALQTIPEDLDLRDDSLLKNLDIRCIRSLNGCRVTDEILHLVPNIDNFRLTLRAIKLWAKRHNIYSNILGFLGGVSWAMLVARTCQLYPNAIASTLVHKFFLVFSKWEWPNPVLLKQPEECNLNLPVWDPRVNPSDRYHLMPIITPAYPQQNSTYNVSVSTRMVMVEEFKQGLAITDEILLSKAEWSKLFEAPNFFQKYKHYIVLLASAPTEKQRLEWVGLVESKIRILVGSLEKNEFITLAHVNPQSFPAPKESPDREEFRTMWVIGLVFKKTENSENLSVDLTYDIQSFTDTVYRQAINSKMFELDMKIAAMHVKRKQLHQLLPSHVLQKRKKHSTEGVKLTALNDSSLDLSMDSDNSMSVPSPTSAMKTSPLNSSGSSQGRNSPAPAVTAASVTSIQASEVSVPQANSSESPGGPSSESIPQTATQPAIAPPPKPTVSRVVSSTRLVNPSPRPSGNTATKVPNPIVGVKRTSAPNKEEAPRRTKTEEDETSEDANCLALSGHDKTETKEQVDLETSAVQSETVPASASLLASQKTSSTDLSDIPALPANPIPVIKNSIKLRLNRGSGPRPLLAIHPTEARHKQKIVAPVKQTLNFDLLKLAGDVESNPGPASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSQDLHAIQLQLEEEMFNGGIRRFEADQQRQIASGNESDTAWNRRLLSELIAPMAEGIQAYKEEYEGKRGRAPRALAFINCVGNEVAAYITMKIVMDMLNTDVTLQAIAMNVADRIEDQVRFSKLEGHAAKYFEKVKKSLKASKTKSYRHAHNVAVVAEKSVADRDADFSRWEAWPKDTLLQIGMTLLEILENSVFFNGQPVFLRTLRTNGGKHGVYYLQTSEHVGEWITAFKEHVAQLSPAYAPCVIPPRPWVSPFNGGFHTEKVASRIRLVKGNREHVRKLTKKQMPAVYKAVNALQATKWQVNKEVLQVVEDVIRLDLGYGVPSFKPLIDRENKPANPVPLEFQHLRGRELKEMLTPEQWQAFINWKGECTKLYTAETKRGSKSAATVRMVGQARKYSQFDAIYFVYALDSRSRVYAQSSTLSPQSNDLGKALLRFTEGQRLDSAEALKWFLVNGANNWGWDKKTFDVRTANVLDSEFQDMCRDIAADPLTFTQWVNADSPYGFLAWCFEYARYLDALDEGTQDQFMTHLPVHQDGSCSGIQHYSAMLSDAVGAKAVNLKPSDSPQDIYGAVAQVVIQKNYAYMNAEDAETFTSGSVTLTGAELRSMASAWDMIGITRGLTKKPVMTLPYGSTRLTCRESVIDYIVDLEEKEAQRAIAEGRTANPVHPFDNDRKDSLTPSAAYNYMTALIWPSISEVVKAPIVAMKMIRQLARFAAKRNEGLEYPLPTGFILQQKIMATDMLRVSTCLMGEIKMSLQIETDVVDETAMMGAAAPNFVHGHDASHLILTVCDLVDKGITSVAVIHDSFGTHAGRTADLRDSLREEMVKMYQNHNALQNLLDVHEERWLVDTGIQVPEQGEFDLNEILVSDYCFA

SEQ ID NO: 27 - DNA, 8394 bp, Artificial Sequence
  recombinant DNA open-reading frame from pC3P3- G3e plasmid
atgagcaagatctacatcgacgagagaagcgacgccgagatcgtgtgcgccgccatcaagaacatcggcatcgagggcgccaccgccgcccagctgaccagacagctgaacatggagaagagagaggtgaacaaggccctgtacgacctgcagagaagcgccatggtgtacagcagcgacgacatcccccccagatggttcatgaccaccgaggccgacaagcccgacgccgacgccatggccgacgtgatcatcgacgacgtgagcagagagaagagcatgagagaggaccacaagagcttcgacgacgtgatccccgccaagaagatcatcgactggaaggacgccgaccccaatacagtctcctcttttcaagtggactgttttttgtggcatgttcgaaagcgggttgctgaccaagagctgggggatgccccgtttctagaccggctacggagagaccaaaagagcttgcgtgggagaggcagtaccctgggcctcgacatcgagaccgctacacgcgccggcaagcagatcgtggaacgcatcttaaaagaggagtccggaggcgggggaagtggagggggcggcagtaggatggctcagttggaggctaaggtagaggaactattgtccaaaaattggaatctcgagaacgaagtagctcgcctcaagaaactggtcggcgagcgcggaggaggaggctctggtggtggcgggagcgatgctcagaccagacgcagagaacggcgggcagagaagcaggcacagtggaaggccgctaatccgtttccagttacaacgcagggatcacaacaaacgcagccaccacagaggcactatggcattacctctcctatcagcttagcggcccccaaggagactgactgcctactcacacagaagctcatcgagacgctgaagccctttggggtttttgaagaagaagaggaactgcagcgcaggattttaattttgggaaaattaaataacctggtgaaagaatggattcgagaaatcagtgaaagcaagaatctcccacaatctgtaattgaaaatgttggagggaagatttttacatttggatcttacagactaggagtccacacgaaaggtgctgatattgatgcgttgtgtgttgcaccaagacatgttgatcgaagtgactttttcacctcattctatgataaattgaaattacaagaagaagtgaaagatttaagagctgttgaagaggcatttgtaccagttatcaaactctgttttgatggaatagagattgatattttgtttgcaagattagcactgcagactattccagaagatttggacctacgagatgacagtctgcttaaaaacctagatataagatgcataagaagccttaatggttgcagggtaaccgatgaaattttacatctagtaccaaacattgacaacttcaggttaactctgagagccatcaaactgtgggccaaacggcacaacatctattccaatatattaggtttcctcggtggtgtttcctgggctatgctagtagcaagaacttgccagctttatccaaatgcaatagcatcaactcttgtacataaatttttcttggtattttctaaatgggaatggccaaatccagtgctattgaaacagcctgaagaatgcaatcttaatttgcctgtgtgggacccaagggtaaaccccagtgataggtaccatcttatgcctataattacaccagcatacccacagcagaactccacgtacaatgtgtccgtttcaacacggatggtcatggttgaggagtttaaacaaggtcttgctatcacagatgaaattttgctgagtaaggcagagtggtccaaactttttgaagctccaaacttctttcagaagtacaagcattatattgtacttctagcaagtgcgcccacggaaaagcagcgtctggaatgggtgggcttggtggaatcaaaaatccgcatcctggttggaagcttggagaagaatgagtttattacactggctcatgtgaatccccagtcatttccagcccccaaagaaagtcctgacagggaagaatttcgcacaatgtgggtgattgggttagtgtttaaaaaaactgaaaactctgaaaatctcagtgtcgacctcacctatgatatccagtctttcacagacacagtttataggcaagcaataaacagcaaaatgtttgagttggatatgaagattgcagcaatgcatgtgaagagaaagcaactccatcagctgctgcctagtcacgtgcttcagaagaggaagaagcattcaacagaaggagtcaagttaacagctctgaatgacagcagccttgacttgtctatggacagtgataacagcatgtctgtgccttcacccaccagtgctatgaagaccagtccattgaatagttctggcagctcccagggcagaaacagtcctgctccagctgtgaccgcagcatctgtgaccagcatccaggcttctgaggtttctgtaccgcaagcaaattccagtgaaagcccagggggtccatcgagcgaaagcattcctcaaactgccacacagccagccattgccccaccaccaaagcctacagtctccagagttgtctcctcaacacgactggtaaacccatcgcctagaccttcaggaaacacagcaacaaaagtccctaatcctatagtaggagtcaagagaacgtccgcccccaataaagaagaagcccctagaaggaccaaaacagaagaggatgaaacaagtgaagatgctaactgtcttgctttgagtggacatgataaaacagagacaaaggaacaagttgatctggagacaagtgcggttcaatcagaaactgttccggcatcggcttctctgttggcctctcagaaaacatccagtacagacctttctgatatccctgctctccctgcaaatcctattcctgttatcaagaactcaataaaactgagactgaatcggggatcaggccctcgcccactgctcgcaatccaccctactgaagcaaggcacaagcaaaaaattgtggctcctgtgaagcagacgctgaactttgatctgctgaagctcgctggcgacgtggagtcgaaccctggccctgccagcctggacaacctggtggccagataccagcggtgcttcaacgaccagagcctgaagaacagcaccatcgagctggaaatccggttccagcagatcaacttcctgctgttcaagaccgtgtacgaggccctggtcgcccaggaaatccccagcaccatcagccacagcatccggtgcatcaagaaggtgcaccacgagaaccactgccgggagaagatcctgcccagcgagaacctgtacttcaagaaacagcccctgatgttcttcaagttcagcgagcccgccagcctgggctgtaaagtgtccctggccatcgagcagcccatccggaagttcatcctggacagcagcgtgctggtccggctgaagaaccggaccaccttccgggtgtccgagctgtggaagatcgagctgaccatcgtgaagcagctgatgggcagcgaggtgtcagccaagctggccgccttcaagaccctgctgttcgacacccccgagcagcagaccaccaagaacatgatgaccctgatcaaccccgacgacgagtacctgtacgagatcgagatcgagtacaccggcaagcctgagagcctgacagccgccgacgtgatcaagatcaagaacaccgtgctgacactgatcagccccaaccacctgatgctgaccgcctaccaccaggccatcgagtttatcgccagccacatcctgagcagcgagatcctgctggcccggatcaagagcggcaagtggggcctgaagagactgctgccccaggtcaagtccatgaccaaggccgactacatgaagttctacccccccgtgggctactacgtgaccgacaaggccgacggcatccggggcattgccgtgatccaggacacccagatctacgtggtggccgaccagctgtacagcctgggcaccaccggcatcgagcccctgaagcccaccatcctggacggcgagttcatgcccgagaagaaagagttctacggctttgacgtgatcatgtacgagggcaacctgctgacccagcagggcttcgagacacggatcgagagcctgagcaagggcatcaaggtgctgcaggccttcaacatcaaggccgagatgaagcccttcatcagcctgacctccgccgaccccaacgtgctgctgaagaatttcgagagcatcttcaagaagaaaacccggccctacagcatcgacggcatcatcctggtggagcccggcaacagctacctgaacaccaacaccttcaagtggaagcccacctgggacaacaccctggactttctggtccggaagtgccccgagtccctgaacgtgcccgagtacgcccccaagaagggcttcagcctgcatctgctgttcgtgggcatcagcggcgagctgtttaagaagctggccctgaactggtgccccggctacaccaagctgttccccgtgacccagcggaaccagaactacttccccgtgcagttccagcccagcgacttccccctggccttcctgtactaccaccccgacaccagcagcttcagcaacatcgatggcaaggtgctggaaatgcggtgcctgaagcgggagatcaactacgtgcgctgggagatcgtgaagatccgggaggaccggcagcaggatctgaaaaccggcggctacttcggcaacgacttcaagaccgccgagctgacctggctgaactacatggaccccttcagcttcgaggaactggccaagggacccagcggcatgtacttcgctggcgccaagaccggcatctacagagcccagaccgccctgatcagcttcatcaagcaggaaatcatccagaagatcagccaccagagctgggtgatcgacctgggcatcggcaagggccaggacctgggcagatacctggacgccggcgtgagacacctggtcggcatcgataaggaccagacagccctggccgagctggtgtaccggaagttctcccacgccaccaccagacagcacaagcacgccaccaacatctacgtgctgcaccaggatctggccgagcctgccaaagaaatcagcgagaaagtgcaccagatctatggcttccccaaagagggcgccagcagcatcgtgtccaacctgttcatccactacctgatgaagaacacccagcaggtcgagaacctggctgtgctgtgccacaagctgctgcagcctggcggcatggtctggttcaccaccatgctgggcgaacaggtgctggaactgctgcacgagaaccggatcgaactgaacgaagtgtgggaggcccgggagaacgaggtggtcaagttcgccatcaagcggctgttcaaagaggacatcctgcaggaaaccggccaggaaatcggcgtcctgctgcccttcagcaacggcgacttctacaatgagtacctggtcaacaccgcctttctgatcaagattttcaagcaccatggctttagcctcgtgcagaagcagagcttcaaggactggatccccgagttccagaacttcagcaagagcctgtacaagatcctgaccgaggccgacaagacctggaccagcctgttcggcttcatctgcctgcggaagaacggaggcgggggaagtggagggggcggcagtcaggacctgcacgccatccagctgcagctcgaagaggaaatgttcaacggcggcatcagaagattcgaggccgaccagcagagacagatcgcctctggcaacgagagcgacaccgcctggaatagaaggctgctgtctgagctgatcgcccctatggccgaaggcatccaggcctacaaagaggaatacgagggcaagagaggcagagcccctagagccctggccttcatcaactgtgtgggcaatgaggtggccgcctacatcaccatgaagatcgtgatggacatgctgaacaccgacgtgaccctgcaggccattgccatgaacgtggccgacagaatcgaggaccaggtccgattcagcaagctggaaggacacgccgccaagtacttcgagaaagtgaagaagtccctgaaggccagcaagaccaagagctacagacacgcccacaacgtggccgtggtggccgaaaaatctgtggccgatagggacgccgacttctctagatgggaggcctggcctaaggacaccctgctgcagatcggcatgaccctgctggaaatcctggaaaacagcgtgttcttcaacggccagcccgtgttcctgagaaccctgaggacaaatggcggcaagcacggcgtgtactacctgcagacatctgagcacgtgggcgagtggatcaccgccttcaaagaacatgtggcccagctgagccctgcctatgccccttgtgtgatccctcctagaccctgggtgtcccctttcaatggcggctttcacaccgagaaggtggccagcagaatcagactggtcaagggcaaccgggaacacgtgcggaagctgaccaagaaacagatgcccgccgtgtacaaggccgtgaatgctctgcaggccaccaagtggcaggtcaacaaagaggtgctgcaggtcgtcgaggacgtgatcagactggatctgggctacggcgtgccaagctttaagcccctgatcgacagagagaacaagcccgccaaccctgtgcccctggaatttcagcacctgagaggccgcgagctgaaagagatgctgacacctgaacagtggcaggcctttatcaattggaagggcgagtgcaccaagctgtacaccgccgagacaaagaggggctctaagtctgccgccacagtgcgaatggtcggacaggccagaaagtacagccagttcgacgccatctacttcgtgtacgccctggacagccggtctagagtgtatgcccagagcagcacactgagcccccagtctaacgatctgggaaaggccctgctgagattcaccgagggccagagactggattctgccgaagccctgaagtggttcctggtcaacggcgccaacaactggggctgggacaagaaaaccttcgatgtgcggaccgccaacgtgctggatagcgagttccaggacatgtgcagagatatcgccgccgaccctctgacctttacccagtgggtcaacgccgatagcccctatggattcctggcctggtgcttcgagtacgccagatacctggacgccctggatgagggaacccaggatcagttcatgacccatctgcccgtgcaccaggatggctcttgttctggcatccagcactacagcgccatgctgagcgatgccgtgggagccaaagccgtgaacctgaagcctagcgacagcccccaggatatctatggcgctgtggcccaggtggtcatccagaaaaactacgcctacatgaacgccgaggacgccgagacattcacaagcggaagcgtgacactgacaggcgccgagctgagatctatggcctctgcctgggacatgatcggcatcacacggggcctgaccaaaaagcctgtgatgacactgccctacggcagcaccagactgacctgtagagaaagcgtgatcgactacatcgtggacctggaagagaaagaggcccagagagccattgccgagggcagaacagccaatcctgtgcaccccttcgacaacgaccggaaggatagcctgacacctagcgccgcctacaactacatgaccgccctgatctggcccagcatctctgaagtggtcaaggcccctatcgtggccatgaagatgatcagacagctggccagattcgccgccaagagaaatgagggcctggaataccctctgcccaccggctttatcctgcagcagaaaatcatggccaccgacatgctgcgggtgtccacatgtctgatgggcgagatcaagatgagcctgcagatcgagacagacgtggtggacgagacagccatgatgggagccgccgctcctaattttgtgcacggacacgatgccagccacctgatcctgaccgtgtgcgatctggtggacaagggcatcactagcgtggccgtgatccacgatagctttggaacacacgccggcagaaccgccgacctgagagattctctgcgggaagagatggtcaagatgtaccagaaccacaacgccctgcagaacctgctggacgtgcacgaagaaagatggctggtggacaccggcatccaggtgccagaacagggagagttcgacctgaacgagatcctggtgtccgactactgcttcgcctga

SEQ ID NO: 28 - protein, 2797 aa, Artificial Sequence
  recombinant protein encocded by pC3P3-G3e plasmid
MSKIYIDERSDAEIVCAAIKNIGIEGATAAQLTRQLNMEKREVNKALYDLQRSAMVYSSDDIPPRWFMTTEADKPDADAMADVIIDDVSREKSMREDHKSFDDVIPAKKIIDWKDADPNTVSSFQVDCFLWHVRKRVADQELGDAPFLDRLRRDQKSLRGRGSTLGLDIETATRAGKQIVERILKEESGGGGSGGGGSRMAQLEAKVEELLSKNWNLENEVARLKKLVGERGGGGSGGGGSDAQTRRRERRAEKQAQWKAANPFPVTTQGSQQTQPPQRHYGITSPISLAAPKETDCLLTQKLIETLKPFGVFEEEEELQRRILILGKLNNLVKEWIREISESKNLPQSVIENVGGKIFTFGSYRLGVHTKGADIDALCVAPRHVDRSDFFTSFYDKLKLQEEVKDLRAVEEAFVPVIKLCFDGIEIDILFARLALQTIPEDLDLRDDSLLKNLDIRCIRSLNGCRVTDEILHLVPNIDNFRLTLRAIKLWAKRHNIYSNILGFLGGVSWAMLVARTCQLYPNAIASTLVHKFFLVFSKWEWPNPVLLKQPEECNLNLPVWDPRVNPSDRYHLMPIITPAYPQQNSTYNVSVSTRMVMVEEFKQGLAITDEILLSKAEWSKLFEAPNFFQKYKHYIVLLASAPTEKQRLEWVGLVESKIRILVGSLEKNEFITLAHVNPQSFPAPKESPDREEFRTMWVIGLVFKKTENSENLSVDLTYDIQSFTDTVYRQAINSKMFELDMKIAAMHVKRKQLHQLLPSHVLQKRKKHSTEGVKLTALNDSSLDLSMDSDNSMSVPSPTSAMKTSPLNSSGSSQGRNSPAPAVTAASVTSIQASEVSVPQANSSESPGGPSSESIPQTATQPAIAPPPKPTVSRVVSSTRLVNPSPRPSGNTATKVPNPIVGVKRTSAPNKEEAPRRTKTEEDETSEDANCLALSGHDKTETKEQVDLETSAVQSETVPASASLLASQKTSSTDLSDIPALPANPIPVIKNSIKLRLNRGSGPRPLLAIHPTEARHKQKIVAPVKQTLNFDLLKLAGDVESNPGPASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSQDLHAIQLQLEEEMFNGGIRRFEADQQRQIASGNESDTAWNRRLLSELIAPMAEGIQAYKEEYEGKRGRAPRALAFINCVGNEVAAYITMKIVMDMLNTDVTLQAIAMNVADRIEDQVRFSKLEGHAAKYFEKVKKSLKASKTKSYRHAHNVAVVAEKSVADRDADFSRWEAWPKDTLLQIGMTLLEILENSVFFNGQPVFLRTLRTNGGKHGVYYLQTSEHVGEWITAFKEHVAQLSPAYAPCVIPPRPWVSPFNGGFHTEKVASRIRLVKGNREHVRKLTKKQMPAVYKAVNALQATKWQVNKEVLQVVEDVIRLDLGYGVPSFKPLIDRENKPANPVPLEFQHLRGRELKEMLTPEQWQAFINWKGECTKLYTAETKRGSKSAATVRMVGQARKYSQFDAIYFVYALDSRSRVYAQSSTLSPQSNDLGKALLRFTEGQRLDSAEALKWFLVNGANNWGWDKKTFDVRTANVLDSEFQDMCRDIAADPLTFTQWVNADSPYGFLAWCFEYARYLDALDEGTQDQFMTHLPVHQDGSCSGIQHYSAMLSDAVGAKAVNLKPSDSPQDIYGAVAQVVIQKNYAYMNAEDAETFTSGSVTLTGAELRSMASAWDMIGITRGLTKKPVMTLPYGSTRLTCRESVIDYIVDLEEKEAQRAIAEGRTANPVHPFDNDRKDSLTPSAAYNYMTALIWPSISEVVKAPIVAMKMIRQLARFAAKRNEGLEYPLPTGFILQQKIMATDMLRVSTCLMGEIKMSLQIETDVVDETAMMGAAAPNFVHGHDASHLILTVCDLVDKGITSVAVIHDSFGTHAGRTADLRDSLREEMVKMYQNHNALQNLLDVHEERWLVDTGIQVPEQGEFDLNEILVSDYCFA

SEQ ID NO: 29 - DNA, 66 bp, Enterobacteria phage lambda
atggatgctcagaccagacgcagagaacggcgggcagagaagcaggcacagtggaaggccgctaat

SEQ ID NO: 30 - protein, 22 aa, Enterobacteria phage lambda
MDAQTRRRERRAEKQAQWKAAN

SEQ ID NO: 31 - DNA, 15 bp, Artificial Sequence
  boxBL
gccctgaaaaagggc

SEQ ID NO: 32 - DNA, 19 bp, Artificial Sequence
  BoxBR
acatgaggatcacccatgt

SEQ ID NO: 33 - protein, 5 aa, Artificial Sequence
  Linker G4S
GGGGS

SEQ ID NO: 34 - protein, 10 aa, Artificial Sequence
  Linker (G4S)2
GGGGSGGGGS

SEQ ID NO: 35 - DNA, 8889 bp, Artificial Sequence
  recombinant DNA open-reading frame from pC3P3f plasmid
atggctggcgatctgagcgccggttttttcatggaagaactcaatacttatagacaaaaacagggagtggtccttaagtaccaggagcttcctaattcaggtcccccccacgatcgaagattcacgtttcaggtgatcatcgatggcagagaattccccgaaggcgagggacgctcaaagaaagaggctaagaatgctgcggccaagcttgccgtcgaaatcctgaataaggaaaaaaaggcagttagcccgttgctgttgaccaccacgaattcgtcagaaggactatctatgggcaactacatagggttgattaacaggatcgcccagaaaaaacggcttaccgttaattatgagcaatgcgctagtggtgtgcacggcccggaagggttccattacaaatgcaaaatgggtcagaaggagtacagcattgggaccggcagtacaaaacaggaagcaaagcagctggccgccaagctagcatatcttcagattctgtccggaggaggaggctctggtggtggcgggagcagccggcggaacaagcggagccggcggcggcggaagaagcccctgaacaccatccagcccggccccagcaagcccagcgcccaggacgagcccatcaagagcgtgagccaccacagcagcaagatcggcaccaaccccatgctggccttcatcctgggcggcaacgaggacctgagcgacgacagcgactgggacgaggacttcagcctggagaacaccctgatgcccctgaacgaggtgagcctgaagggcaagcacgacagcaagcacttcaacaagggcttcgacaacaacaccgccctgcacgaggtgaacaccaagtgggaggccttctacagcagcgtgaagatccggcagcgggacgtgaaggtgtacttcgccaccgacgacatcctgatcaaggtgcgggaggccgacgacatcgaccggaagggcccctgggagcaggccgccgtggaccggctgcggttccagcggcggatcgccgacaccgagaagatcctgagcgccgtgctgctgcggaagaagctgaaccccatggagcacgagggatcaggcccgcgacccctccttgccatccacccaaccgaggcacggcacaagcagaaaatagtggcacccgttaagcagactctcaactttgatctcctgaagcttgccggcgacgtagagtccaaccccggccccgatgctcagaccagacgcagagaacggcgggcagagaagcaggcacagtggaaggccgctaatccgtttccagttacaacgcagggatcacaacaaacgcagccaccacagaggcactatggcattacctctcctatcagcttagcggcccccaaggagactgactgcctactcacacagaagctcatcgagacgctgaagccctttggggtttttgaagaagaagaggaactgcagcgcaggattttaattttgggaaaattaaataacctggtgaaagaatggattcgagaaatcagtgaaagcaagaatctcccacaatctgtaattgaaaatgttggagggaagatttttacatttggatcttacagactaggagtccacacgaaaggtgctgatattgatgcgttgtgtgttgcaccaagacatgttgatcgaagtgactttttcacctcattctatgataaattgaaattacaagaagaagtgaaagatttaagagctgttgaagaggcatttgtaccagttatcaaactctgttttgatggaatagagattgatattttgtttgcaagattagcactgcagactattccagaagatttggacctacgagatgacagtctgcttaaaaacctagatataagatgcataagaagccttaatggttgcagggtaaccgatgaaattttacatctagtaccaaacattgacaacttcaggttaactctgagagccatcaaactgtgggccaaacggcacaacatctattccaatatattaggtttcctcggtggtgtttcctgggctatgctagtagcaagaacttgccagctttatccaaatgcaatagcatcaactcttgtacataaatttttcttggtattttctaaatgggaatggccaaatccagtgctattgaaacagcctgaagaatgcaatcttaatttgcctgtgtgggacccaagggtaaaccccagtgataggtaccatcttatgcctataattacaccagcatacccacagcagaactccacgtacaatgtgtccgtttcaacacggatggtcatggttgaggagtttaaacaaggtcttgctatcacagatgaaattttgctgagtaaggcagagtggtccaaactttttgaagctccaaacttctttcagaagtacaagcattatattgtacttctagcaagtgcgcccacggaaaagcagcgtctggaatgggtgggcttggtggaatcaaaaatccgcatcctggttggaagcttggagaagaatgagtttattacactggctcatgtgaatccccagtcatttccagcccccaaagaaagtcctgacagggaagaatttcgcacaatgtgggtgattgggttagtgtttaaaaaaactgaaaactctgaaaatctcagtgtcgacctcacctatgatatccagtctttcacagacacagtttataggcaagcaataaacagcaaaatgtttgagttggatatgaagattgcagcaatgcatgtgaagagaaagcaactccatcagctgctgcctagtcacgtgcttcagaagaggaagaagcattcaacagaaggagtcaagttaacagctctgaatgacagcagccttgacttgtctatggacagtgataacagcatgtctgtgccttcacccaccagtgctatgaagaccagtccattgaatagttctggcagctcccagggcagaaacagtcctgctccagctgtgaccgcagcatctgtgaccagcatccaggcttctgaggtttctgtaccgcaagcaaattccagtgaaagcccagggggtccatcgagcgaaagcattcctcaaactgccacacagccagccattgccccaccaccaaagcctacagtctccagagttgtctcctcaacacgactggtaaacccatcgcctagaccttcaggaaacacagcaacaaaagtccctaatcctatagtaggagtcaagagaacgtccgcccccaataaagaagaagcccctagaaggaccaaaacagaagaggatgaaacaagtgaagatgctaactgtcttgctttgagtggacatgataaaacagagacaaaggaacaagttgatctggagacaagtgcggttcaatcagaaactgttccggcatcggcttctctgttggcctctcagaaaacatccagtacagacctttctgatatccctgctctccctgcaaatcctattcctgttatcaagaactcaataaaactgagactgaatcggggatcaggcccgcgacccctccttgccatccacccaaccgaggcacggcacaagcagaaaatagtggcacccgttaagcagactctcaactttgatctcctgaagcttgccggcgacgtagagtccaaccccggccccgccagcctggacaacctggtggccagataccagcggtgcttcaacgaccagagcctgaagaacagcaccatcgagctggaaatccggttccagcagatcaacttcctgctgttcaagaccgtgtacgaggccctggtcgcccaggaaatccccagcaccatcagccacagcatccggtgcatcaagaaggtgcaccacgagaaccactgccgggagaagatcctgcccagcgagaacctgtacttcaagaaacagcccctgatgttcttcaagttcagcgagcccgccagcctgggctgtaaagtgtccctggccatcgagcagcccatccggaagttcatcctggacagcagcgtgctggtccggctgaagaaccggaccaccttccgggtgtccgagctgtggaagatcgagctgaccatcgtgaagcagctgatgggcagcgaggtgtcagccaagctggccgccttcaagaccctgctgttcgacacccccgagcagcagaccaccaagaacatgatgaccctgatcaaccccgacgacgagtacctgtacgagatcgagatcgagtacaccggcaagcctgagagcctgacagccgccgacgtgatcaagatcaagaacaccgtgctgacactgatcagccccaaccacctgatgctgaccgcctaccaccaggccatcgagtttatcgccagccacatcctgagcagcgagatcctgctggcccggatcaagagcggcaagtggggcctgaagagactgctgccccaggtcaagtccatgaccaaggccgactacatgaagttctacccccccgtgggctactacgtgaccgacaaggccgacggcatccggggcattgccgtgatccaggacacccagatctacgtggtggccgaccagctgtacagcctgggcaccaccggcatcgagcccctgaagcccaccatcctggacggcgagttcatgcccgagaagaaagagttctacggctttgacgtgatcatgtacgagggcaacctgctgacccagcagggcttcgagacacggatcgagagcctgagcaagggcatcaaggtgctgcaggccttcaacatcaaggccgagatgaagcccttcatcagcctgacctccgccgaccccaacgtgctgctgaagaatttcgagagcatcttcaagaagaaaacccggccctacagcatcgacggcatcatcctggtggagcccggcaacagctacctgaacaccaacaccttcaagtggaagcccacctgggacaacaccctggactttctggtccggaagtgccccgagtccctgaacgtgcccgagtacgcccccaagaagggcttcagcctgcatctgctgttcgtgggcatcagcggcgagctgtttaagaagctggccctgaactggtgccccggctacaccaagctgttccccgtgacccagcggaaccagaactacttccccgtgcagttccagcccagcgacttccccctggccttcctgtactaccaccccgacaccagcagcttcagcaacatcgatggcaaggtgctggaaatgcggtgcctgaagcgggagatcaactacgtgcgctgggagatcgtgaagatccgggaggaccggcagcaggatctgaaaaccggcggctacttcggcaacgacttcaagaccgccgagctgacctggctgaactacatggaccccttcagcttcgaggaactggccaagggacccagcggcatgtacttcgctggcgccaagaccggcatctacagagcccagaccgccctgatcagcttcatcaagcaggaaatcatccagaagatcagccaccagagctgggtgatcgacctgggcatcggcaagggccaggacctgggcagatacctggacgccggcgtgagacacctggtcggcatcgataaggaccagacagccctggccgagctggtgtaccggaagttctcccacgccaccaccagacagcacaagcacgccaccaacatctacgtgctgcaccaggatctggccgagcctgccaaagaaatcagcgagaaagtgcaccagatctatggcttccccaaagagggcgccagcagcatcgtgtccaacctgttcatccactacctgatgaagaacacccagcaggtcgagaacctggctgtgctgtgccacaagctgctgcagcctggcggcatggtctggttcaccaccatgctgggcgaacaggtgctggaactgctgcacgagaaccggatcgaactgaacgaagtgtgggaggcccgggagaacgaggtggtcaagttcgccatcaagcggctgttcaaagaggacatcctgcaggaaaccggccaggaaatcggcgtcctgctgcccttcagcaacggcgacttctacaatgagtacctggtcaacaccgcctttctgatcaagattttcaagcaccatggctttagcctcgtgcagaagcagagcttcaaggactggatccccgagttccagaacttcagcaagagcctgtacaagatcctgaccgaggccgacaagacctggaccagcctgttcggcttcatctgcctgcggaagaacggaggcgggggaagtggagggggcggcagtcaggacctgcacgccatccagctgcagctcgaagaggaaatgttcaacggcggcatcagaagattcgaggccgaccagcagagacagatcgcctctggcaacgagagcgacaccgcctggaatagaaggctgctgtctgagctgatcgcccctatggccgaaggcatccaggcctacaaagaggaatacgagggcaagagaggcagagcccctagagccctggccttcatcaactgtgtgggcaatgaggtggccgcctacatcaccatgaagatcgtgatggacatgctgaacaccgacgtgaccctgcaggccattgccatgaacgtggccgacagaatcgaggaccaggtccgattcagcaagctggaaggacacgccgccaagtacttcgagaaagtgaagaagtccctgaaggccagcaagaccaagagctacagacacgcccacaacgtggccgtggtggccgaaaaatctgtggccgatagggacgccgacttctctagatgggaggcctggcctaaggacaccctgctgcagatcggcatgaccctgctggaaatcctggaaaacagcgtgttcttcaacggccagcccgtgttcctgagaaccctgaggacaaatggcggcaagcacggcgtgtactacctgcagacatctgagcacgtgggcgagtggatcaccgccttcaaagaacatgtggcccagctgagccctgcctatgccccttgtgtgatccctcctagaccctgggtgtcccctttcaatggcggctttcacaccgagaaggtggccagcagaatcagactggtcaagggcaaccgggaacacgtgcggaagctgaccaagaaacagatgcccgccgtgtacaaggccgtgaatgctctgcaggccaccaagtggcaggtcaacaaagaggtgctgcaggtcgtcgaggacgtgatcagactggatctgggctacggcgtgccaagctttaagcccctgatcgacagagagaacaagcccgccaaccctgtgcccctggaatttcagcacctgagaggccgcgagctgaaagagatgctgacacctgaacagtggcaggcctttatcaattggaagggcgagtgcaccaagctgtacaccgccgagacaaagaggggctctaagtctgccgccacagtgcgaatggtcggacaggccagaaagtacagccagttcgacgccatctacttcgtgtacgccctggacagccggtctagagtgtatgcccagagcagcacactgagcccccagtctaacgatctgggaaaggccctgctgagattcaccgagggccagagactggattctgccgaagccctgaagtggttcctggtcaacggcgccaacaactggggctgggacaagaaaaccttcgatgtgcggaccgccaacgtgctggatagcgagttccaggacatgtgcagagatatcgccgccgaccctctgacctttacccagtgggtcaacgccgatagcccctatggattcctggcctggtgcttcgagtacgccagatacctggacgccctggatgagggaacccaggatcagttcatgacccatctgcccgtgcaccaggatggctcttgttctggcatccagcactacagcgccatgctgagcgatgccgtgggagccaaagccgtgaacctgaagcctagcgacagcccccaggatatctatggcgctgtggcccaggtggtcatccagaaaaactacgcctacatgaacgccgaggacgccgagacattcacaagcggaagcgtgacactgacaggcgccgagctgagatctatggcctctgcctgggacatgatcggcatcacacggggcctgaccaaaaagcctgtgatgacactgccctacggcagcaccagactgacctgtagagaaagcgtgatcgactacatcgtggacctggaagagaaagaggcccagagagccattgccgagggcagaacagccaatcctgtgcaccccttcgacaacgaccggaaggatagcctgacacctagcgccgcctacaactacatgaccgccctgatctggcccagcatctctgaagtggtcaaggcccctatcgtggccatgaagatgatcagacagctggccagattcgccgccaagagaaatgagggcctggaataccctctgcccaccggctttatcctgcagcagaaaatcatggccaccgacatgctgcgggtgtccacatgtctgatgggcgagatcaagatgagcctgcagatcgagacagacgtggtggacgagacagccatgatgggagccgccgctcctaattttgtgcacggacacgatgccagccacctgatcctgaccgtgtgcgatctggtggacaagggcatcactagcgtggccgtgatccacgatagctttggaacacacgccggcagaaccgccgacctgagagattctctgcgggaagagatggtcaagatgtaccagaaccacaacgccctgcagaacctgctggacgtgcacgaagaaagatggctggtggacaccggcatccaggtgccagaacagggagagttcgacctgaacgagatcctggtgtccgactactgcttcgcctga

SEQ ID NO: 36 - protein, 2962 aa, Artificial Sequence
  recombinant protein encoded by pC3P3-G3f plasmid
MAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSGGGGSGGGGSSRRNKRSRRRRKKPLNTIQPGPSKPSAQDEPIKSVSHHSSKIGTNPMLAFILGGNEDLSDDSDWDEDFSLENTLMPLNEVSLKGKHDSKHFNKGFDNNTALHEVNTKWEAFYSSVKIRQRDVKVYFATDDILIKVREADDIDRKGPWEQAAVDRLRFQRRIADTEKILSAVLLRKKLNPMEHEGSGPRPLLAIHPTEARHKQKIVAPVKQTLNFDLLKLAGDVESNPGPDAQTRRRERRAEKQAQWKAANPFPVTTQGSQQTQPPQRHYGITSPISLAAPKETDCLLTQKLIETLKPFGVFEEEEELQRRILILGKLNNLVKEWIREISESKNLPQSVIENVGGKIFTFGSYRLGVHTKGADIDALCVAPRHVDRSDFFTSFYDKLKLQEEVKDLRAVEEAFVPVIKLCFDGIEIDILFARLALQTIPEDLDLRDDSLLKNLDIRCIRSLNGCRVTDEILHLVPNIDNFRLTLRAIKLWAKRHNIYSNILGFLGGVSWAMLVARTCQLYPNAIASTLVHKFFLVFSKWEWPNPVLLKQPEECNLNLPVWDPRVNPSDRYHLMPIITPAYPQQNSTYNVSVSTRMVMVEEFKQGLAITDEILLSKAEWSKLFEAPNFFQKYKHYIVLLASAPTEKQRLEWVGLVESKIRILVGSLEKNEFITLAHVNPQSFPAPKESPDREEFRTMWVIGLVFKKTENSENLSVDLTYDIQSFTDTVYRQAINSKMFELDMKIAAMHVKRKQLHQLLPSHVLQKRKKHSTEGVKLTALNDSSLDLSMDSDNSMSVPSPTSAMKTSPLNSSGSSQGRNSPAPAVTAASVTSIQASEVSVPQANSSESPGGPSSESIPQTATQPAIAPPPKPTVSRVVSSTRLVNPSPRPSGNTATKVPNPIVGVKRTSAPNKEEAPRRTKTEEDETSEDANCLALSGHDKTETKEQVDLETSAVQSETVPASASLLASQKTSSTDLSDIPALPANPIPVIKNSIKLRLNRGSGPRPLLAIHPTEARHKQKIVAPVKQTLNFDLLKLAGDVESNPGPASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSQDLHAIQLQLEEEMFNGGIRRFEADQQRQIASGNESDTAWNRRLLSELIAPMAEGIQAYKEEYEGKRGRAPRALAFINCVGNEVAAYITMKIVMDMLNTDVTLQAIAMNVADRIEDQVRFSKLEGHAAKYFEKVKKSLKASKTKSYRHAHNVAVVAEKSVADRDADFSRWEAWPKDTLLQIGMTLLEILENSVFFNGQPVFLRTLRTNGGKHGVYYLQTSEHVGEWITAFKEHVAQLSPAYAPCVIPPRPWVSPFNGGFHTEKVASRIRLVKGNREHVRKLTKKQMPAVYKAVNALQATKWQVNKEVLQVVEDVIRLDLGYGVPSFKPLIDRENKPANPVPLEFQHLRGRELKEMLTPEQWQAFINWKGECTKLYTAETKRGSKSAATVRMVGQARKYSQFDAIYFVYALDSRSRVYAQSSTLSPQSNDLGKALLRFTEGQRLDSAEALKWFLVNGANNWGWDKKTFDVRTANVLDSEFQDMCRDIAADPLTFTQWVNADSPYGFLAWCFEYARYLDALDEGTQDQFMTHLPVHQDGSCSGIQHYSAMLSDAVGAKAVNLKPSDSPQDIYGAVAQVVIQKNYAYMNAEDAETFTSGSVTLTGAELRSMASAWDMIGITRGLTKKPVMTLPYGSTRLTCRESVIDYIVDLEEKEAQRAIAEGRTANPVHPFDNDRKDSLTPSAAYNYMTALIWPSISEVVKAPIVAMKMIRQLARFAAKRNEGLEYPLPTGFILQQKIMATDMLRVSTCLMGEIKMSLQIETDVVDETAMMGAAAPNFVHGHDASHLILTVCDLVDKGITSVAVIHDSFGTHAGRTADLRDSLREEMVKMYQNHNALQNLLDVHEERWLVDTGIQVPEQGEFDLNEILVSDYCFA

SEQ ID NO: 37 - DNA, 8997 bp, Artificial Sequence
  recombinant DNA open-reading frame from pC3P3- G3g plasmid
atggctggcgatctgagcgccggttttttcatggaagaactcaatacttatagacaaaaacagggagtggtccttaagtaccaggagcttcctaattcaggtcccccccacgatcgaagattcacgtttcaggtgatcatcgatggcagagaattccccgaaggcgagggacgctcaaagaaagaggctaagaatgctgcggccaagcttgccgtcgaaatcctgaataaggaaaaaaaggcagttagcccgttgctgttgaccaccacgaattcgtcagaaggactatctatgggcaactacatagggttgattaacaggatcgcccagaaaaaacggcttaccgttaattatgagcaatgcgctagtggtgtgcacggcccggaagggttccattacaaatgcaaaatgggtcagaaggagtacagcattgggaccggcagtacaaaacaggaagcaaagcagctggccgccaagctagcatatcttcagattctgtccggatcaggcccgcgacccctccttgccatccacccaaccgaggcacggcacaagcagaaaatagtggcacccgttaagcagactctcaactttgatctcctgaagcttgccggcgacgtagagtccaaccccggccccagccggcggaacaagcggagccggcggcggcggaagaagcccctgaacaccatccagcccggccccagcaagcccagcgcccaggacgagcccatcaagagcgtgagccaccacagcagcaagatcggcaccaaccccatgctggccttcatcctgggcggcaacgaggacctgagcgacgacagcgactgggacgaggacttcagcctggagaacaccctgatgcccctgaacgaggtgagcctgaagggcaagcacgacagcaagcacttcaacaagggcttcgacaacaacaccgccctgcacgaggtgaacaccaagtgggaggccttctacagcagcgtgaagatccggcagcgggacgtgaaggtgtacttcgccaccgacgacatcctgatcaaggtgcgggaggccgacgacatcgaccggaagggcccctgggagcaggccgccgtggaccggctgcggttccagcggcggatcgccgacaccgagaagatcctgagcgccgtgctgctgcggaagaagctgaaccccatggagcacgagggatcaggcccgcgacccctccttgccatccacccaaccgaggcacggcacaagcagaaaatagtggcacccgttaagcagactctcaactttgatctcctgaagcttgccggcgacgtagagtccaaccccggccccgatgctcagaccagacgcagagaacggcgggcagagaagcaggcacagtggaaggccgctaatccgtttccagttacaacgcagggatcacaacaaacgcagccaccacagaggcactatggcattacctctcctatcagcttagcggcccccaaggagactgactgcctactcacacagaagctcatcgagacgctgaagccctttggggtttttgaagaagaagaggaactgcagcgcaggattttaattttgggaaaattaaataacctggtgaaagaatggattcgagaaatcagtgaaagcaagaatctcccacaatctgtaattgaaaatgttggagggaagatttttacatttggatcttacagactaggagtccacacgaaaggtgctgatattgatgcgttgtgtgttgcaccaagacatgttgatcgaagtgactttttcacctcattctatgataaattgaaattacaagaagaagtgaaagatttaagagctgttgaagaggcatttgtaccagttatcaaactctgttttgatggaatagagattgatattttgtttgcaagattagcactgcagactattccagaagatttggacctacgagatgacagtctgcttaaaaacctagatataagatgcataagaagccttaatggttgcagggtaaccgatgaaattttacatctagtaccaaacattgacaacttcaggttaactctgagagccatcaaactgtgggccaaacggcacaacatctattccaatatattaggtttcctcggtggtgtttcctgggctatgctagtagcaagaacttgccagctttatccaaatgcaatagcatcaactcttgtacataaatttttcttggtattttctaaatgggaatggccaaatccagtgctattgaaacagcctgaagaatgcaatcttaatttgcctgtgtgggacccaagggtaaaccccagtgataggtaccatcttatgcctataattacaccagcatacccacagcagaactccacgtacaatgtgtccgtttcaacacggatggtcatggttgaggagtttaaacaaggtcttgctatcacagatgaaattttgctgagtaaggcagagtggtccaaactttttgaagctccaaacttctttcagaagtacaagcattatattgtacttctagcaagtgcgcccacggaaaagcagcgtctggaatgggtgggcttggtggaatcaaaaatccgcatcctggttggaagcttggagaagaatgagtttattacactggctcatgtgaatccccagtcatttccagcccccaaagaaagtcctgacagggaagaatttcgcacaatgtgggtgattgggttagtgtttaaaaaaactgaaaactctgaaaatctcagtgtcgacctcacctatgatatccagtctttcacagacacagtttataggcaagcaataaacagcaaaatgtttgagttggatatgaagattgcagcaatgcatgtgaagagaaagcaactccatcagctgctgcctagtcacgtgcttcagaagaggaagaagcattcaacagaaggagtcaagttaacagctctgaatgacagcagccttgacttgtctatggacagtgataacagcatgtctgtgccttcacccaccagtgctatgaagaccagtccattgaatagttctggcagctcccagggcagaaacagtcctgctccagctgtgaccgcagcatctgtgaccagcatccaggcttctgaggtttctgtaccgcaagcaaattccagtgaaagcccagggggtccatcgagcgaaagcattcctcaaactgccacacagccagccattgccccaccaccaaagcctacagtctccagagttgtctcctcaacacgactggtaaacccatcgcctagaccttcaggaaacacagcaacaaaagtccctaatcctatagtaggagtcaagagaacgtccgcccccaataaagaagaagcccctagaaggaccaaaacagaagaggatgaaacaagtgaagatgctaactgtcttgctttgagtggacatgataaaacagagacaaaggaacaagttgatctggagacaagtgcggttcaatcagaaactgttccggcatcggcttctctgttggcctctcagaaaacatccagtacagacctttctgatatccctgctctccctgcaaatcctattcctgttatcaagaactcaataaaactgagactgaatcggggatcaggcccgcgacccctccttgccatccacccaaccgaggcacggcacaagcagaaaatagtggcacccgttaagcagactctcaactttgatctcctgaagcttgccggcgacgtagagtccaaccccggccccgccagcctggacaacctggtggccagataccagcggtgcttcaacgaccagagcctgaagaacagcaccatcgagctggaaatccggttccagcagatcaacttcctgctgttcaagaccgtgtacgaggccctggtcgcccaggaaatccccagcaccatcagccacagcatccggtgcatcaagaaggtgcaccacgagaaccactgccgggagaagatcctgcccagcgagaacctgtacttcaagaaacagcccctgatgttcttcaagttcagcgagcccgccagcctgggctgtaaagtgtccctggccatcgagcagcccatccggaagttcatcctggacagcagcgtgctggtccggctgaagaaccggaccaccttccgggtgtccgagctgtggaagatcgagctgaccatcgtgaagcagctgatgggcagcgaggtgtcagccaagctggccgccttcaagaccctgctgttcgacacccccgagcagcagaccaccaagaacatgatgaccctgatcaaccccgacgacgagtacctgtacgagatcgagatcgagtacaccggcaagcctgagagcctgacagccgccgacgtgatcaagatcaagaacaccgtgctgacactgatcagccccaaccacctgatgctgaccgcctaccaccaggccatcgagtttatcgccagccacatcctgagcagcgagatcctgctggcccggatcaagagcggcaagtggggcctgaagagactgctgccccaggtcaagtccatgaccaaggccgactacatgaagttctacccccccgtgggctactacgtgaccgacaaggccgacggcatccggggcattgccgtgatccaggacacccagatctacgtggtggccgaccagctgtacagcctgggcaccaccggcatcgagcccctgaagcccaccatcctggacggcgagttcatgcccgagaagaaagagttctacggctttgacgtgatcatgtacgagggcaacctgctgacccagcagggcttcgagacacggatcgagagcctgagcaagggcatcaaggtgctgcaggccttcaacatcaaggccgagatgaagcccttcatcagcctgacctccgccgaccccaacgtgctgctgaagaatttcgagagcatcttcaagaagaaaacccggccctacagcatcgacggcatcatcctggtggagcccggcaacagctacctgaacaccaacaccttcaagtggaagcccacctgggacaacaccctggactttctggtccggaagtgccccgagtccctgaacgtgcccgagtacgcccccaagaagggcttcagcctgcatctgctgttcgtgggcatcagcggcgagctgtttaagaagctggccctgaactggtgccccggctacaccaagctgttccccgtgacccagcggaaccagaactacttccccgtgcagttccagcccagcgacttccccctggccttcctgtactaccaccccgacaccagcagcttcagcaacatcgatggcaaggtgctggaaatgcggtgcctgaagcgggagatcaactacgtgcgctgggagatcgtgaagatccgggaggaccggcagcaggatctgaaaaccggcggctacttcggcaacgacttcaagaccgccgagctgacctggctgaactacatggaccccttcagcttcgaggaactggccaagggacccagcggcatgtacttcgctggcgccaagaccggcatctacagagcccagaccgccctgatcagcttcatcaagcaggaaatcatccagaagatcagccaccagagctgggtgatcgacctgggcatcggcaagggccaggacctgggcagatacctggacgccggcgtgagacacctggtcggcatcgataaggaccagacagccctggccgagctggtgtaccggaagttctcccacgccaccaccagacagcacaagcacgccaccaacatctacgtgctgcaccaggatctggccgagcctgccaaagaaatcagcgagaaagtgcaccagatctatggcttccccaaagagggcgccagcagcatcgtgtccaacctgttcatccactacctgatgaagaacacccagcaggtcgagaacctggctgtgctgtgccacaagctgctgcagcctggcggcatggtctggttcaccaccatgctgggcgaacaggtgctggaactgctgcacgagaaccggatcgaactgaacgaagtgtgggaggcccgggagaacgaggtggtcaagttcgccatcaagcggctgttcaaagaggacatcctgcaggaaaccggccaggaaatcggcgtcctgctgcccttcagcaacggcgacttctacaatgagtacctggtcaacaccgcctttctgatcaagattttcaagcaccatggctttagcctcgtgcagaagcagagcttcaaggactggatccccgagttccagaacttcagcaagagcctgtacaagatcctgaccgaggccgacaagacctggaccagcctgttcggcttcatctgcctgcggaagaacggaggcgggggaagtggagggggcggcagtcaggacctgcacgccatccagctgcagctcgaagaggaaatgttcaacggcggcatcagaagattcgaggccgaccagcagagacagatcgcctctggcaacgagagcgacaccgcctggaatagaaggctgctgtctgagctgatcgcccctatggccgaaggcatccaggcctacaaagaggaatacgagggcaagagaggcagagcccctagagccctggccttcatcaactgtgtgggcaatgaggtggccgcctacatcaccatgaagatcgtgatggacatgctgaacaccgacgtgaccctgcaggccattgccatgaacgtggccgacagaatcgaggaccaggtccgattcagcaagctggaaggacacgccgccaagtacttcgagaaagtgaagaagtccctgaaggccagcaagaccaagagctacagacacgcccacaacgtggccgtggtggccgaaaaatctgtggccgatagggacgccgacttctctagatgggaggcctggcctaaggacaccctgctgcagatcggcatgaccctgctggaaatcctggaaaacagcgtgttcttcaacggccagcccgtgttcctgagaaccctgaggacaaatggcggcaagcacggcgtgtactacctgcagacatctgagcacgtgggcgagtggatcaccgccttcaaagaacatgtggcccagctgagccctgcctatgccccttgtgtgatccctcctagaccctgggtgtcccctttcaatggcggctttcacaccgagaaggtggccagcagaatcagactggtcaagggcaaccgggaacacgtgcggaagctgaccaagaaacagatgcccgccgtgtacaaggccgtgaatgctctgcaggccaccaagtggcaggtcaacaaagaggtgctgcaggtcgtcgaggacgtgatcagactggatctgggctacggcgtgccaagctttaagcccctgatcgacagagagaacaagcccgccaaccctgtgcccctggaatttcagcacctgagaggccgcgagctgaaagagatgctgacacctgaacagtggcaggcctttatcaattggaagggcgagtgcaccaagctgtacaccgccgagacaaagaggggctctaagtctgccgccacagtgcgaatggtcggacaggccagaaagtacagccagttcgacgccatctacttcgtgtacgccctggacagccggtctagagtgtatgcccagagcagcacactgagcccccagtctaacgatctgggaaaggccctgctgagattcaccgagggccagagactggattctgccgaagccctgaagtggttcctggtcaacggcgccaacaactggggctgggacaagaaaaccttcgatgtgcggaccgccaacgtgctggatagcgagttccaggacatgtgcagagatatcgccgccgaccctctgacctttacccagtgggtcaacgccgatagcccctatggattcctggcctggtgcttcgagtacgccagatacctggacgccctggatgagggaacccaggatcagttcatgacccatctgcccgtgcaccaggatggctcttgttctggcatccagcactacagcgccatgctgagcgatgccgtgggagccaaagccgtgaacctgaagcctagcgacagcccccaggatatctatggcgctgtggcccaggtggtcatccagaaaaactacgcctacatgaacgccgaggacgccgagacattcacaagcggaagcgtgacactgacaggcgccgagctgagatctatggcctctgcctgggacatgatcggcatcacacggggcctgaccaaaaagcctgtgatgacactgccctacggcagcaccagactgacctgtagagaaagcgtgatcgactacatcgtggacctggaagagaaagaggcccagagagccattgccgagggcagaacagccaatcctgtgcaccccttcgacaacgaccggaaggatagcctgacacctagcgccgcctacaactacatgaccgccctgatctggcccagcatctctgaagtggtcaaggcccctatcgtggccatgaagatgatcagacagctggccagattcgccgccaagagaaatgagggcctggaataccctctgcccaccggctttatcctgcagcagaaaatcatggccaccgacatgctgcgggtgtccacatgtctgatgggcgagatcaagatgagcctgcagatcgagacagacgtggtggacgagacagccatgatgggagccgccgctcctaattttgtgcacggacacgatgccagccacctgatcctgaccgtgtgcgatctggtggacaagggcatcactagcgtggccgtgatccacgatagctttggaacacacgccggcagaaccgccgacctgagagattctctgcgggaagagatggtcaagatgtaccagaaccacaacgccctgcagaacctgctggacgtgcacgaagaaagatggctggtggacaccggcatccaggtgccagaacagggagagttcgacctgaacgagatcctggtgtccgactactgcttcgcctga

SEQ ID NO: 38 - protein, 2998 aa, Artificial Sequence
  recombinant protein encoded by pC3P3-G3g plasmid
MAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSGSGPRPLLAIHPTEARHKQKIVAPVKQTLNFDLLKLAGDVESNPGPSRRNKRSRRRRKKPLNTIQPGPSKPSAQDEPIKSVSHHSSKIGTNPMLAFILGGNEDLSDDSDWDEDFSLENTLMPLNEVSLKGKHDSKHFNKGFDNNTALHEVNTKWEAFYSSVKIRQRDVKVYFATDDILIKVREADDIDRKGPWEQAAVDRLRFQRRIADTEKILSAVLLRKKLNPMEHEGSGPRPLLAIHPTEARHKQKIVAPVKQTLNFDLLKLAGDVESNPGPDAQTRRRERRAEKQAQWKAANPFPVTTQGSQQTQPPQRHYGITSPISLAAPKETDCLLTQKLIETLKPFGVFEEEEELQRRILILGKLNNLVKEWIREISESKNLPQSVIENVGGKIFTFGSYRLGVHTKGADIDALCVAPRHVDRSDFFTSFYDKLKLQEEVKDLRAVEEAFVPVIKLCFDGIEIDILFARLALQTIPEDLDLRDDSLLKNLDIRCIRSLNGCRVTDEILHLVPNIDNFRLTLRAIKLWAKRHNIYSNILGFLGGVSWAMLVARTCQLYPNAIASTLVHKFFLVFSKWEWPNPVLLKQPEECNLNLPVWDPRVNPSDRYHLMPIITPAYPQQNSTYNVSVSTRMVMVEEFKQGLAITDEILLSKAEWSKLFEAPNFFQKYKHYIVLLASAPTEKQRLEWVGLVESKIRILVGSLEKNEFITLAHVNPQSFPAPKESPDREEFRTMWVIGLVFKKTENSENLSVDLTYDIQSFTDTVYRQAINSKMFELDMKIAAMHVKRKQLHQLLPSHVLQKRKKHSTEGVKLTALNDSSLDLSMDSDNSMSVPSPTSAMKTSPLNSSGSSQGRNSPAPAVTAASVTSIQASEVSVPQANSSESPGGPSSESIPQTATQPAIAPPPKPTVSRVVSSTRLVNPSPRPSGNTATKVPNPIVGVKRTSAPNKEEAPRRTKTEEDETSEDANCLALSGHDKTETKEQVDLETSAVQSETVPASASLLASQKTSSTDLSDIPALPANPIPVIKNSIKLRLNRGSGPRPLLAIHPTEARHKQKIVAPVKQTLNFDLLKLAGDVESNPGPASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSQDLHAIQLQLEEEMFNGGIRRFEADQQRQIASGNESDTAWNRRLLSELIAPMAEGIQAYKEEYEGKRGRAPRALAFINCVGNEVAAYITMKIVMDMLNTDVTLQAIAMNVADRIEDQVRFSKLEGHAAKYFEKVKKSLKASKTKSYRHAHNVAVVAEKSVADRDADFSRWEAWPKDTLLQIGMTLLEILENSVFFNGQPVFLRTLRTNGGKHGVYYLQTSEHVGEWITAFKEHVAQLSPAYAPCVIPPRPWVSPFNGGFHTEKVASRIRLVKGNREHVRKLTKKQMPAVYKAVNALQATKWQVNKEVLQVVEDVIRLDLGYGVPSFKPLIDRENKPANPVPLEFQHLRGRELKEMLTPEQWQAFINWKGECTKLYTAETKRGSKSAATVRMVGQARKYSQFDAIYFVYALDSRSRVYAQSSTLSPQSNDLGKALLRFTEGQRLDSAEALKWFLVNGANNWGWDKKTFDVRTANVLDSEFQDMCRDIAADPLTFTQWVNADSPYGFLAWCFEYARYLDALDEGTQDQFMTHLPVHQDGSCSGIQHYSAMLSDAVGAKAVNLKPSDSPQDIYGAVAQVVIQKNYAYMNAEDAETFTSGSVTLTGAELRSMASAWDMIGITRGLTKKPVMTLPYGSTRLTCRESVIDYIVDLEEKEAQRAIAEGRTANPVHPFDNDRKDSLTPSAAYNYMTALIWPSISEVVKAPIVAMKMIRQLARFAAKRNEGLEYPLPTGFILQQKIMATDMLRVSTCLMGEIKMSLQIETDVVDETAMMGAAAPNFVHGHDASHLILTVCDLVDKGITSVAVIHDSFGTHAGRTADLRDSLREEMVKMYQNHNALQNLLDVHEERWLVDTGIQVPEQGEFDLNEILVSDYCFA

SEQ ID NO: 39 - DNA, 8889 bp, Artificial Sequence
  recombinant DNA open-reading frame from pC3P3- G3h plasmid
atggatgctcagaccagacgcagagaacggcgggcagagaagcaggcacagtggaaggccgctaatccgtttccagttacaacgcagggatcacaacaaacgcagccaccacagaggcactatggcattacctctcctatcagcttagcggcccccaaggagactgactgcctactcacacagaagctcatcgagacgctgaagccctttggggtttttgaagaagaagaggaactgcagcgcaggattttaattttgggaaaattaaataacctggtgaaagaatggattcgagaaatcagtgaaagcaagaatctcccacaatctgtaattgaaaatgttggagggaagatttttacatttggatcttacagactaggagtccacacgaaaggtgctgatattgatgcgttgtgtgttgcaccaagacatgttgatcgaagtgactttttcacctcattctatgataaattgaaattacaagaagaagtgaaagatttaagagctgttgaagaggcatttgtaccagttatcaaactctgttttgatggaatagagattgatattttgtttgcaagattagcactgcagactattccagaagatttggacctacgagatgacagtctgcttaaaaacctagatataagatgcataagaagccttaatggttgcagggtaaccgatgaaattttacatctagtaccaaacattgacaacttcaggttaactctgagagccatcaaactgtgggccaaacggcacaacatctattccaatatattaggtttcctcggtggtgtttcctgggctatgctagtagcaagaacttgccagctttatccaaatgcaatagcatcaactcttgtacataaatttttcttggtattttctaaatgggaatggccaaatccagtgctattgaaacagcctgaagaatgcaatcttaatttgcctgtgtgggacccaagggtaaaccccagtgataggtaccatcttatgcctataattacaccagcatacccacagcagaactccacgtacaatgtgtccgtttcaacacggatggtcatggttgaggagtttaaacaaggtcttgctatcacagatgaaattttgctgagtaaggcagagtggtccaaactttttgaagctccaaacttctttcagaagtacaagcattatattgtacttctagcaagtgcgcccacggaaaagcagcgtctggaatgggtgggcttggtggaatcaaaaatccgcatcctggttggaagcttggagaagaatgagtttattacactggctcatgtgaatccccagtcatttccagcccccaaagaaagtcctgacagggaagaatttcgcacaatgtgggtgattgggttagtgtttaaaaaaactgaaaactctgaaaatctcagtgtcgacctcacctatgatatccagtctttcacagacacagtttataggcaagcaataaacagcaaaatgtttgagttggatatgaagattgcagcaatgcatgtgaagagaaagcaactccatcagctgctgcctagtcacgtgcttcagaagaggaagaagcattcaacagaaggagtcaagttaacagctctgaatgacagcagccttgacttgtctatggacagtgataacagcatgtctgtgccttcacccaccagtgctatgaagaccagtccattgaatagttctggcagctcccagggcagaaacagtcctgctccagctgtgaccgcagcatctgtgaccagcatccaggcttctgaggtttctgtaccgcaagcaaattccagtgaaagcccagggggtccatcgagcgaaagcattcctcaaactgccacacagccagccattgccccaccaccaaagcctacagtctccagagttgtctcctcaacacgactggtaaacccatcgcctagaccttcaggaaacacagcaacaaaagtccctaatcctatagtaggagtcaagagaacgtccgcccccaataaagaagaagcccctagaaggaccaaaacagaagaggatgaaacaagtgaagatgctaactgtcttgctttgagtggacatgataaaacagagacaaaggaacaagttgatctggagacaagtgcggttcaatcagaaactgttccggcatcggcttctctgttggcctctcagaaaacatccagtacagacctttctgatatccctgctctccctgcaaatcctattcctgttatcaagaactcaataaaactgagactgaatcggggatcaggcccgcgacccctccttgccatccacccaaccgaggcacggcacaagcagaaaatagtggcacccgttaagcagactctcaactttgatctcctgaagcttgccggcgacgtagagtccaaccccggccccgctggcgatctgagcgccggttttttcatggaagaactcaatacttatagacaaaaacagggagtggtccttaagtaccaggagcttcctaattcaggtcccccccacgatcgaagattcacgtttcaggtgatcatcgatggcagagaattccccgaaggcgagggacgctcaaagaaagaggctaagaatgctgcggccaagcttgccgtcgaaatcctgaataaggaaaaaaaggcagttagcccgttgctgttgaccaccacgaattcgtcagaaggactatctatgggcaactacatagggttgattaacaggatcgcccagaaaaaacggcttaccgttaattatgagcaatgcgctagtggtgtgcacggcccggaagggttccattacaaatgcaaaatgggtcagaaggagtacagcattgggaccggcagtacaaaacaggaagcaaagcagctggccgccaagctagcatatcttcagattctgtccggaggaggaggctctggtggtggcgggagcagccggcggaacaagcggagccggcggcggcggaagaagcccctgaacaccatccagcccggccccagcaagcccagcgcccaggacgagcccatcaagagcgtgagccaccacagcagcaagatcggcaccaaccccatgctggccttcatcctgggcggcaacgaggacctgagcgacgacagcgactgggacgaggacttcagcctggagaacaccctgatgcccctgaacgaggtgagcctgaagggcaagcacgacagcaagcacttcaacaagggcttcgacaacaacaccgccctgcacgaggtgaacaccaagtgggaggccttctacagcagcgtgaagatccggcagcgggacgtgaaggtgtacttcgccaccgacgacatcctgatcaaggtgcgggaggccgacgacatcgaccggaagggcccctgggagcaggccgccgtggaccggctgcggttccagcggcggatcgccgacaccgagaagatcctgagcgccgtgctgctgcggaagaagctgaaccccatggagcacgagggatcaggcccgcgacccctccttgccatccacccaaccgaggcacggcacaagcagaaaatagtggcacccgttaagcagactctcaactttgatctcctgaagcttgccggcgacgtagagtccaaccccggccccgccagcctggacaacctggtggccagataccagcggtgcttcaacgaccagagcctgaagaacagcaccatcgagctggaaatccggttccagcagatcaacttcctgctgttcaagaccgtgtacgaggccctggtcgcccaggaaatccccagcaccatcagccacagcatccggtgcatcaagaaggtgcaccacgagaaccactgccgggagaagatcctgcccagcgagaacctgtacttcaagaaacagcccctgatgttcttcaagttcagcgagcccgccagcctgggctgtaaagtgtccctggccatcgagcagcccatccggaagttcatcctggacagcagcgtgctggtccggctgaagaaccggaccaccttccgggtgtccgagctgtggaagatcgagctgaccatcgtgaagcagctgatgggcagcgaggtgtcagccaagctggccgccttcaagaccctgctgttcgacacccccgagcagcagaccaccaagaacatgatgaccctgatcaaccccgacgacgagtacctgtacgagatcgagatcgagtacaccggcaagcctgagagcctgacagccgccgacgtgatcaagatcaagaacaccgtgctgacactgatcagccccaaccacctgatgctgaccgcctaccaccaggccatcgagtttatcgccagccacatcctgagcagcgagatcctgctggcccggatcaagagcggcaagtggggcctgaagagactgctgccccaggtcaagtccatgaccaaggccgactacatgaagttctacccccccgtgggctactacgtgaccgacaaggccgacggcatccggggcattgccgtgatccaggacacccagatctacgtggtggccgaccagctgtacagcctgggcaccaccggcatcgagcccctgaagcccaccatcctggacggcgagttcatgcccgagaagaaagagttctacggctttgacgtgatcatgtacgagggcaacctgctgacccagcagggcttcgagacacggatcgagagcctgagcaagggcatcaaggtgctgcaggccttcaacatcaaggccgagatgaagcccttcatcagcctgacctccgccgaccccaacgtgctgctgaagaatttcgagagcatcttcaagaagaaaacccggccctacagcatcgacggcatcatcctggtggagcccggcaacagctacctgaacaccaacaccttcaagtggaagcccacctgggacaacaccctggactttctggtccggaagtgccccgagtccctgaacgtgcccgagtacgcccccaagaagggcttcagcctgcatctgctgttcgtgggcatcagcggcgagctgtttaagaagctggccctgaactggtgccccggctacaccaagctgttccccgtgacccagcggaaccagaactacttccccgtgcagttccagcccagcgacttccccctggccttcctgtactaccaccccgacaccagcagcttcagcaacatcgatggcaaggtgctggaaatgcggtgcctgaagcgggagatcaactacgtgcgctgggagatcgtgaagatccgggaggaccggcagcaggatctgaaaaccggcggctacttcggcaacgacttcaagaccgccgagctgacctggctgaactacatggaccccttcagcttcgaggaactggccaagggacccagcggcatgtacttcgctggcgccaagaccggcatctacagagcccagaccgccctgatcagcttcatcaagcaggaaatcatccagaagatcagccaccagagctgggtgatcgacctgggcatcggcaagggccaggacctgggcagatacctggacgccggcgtgagacacctggtcggcatcgataaggaccagacagccctggccgagctggtgtaccggaagttctcccacgccaccaccagacagcacaagcacgccaccaacatctacgtgctgcaccaggatctggccgagcctgccaaagaaatcagcgagaaagtgcaccagatctatggcttccccaaagagggcgccagcagcatcgtgtccaacctgttcatccactacctgatgaagaacacccagcaggtcgagaacctggctgtgctgtgccacaagctgctgcagcctggcggcatggtctggttcaccaccatgctgggcgaacaggtgctggaactgctgcacgagaaccggatcgaactgaacgaagtgtgggaggcccgggagaacgaggtggtcaagttcgccatcaagcggctgttcaaagaggacatcctgcaggaaaccggccaggaaatcggcgtcctgctgcccttcagcaacggcgacttctacaatgagtacctggtcaacaccgcctttctgatcaagattttcaagcaccatggctttagcctcgtgcagaagcagagcttcaaggactggatccccgagttccagaacttcagcaagagcctgtacaagatcctgaccgaggccgacaagacctggaccagcctgttcggcttcatctgcctgcggaagaacggaggcgggggaagtggagggggcggcagtcaggacctgcacgccatccagctgcagctcgaagaggaaatgttcaacggcggcatcagaagattcgaggccgaccagcagagacagatcgcctctggcaacgagagcgacaccgcctggaatagaaggctgctgtctgagctgatcgcccctatggccgaaggcatccaggcctacaaagaggaatacgagggcaagagaggcagagcccctagagccctggccttcatcaactgtgtgggcaatgaggtggccgcctacatcaccatgaagatcgtgatggacatgctgaacaccgacgtgaccctgcaggccattgccatgaacgtggccgacagaatcgaggaccaggtccgattcagcaagctggaaggacacgccgccaagtacttcgagaaagtgaagaagtccctgaaggccagcaagaccaagagctacagacacgcccacaacgtggccgtggtggccgaaaaatctgtggccgatagggacgccgacttctctagatgggaggcctggcctaaggacaccctgctgcagatcggcatgaccctgctggaaatcctggaaaacagcgtgttcttcaacggccagcccgtgttcctgagaaccctgaggacaaatggcggcaagcacggcgtgtactacctgcagacatctgagcacgtgggcgagtggatcaccgccttcaaagaacatgtggcccagctgagccctgcctatgccccttgtgtgatccctcctagaccctgggtgtcccctttcaatggcggctttcacaccgagaaggtggccagcagaatcagactggtcaagggcaaccgggaacacgtgcggaagctgaccaagaaacagatgcccgccgtgtacaaggccgtgaatgctctgcaggccaccaagtggcaggtcaacaaagaggtgctgcaggtcgtcgaggacgtgatcagactggatctgggctacggcgtgccaagctttaagcccctgatcgacagagagaacaagcccgccaaccctgtgcccctggaatttcagcacctgagaggccgcgagctgaaagagatgctgacacctgaacagtggcaggcctttatcaattggaagggcgagtgcaccaagctgtacaccgccgagacaaagaggggctctaagtctgccgccacagtgcgaatggtcggacaggccagaaagtacagccagttcgacgccatctacttcgtgtacgccctggacagccggtctagagtgtatgcccagagcagcacactgagcccccagtctaacgatctgggaaaggccctgctgagattcaccgagggccagagactggattctgccgaagccctgaagtggttcctggtcaacggcgccaacaactggggctgggacaagaaaaccttcgatgtgcggaccgccaacgtgctggatagcgagttccaggacatgtgcagagatatcgccgccgaccctctgacctttacccagtgggtcaacgccgatagcccctatggattcctggcctggtgcttcgagtacgccagatacctggacgccctggatgagggaacccaggatcagttcatgacccatctgcccgtgcaccaggatggctcttgttctggcatccagcactacagcgccatgctgagcgatgccgtgggagccaaagccgtgaacctgaagcctagcgacagcccccaggatatctatggcgctgtggcccaggtggtcatccagaaaaactacgcctacatgaacgccgaggacgccgagacattcacaagcggaagcgtgacactgacaggcgccgagctgagatctatggcctctgcctgggacatgatcggcatcacacggggcctgaccaaaaagcctgtgatgacactgccctacggcagcaccagactgacctgtagagaaagcgtgatcgactacatcgtggacctggaagagaaagaggcccagagagccattgccgagggcagaacagccaatcctgtgcaccccttcgacaacgaccggaaggatagcctgacacctagcgccgcctacaactacatgaccgccctgatctggcccagcatctctgaagtggtcaaggcccctatcgtggccatgaagatgatcagacagctggccagattcgccgccaagagaaatgagggcctggaataccctctgcccaccggctttatcctgcagcagaaaatcatggccaccgacatgctgcgggtgtccacatgtctgatgggcgagatcaagatgagcctgcagatcgagacagacgtggtggacgagacagccatgatgggagccgccgctcctaattttgtgcacggacacgatgccagccacctgatcctgaccgtgtgcgatctggtggacaagggcatcactagcgtggccgtgatccacgatagctttggaacacacgccggcagaaccgccgacctgagagattctctgcgggaagagatggtcaagatgtaccagaaccacaacgccctgcagaacctgctggacgtgcacgaagaaagatggctggtggacaccggcatccaggtgccagaacagggagagttcgacctgaacgagatcctggtgtccgactactgcttcgcctga

SEQ ID NO: 40 - protein, 2962 aa, Artificial Sequence
  recombinant protein encoded by pC3P3-G3h plasmid
MDAQTRRRERRAEKQAQWKAANPFPVTTQGSQQTQPPQRHYGITSPISLAAPKETDCLLTQKLIETLKPFGVFEEEEELQRRILILGKLNNLVKEWIREISESKNLPQSVIENVGGKIFTFGSYRLGVHTKGADIDALCVAPRHVDRSDFFTSFYDKLKLQEEVKDLRAVEEAFVPVIKLCFDGIEIDILFARLALQTIPEDLDLRDDSLLKNLDIRCIRSLNGCRVTDEILHLVPNIDNFRLTLRAIKLWAKRHNIYSNILGFLGGVSWAMLVARTCQLYPNAIASTLVHKFFLVFSKWEWPNPVLLKQPEECNLNLPVWDPRVNPSDRYHLMPIITPAYPQQNSTYNVSVSTRMVMVEEFKQGLAITDEILLSKAEWSKLFEAPNFFQKYKHYIVLLASAPTEKQRLEWVGLVESKIRILVGSLEKNEFITLAHVNPQSFPAPKESPDREEFRTMWVIGLVFKKTENSENLSVDLTYDIQSFTDTVYRQAINSKMFELDMKIAAMHVKRKQLHQLLPSHVLQKRKKHSTEGVKLTALNDSSLDLSMDSDNSMSVPSPTSAMKTSPLNSSGSSQGRNSPAPAVTAASVTSIQASEVSVPQANSSESPGGPSSESIPQTATQPAIAPPPKPTVSRVVSSTRLVNPSPRPSGNTATKVPNPIVGVKRTSAPNKEEAPRRTKTEEDETSEDANCLALSGHDKTETKEQVDLETSAVQSETVPASASLLASQKTSSTDLSDIPALPANPIPVIKNSIKLRLNRGSGPRPLLAIHPTEARHKQKIVAPVKQTLNFDLLKLAGDVESNPGPAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSGGGGSGGGGSSRRNKRSRRRRKKPLNTIQPGPSKPSAQDEPIKSVSHHSSKIGTNPMLAFILGGNEDLSDDSDWDEDFSLENTLMPLNEVSLKGKHDSKHFNKGFDNNTALHEVNTKWEAFYSSVKIRQRDVKVYFATDDILIKVREADDIDRKGPWEQAAVDRLRFQRRIADTEKILSAVLLRKKLNPMEHEGSGPRPLLAIHPTEARHKQKIVAPVKQTLNFDLLKLAGDVESNPGPASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSQDLHAIQLQLEEEMFNGGIRRFEADQQRQIASGNESDTAWNRRLLSELIAPMAEGIQAYKEEYEGKRGRAPRALAFINCVGNEVAAYITMKIVMDMLNTDVTLQAIAMNVADRIEDQVRFSKLEGHAAKYFEKVKKSLKASKTKSYRHAHNVAVVAEKSVADRDADFSRWEAWPKDTLLQIGMTLLEILENSVFFNGQPVFLRTLRTNGGKHGVYYLQTSEHVGEWITAFKEHVAQLSPAYAPCVIPPRPWVSPFNGGFHTEKVASRIRLVKGNREHVRKLTKKQMPAVYKAVNALQATKWQVNKEVLQVVEDVIRLDLGYGVPSFKPLIDRENKPANPVPLEFQHLRGRELKEMLTPEQWQAFINWKGECTKLYTAETKRGSKSAATVRMVGQARKYSQFDAIYFVYALDSRSRVYAQSSTLSPQSNDLGKALLRFTEGQRLDSAEALKWFLVNGANNWGWDKKTFDVRTANVLDSEFQDMCRDIAADPLTFTQWVNADSPYGFLAWCFEYARYLDALDEGTQDQFMTHLPVHQDGSCSGIQHYSAMLSDAVGAKAVNLKPSDSPQDIYGAVAQVVIQKNYAYMNAEDAETFTSGSVTLTGAELRSMASAWDMIGITRGLTKKPVMTLPYGSTRLTCRESVIDYIVDLEEKEAQRAIAEGRTANPVHPFDNDRKDSLTPSAAYNYMTALIWPSISEVVKAPIVAMKMIRQLARFAAKRNEGLEYPLPTGFILQQKIMATDMLRVSTCLMGEIKMSLQIETDVVDETAMMGAAAPNFVHGHDASHLILTVCDLVDKGITSVAVIHDSFGTHAGRTADLRDSLREEMVKMYQNHNALQNLLDVHEERWLVDTGIQVPEQGEFDLNEILVSDYCFA

SEQ ID NO: 41 - DNA, 8997 bp, Artificial Sequence
  recombinant DNA open-reading frame from pC3P3- G3i plasmid
atggatgctcagaccagacgcagagaacggcgggcagagaagcaggcacagtggaaggccgctaatccgtttccagttacaacgcagggatcacaacaaacgcagccaccacagaggcactatggcattacctctcctatcagcttagcggcccccaaggagactgactgcctactcacacagaagctcatcgagacgctgaagccctttggggtttttgaagaagaagaggaactgcagcgcaggattttaattttgggaaaattaaataacctggtgaaagaatggattcgagaaatcagtgaaagcaagaatctcccacaatctgtaattgaaaatgttggagggaagatttttacatttggatcttacagactaggagtccacacgaaaggtgctgatattgatgcgttgtgtgttgcaccaagacatgttgatcgaagtgactttttcacctcattctatgataaattgaaattacaagaagaagtgaaagatttaagagctgttgaagaggcatttgtaccagttatcaaactctgttttgatggaatagagattgatattttgtttgcaagattagcactgcagactattccagaagatttggacctacgagatgacagtctgcttaaaaacctagatataagatgcataagaagccttaatggttgcagggtaaccgatgaaattttacatctagtaccaaacattgacaacttcaggttaactctgagagccatcaaactgtgggccaaacggcacaacatctattccaatatattaggtttcctcggtggtgtttcctgggctatgctagtagcaagaacttgccagctttatccaaatgcaatagcatcaactcttgtacataaatttttcttggtattttctaaatgggaatggccaaatccagtgctattgaaacagcctgaagaatgcaatcttaatttgcctgtgtgggacccaagggtaaaccccagtgataggtaccatcttatgcctataattacaccagcatacccacagcagaactccacgtacaatgtgtccgtttcaacacggatggtcatggttgaggagtttaaacaaggtcttgctatcacagatgaaattttgctgagtaaggcagagtggtccaaactttttgaagctccaaacttctttcagaagtacaagcattatattgtacttctagcaagtgcgcccacggaaaagcagcgtctggaatgggtgggcttggtggaatcaaaaatccgcatcctggttggaagcttggagaagaatgagtttattacactggctcatgtgaatccccagtcatttccagcccccaaagaaagtcctgacagggaagaatttcgcacaatgtgggtgattgggttagtgtttaaaaaaactgaaaactctgaaaatctcagtgtcgacctcacctatgatatccagtctttcacagacacagtttataggcaagcaataaacagcaaaatgtttgagttggatatgaagattgcagcaatgcatgtgaagagaaagcaactccatcagctgctgcctagtcacgtgcttcagaagaggaagaagcattcaacagaaggagtcaagttaacagctctgaatgacagcagccttgacttgtctatggacagtgataacagcatgtctgtgccttcacccaccagtgctatgaagaccagtccattgaatagttctggcagctcccagggcagaaacagtcctgctccagctgtgaccgcagcatctgtgaccagcatccaggcttctgaggtttctgtaccgcaagcaaattccagtgaaagcccagggggtccatcgagcgaaagcattcctcaaactgccacacagccagccattgccccaccaccaaagcctacagtctccagagttgtctcctcaacacgactggtaaacccatcgcctagaccttcaggaaacacagcaacaaaagtccctaatcctatagtaggagtcaagagaacgtccgcccccaataaagaagaagcccctagaaggaccaaaacagaagaggatgaaacaagtgaagatgctaactgtcttgctttgagtggacatgataaaacagagacaaaggaacaagttgatctggagacaagtgcggttcaatcagaaactgttccggcatcggcttctctgttggcctctcagaaaacatccagtacagacctttctgatatccctgctctccctgcaaatcctattcctgttatcaagaactcaataaaactgagactgaatcggggatcaggcccgcgacccctccttgccatccacccaaccgaggcacggcacaagcagaaaatagtggcacccgttaagcagactctcaactttgatctcctgaagcttgccggcgacgtagagtccaaccccggccccgctggcgatctgagcgccggttttttcatggaagaactcaatacttatagacaaaaacagggagtggtccttaagtaccaggagcttcctaattcaggtcccccccacgatcgaagattcacgtttcaggtgatcatcgatggcagagaattccccgaaggcgagggacgctcaaagaaagaggctaagaatgctgcggccaagcttgccgtcgaaatcctgaataaggaaaaaaaggcagttagcccgttgctgttgaccaccacgaattcgtcagaaggactatctatgggcaactacatagggttgattaacaggatcgcccagaaaaaacggcttaccgttaattatgagcaatgcgctagtggtgtgcacggcccggaagggttccattacaaatgcaaaatgggtcagaaggagtacagcattgggaccggcagtacaaaacaggaagcaaagcagctggccgccaagctagcatatcttcagattctgtccggatcaggcccgcgacccctccttgccatccacccaaccgaggcacggcacaagcagaaaatagtggcacccgttaagcagactctcaactttgatctcctgaagcttgccggcgacgtagagtccaaccccggccccagccggcggaacaagcggagccggcggcggcggaagaagcccctgaacaccatccagcccggccccagcaagcccagcgcccaggacgagcccatcaagagcgtgagccaccacagcagcaagatcggcaccaaccccatgctggccttcatcctgggcggcaacgaggacctgagcgacgacagcgactgggacgaggacttcagcctggagaacaccctgatgcccctgaacgaggtgagcctgaagggcaagcacgacagcaagcacttcaacaagggcttcgacaacaacaccgccctgcacgaggtgaacaccaagtgggaggccttctacagcagcgtgaagatccggcagcgggacgtgaaggtgtacttcgccaccgacgacatcctgatcaaggtgcgggaggccgacgacatcgaccggaagggcccctgggagcaggccgccgtggaccggctgcggttccagcggcggatcgccgacaccgagaagatcctgagcgccgtgctgctgcggaagaagctgaaccccatggagcacgagggatcaggcccgcgacccctccttgccatccacccaaccgaggcacggcacaagcagaaaatagtggcacccgttaagcagactctcaactttgatctcctgaagcttgccggcgacgtagagtccaaccccggccccgccagcctggacaacctggtggccagataccagcggtgcttcaacgaccagagcctgaagaacagcaccatcgagctggaaatccggttccagcagatcaacttcctgctgttcaagaccgtgtacgaggccctggtcgcccaggaaatccccagcaccatcagccacagcatccggtgcatcaagaaggtgcaccacgagaaccactgccgggagaagatcctgcccagcgagaacctgtacttcaagaaacagcccctgatgttcttcaagttcagcgagcccgccagcctgggctgtaaagtgtccctggccatcgagcagcccatccggaagttcatcctggacagcagcgtgctggtccggctgaagaaccggaccaccttccgggtgtccgagctgtggaagatcgagctgaccatcgtgaagcagctgatgggcagcgaggtgtcagccaagctggccgccttcaagaccctgctgttcgacacccccgagcagcagaccaccaagaacatgatgaccctgatcaaccccgacgacgagtacctgtacgagatcgagatcgagtacaccggcaagcctgagagcctgacagccgccgacgtgatcaagatcaagaacaccgtgctgacactgatcagccccaaccacctgatgctgaccgcctaccaccaggccatcgagtttatcgccagccacatcctgagcagcgagatcctgctggcccggatcaagagcggcaagtggggcctgaagagactgctgccccaggtcaagtccatgaccaaggccgactacatgaagttctacccccccgtgggctactacgtgaccgacaaggccgacggcatccggggcattgccgtgatccaggacacccagatctacgtggtggccgaccagctgtacagcctgggcaccaccggcatcgagcccctgaagcccaccatcctggacggcgagttcatgcccgagaagaaagagttctacggctttgacgtgatcatgtacgagggcaacctgctgacccagcagggcttcgagacacggatcgagagcctgagcaagggcatcaaggtgctgcaggccttcaacatcaaggccgagatgaagcccttcatcagcctgacctccgccgaccccaacgtgctgctgaagaatttcgagagcatcttcaagaagaaaacccggccctacagcatcgacggcatcatcctggtggagcccggcaacagctacctgaacaccaacaccttcaagtggaagcccacctgggacaacaccctggactttctggtccggaagtgccccgagtccctgaacgtgcccgagtacgcccccaagaagggcttcagcctgcatctgctgttcgtgggcatcagcggcgagctgtttaagaagctggccctgaactggtgccccggctacaccaagctgttccccgtgacccagcggaaccagaactacttccccgtgcagttccagcccagcgacttccccctggccttcctgtactaccaccccgacaccagcagcttcagcaacatcgatggcaaggtgctggaaatgcggtgcctgaagcgggagatcaactacgtgcgctgggagatcgtgaagatccgggaggaccggcagcaggatctgaaaaccggcggctacttcggcaacgacttcaagaccgccgagctgacctggctgaactacatggaccccttcagcttcgaggaactggccaagggacccagcggcatgtacttcgctggcgccaagaccggcatctacagagcccagaccgccctgatcagcttcatcaagcaggaaatcatccagaagatcagccaccagagctgggtgatcgacctgggcatcggcaagggccaggacctgggcagatacctggacgccggcgtgagacacctggtcggcatcgataaggaccagacagccctggccgagctggtgtaccggaagttctcccacgccaccaccagacagcacaagcacgccaccaacatctacgtgctgcaccaggatctggccgagcctgccaaagaaatcagcgagaaagtgcaccagatctatggcttccccaaagagggcgccagcagcatcgtgtccaacctgttcatccactacctgatgaagaacacccagcaggtcgagaacctggctgtgctgtgccacaagctgctgcagcctggcggcatggtctggttcaccaccatgctgggcgaacaggtgctggaactgctgcacgagaaccggatcgaactgaacgaagtgtgggaggcccgggagaacgaggtggtcaagttcgccatcaagcggctgttcaaagaggacatcctgcaggaaaccggccaggaaatcggcgtcctgctgcccttcagcaacggcgacttctacaatgagtacctggtcaacaccgcctttctgatcaagattttcaagcaccatggctttagcctcgtgcagaagcagagcttcaaggactggatccccgagttccagaacttcagcaagagcctgtacaagatcctgaccgaggccgacaagacctggaccagcctgttcggcttcatctgcctgcggaagaacggaggcgggggaagtggagggggcggcagtcaggacctgcacgccatccagctgcagctcgaagaggaaatgttcaacggcggcatcagaagattcgaggccgaccagcagagacagatcgcctctggcaacgagagcgacaccgcctggaatagaaggctgctgtctgagctgatcgcccctatggccgaaggcatccaggcctacaaagaggaatacgagggcaagagaggcagagcccctagagccctggccttcatcaactgtgtgggcaatgaggtggccgcctacatcaccatgaagatcgtgatggacatgctgaacaccgacgtgaccctgcaggccattgccatgaacgtggccgacagaatcgaggaccaggtccgattcagcaagctggaaggacacgccgccaagtacttcgagaaagtgaagaagtccctgaaggccagcaagaccaagagctacagacacgcccacaacgtggccgtggtggccgaaaaatctgtggccgatagggacgccgacttctctagatgggaggcctggcctaaggacaccctgctgcagatcggcatgaccctgctggaaatcctggaaaacagcgtgttcttcaacggccagcccgtgttcctgagaaccctgaggacaaatggcggcaagcacggcgtgtactacctgcagacatctgagcacgtgggcgagtggatcaccgccttcaaagaacatgtggcccagctgagccctgcctatgccccttgtgtgatccctcctagaccctgggtgtcccctttcaatggcggctttcacaccgagaaggtggccagcagaatcagactggtcaagggcaaccgggaacacgtgcggaagctgaccaagaaacagatgcccgccgtgtacaaggccgtgaatgctctgcaggccaccaagtggcaggtcaacaaagaggtgctgcaggtcgtcgaggacgtgatcagactggatctgggctacggcgtgccaagctttaagcccctgatcgacagagagaacaagcccgccaaccctgtgcccctggaatttcagcacctgagaggccgcgagctgaaagagatgctgacacctgaacagtggcaggcctttatcaattggaagggcgagtgcaccaagctgtacaccgccgagacaaagaggggctctaagtctgccgccacagtgcgaatggtcggacaggccagaaagtacagccagttcgacgccatctacttcgtgtacgccctggacagccggtctagagtgtatgcccagagcagcacactgagcccccagtctaacgatctgggaaaggccctgctgagattcaccgagggccagagactggattctgccgaagccctgaagtggttcctggtcaacggcgccaacaactggggctgggacaagaaaaccttcgatgtgcggaccgccaacgtgctggatagcgagttccaggacatgtgcagagatatcgccgccgaccctctgacctttacccagtgggtcaacgccgatagcccctatggattcctggcctggtgcttcgagtacgccagatacctggacgccctggatgagggaacccaggatcagttcatgacccatctgcccgtgcaccaggatggctcttgttctggcatccagcactacagcgccatgctgagcgatgccgtgggagccaaagccgtgaacctgaagcctagcgacagcccccaggatatctatggcgctgtggcccaggtggtcatccagaaaaactacgcctacatgaacgccgaggacgccgagacattcacaagcggaagcgtgacactgacaggcgccgagctgagatctatggcctctgcctgggacatgatcggcatcacacggggcctgaccaaaaagcctgtgatgacactgccctacggcagcaccagactgacctgtagagaaagcgtgatcgactacatcgtggacctggaagagaaagaggcccagagagccattgccgagggcagaacagccaatcctgtgcaccccttcgacaacgaccggaaggatagcctgacacctagcgccgcctacaactacatgaccgccctgatctggcccagcatctctgaagtggtcaaggcccctatcgtggccatgaagatgatcagacagctggccagattcgccgccaagagaaatgagggcctggaataccctctgcccaccggctttatcctgcagcagaaaatcatggccaccgacatgctgcgggtgtccacatgtctgatgggcgagatcaagatgagcctgcagatcgagacagacgtggtggacgagacagccatgatgggagccgccgctcctaattttgtgcacggacacgatgccagccacctgatcctgaccgtgtgcgatctggtggacaagggcatcactagcgtggccgtgatccacgatagctttggaacacacgccggcagaaccgccgacctgagagattctctgcgggaagagatggtcaagatgtaccagaaccacaacgccctgcagaacctgctggacgtgcacgaagaaagatggctggtggacaccggcatccaggtgccagaacagggagagttcgacctgaacgagatcctggtgtccgactactgcttcgcctga

SEQ ID NO: 42 - protein, 2998 aa, Artificial Sequence
  recombinant protein encoded by pC3P3-G3i plasmid
MDAQTRRRERRAEKQAQWKAANPFPVTTQGSQQTQPPQRHYGITSPISLAAPKETDCLLTQKLIETLKPFGVFEEEEELQRRILILGKLNNLVKEWIREISESKNLPQSVIENVGGKIFTFGSYRLGVHTKGADIDALCVAPRHVDRSDFFTSFYDKLKLQEEVKDLRAVEEAFVPVIKLCFDGIEIDILFARLALQTIPEDLDLRDDSLLKNLDIRCIRSLNGCRVTDEILHLVPNIDNFRLTLRAIKLWAKRHNIYSNILGFLGGVSWAMLVARTCQLYPNAIASTLVHKFFLVFSKWEWPNPVLLKQPEECNLNLPVWDPRVNPSDRYHLMPIITPAYPQQNSTYNVSVSTRMVMVEEFKQGLAITDEILLSKAEWSKLFEAPNFFQKYKHYIVLLASAPTEKQRLEWVGLVESKIRILVGSLEKNEFITLAHVNPQSFPAPKESPDREEFRTMWVIGLVFKKTENSENLSVDLTYDIQSFTDTVYRQAINSKMFELDMKIAAMHVKRKQLHQLLPSHVLQKRKKHSTEGVKLTALNDSSLDLSMDSDNSMSVPSPTSAMKTSPLNSSGSSQGRNSPAPAVTAASVTSIQASEVSVPQANSSESPGGPSSESIPQTATQPAIAPPPKPTVSRVVSSTRLVNPSPRPSGNTATKVPNPIVGVKRTSAPNKEEAPRRTKTEEDETSEDANCLALSGHDKTETKEQVDLETSAVQSETVPASASLLASQKTSSTDLSDIPALPANPIPVIKNSIKLRLNRGSGPRPLLAIHPTEARHKQKIVAPVKQTLNFDLLKLAGDVESNPGPAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSGSGPRPLLAIHPTEARHKQKIVAPVKQTLNFDLLKLAGDVESNPGPSRRNKRSRRRRKKPLNTIQPGPSKPSAQDEPIKSVSHHSSKIGTNPMLAFILGGNEDLSDDSDWDEDFSLENTLMPLNEVSLKGKHDSKHFNKGFDNNTALHEVNTKWEAFYSSVKIRQRDVKVYFATDDILIKVREADDIDRKGPWEQAAVDRLRFQRRIADTEKILSAVLLRKKLNPMEHEGSGPRPLLAIHPTEARHKQKIVAPVKQTLNFDLLKLAGDVESNPGPASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSVLVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINYVRWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGVRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKNGGGGSGGGGSQDLHAIQLQLEEEMFNGGIRRFEADQQRQIASGNESDTAWNRRLLSELIAPMAEGIQAYKEEYEGKRGRAPRALAFINCVGNEVAAYITMKIVMDMLNTDVTLQAIAMNVADRIEDQVRFSKLEGHAAKYFEKVKKSLKASKTKSYRHAHNVAVVAEKSVADRDADFSRWEAWPKDTLLQIGMTLLEILENSVFFNGQPVFLRTLRTNGGKHGVYYLQTSEHVGEWITAFKEHVAQLSPAYAPCVIPPRPWVSPFNGGFHTEKVASRIRLVKGNREHVRKLTKKQMPAVYKAVNALQATKWQVNKEVLQVVEDVIRLDLGYGVPSFKPLIDRENKPANPVPLEFQHLRGRELKEMLTPEQWQAFINWKGECTKLYTAETKRGSKSAATVRMVGQARKYSQFDAIYFVYALDSRSRVYAQSSTLSPQSNDLGKALLRFTEGQRLDSAEALKWFLVNGANNWGWDKKTFDVRTANVLDSEFQDMCRDIAADPLTFTQWVNADSPYGFLAWCFEYARYLDALDEGTQDQFMTHLPVHQDGSCSGIQHYSAMLSDAVGAKAVNLKPSDSPQDIYGAVAQVVIQKNYAYMNAEDAETFTSGSVTLTGAELRSMASAWDMIGITRGLTKKPVMTLPYGSTRLTCRESVIDYIVDLEEKEAQRAIAEGRTANPVHPFDNDRKDSLTPSAAYNYMTALIWPSISEVVKAPIVAMKMIRQLARFAAKRNEGLEYPLPTGFILQQKIMATDMLRVSTCLMGEIKMSLQIETDVVDETAMMGAAAPNFVHGHDASHLILTVCDLVDKGITSVAVIHDSFGTHAGRTADLRDSLREEMVKMYQNHNALQNLLDVHEERWLVDTGIQVPEQGEFDLNEILVSDYCFA